(19) United States
(12) Patent Application Publication
(10) Pub. No.: US 2017/0283809 A1
(43) Pub. Date: Oct. 5, 2017

(54) RECOMBINANT MICROORGANISM PRODUCING ALKENES FROM ACETYL-COA

(71) Applicant: GLOBAL BIOENERGIES, Evry Cedex (FR)

(72) Inventor: Bernhard GÜNTNER, Vaals (NL)

(73) Assignee: Global Bioenergies, Evry Cedex (FR)

(21) Appl. No.: 15/508,046

(22) PCT Filed: Sep. 3, 2015
(86) PCT No.: PCT/EP2015/070194

§ 371 Date: Mar. 1, 2017

Related U.S. Application Data
(60) Provisional application No. 62/047,827, filed on Sep. 9, 2014.
(60) Provisional application No. 62/045,083, filed on Sep. 3, 2014.

(30) Foreign Application Priority Data
Sep. 26, 2014 (EP) 14186574.1
Sep. 26, 2014 (EP) 14186690.5

(51) Int. Cl. C12N 15/52; C12N 9/10; C12N 9/04; C12N 9/02; C12N 15/63; C12P 7/02

(52) U.S. Cl. CPC C12N 15/52; C12N 15/63; C12P 7/02; C12N 9/0006; C12N 9/001; C12N 9/1025; C12N 1/00

(57) ABSTRACT

Disclosed is a recombinant microorganism comprising endogenous enzymes that convert CO and/or CO₂ to acetyl-CoA. The recombinant microorganism contains a heterologous nucleic acid sequence encoding one or more enzymes that allow the conversion of acetyl-CoA to an alkene with a main chain of 1 to 5 carbon atoms. The heterologous nucleic acid sequence comprises one or more coding sequences encoding one or more enzymes that catalyse the conversion of acetyl-CoA to crotonyl-CoA, and that further catalyse the conversion of crotonyl-CoA to an alkene; or one or more coding sequences encoding one or more enzymes that catalyse the conversion of acetyl-CoA to 3-methylcrotonyl-CoA, and that further catalyse the conversion of 3-methylcrotonyl-CoA to an alkene; or one or more coding sequences encoding one or more enzymes that catalyse the conversion of acetyl-CoA to propionyl-CoA, and that further catalyse the conversion of propionyl-CoA to an alkene. Each coding sequence is operationally linked to a transcriptional promoter.

### CROSS-REFERENCE TO RELATED APPLICATIONS

The present application claims the benefit of and the priority to an application for “Genetically engineered microorganism and process for converting gaseous C1-carbon sources and/or gaseous C1-carbon sources and electrons into alkenes” filed on 3 Sep. 2014 with the USPTO, and there duly assigned serial number U.S. 62/045,083. The present application also claims the benefit of and the priority to an application for “Genetically engineered microorganism and process for converting gaseous C1-carbon sources and/or gaseous C1-carbon sources and electrons into 1,3-butadiene” filed on 9 Sep. 2014 with the USPTO, and there duly assigned serial number U.S. 62/047,827. The present application also claims the benefit of and the priority to an application for “Genetically engineered microorganism and process for converting gaseous C1-carbon sources and/or gaseous C1-carbon sources and electrons into 2-methyl-1,3-butadiene and 1,3-butadiene” filed on 26 Sep. 2014 with the European Patent Office, and there duly assigned serial number EP 14 186 574. The present application also claims the benefit of and the priority to an application for “Genetically engineered microorganism and process for converting gaseous C1-carbon sources and/or gaseous C1-carbon sources and electrons into alkenes” filed on 26 Sep. 2014 with the European Patent Office, and there duly assigned serial number EP 14 186 690. The contents of said applications U.S. 62/045,083, U.S. 62/047,827, EP 14 186 574, and EP 14 186 690 are incorporated herein by reference for all purposes in their entirety including all tables, figures, and claims—as well as including an incorporation of any element or part of the description, claims or drawings not contained herein and referred to in Rule 20.5(a) of the PCT, pursuant to Rule 4.18 of the PCT.

### REFERENCE TO A SEQUENCE LISTING

This application includes a sequence listing.

### FIELD OF THE DISCLOSURE

A recombinant microorganism is provided. Also provided is a method in which the recombinant microorganism is being used. The method is a method of converting a C₁-carbon source into one or more alkanes and/or alkenes with a main chain of 1 to 6 carbon atoms.

### BACKGROUND

The following discussion of the background is merely provided to aid the reader in understanding the microorganisms, method and uses disclosed in the following, and is not admitted to describe or constitute prior art.

Increased demand for energy and bulk chemicals by the global economy has put increasing pressure on the cost of hydrocarbons. Many industries, including the chemical and plastics industry rely heavily on fossil hydrocarbon sources being available as feedstock for their products. Alkanes are the major constituents of gasoline, diesel, and jet fuel. Alkenes are commonly produced by cracking the alkanes found in crude oil. The rapid depletion of fossil fuels has been a driving force to identify alternative sources for the production of alkanes. Many long chain alkanes are naturally produced by diverse species. However, for alkanes and alkenes with a chain length of less than about 10 atoms a natural biosynthesis pathway is only known for methane. Such short chain alkanes and alkenes are used not only as fuel, but are also important in the production of bulk chemicals. They for instance serve as building blocks for plastics, poylesters, solvents, fuels, adhesives, or paints. Propene (propylene) for instance is a terminal olefin which is used to manufacture polyethylene, polypropylene, alpha olefins, styrene, polyesters, acrylics, ethylene glycol antifreeze, polyvinyl chloride (PVC), propylene oxide, keto alcohols, and isopropanol. Propene is traditionally derived from fractional distillation from hydrocarbon mixtures obtained from cracking and other refining processes. Ethylene also serves as a starting material, e.g. as a monomer for the production of polyurethanes, rubber products, laboratory chemicals, industrial fluids, and anti-freeze compounds. It is also used to produce fuel and fuel additives.

1,3-butadiene is a colorless gas that condenses to a liquid at minus 4.5 degrees centigrade. Using an extractive distillation process, butadiene is derived from the crude C4 stream, one of the cracker by-products of ethylene and propylene production. The largest single use of butadiene is in the production of styrene-butadiene rubber (SBR) which, in turn, is principally used in the manufacture of automobile tires. SBR is also used in adhesives, sealants, coatings and in rubber articles like shoe soles. Polybutadiene is also used in tires and can be used as an intermediate in the production of acrylonitrile-butadiene-styrene (ABS). ABS is widely used in items such as telephones, computer casings and other appliances. Other polymers made from butadiene include styrene-butadiene latex, used for example in carpet backings and adhesives; nitrile rubber, used in hoses, fuel lines, gasket seals, gloves and footwear; and styrene-butadiene block copolymers which are used in many end-uses ranging from asphalt modifiers (road and roofing construction applications), to adhesives, footwear and toys. Chemical intermediates made from butadiene include adiponitrile and chloroprene which are used, respectively, in the manufacture of nylon and neoprene. Approximately 10 million tons of butadiene are currently produced annually. The sales of 1,3-butadiene exceed a value of over 10 billion US dollars worldwide. The ability to manufacture butadiene from alternative and/or renewable feedstocks represents a major advance in the quest for more sustainable chemical production processes.

2-methyl-1,3-butadiene or isoprene, is a colorless volatile liquid, which is naturally produced by many plants, animals and bacteria. 2-methyl-1,3-butadiene serves as a key starting material for the synthesis of a variety of synthetic polymers, in particular of synthetic elastomers. Most of the 2-methyl-1,3-butadiene today available and needed for the production of synthetic elastomers is produced by thermal cracking of petroleum or oil. Elastomeres, or rubbers, are needed not only in tire production, but also in the rubber industry, where they are used for a wide variety of consumer products such as medical gloves, fittings, rubber bands, shoes, sporting goods, etc. Synthetic rubber is based primarily on butadiene polymers, which are obtained as a by-product from ethylene and propylene production. Although 2-methyl-1,3-butadiene can be obtained by fractionating petroleum, the purification of petroleum is expensive and time-consuming, with the yield of the obtained 2-methyl-1,3-butadiene being low when compared to the efforts required. The supply of 2-methyl-1,3-butadiene is furthermore at risk, as availability of natural rubber tightens and its price increases. Also, due to a more profound ecological awareness, acceptance of crude C5 feed streams from olefine cracking is declining. Thus, recently, more economical methods for producing 2-methyl-1,3-butadiene have been endeavored, and in particular biobased processes. Although the biological resources for 2-methyl-1,3-butadiene, i.e. certain plants, are available as such, a commercial process to produce and recover 2-methyl-1,3-butadiene from plants that would be economically and practically feasible has not been developed so far. Attempts of isoprene production from synthesis using bacteria gas have been made (U.S. patent applications US 2014/0234926, US2013/0323820, international patent applications WO 2014/065271, WO 2013/180584, WO2013/181647), the strains used do not meet the requirements of industrial production in terms of yield, purity and stability.

Available technologies for syngas conversion to liquid fuels or chemicals include chemical catalytic processes such as the Fischer-Tropsch process as well as processes for the synthesis of methanol or other mixed alcohols, and biological gas fermentation processes. The Fischer-Tropsch process is in use since almost one hundred years and relies on metal-based, inorganic catalysts for the conversion of syngas into longer chain hydrocarbons.

Biologic systems that fix carbon through natural biochemical metabolic processes are known. Algal systems have been developed to create hydrocarbons through photosynthetic reactions, as well as heterotrophic reactions fed by sugar that indirectly depend upon photosynthesis. Bacterial cells have been genetically engineered to process sugar feedstocks into useful hydrocarbons in heterotrophic fermentation systems.

Biological pathways to produce for instance alkenes directly or indirectly have been disclosed (US 2011/0269204, US 2011/0091952, US 2011/0269204, WO 2012/058606, US 2012/0329119, US 2013/0224808, US 2013/0316425, WO 2013/148348, US 2014/0039143, WO 2014/047209). However, the methods of these disclosures have limitations that render them inter alia inefficient or unsuitable for large scale industrial processes. Several publications (Lee, C. C., et al., Science (2010) 329, 5992, 642; Yang, Z.-Y, et al., Journal of Biological Chemistry (2011) 286, 22, 19417-19421) show that purified vanadium as well as molybdenum nitrogenases are able to catalyze the conversion of CO or CO₂ or CN to form hydrocarbons in vitro.

### SUMMARY

A recombinant microorganism is provided. The recombinant microorganism is typically capable of producing one or more alkenes with a main chain of 2 to 5 carbon atoms from a C₁-carbon compound, in particular carbon monoxide and/or carbon dioxide. The recombinant microorganism may also be capable of producing one or more alkanes and/or alkenes with a main chain of 1 to 6 carbon atoms from a C₁-carbon such as carbon monoxide, carbon dioxide and/or cyanide. In some embodiments the recombinant microorganism may be capable of producing one or more alkanes and/or alkenes with a main chain of 2 or 3 carbon atoms from carbon monoxide or carbon dioxide. In some embodiments the recombinant microorganism may be capable of producing one or more alkanes and/or alkenes with a main chain of 4 or 5 carbon atoms from carbon monoxide or carbon dioxide. In some embodiments the recombinant microorganism is not capable of carrying out photosynthesis. In some embodiments the recombinant microorganism has endogenous enzymes that allow the recombinant microorganism to perform a biosynthesis of acetyl-CoA from CO, CO₂ and/or cyanide. In some embodiments the recombinant microorganism has homologous enzymes that allow the recombinant microorganism to perform a biosynthesis of acetyl-CoA from CO, CO₂ and/or cyanide.

The cells described herein contain artificial and efficient pathways for the biosynthesis of alkanes and/or alkenes. In certain embodiments the respective biosynthesis genes are stably integrated into the host chromosome. Provided are also processes by means of which 2 alkanes and/or alkenes can be produced with high yield and purity that are sufficient to meet the demands of a robust commercial process. These processes represent a very clean and efficient (in terms of cost- and time-saving) and industrially feasible way of producing alkanes and/or alkenes of interest.

According to a first aspect, a recombinant microorganism is provided. The recombinant microorganism contains a heterologous nucleic acid sequence, which encodes one or more enzymes that catalyse the conversion of acetyl-CoA to one or more alkanes and/or alkenes with a main chain of 1 to 5 carbon atoms. The heterologous nucleic acid sequence includes a coding sequence or a combination of coding sequences that encode one or more enzymes that catalyse the conversion of acetyl-CoA to one or more alkanes and/or alkenes with a main chain of 1 to 5 carbon atoms. Each coding sequence is operationally linked to a transcriptional promoter.

In some embodiments the recombinant microorganism contains a heterologous nucleic acid sequence that encodes one or more enzymes that allow the conversion of acetyl-CoA to one or more alkenes with a main chain of 2 to 5 carbon atoms. In some embodiments the recombinant microorganism furthermore contains one or more endogenous enzymes that convert a C₁-carbon source to acetyl-CoA. The C₁-carbon source is in some embodiments at least one of carbon monoxide, carbon dioxide and cyanide.

In some embodiments the recombinant microorganism does not include at least one coding sequence or a combination of coding sequences that encode one or more enzymes that catalyse the conversion of acetyl-CoA to one or more alkanes and/or alkenes with a main chain of 1 to 8 carbon atoms. In embodiments where the recombinant microorganism includes more than one coding sequences or a combination of more than one coding sequences that encode one or more enzymes that catalyse the conversion of acetyl-CoA to one or more alkanes and/or alkenes with a main chain of 1 to 8 carbon atoms, the recombinant microorganism may not include at least one of the respective coding sequences.

According to some embodiments of the recombinant microorganism according to the first aspect the heterologous nucleic acid sequence includes a coding sequence that encodes a nitrogenase enzyme EC 1.18.6.1 or EC 1.19.6.1.

According to some embodiments of the recombinant microorganism according to the first aspect the heterologous nucleic acid sequence includes one or more coding sequences encoding one or more enzymes that catalyse the conversion of acetyl-CoA to crotonyl-CoA, and that further catalyse the conversion of crotonyl-CoA to an alkene.

According to some embodiments of the recombinant microorganism according to the first aspect the heterologous nucleic acid sequence includes one or more coding sequences encoding one or more enzymes that catalyse the conversion of acetyl-CoA to 3-methylcrotonyl-CoA, and that further catalyse the conversion of 3-methylcrotonyl-CoA to an alkene.

According to some embodiments of the recombinant microorganism according to the first aspect the heterologous nucleic acid sequence includes one or more coding sequences encoding one or more enzymes that catalyse the conversion of acetyl-CoA to propionyl-CoA, and that further catalyse the conversion of propionyl-CoA to an alkene.

According to some embodiments of the recombinant microorganism according to the first aspect the heterologous nucleic acid sequence includes one or more coding sequences encoding one or more enzymes that catalyse the conversion of acetyl-CoA to acryloyl-CoA, and that further catalyse the conversion of acryloyl-CoA to an alkene.

According to some embodiments of the recombinant microorganism according to the first aspect the one or more enzymes that catalyse the conversion of acetyl-CoA to crotonyl-CoA include acetyl-CoA carboxylase (EC 6.4.1.2); acetoacetyl-CoA synthase (EC 2.3.1.194); acetoacetyl-CoA reductase (EC 1.1.1.36); and enoyl-CoA hydratase 2 (EC 4.2.1.119). According to some embodiments of the recombinant microorganism according to the first aspect the one or more enzymes that catalyse the conversion of acetyl-CoA to crotonyl-CoA include acetyl-CoA C-acetyltransferase (EC 2.3.1.9); 3-hydroxybutyryl-CoA dehydrogenase (EC 1.1.1.157); and 3-hydroxybutyryl-CoA dehydratase. According to some embodiments of the recombinant microorganism according to the first aspect the one or more enzymes that catalyse the conversion of acetyl-CoA to crotonyl-CoA include 4-aminobutyrate aminotransferase (EC 2.6.1.19); glutamate dehydrogenase (EC 1.4.1.2); 2-hydroxyglutarate dehydrogenase (EC 1.1.99.2); glutaconate CoA-transferase (EC 2.8.3.12); 2-hydroxyglutaryl-CoA dehydratase (EC 4.2.1.-); and glutaconyl-CoA decarboxylase (EC 4.1.1.70). According to some embodiments of the recombinant microorganism according to the first aspect the one or more enzymes that catalyse the conversion of acetyl-CoA to crotonyl-CoA include 4-aminobutyrate aminotransferase (EC 2.6.1.19); 4-hydroxybutyrate dehydrogenase (EC 1.1.1.61); 4-hydroxybutyrate CoA-transferase (EC 2.8.3.-); vinylacetyl-CoA-Delta-isomerase (EC 4.2.1.120); and 4-hydroxybutyryl-CoA dehydratase (EC 5.3.3.3).

According to some embodiments of the recombinant microorganism according to the first aspect the one or more enzymes that catalyse the conversion of acetyl-CoA to 3-methylcrotonyl-CoA include acetyl-CoA C-acetyltransferase (EC 2.3.1.9); 3-hydroxy-3-methylglutaryl-CoA synthase (EC 2.3.3.10); and 3-methylglutaconyl-CoA hydratase (EC 4.2.1.18). The one or more enzymes that catalyse the conversion of acetyl-CoA to 3-methylcrotonyl-CoA may also include 3-methylcrotonyl-CoA carboxylase (EC 6.4.1.4). The one or more enzymes that catalyse the conversion of acetyl-CoA to 3-methylcrotonyl-CoA may in some embodiments include acetyl-CoA carboxylase (EC 6.4.1.2); acetoacetyl-CoA synthase (EC 2.3.1.194); 3-hydroxy-3-methylglutaryl-CoA synthase (EC 2.3.3.10); 3-methylglutaconyl-CoA hydratase (EC 4.2.1.18); and 3-methylcrotonyl-CoA carboxylase (EC 6.4.1.4). In some embodiments the one or more enzymes that catalyse the conversion of acetyl-CoA to 3-methylcrotonyl-CoA include acetyl-CoA C-acetyltransferase (EC 2.3.1.9); acetyl-CoA carboxylase (EC 6.4.1.2); acetoacetyl-CoA synthase (EC 2.3.1.194); 3-hydroxy-3-methylglutaryl-CoA synthase (EC 2.3.3.10); 3-methylglutaconyl-CoA hydratase (EC 4.2.1.18); and 3-methylcrotonyl-CoA carboxylase (EC 6.4.1.4).

According to some embodiments of the recombinant microorganism according to the first aspect the one or more enzymes that catalyse the conversion of acetyl-CoA to propionyl-CoA include acetyl-CoA carboxylase (EC 6.4.1.2); malonyl CoA reductase (EC 1.2.1.75); malonyl CoA reductase (EC 1.1.1.298); propionyl-CoA synthase (EC 6.2.1.36); propionyl-CoA synthase/acryloyl-CoA synthase (EC 4.2.1.116); and propionyl-CoA synthase (EC 1.3.1.84). According to some embodiments of the recombinant microorganism according to the first aspect the one or more enzymes that catalyse the conversion of acetyl-CoA to propionyl-CoA include acetyl-CoA carboxylase (EC 6.4.1.2) and methylmalonyl-CoA decarboxylase (EC 4.1.1.41). The one or more enzymes that catalyse the conversion of acetyl-CoA to propionyl-CoA may for example include acetyl-CoA carboxylase (EC 6.4.1.2); methylmalonyl-CoA mutase (EC 5.4.99.2) and methylmalonyl-CoA decarboxylase (EC 4.1.1.41). According to some embodiments of the recombinant microorganism according to the first aspect the one or more enzymes that catalyse the conversion of acetyl-CoA to propionyl-CoA include 2-oxoglutarate/butyrate ferredoxin oxidoreductase (EC 1.2.7.3) and/or pyruvate/2-oxobutyrate ferredoxin oxidoreductase (EC 1.2.7.1). According to some embodiments of the recombinant microorganism according to the first aspect the one or more enzymes that catalyse the conversion of acetyl-CoA to propionyl-CoA include lactate CoA-transferase (EC 2.8.3.-); lactoyl-CoA dehydratase (EC 4.2.1.54); and propionyl-CoA synthase (EC 1.3.1.84).

According to some embodiments of the recombinant microorganism according to the first aspect the one or more enzymes that catalyse the conversion of acetyl-CoA to acryloyl-CoA include acetyl-CoA carboxylase (EC 6.4.1.2); malonyl CoA reductase (EC 1.2.1.75); malonyl CoA reductase (EC 1.1.1.298); propionyl-CoA synthase (EC 6.2.1.36); and propionyl-CoA synthase/acryloyl-CoA synthase (EC 4.2.1.116). In some embodiments the one or more enzymes that catalyse the conversion of acetyl-CoA to acryloyl-CoA include lactate CoA-transferase (EC 2.8.3.-); and lactoyl-CoA dehydratase (EC 4.2.1.54).

In some embodiments the one or more enzymes that catalyse the conversion of crotonyl-CoA to an alkene include acyl-CoA thioesterase (EC 3.1.2.-) and phenyl acrylic acid decarboxylase (EC 4.1.1.-). In some embodiments the one or more enzymes that catalyse the conversion of crotonyl-CoA to an alkene include phosphate butyryltransferase (EC 2.3.1.19); butyrate kinase (EC 2.7.2.7), and phenyl acrylic acid decarboxylase (EC 4.1.1.-). In some embodiments the one or more enzymes that catalyse the conversion of crotonyl-CoA to an alkene include trans-2-enoyl-CoA reductase (EC1.1.1.36); acetyl-CoA C-acetyltransferase (EC 2.3.1.9); 3-hydroxybutyryl-CoA dehydrogenase (EC 1.1.1.157); 3-hydroxybutyryl-CoA dehydratase (EC 4.2.1.55); acyl-CoA thioesterase (EC 3.1.2.-); and a phenyl acrylic acid decarboxylase enzyme (EC 4.1.1.-). In some embodiments the one or more enzymes that catalyse the conversion of crotonyl-CoA to an alkene include trans-2-enoyl-CoA reductase (EC1.1.1.36); acetyl-CoA C-acetyltransferase (EC 2.3.1.9); 3-hydroxybutyryl-CoA dehydrogenase (EC 1.1.1.157); 3-hydroxybutyryl-CoA dehydratase (EC 4.2.1.55); phosphate butyryltransferase (EC 2.3.1.19); butyrate kinase (EC 2.7.2.7); and a phenyl acrylic acid decarboxylase enzyme (EC 4.1.1.-).

In some embodiments the one or more enzymes that catalyse the conversion of crotonyl-CoA to an alkene include aldehyde dehydrogenase (EC 1.2.1.-); aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127).

In some embodiments the one or more enzymes that catalyse the conversion of crotonyl-CoA to an alkene include aldehyde dehydrogenase (EC 1.2.1.-); aldehyde dehydrogenase (EC 1.2.1.-) and/or alcohol dehydrogenase (EC 1.1.1.-); farnesol kinase (EC 2.7.1.-), geranylgeraniol kinase (EC 2.7.1.B19), hydroxyethylthiazole kinase (EC 2.7.1.50) and/or undecaprenol kinase (EC 2.7.1.66); and 2-methyl-1,3-butadiene synthase (EC 4.2.3.27) and/or methylbutenol synthase.

In some embodiments the one or more enzymes that catalyse the conversion of crotonyl-CoA to an alkene include alcohol dehydrogenase (EC 1.1.1.-); aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127).

In some embodiments the one or more enzymes that catalyse the conversion of crotonyl-CoA to an alkene include alcohol dehydrogenase (EC 1.1.1.-); aldehyde dehydrogenase (EC 1.2.1.-) and/or alcohol dehydrogenase (EC 1.1.1.-); farnesol kinase (EC 2.7.1.-), geranylgeraniol kinase (EC 2.7.1.B19), hydroxyethylthiazole kinase (EC 2.7.1.50) and/or undecaprenol kinase (EC 2.7.1.66); and 2-methyl-1,3-butadiene synthase (EC 4.2.3.27) and/or methylbutenol synthase.

In some embodiments the one or more enzymes that catalyse the conversion of crotonyl-CoA to an alkene include acyl-CoA thioesterase (EC 3.1.2.-); aldehyde dehydrogenase (EC 1.2.1.-) and/or or alcohol dehydrogenase (EC 1.1.1.-); aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127).

In some embodiments the one or more enzymes that catalyse the conversion of crotonyl-CoA to an alkene include acyl-CoA thioesterase (EC 3.1.2.-); aldehyde dehydrogenase (EC 1.2.1.-) and/or or alcohol dehydrogenase (EC 1.1.1.-); aldehyde dehydrogenase (EC 1.2.1.-) and/or alcohol dehydrogenase (EC 1.1.1.-); farnesol kinase (EC 2.7.1.-), geranylgeraniol kinase (EC 2.7.1.B19), hydroxyethylthiazole kinase (EC 2.7.1.50) and/or undecaprenol kinase (EC 2.7.1.66); and 2-methyl-1,3-butadiene synthase (EC 4.2.3.27) and/or methylbutenol synthase.

In some embodiments the one or more enzymes that catalyse the conversion of crotonyl-CoA to an alkene include aldehyde ferredoxin oxidoreductase (EC 1.2.7.5); acyl-CoA thioesterase (EC 3.1.2.-); aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127).

In some embodiments the one or more enzymes that catalyse the conversion of crotonyl-CoA to an alkene include aldehyde ferredoxin oxidoreductase (EC 1.2.7.5); acyl-CoA thioesterase (EC 3.1.2.-); aldehyde dehydrogenase (EC 1.2.1.-) and/or alcohol dehydrogenase (EC 1.1.1.-); farnesol kinase (EC 2.7.1.-), geranylgeraniol kinase (EC 2.7.1.B19), hydroxyethylthiazole kinase (EC 2.7.1.50) and/or undecaprenol kinase (EC 2.7.1.66); and 2-methyl-1,3-butadiene synthase (EC 4.2.3.27) and/or methylbutenol synthase.

In some embodiments the one or more enzymes that catalyse the conversion of crotonyl-CoA to an alkene include phosphate butyryltransferase (EC 2.3.1.19); butyrate kinase (EC 2.7.2.7); aldehyde dehydrogenase (EC 1.2.1.-) or alcohol dehydrogenase (EC 1.1.1.-); aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127).

In some embodiments the one or more enzymes that catalyse the conversion of crotonyl-CoA to an alkene include phosphate butyryltransferase (EC 2.3.1.19); butyrate kinase (EC 2.7.2.7); aldehyde dehydrogenase (EC 1.2.1.-) or alcohol dehydrogenase (EC 1.1.1.-); aldehyde dehydrogenase (EC 1.2.1.-) and/or alcohol dehydrogenase (EC 1.1.1.-); farnesol kinase (EC 2.7.1.-), geranylgeraniol kinase (EC 2.7.1.B19), hydroxyethylthiazole kinase (EC 2.7.1.50) and/or undecaprenol kinase (EC 2.7.1.66); and 2-methyl-1,3-butadiene synthase (EC 4.2.3.27) and/or methylbutenol synthase.

In some embodiments the one or more enzymes that catalyse the conversion of crotonyl-CoA to an alkene include phosphate butyryltransferase (EC 2.3.1.19); butyrate kinase (EC 2.7.2.7); aldehyde ferredoxin oxidoreductase (EC 1.2.7.5); aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127).

In some embodiments the one or more enzymes that catalyse the conversion of crotonyl-CoA to an alkene include phosphate butyryltransferase (EC 2.3.1.19); butyrate kinase (EC 2.7.2.7); aldehyde ferredoxin oxidoreductase (EC 1.2.7.5); aldehyde dehydrogenase (EC 1.2.1.-) and/or alcohol dehydrogenase (EC 1.1.1.-); farnesol kinase (EC 2.7.1.-), geranylgeraniol kinase (EC 2.7.1.B19), hydroxyethylthiazole kinase (EC 2.7.1.50) and/or undecaprenol kinase (EC 2.7.1.66); and 2-methyl-1,3-butadiene synthase (EC 4.2.3.27) and/or methylbutenol synthase.

In some embodiments the one or more enzymes that catalyse the conversion of crotonyl-CoA to an alkene include phenyl acrylic acid decarboxylase enzyme (EC 4.1.1.-); and acyl-CoA thioesterase (EC 3.1.2.-). In some embodiments the one or more enzymes that catalyse the conversion of crotonyl-CoA to an alkene include phenyl acrylic acid decarboxylase enzyme (EC 4.1.1.-); phosphate butyryltransferase (EC 2.3.1.19); and butyrate kinase (EC 2.7.2.7). In some embodiments the one or more enzymes that catalyse the conversion of crotonyl-CoA to an alkene include phenyl acrylic acid decarboxylase enzyme (EC 4.1.1.-); acyl-CoA thioesterase (EC 3.1.2.-); phosphate butyryltransferase (EC 2.3.1.19); and butyrate kinase (EC 2.7.2.7).

In some embodiments the one or more enzymes that catalyse the conversion of crotonyl-CoA to an alkene include aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127).

In some embodiments the one or more enzymes that catalyse the conversion of crotonyl-CoA to an alkene include aldehyde dehydrogenase (EC 1.2.1.-) and/or alcohol dehydrogenase (EC 1.1.1.-); farnesol kinase (EC 2.7.1.-), geranylgeraniol kinase (EC 2.7.1.B19), hydroxyethylthiazole kinase (EC 2.7.1.50) and/or undecaprenol kinase (EC 2.7.1.66); 2-methyl-1,3-butadiene synthase (EC 4.2.3.27) and/or methylbutenol synthase.

In some embodiments the one or more enzymes that catalyse the conversion of crotonyl-CoA to an alkene include acyl-CoA thioesterase (EC 3.1.2.-); aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127).

In some embodiments the one or more enzymes that catalyse the conversion of crotonyl-CoA to an alkene include acyl-CoA thioesterase (EC 3.1.2.-); aldehyde dehydrogenase (EC 1.2.1.-) and/or alcohol dehydrogenase (EC 1.1.1.-); farnesol kinase (EC 2.7.1.-), geranylgeraniol kinase (EC 2.7.1.B19), hydroxyethylthiazole kinase (EC 2.7.1.50) and/or undecaprenol kinase (EC 2.7.1.66); 2-methyl-1,3-butadiene synthase (EC 4.2.3.27) and/or methylbutenol synthase.

In some embodiments the one or more enzymes that catalyse the conversion of crotonyl-CoA to an alkene include aldehyde ferredoxin oxidoreductase (EC 1.2.7.5); acyl-CoA thioesterase (EC 3.1.2.-); aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127).

In some embodiments the one or more enzymes that catalyse the conversion of crotonyl-CoA to an alkene include aldehyde ferredoxin oxidoreductase (EC 1.2.7.5); acyl-CoA thioesterase (EC 3.1.2.-); aldehyde dehydrogenase (EC 1.2.1.-) and/or alcohol dehydrogenase (EC 1.1.1.-); farnesol kinase (EC 2.7.1.-), geranylgeraniol kinase (EC 2.7.1.B19), hydroxyethylthiazole kinase (EC 2.7.1.50) and/or undecaprenol kinase (EC 2.7.1.66); 2-methyl-1,3-butadiene synthase (EC 4.2.3.27) and/or methylbutenol synthase.

In some embodiments the one or more enzymes that catalyse the conversion of crotonyl-CoA to an alkene include phosphate butyryltransferase (EC 2.3.1.19); butyrate kinase (EC 2.7.2.7); aldehyde dehydrogenase (EC 1.2.1.-) and/or alcohol dehydrogenase (EC 1.1.1.-); geraniol isomerase (EC 5.4.4.4), which is also linalool dehydratase (EC 4.2.1.127).

In some embodiments the one or more enzymes that catalyse the conversion of crotonyl-CoA to an alkene include phosphate butyryltransferase (EC 2.3.1.19); butyrate kinase (EC 2.7.2.7); aldehyde dehydrogenase (EC 1.2.1.-) and/or alcohol dehydrogenase (EC 1.1.1.-); farnesol kinase (EC 2.7.1.-), geranylgeraniol kinase (EC 2.7.1.B19), hydroxyethylthiazole kinase (EC 2.7.1.50) and/or undecaprenol kinase (EC 2.7.1.66); 2-methyl-1,3-butadiene synthase (EC 4.2.3.27) and/or methylbutenol synthase.

In some embodiments the one or more enzymes that catalyse the conversion of crotonyl-CoA to an alkene include phosphate butyryltransferase (EC 2.3.1.19); butyrate kinase (EC 2.7.2.7); aldehyde ferredoxin oxidoreductase (EC 1.2.7.5); aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127).

In some embodiments the one or more enzymes that catalyse the conversion of crotonyl-CoA to an alkene include phosphate butyryltransferase (EC 2.3.1.19); butyrate kinase (EC 2.7.2.7); aldehyde ferredoxin oxidoreductase (EC 1.2.7.5); aldehyde dehydrogenase (EC 1.2.1.-) and/or alcohol dehydrogenase (EC 1.1.1.-); farnesol kinase (EC 2.7.1.-), geranylgeraniol kinase (EC 2.7.1.B19), hydroxyethylthiazole kinase (EC 2.7.1.50) and/or undecaprenol kinase (EC 2.7.1.66); 2-methyl-1,3-butadiene synthase (EC 4.2.3.27) and/or methylbutenol synthase.

In some embodiments the one or more enzymes that catalyse the conversion of propionyl-CoA to an alkene include acetyl-CoA C-acetyltransferase (EC 2.3.1.9); acetoacetyl-CoA reductase (EC 1.1.1.36); enoyl-CoA hydratase 2 (EC 4.2.1.119); acyl-CoA thioesterase (EC 3.1.2.-); and phenyl acrylic acid decarboxylase (EC 4.1.1.-).

In some embodiments the one or more enzymes that catalyse the conversion of propionyl-CoA to an alkene include acetyl-CoA C-acetyltransferase (EC 2.3.1.9); acetoacetyl-CoA reductase (EC 1.1.1.36); enoyl-CoA hydratase 2 (EC 4.2.1.119); phosphate butyryltransferase (EC 2.3.1.19); butyrate kinase (EC 2.7.2.7); and phenyl acrylic acid decarboxylase (EC 4.1.1.-).

In some embodiments the one or more enzymes that catalyse the conversion of propionyl-CoA to an alkene include acetyl-CoA C-acetyltransferase (EC 2.3.1.9); 3-hydroxybutyryl-CoA dehydrogenase (EC 1.1.1.157); 3-hydroxybutyryl-CoA dehydratase (EC 4.2.1.55); acyl-CoA thioesterase (EC 3.1.2.-); and phenyl acrylic acid decarboxylase (EC 4.1.1.-).

In some embodiments the one or more enzymes that catalyse the conversion of propionyl-CoA to an alkene include acetyl-CoA C-acetyltransferase (EC 2.3.1.9); 3-hydroxybutyryl-CoA dehydrogenase (EC 1.1.1.157); 3-hydroxybutyryl-CoA dehydratase (EC 4.2.1.55); phosphate butyryltransferase (EC 2.3.1.19); butyrate kinase (EC 2.7.2.7); and phenyl acrylic acid decarboxylase (EC 4.1.1.-).

In some embodiments the one or more enzymes that catalyse the conversion of acryloyl-CoA to an alkene include acyl-CoA thioesterase (EC 3.1.2.-); and phenyl acrylic acid decarboxylase (EC 4.1.1.-).

In some embodiments the one or more enzymes that catalyse the conversion of acryloyl-CoA to an alkene include phosphate butyryltransferase (EC 2.3.1.19); butyrate kinase (EC 2.7.2.7); and phenyl acrylic acid decarboxylase (EC 4.1.1.-).

According to some embodiments of the recombinant microorganism according to the first aspect the heterologous nucleic acid sequence includes coding sequences that encode the enzyme acetyl-CoA C-acetyltransferase (EC 2.3.1.9); the enzyme 3-hydroxybutyryl-CoA dehydrogenase (EC 1.1.1.157); the enzyme 3-hydroxybutyryl-CoA dehydratase (EC 4.2.1.55); an acyl-CoA thioesterase enzyme (EC 3.1.2.-); an aldehyde dehydrogenase (EC 1.2.1.-) or an alcohol dehydrogenase enzyme (EC 1.1.1.-); and the enzyme geraniol isomerase (EC 5.4.4.4), which is also linalool dehydratase (EC 4.2.1.127).

According to some embodiments of the recombinant microorganism according to the first aspect, the heterologous nucleic acid sequence includes coding sequences that encode the enzyme Acetyl-CoA carboxylase (EC 6.4.1.2); the enzyme acetoacetyl-CoA synthase (EC 2.3.1.194); the enzyme acetoacetyl-CoA reductase (EC 1.1.1.36); the enzyme enoyl-CoA hydratase 2 (EC 4.2.1.119); an acyl-CoA thioesterase enzyme (EC 3.1.2.-); an aldehyde dehydrogenase (EC 1.2.1.-) or an alcohol dehydrogenase enzyme (EC 1.1.1.-); and the enzyme geraniol isomerase (EC 5.4.4.4), which is also linalool dehydratase (EC 4.2.1.127).

According to some embodiments of the recombinant microorganism according to the first aspect, the heterologous nucleic acid sequence includes coding sequences that encode the enzyme acetyl-CoA carboxylase (EC 6.4.1.2); a lactate CoA-transferase enzyme (EC 2.8.3.-); the enzyme lactoyl-CoA dehydratase (EC 4.2.1.54); an acyl-CoA thioesterase enzyme (EC 3.1.2.-); and a phenyl acrylic acid decarboxylase enzyme (EC 4.1.1.-).

According to some embodiments of the recombinant microorganism according to the first aspect, the heterologous nucleic acid sequence includes coding sequences that encode the enzyme 4-aminobutyrate aminotransferase (EC 2.6.1.19); the enzyme glutamate dehydrogenase (EC 1.4.1.2); the enzyme 2-hydroxyglutarate dehydrogenase (EC 1.1.99.2); the enzyme glutaconate CoA-transferase (EC 2.8.3.12); a 2-hydroxyglutaryl-CoA dehydratase enzyme (EC 4.2.1.-); the enzyme glutaconyl-CoA decarboxylase (EC 4.1.1.70); an acyl-CoA thioesterase enzyme (EC 3.1.2.-); an aldehyde dehydrogenase (EC 1.2.1.-) or an alcohol dehydrogenase enzyme (EC 1.1.1.-); and the enzyme geraniol isomerase (EC 5.4.4.4), which is also linalool dehydratase (EC 4.2.1.127).

According to some embodiments of the recombinant microorganism according to the first aspect, the heterologous nucleic acid sequence includes coding sequences that encode the enzyme 4-aminobutyrate aminotransferase (EC 2.6.1.19); the enzyme 4-hydroxybutyrate dehydrogenase (EC 1.1.1.61); a 4-hydroxybutyrate CoA-transferase enzyme (EC 2.8.3.-); the enzyme vinylacetyl-CoA-Delta-isomerase (EC 4.2.1.120); the enzyme 4-hydroxybutyryl-CoA dehydratase (EC 5.3.3.3); an acyl-CoA thioesterase enzyme (EC 3.1.2.-); an aldehyde/alcohol dehydrogenase enzyme (EC 1.1.1.- and/or EC 1.2.1.-); and the enzyme geraniol isomerase (EC 5.4.4.4), which is also linalool dehydratase (EC 4.2.1.127).

According to some embodiments of the recombinant microorganism according to the first aspect, the heterologous nucleic acid sequence includes coding sequences that encode the enzyme 4-aminobutyrate aminotransferase (EC 2.6.1.19); the enzyme glutamate dehydrogenase (EC 1.4.1.2); the enzyme 2-hydroxyglutarate dehydrogenase (EC 1.1.99.2); the enzyme glutaconate CoA-transferase (EC 2.8.3.12); a 2-hydroxyglutaryl-CoA dehydratase enzyme (EC 4.2.1.-); the enzyme glutaconyl-CoA decarboxylase (EC 4.1.1.70); the enzyme 4-hydroxybutyrate dehydrogenase (EC 1.1.1.61); a 4-hydroxybutyrate CoA-transferase enzyme (EC 2.8.3.-); the enzyme vinylacetyl-CoA-Delta-isomerase (EC 4.2.1.120); the enzyme 4-hydroxybutyryl-CoA dehydratase (EC 5.3.3.3); an acyl-CoA thioesterase enzyme (EC 3.1.2.-); an aldehyde dehydrogenase (EC 1.2.1.-) or an alcohol dehydrogenase enzyme (EC 1.1.1.-); and the enzyme geraniol isomerase (EC 5.4.4.4), which is also linalool dehydratase (EC 4.2.1.127).

According to some embodiments of the recombinant microorganism according to the first aspect, the heterologous nucleic acid sequence includes coding sequences that encode the enzyme 4-aminobutyrate aminotransferase (EC 2.6.1.19); the enzyme 4-hydroxybutyrate dehydrogenase (EC 1.1.1.61); a 4-hydroxybutyrate CoA-transferase enzyme (EC 2.8.3.-); the enzyme vinylacetyl-CoA-delta-isomerase (EC 4.2.1.120); the enzyme glutaconyl-CoA decarboxylase (EC 4.1.1.70); the enzyme 4-hydroxybutyryl-CoA dehydratase (EC 5.3.3.3); the enzyme trans-2-enoyl-CoA reductase (EC1.1.1.36); the enzyme acetyl-CoA C-acetyltransferase (EC 2.3.1.9); the enzyme 3-hydroxybutyryl-CoA dehydrogenase (EC 1.1.1.157); the enzyme 3-hydroxybutyryl-CoA dehydratase (EC 4.2.1.55); an acyl-CoA thioesterase enzyme (EC 3.1.2.-); and a phenyl acrylic acid decarboxylase enzyme (EC 4.1.1.-).

According to some embodiments of the recombinant microorganism according to the first aspect, the heterologous nucleic acid sequence includes coding sequences that encode the enzyme acetyl-CoA C-acetyltransferase (EC 2.3.1.9), the enzyme 3-hydroxy-3-methylglutaryl-CoA synthase (EC 2.3.3.10), the enzyme 3-methylglutaconyl-CoA hydratase (EC 4.2.1.18), and the enzyme 3-methylcrotonyl-CoA carboxylase (EC 6.4.1.4), an aldehyde dehydrogenase (EC 1.2.1.-) or an alcohol dehydrogenase enzyme (EC 1.1.1.-); and the enzyme geraniol isomerase (EC 5.4.4.4), which is also linalool dehydratase (EC 4.2.1.127).

According to some embodiments of the recombinant microorganism according to the first aspect, the heterologous nucleic acid sequence includes coding sequences that encode the enzyme acetyl-CoA C-acetyltransferase (EC 2.3.1.9), the enzyme 3-hydroxy-3-methylglutaryl-CoA synthase (EC 2.3.3.10), the enzyme 3-methylglutaconyl-CoA hydratase (EC 4.2.1.18), and the enzyme 3-methylcrotonyl-CoA carboxylase (EC 6.4.1.4), an aldehyde dehydrogenase (EC 1.2.1.-) or an alcohol dehydrogenase enzyme (EC 1.1.1.-); the enzyme farnesol kinase (EC 2.7.1.-), and/or the enzyme geranylgeraniol kinase (EC 2.7.1.B19), and/or the enzyme hydroxyethylthiazole kinase (EC 2.7.1.50) and/or the enzyme undecaprenol kinase (EC 2.7.1.66); and the enzyme 2-methyl-1,3-butadiene synthase (EC 4.2.3.27) and/or the enzyme methylbutenol synthase.

According to some embodiments of the recombinant microorganism according to the first aspect, the heterologous nucleic acid sequence includes coding sequences that encode the enzyme acetyl-CoA carboxylase (EC 6.4.1.2), the enzyme acetoacetyl-CoA synthase (EC 2.3.1.194), the enzyme 3-hydroxy-3-methylglutaryl-CoA synthase (EC 2.3.3.10), the enzyme 3-methylglutaconyl-CoA hydratase (EC 4.2.1.18), the enzyme 3-methylcrotonyl-CoA carboxylase (EC 6.4.1.4), an aldehyde dehydrogenase (EC 1.2.1.-) or an alcohol dehydrogenase enzyme (EC 1.1.1.-); and the enzyme geraniol isomerase (EC 5.4.4.4), which is also linalool dehydratase (EC 4.2.1.127).

According to some embodiments of the recombinant microorganism according to the first aspect, the heterologous nucleic acid sequence includes coding sequences that encode the enzyme acetyl-CoA carboxylase (EC 6.4.1.2), the enzyme acetoacetyl-CoA synthase (EC 2.3.1.194), the enzyme 3-hydroxy-3-methylglutaryl-CoA synthase (EC 2.3.3.10), the enzyme 3-methylglutaconyl-CoA hydratase (EC 4.2.1.18), the enzyme 3-methylcrotonyl-CoA carboxylase (EC 6.4.1.4), an aldehyde dehydrogenase (EC 1.2.1.-) or an alcohol dehydrogenase enzyme (EC 1.1.1.-); the enzyme farnesol kinase (EC 2.7.1.-), and/or the enzyme geranylgeraniol kinase (EC 2.7.1.B19), and/or the enzyme hydroxyethylthiazole kinase (EC 2.7.1.50) and/or the enzyme undecaprenol kinase (EC 2.7.1.66); and the enzyme 2-methyl-1,3-butadiene synthase (EC 4.2.3.27) and/or the enzyme methylbutenol synthase.

According to some embodiments of the recombinant microorganism according to the first aspect, the heterologous nucleic acid sequence includes coding sequences that encode the enzyme acetyl-CoA C-acetyltransferase (EC 2.3.1.9), the enzyme 3-hydroxybutyryl-CoA dehydrogenase (EC 1.1.1.157), the enzyme 3-hydroxybutyryl-CoA dehydratase (EC 4.2.1.55), an aldehyde dehydrogenase (EC 1.2.1.-) or an alcohol dehydrogenase enzyme (EC 1.1.1.-); and the geraniol isomerase (EC 5.4.4.4), which is also linalool dehydratase (EC 4.2.1.127).

According to some embodiments of the recombinant microorganism according to the first aspect, the heterologous nucleic acid sequence includes coding sequences that encode the enzyme acetyl-CoA C-acetyltransferase (EC 2.3.1.9), the enzyme 3-hydroxybutyryl-CoA dehydrogenase (EC 1.1.1.157), the enzyme 3-hydroxybutyryl-CoA dehydratase (EC 4.2.1.55), an aldehyde dehydrogenase (EC 1.2.1.-) or an alcohol dehydrogenase enzyme (EC 1.1.1.-); the enzyme farnesol kinase (EC 2.7.1.-), and/or the enzyme geranylgeraniol kinase (EC 2.7.1.B19), and/or the enzyme hydroxyethylthiazole kinase (EC 2.7.1.50) and/or the enzyme undecaprenol kinase (EC 2.7.1.66); and the enzyme 2-methyl-1,3-butadiene synthase (EC 4.2.3.27) and/or the enzyme methylbutenol synthase.

According to some embodiments of the recombinant microorganism according to the first aspect, the heterologous nucleic acid sequence includes coding sequences that encode the enzyme Acetyl-CoA carboxylase (EC 6.4.1.2), the enzyme acetoacetyl-CoA synthase (EC 2.3.1.194), the enzyme acetoacetyl-CoA reductase (EC 1.1.1.36), the enzyme enoyl-CoA hydratase 2 (EC 4.2.1.119), an aldehyde dehydrogenase (EC 1.2.1.-) or an alcohol dehydrogenase enzyme (EC 1.1.1.-); and the enzyme geraniol isomerase (EC 5.4.4.4), which is also linalool dehydratase (EC 4.2.1.127).

According to some embodiments of the recombinant microorganism according to the first aspect, the heterologous nucleic acid sequence includes coding sequences that encode the enzyme Acetyl-CoA carboxylase (EC 6.4.1.2), the enzyme acetoacetyl-CoA synthase (EC 2.3.1.194), the enzyme acetoacetyl-CoA reductase (EC 1.1.1.36), the enzyme enoyl-CoA hydratase 2 (EC 4.2.1.119), an aldehyde dehydrogenase (EC 1.2.1.-) or an alcohol dehydrogenase enzyme (EC 1.1.1.-); the enzyme farnesol kinase (EC 2.7.1.-), and/or the enzyme geranylgeraniol kinase (EC 2.7.1.B19), and/or the enzyme hydroxyethylthiazole kinase (EC 2.7.1.50) and/or the enzyme undecaprenol kinase (EC 2.7.1.66); and the enzyme 2-methyl-1,3-butadiene synthase (EC 4.2.3.27) and/or the enzyme methylbutenol synthase.

According to some embodiments of the recombinant microorganism according to the first aspect the heterologous nucleic acid sequence includes coding sequences that encode the enzyme acetyl-CoA C-acetyltransferase (EC 2.3.1.9); the enzyme 3-hydroxy-3-methylglutaryl-CoA synthase (EC 2.3.3.10); the enzyme 3-methylglutaconyl-CoA hydratase (EC 4.2.1.18); the enzyme 3-methyl-glutaconyl-CoA decarboxylase; an aldehyde/alcohol dehydrogenase enzyme (EC 1.1.1.- or EC 1.2.1.-); and the enzyme geraniol isomerase (EC 5.4.4.4), which is also linalool dehydratase (EC 4.2.1.127).

According to some embodiments of the recombinant microorganism according to the first aspect the heterologous nucleic acid sequence includes coding sequences that encode the enzyme acetyl-CoA carboxylase (EC 6.4.1.2); the enzyme acetoacetyl-CoA synthase (EC 2.3.1.194); the enzyme 3-hydroxy-3-methylglutaryl-CoA synthase (EC 2.3.3.10); the enzyme 3-methylglutaconyl-CoA hydratase (EC 4.2.1.18); the enzyme 3-methylglutaconyl-CoA decarboxylase; an aldehyde/alcohol dehydrogenase enzyme (EC 1.1.1.- or EC 1.2.1.-); and the enzyme geraniol isomerase (EC 5.4.4.4), which is also linalool dehydratase (EC 4.2.1.127).

According to some embodiments of the recombinant microorganism according to the first aspect the heterologous nucleic acid sequence includes coding sequences that encode the enzyme acetyl-CoA C-acetyltransferase (EC 2.3.1.9); the enzyme 3-hydroxy-3-methylglutaryl-CoA synthase (EC 2.3.3.10); the enzyme 3-methylglutaconyl-CoA hydratase (EC 4.2.1.18); the enzyme 3-methylglutaconyl-CoA decarboxylase; the enzyme farnesol kinase (EC 2.7.1.-), and/or the enzyme geranylgeraniol kinase (EC 2.7.1.B19) and/or the enzyme hydroxyethylthiazole kinase (EC 2.7.1.50) and/or the enzyme undecaprenol kinase (EC 2.7.1.66); and the enzyme 2-methyl-1,3-butadiene synthase (EC 4.2.3.27) and/or the enzyme methylbutenol synthase.

According to some embodiments of the recombinant microorganism according to the first aspect the heterologous nucleic acid sequence includes coding sequences that encode the enzyme acetyl-CoA carboxylase (EC 6.4.1.2); the enzyme acetoacetyl-CoA synthase (EC 2.3.1.194); the enzyme 3-hydroxy-3-methylglutaryl-CoA synthase (EC 2.3.3.10); the enzyme 3-methylglutaconyl-CoA hydratase (EC 4.2.1.18); the enzyme 3-methylglutaconyl-CoA decarboxylase; the enzyme farnesol kinase (EC 2.7.1.-), and/or the enzyme geranylgeraniol kinase (EC 2.7.1.B19) and/or the enzyme hydroxyethylthiazole kinase (EC 2.7.1.50) and/or the enzyme undecaprenol kinase (EC 2.7.1.66); and the enzyme 2-methyl-1,3-butadiene synthase (EC 4.2.3.27) and/or the enzyme methylbutenol synthase.

According to some embodiments of the recombinant microorganism according to the first aspect, the heterologous nucleic acid sequence includes coding sequences that encode the enzyme acetyl-CoA C-acetyltransferase (EC 2.3.1.9); the enzyme 3-hydroxybutyryl-CoA dehydrogenase (EC 1.1.1.157); the enzyme 3-hydroxybutyryl-CoA dehydratase (EC 4.2.1.55); an acyl-CoA thioesterase enzyme (EC 3.1.2.-); and a phenyl acrylic acid decarboxylase enzyme (EC 4.1.1.-).

According to some embodiments of the recombinant microorganism according to the first aspect, the heterologous nucleic acid sequence includes coding sequences that encode the enzyme Acetyl-CoA carboxylase (EC 6.4.1.2); the enzyme acetoacetyl-CoA synthase (EC 2.3.1.194); the enzyme acetoacetyl-CoA reductase (EC 1.1.1.36); the enzyme enoyl-CoA hydratase 2 (EC 4.2.1.119); an acyl-CoA thioesterase enzyme (EC 3.1.2.-); and a phenyl acrylic acid decarboxylase enzyme (EC 4.1.1.-).

According to some embodiments of the recombinant microorganism according to the first aspect, the heterologous nucleic acid sequence includes coding sequences that encode the enzyme acetyl-CoA C-acetyltransferase (EC 2.3.1.9); the enzyme 3-hydroxy-3-methylglutaryl-CoA synthase (EC 2.3.3.10); the enzyme 3-methylglutaconyl-CoA hydratase (EC 4.2.1.18); the enzyme 3-methylglutaconyl-CoA decarboxylase; a acyl-CoA thioesterase enzyme (EC 3.1.2.-); and the enzyme phenyl acrylic acid decarboxylase (EC 4.1.1.-).

According to some embodiments of the recombinant microorganism according to the first aspect, the heterologous nucleic acid sequence includes coding sequences that encode the enzyme Acetyl-CoA carboxylase (EC 6.4.1.2); the enzyme acetoacetyl-CoA synthase (EC 2.3.1.194); the enzyme 3-hydroxy-3-methylglutaryl-CoA synthase (EC 2.3.3.10); the enzyme 3-methylglutaconyl-CoA hydratase (EC 4.2.1.18); the enzyme 3-methylglutaconyl-CoA decarboxylase; a acyl-CoA thioesterase enzyme (EC 3.1.2.-); and the enzyme phenyl acrylic acid decarboxylase (EC 4.1.1.-).

According to some embodiments of the recombinant microorganism according to the first aspect, the heterologous nucleic acid sequence includes coding sequences that encode the enzyme 3-hydroxybutyryl-CoA dehydrogenase (EC 1.1.1.157); the enzyme 3-hydroxybutyryl-CoA dehydratase (EC 4.2.1.55); the enzyme trans-2-enoyl-CoA reductase (EC1.1.1.36); the enzyme acetyl-CoA C-acetyltransferase (EC 2.3.1.9); the enzyme 3-hydroxybutyryl-CoA dehydrogenase (EC 1.1.1.157); the enzyme 3-hydroxybutyryl-CoA dehydratase (EC 4.2.1.55); an acyl-CoA thioesterase enzyme (EC 3.1.2.-); and a phenyl acrylic acid decarboxylase enzyme (EC 4.1.1.-).

According to some embodiments of the recombinant microorganism according to the first aspect, the heterologous nucleic acid sequence includes coding sequences that encode the enzyme Acetyl-CoA carboxylase (EC 6.4.1.2); the enzyme acetoacetyl-CoA synthase (EC 2.3.1.194); the enzyme acetoacetyl-CoA reductase (EC 1.1.1.36); the enzyme enoyl-CoA hydratase 2 (EC 4.2.1.119); the enzyme trans-2-enoyl-CoA reductase (EC1.1.1.36); the enzyme acetyl-CoA C-acetyltransferase (EC 2.3.1.9); the enzyme 3-hydroxybutyryl-CoA dehydrogenase (EC 1.1.1.157); the enzyme 3-hydroxybutyryl-CoA dehydratase (EC 4.2.1.55); an acyl-CoA thioesterase enzyme (EC 3.1.2.-); and a phenyl acrylic acid decarboxylase enzyme (EC 4.1.1.-).

According to some embodiments of the recombinant microorganism according to the first aspect, the heterologous nucleic acid sequence includes coding sequences that encode the enzyme Acetyl-CoA carboxylase (EC 6.4.1.2); the enzyme acetoacetyl-CoA synthase (EC 2.3.1.194); the enzyme acetoacetyl-CoA reductase (EC 1.1.1.36); the enzyme enoyl-CoA hydratase 2 (EC 4.2.1.119); the enzyme trans-2-enoyl-CoA reductase (EC1.1.1.36); the enzyme acetyl-CoA C-acetyltransferase (EC 2.3.1.9); the enzyme 3-hydroxybutyryl-CoA dehydrogenase (EC 1.1.1.157); the enzyme 3-hydroxybutyryl-CoA dehydratase (EC 4.2.1.55); an acyl-CoA thioesterase enzyme (EC 3.1.2.-); and a phenyl acrylic acid decarboxylase enzyme (EC 4.1.1.-).

According to some embodiments of the recombinant microorganism according to the first aspect, the heterologous nucleic acid sequence includes coding sequences that encode the enzyme 3-hydroxybutyryl-CoA dehydrogenase (EC 1.1.1.157); the enzyme 3-hydroxybutyryl-CoA dehydratase (EC 4.2.1.55); the enzyme trans-2-enoyl-CoA reductase (EC1.1.1.36); the enzyme acetyl-CoA C-acetyltransferase (EC 2.3.1.9); the enzyme 3-hydroxybutyryl-CoA dehydrogenase (EC 1.1.1.157); the enzyme 3-hydroxybutyryl-CoA dehydratase (EC 4.2.1.55); a acyl-CoA thioesterase enzyme (EC 3.1.2.-); and a phenyl acrylic acid decarboxylase enzyme (EC 4.1.1.-).

According to some embodiments of the recombinant microorganism according to the first aspect, the heterologous nucleic acid sequence includes coding sequences that encode the enzyme 4-aminobutyrate aminotransferase (EC 2.6.1.19); the enzyme 4-hydroxybutyrate dehydrogenase (EC 1.1.1.61); a 4-hydroxybutyrate CoA-transferase enzyme (EC 2.8.3.-); the enzyme vinylacetyl-CoA-delta-isomerase (EC 4.2.1.120); the enzyme 4-hydroxybutyryl-CoA dehydratase (EC 5.3.3.3); an acyl-CoA thioesterase enzyme (EC 3.1.2.-); and the enzyme phenyl acrylic acid decarboxylase (EC 4.1.1.-).

According to some embodiments of the recombinant microorganism according to the first aspect, the heterologous nucleic acid sequence includes coding sequences that encode the enzyme 4-aminobutyrate aminotransferase (EC 2.6.1.19); the enzyme glutamate dehydrogenase (EC 1.4.1.2); the enzyme 2-hydroxyglutarate dehydrogenase (EC 1.1.99.2); the enzyme glutaconate CoA-transferase (EC 2.8.3.12); the enzyme 2-hydroxyglutaryl-CoA dehydratase (EC 4.2.1.-); the enzyme glutaconyl-CoA decarboxylase (EC 4.1.1.70); an acyl-CoA thioesterase enzyme (EC 3.1.2.-); and the enzyme phenyl acrylic acid decarboxylase (EC 4.1.1.-).

According to some embodiments of the recombinant microorganism according to the first aspect, the heterologous nucleic acid sequence includes coding sequences that encode the enzyme 2-oxoglutarate/butyrate ferredoxin oxidoreductase (EC 1.2.7.3); the enzyme pyruvate/2-oxobutyrate ferredoxin oxidoreductase (EC 1.2.7.1); the enzyme acetyl-CoA C-acetyltransferase (EC 2.3.1.9); the enzyme 3-hydroxybutyryl-CoA dehydrogenase (EC 1.1.1.157); the enzyme 3-hydroxybutyryl-CoA dehydratase (EC 4.2.1.55); an acyl-CoA thioesterase enzyme (EC 3.1.2.-); and a phenyl acrylic acid decarboxylase enzyme (EC 4.1.1.-).

According to some embodiments of the recombinant microorganism according to the first aspect, the heterologous nucleic acid sequence includes coding sequences that encode the enzyme 2-oxoglutarate/butyrate ferredoxin oxidoreductase (EC 1.2.7.3); the enzyme pyruvate/2-oxobutyrate ferredoxin oxidoreductase (EC 1.2.7.1); the enzyme acetyl-CoA C-acetyltransferase (EC 2.3.1.9); the enzyme acetoacetyl-CoA reductase (EC 1.1.1.36); the enzyme enoyl-CoA hydratase 2 (EC 4.2.1.119); an acyl-CoA thioesterase enzyme (EC 3.1.2.-); and a phenyl acrylic acid decarboxylase enzyme (EC 4.1.1.-).

According to some embodiments of the recombinant microorganism according to the first aspect, the heterologous nucleic acid sequence includes coding sequences that encode the enzyme 2-oxoglutarate/butyrate ferredoxin oxidoreductase (EC 1.2.7.3); the enzyme pyruvate/2-oxobutyrate ferredoxin oxidoreductase (EC 1.2.7.1); the enzyme acetyl-CoA C-acetyltransferase (EC 2.3.1.9); the enzyme 3-hydroxybutyryl-CoA dehydrogenase (EC 1.1.1.157); the enzyme 3-hydroxybutyryl-CoA dehydratase (EC 4.2.1.55); the enzyme phosphate butyryltransferase (EC 2.3.1.19); the enzyme butyrate kinase (EC 2.7.2.7); and a phenyl acrylic acid decarboxylase enzyme (EC 4.1.1.-).

According to some embodiments of the recombinant microorganism according to the first aspect, the heterologous nucleic acid sequence includes coding sequences that encode the enzyme 2-oxoglutarate/butyrate ferredoxin oxidoreductase (EC 1.2.7.3); the enzyme pyruvate/2-oxobutyrate ferredoxin oxidoreductase (EC 1.2.7.1); the enzyme acetyl-CoA C-acetyltransferase (EC 2.3.1.9); the enzyme acetoacetyl-CoA reductase (EC 1.1.1.36); the enzyme enoyl-CoA hydratase 2 (EC 4.2.1.119); the enzyme phosphate butyryltransferase (EC 2.3.1.19); the enzyme butyrate kinase (EC 2.7.2.7); and a phenyl acrylic acid decarboxylase enzyme (EC 4.1.1.-).

According to some embodiments of the recombinant microorganism according to the first aspect, the heterologous nucleic acid sequence includes coding sequences that encode the enzyme 2-oxoglutarate/butyrate ferredoxin oxidoreductase (EC 1.2.7.3); the enzyme pyruvate/2-oxobutyrate ferredoxin oxidoreductase (EC 1.2.7.1); the enzyme acetyl-CoA C-acetyltransferase (EC 2.3.1.9); the enzyme 3-hydroxybutyryl-CoA dehydrogenase (EC 1.1.1.157); the enzyme 3-hydroxybutyryl-CoA dehydratase (EC 4.2.1.55); the enzyme phosphate butyryltransferase (EC 2.3.1.19); the enzyme butyrate kinase (EC 2.7.2.7); and a phenyl acrylic acid decarboxylase enzyme (EC 4.1.1.-).

According to some embodiments of the recombinant microorganism according to the first aspect, the heterologous nucleic acid sequence includes coding sequences that encode the enzyme 2-oxoglutarate/butyrate ferredoxin oxidoreductase (EC 1.2.7.3); the enzyme pyruvate/2-oxobutyrate ferredoxin oxidoreductase (EC 1.2.7.1); the enzyme acetyl-CoA C-acetyltransferase (EC 2.3.1.9); the enzyme acetoacetyl-CoA reductase (EC 1.1.1.36); the enzyme enoyl-CoA hydratase 2 (EC 4.2.1.119); the enzyme phosphate butyryltransferase (EC 2.3.1.19); the enzyme butyrate kinase (EC 2.7.2.7); and a phenyl acrylic acid decarboxylase enzyme (EC 4.1.1.-).

According to some embodiments of the recombinant microorganism according to the first aspect, the heterologous nucleic acid sequence includes coding sequences that encode a lactate CoA-transferase enzyme (EC 2.8.3.-); the enzyme lactoyl-CoA dehydratase (EC 4.2.1.54); the enzyme lcdAB; the enzyme propionyl-CoA synthase (EC 1.3.1.84); the enzyme acetyl-CoA C-acetyltransferase (EC 2.3.1.9); the enzyme 3-hydroxybutyryl-CoA dehydrogenase (EC 1.1.1.157); the enzyme 3-hydroxybutyryl-CoA dehydratase (EC 4.2.1.55); an acyl-CoA thioesterase enzyme (EC 3.1.2.-); and a phenyl acrylic acid decarboxylase enzyme (EC 4.1.1.-).

According to some embodiments of the recombinant microorganism according to the first aspect, the heterologous nucleic acid sequence includes coding sequences that encode a lactate CoA-transferase enzyme (EC 2.8.3.-); the enzyme lactoyl-CoA dehydratase (EC 4.2.1.54); the enzyme lcdAB; the enzyme propionyl-CoA synthase (EC 1.3.1.84); the enzyme acetyl-CoA C-acetyltransferase (EC 2.3.1.9); the enzyme acetoacetyl-CoA reductase (EC 1.1.1.36); the enzyme enoyl-CoA hydratase 2 (EC 4.2.1.119); an acyl-CoA thioesterase enzyme (EC 3.1.2.-); and a phenyl acrylic acid decarboxylase enzyme (EC 4.1.1.-).

According to some embodiments of the recombinant microorganism according to the first aspect, the heterologous nucleic acid sequence includes a coding sequences that encode a lactate CoA-transferase enzyme (EC 2.8.3.-); the enzyme lactoyl-CoA dehydratase (EC 4.2.1.54); the enzyme lcdAB; propionyl-CoA synthase (EC 1.3.1.84); the enzyme acetyl-CoA C-acetyltransferase (EC 2.3.1.9); the enzyme acetoacetyl-CoA reductase (EC 1.1.1.36); the enzyme enoyl-CoA hydratase 2 (EC 4.2.1.119); the enzyme phosphate butyryltransferase (EC 2.3.1.19); the enzyme butyrate kinase (EC 2.7.2.7); and a phenyl acrylic acid decarboxylase enzyme (EC 4.1.1.-).

According to some embodiments of the recombinant microorganism according to the first aspect, the heterologous nucleic acid sequence includes coding sequences that encode a lactate CoA-transferase enzyme (EC 2.8.3.-); the enzyme lactoyl-CoA dehydratase (EC 4.2.1.54); the enzyme lcdAB; propionyl-CoA synthase (EC 1.3.1.84); the enzyme acetyl-CoA C-acetyltransferase (EC 2.3.1.9); the enzyme 3-hydroxybutyryl-CoA dehydrogenase (EC 1.1.1.157); the enzyme 3-hydroxybutyryl-CoA dehydratase (EC 4.2.1.55); the enzyme phosphate butyryltransferase (EC 2.3.1.19); the enzyme butyrate kinase (EC 2.7.2.7); and a phenyl acrylic acid decarboxylase enzyme (EC 4.1.1.-).

According to some embodiments of the recombinant microorganism according to the first aspect, the heterologous nucleic acid sequence includes coding sequences that encode a lactate CoA-transferase enzyme (EC 2.8.3.-); the enzyme lactoyl-CoA dehydratase (EC 4.2.1.54); an acyl-CoA thioesterase enzyme (EC 3.1.2.-); and a phenyl acrylic acid decarboxylase enzyme (EC 4.1.1.-).

According to some embodiments of the recombinant microorganism according to the first aspect, the heterologous nucleic acid sequence includes coding sequences that encode the enzyme acetyl-CoA carboxylase (EC 6.4.1.2); the enzyme malonyl CoA reductase (EC 1.2.1.75); the enzyme malonyl CoA reductase (EC 1.1.1.298); the enzyme propionyl-CoA synthase (EC 6.2.1.36); the enzyme propionyl-CoA synthase/acryloyl-CoA synthase (EC 4.2.1.116); the enzyme propionyl-CoA synthase (EC 1.3.1.84); the enzyme acetyl-CoA C-acetyltransferase (EC 2.3.1.9); the enzyme acetoacetyl-CoA reductase (EC 1.1.1.36); the enzyme enoyl-CoA hydratase 2 (EC 4.2.1.119); an acyl-CoA thioesterase enzyme (EC 3.1.2.-); and a phenyl acrylic acid decarboxylase enzyme (EC 4.1.1.-).

According to some embodiments of the recombinant microorganism according to the first aspect, the heterologous nucleic acid sequence includes coding sequences that encode the enzyme acetyl-CoA carboxylase (EC 6.4.1.2); the enzyme malonyl CoA reductase (EC 1.2.1.75); the enzyme malonyl CoA reductase (EC 1.1.1.298); the enzyme propionyl-CoA synthase (EC 6.2.1.36); the enzyme propionyl-CoA synthase/acryloyl-CoA synthase (EC 4.2.1.116); the enzyme propionyl-CoA synthase (EC 1.3.1.84); the enzyme acetyl-CoA C-acetyltransferase (EC 2.3.1.9); the enzyme 3-hydroxybutyryl-CoA dehydrogenase (EC 1.1.1.157); the enzyme 3-hydroxybutyryl-CoA dehydratase (EC 4.2.1.55); an acyl-CoA thioesterase enzyme (EC 3.1.2.-); and a phenyl acrylic acid decarboxylase enzyme (EC 4.1.1.-).

According to some embodiments of the recombinant microorganism according to the first aspect, the heterologous nucleic acid sequence includes coding sequences that encode the enzyme acetyl-CoA carboxylase (EC 6.4.1.2); the enzyme malonyl CoA reductase (EC 1.2.1.75); the enzyme malonyl CoA reductase (EC 1.1.1.298); the enzyme propionyl-CoA synthase (EC 6.2.1.36); the enzyme propionyl-CoA synthase/acryloyl-CoA synthase (EC 4.2.1.116); the enzyme propionyl-CoA synthase (EC 1.3.1.84); the enzyme acetyl-CoA C-acetyltransferase (EC 2.3.1.9); the enzyme acetoacetyl-CoA reductase (EC 1.1.1.36); the enzyme enoyl-CoA hydratase 2 (EC 4.2.1.119); the enzyme phosphate butyryltransferase (EC 2.3.1.19); the enzyme butyrate kinase (EC 2.7.2.7); and a phenyl acrylic acid decarboxylase enzyme (EC 4.1.1.-).

According to some embodiments of the recombinant microorganism according to the first aspect, the heterologous nucleic acid sequence includes coding sequences that encode the enzyme acetyl-CoA carboxylase (EC 6.4.1.2); the enzyme malonyl CoA reductase (EC 1.2.1.75); the enzyme malonyl CoA reductase (EC 1.1.1.298); the enzyme propionyl-CoA synthase (EC 6.2.1.36); the enzyme propionyl-CoA synthase/acryloyl-CoA synthase (EC 4.2.1.116); the enzyme propionyl-CoA synthase (EC 1.3.1.84); the enzyme acetyl-CoA C-acetyltransferase (EC 2.3.1.9); the enzyme 3-hydroxybutyryl-CoA dehydrogenase (EC 1.1.1.157); the enzyme 3-hydroxybutyryl-CoA dehydratase (EC 4.2.1.55); the enzyme phosphate butyryltransferase (EC 2.3.1.19); the enzyme butyrate kinase (EC 2.7.2.7); and a phenyl acrylic acid decarboxylase enzyme (EC 4.1.1.-).

According to some embodiments of the recombinant microorganism according to the first aspect, the heterologous nucleic acid sequence includes coding sequences that encode the enzyme acetyl-CoA carboxylase (EC 6.4.1.2); the enzyme malonyl CoA reductase (malonate semialdehyde-forming) (EC 1.2.1.75); the enzyme 3-hydroxypropionate dehydrogenase (EC 1.1.1.298); the enzyme 3-hydroxypropionyl-CoA synthase (EC 6.2.1.36); the enzyme hydroxypropionyl-CoA dehydratase (EC 4.2.1.116); an acyl-CoA thioesterase enzyme (EC 3.1.2.-); and a phenyl acrylic acid decarboxylase enzyme (EC 4.1.1.-).

According to some embodiments of the recombinant microorganism according to the first aspect, the heterologous nucleic acid sequence includes coding sequences that encode the enzyme acetoacetyl-Coenzyme A synthase thiolase (EC 2.3.1.9); the enzyme phosphate butyryltransferase (EC 2.3.1.19); the enzyme butyrate kinase (EC 2.7.2.7); the enzyme acetyl-CoA carboxylase (EC 6.4.1.2); the enzyme acetoacetyl-CoA synthase (EC 2.3.1.194); the enzyme 3-hydroxy-3-methylglutaryl-CoA synthase (EC 2.3.3.10); the enzyme 3-methylglutaconyl-CoA hydratase (EC 4.2.1.18); the enzyme 3-methylglutaconyl-CoA decarboxylase; an acyl-CoA thioesterase enzyme (EC 3.1.2.-); an aldehyde dehydrogenase (EC 1.2.1.-) or an alcohol dehydrogenase enzyme (EC 1.1.1.-); the enzyme aldehyde ferredoxin oxidoreductase (EC 1.2.7.5); the enzyme geraniol isomerase (EC 5.4.4.4), which is also linalool dehydratase (EC 4.2.1.127); the enzyme farnesol kinase (EC 2.7.1.-); and the enzyme methylbutenol synthase.

In some embodiments a microorganism disclosed herein is anaerobic. The microorganism may be an anaerobic cell. According to some embodiments of the recombinant microorganism according to the first aspect, the endogenous enzymes that convert one or both of carbon monoxide and carbon dioxide to acetyl-CoA are enzymes of the Wood-Ljungdahl pathway. In some embodiments the recombinant microorganism includes sequences encoding endogenous enzymes of all enzymes of the Wood-Ljungdahl pathway. The recombinant microorganism may express endogenous enzymes of the Wood-Ljungdahl pathway that catalyse the conversion of one or both of carbon monoxide and carbon dioxide to acetyl-CoA are enzymes. In some embodiments the recombinant microorganism includes sequences encoding endogenous enzymes of all enzymes of the Wood-Ljungdahl pathway. The recombinant microorganism may express all enzymes of the Wood-Ljungdahl pathway. In some embodiments the recombinant microorganism is caroxyotrop. In some embodiments the recombinant microorganism is acetogen.

According to some embodiments of the recombinant microorganism according to the first aspect, all coding sequences are operationally linked to the same common transcriptional promoter. According to some embodiments the heterologous gene sequence is included in a heterologous nucleic acid molecule or in the microorganism's chromosome. According to some embodiments the microorganism is of the genus Clostridium. In some embodiments the microorganism may be Clostridium ljungdahlii. The microorganism may also be Clostridium autoethanogenum. In some embodiments the microorganism may be Clostridium carboxidivorans. In some embodiments the microorganism may be Clostridium aceticum. The microorganism may in some embodiments be Clostridium drakei. In some embodiments the microorganism may be Clostridium scatologenes. In some embodiments the microorganism may be Clostridium ragsdalei. The microorganism may in some embodiments be Clostridium formicoaceticum. In some embodiments the microorganism may be Clostridium magnum. In some embodiments the microorganism may be Clostridium coccoides. The microorganism may in some embodiments be Clostridium difficile. In some embodiments the microorganism may be Clostridium formicaceticum. The microorganism may in some embodiments be Clostridium fervidus. In some embodiments the microorganism may be Clostridium thermocellum.

According to some embodiments the recombinant microorganism according to the first aspect is of the genus Moorella. In some embodiments the microorganism may be Moorella thermoacetica. In some embodiments the microorganism may be Moorella thermautotrophica. The microorganism may in some embodiments be Moorella mulderi. In some embodiments the microorganism may be Moorella stamsii. In some embodiments the microorganism may be Moorella glycerini. In some embodiments the microorganism is of the genus Acetobacterium. The microorganism may in some embodiments be Acetobacterium woodii. In some embodiments the microorganism may be Acetobacterium bakii. In some embodiments the microorganism may be Acetobacterium carbinolicum. The microorganism may in some embodiments be Acetobacterium dehalogenans. In some embodiments the microorganism may be Acetobacterium malicum. The microorganism may in some embodiments be Acetobacterium paludosum. In some embodiments the microorganism may be Acetobacterium fimetarium. The microorganism may in some embodiments be Acetobacterium tundrae. In some embodiments the microorganism may be Acetobacterium wieringae. In some embodiments the microorganism may be Acetobacterium psammolithicum.

According to some embodiments the microorganism according to the first aspect is of the genus Sporomusa. In some embodiments the microorganism may be Sporomusa ovata. In some embodiments the microorganism may be Sporomusa silvacetica. The microorganism may in some embodiments be Sporomusa sphaeroides. In some embodiments the microorganism may be Sporomusa termitida. The microorganism may in some embodiments be Sporomusa acidovorans. In some embodiments the microorganism may be Sporomusa aerivorans. In some embodiments the microorganism Sporomusa paucivorans acidovorans. According to some embodiments the microorganism is of the genus Blautia. In some embodiments the microorganism may be Blautia producta. In some embodiments the microorganism may be Blautia wexlerae. The microorganism may in some embodiments be Blautia hydrogenotrophica. In some embodiments the microorganism may be Blautia hansenii. According to some embodiments the microorganism is of the genus Carboxydocella. In some embodiments the microorganism may be Carboxydocella sporoproducens. In some embodiments the microorganism may be Carboxydocella ferrireducens. The microorganism may in some embodiments be Carboxydocella thermautotrophica. According to some embodiments the microorganism is of the genus Carboxydocella. In some embodiments the microorganism may be Carboxydocella sporoproducens. According to some embodiments the microorganism is of the genus Carboxydothermus. In some embodiments the microorganism may be Carboxydothermus hydrogenoformans. In some embodiments the microorganism may be Carboxydothermus ferrireducens. The microorganism may in some embodiments be Carboxydothermus pertinax. In some embodiments the microorganism may be Carboxydothermus islandicus. In some embodiments the microorganism may be Carboxydothermus siderophilus. According to some embodiments the microorganism is of the genus Desulfotomaculum. In some embodiments the microorganism may be Desulfotomaculum carboxydivorans. In some embodiments the microorganism may be Desulfotomaculum gibsoniae. The microorganism may in some embodiments be Desulfotomaculum kuznetsovii. In some embodiments the microorganism may be Desulfotomaculum thermobenzoicum subsp. thermosyntrophicum. In some embodiments the microorganism may be Desulfotomaculum acetoxidans. The microorganism may in some embodiments be Desulfotomaculum alcoholivorax. In some embodiments the microorganism may be Desulfotomaculum carboxydivorans. In some embodiments the microorganism may be Desulfotomaculum sapomandens. The microorganism may in some embodiments be Desulfotomaculum thermocisternum.

According to some embodiments the microorganism according to the first aspect is of the genus Desulfovibrio. In some embodiments the microorganism may be Desulfovibrio vulgaris. In some embodiments the microorganism may be Desulfovibrio alaskensis. The microorganism may in some embodiments be Desulfovibrio desulfuricans. In some embodiments the microorganism may be Desulfovibrio vulgaris. According to some embodiments the microorganism is of the genus Mycobacterium. In some embodiments the microorganism may be Mycobacterium smegmatis. In some embodiments the microorganism may be Mycobacterium gordonae. The microorganism may in some embodiments be Mycobacterium tuberculosis. According to some embodiments the microorganism is of the genus Ruminococcus. In some embodiments the microorganism may be Ruminococcus hydrogenotrophicus. The microorganism may in some embodiments be Ruminococcus schinkii. In some embodiments the microorganism may be Ruminococcus productus.

According to some embodiments the recombinant microorganism according to the first aspect is a species of Butyribacterium methylotrophicum, Alkalibaculum bacchii, Aecetoariaerobium riotera, Eubacterium limosum, Desulfitbacterium hafhierise, Peptostreptococcus productus, Rhodospirillum rubrum, Thermoanaerobacter kiuvi, Oxobacter pfennigii, Acetohalobium arabaticum, Carbophilus carboxidus, Cloacibacillus evryensis, Hydrogenophaga pseudoflava, Rhodopseudomonas palustris, Pseudomonas gazotropha, Ralstonia eutropha, Calderihabitans maritimus, Caloribacterium cisternae, Carboxydobrachium pacificum, Desulfurispora thermophila, Dictyoglomus thermophilum, Hydrogenophilus islandicus, Thermincola carboxydiphila, Thermincola ferriacetica, Thermincola potens, Thermoacetogenium phaeum, Thermoanaerobacter kivui, Thermoanaerobacter thermohydrosulfuricus subsp. carboxydovorans, Thermosinus carboxydivorans, Oligotropha carboxidovorans, Desulfosporosinus meridiei, Dehalococcoides mccartyi, Desulfatibacillum aliphaticivorans, Desulfobacterium autotrophicum, Desulfobacula toluolica, Desulfospira joergensenii, Desulfosporosinus orientis, Desulfosporosinus youngiae, Desulfovermiculus halophilus, Desulfurispora thermophila, Holophaga foetida, Methanobrevibacter arboriphilus, Orenia salinaria, Paenibacillus polymyxa, Tindallia californiensis, Anoxybacillus flavithermus, Desulfovirgula thermocuniculi, Thermosediminibacter oceani, Candidatus Scalindua brodae, Acetoanaerobium noterae, Ammonifex degensii, Acetitomaculum ruminis, Acetoanaerobium romashkovil, Acetonema longum, Bryanella formatexigens, Caloramator fervidus, Natroniella acetigena, Natronincola histinovorans, Syntrophococcus sucromutans, Treponema primitia, Pseudomonas carboxydohyrogena, Pseudomonas thermocarboxydovorans, Bradyrhizobium japonicum, Streptomyces thermoautotrophicus, Bacillus schlegelii, Caldanaerobacter subterraneus, Thermolithobacter carboxydivorans, Thermococcus onnurineus, Thermofilum carboxyditrophus, Archaeoglobus fulgidus, Desulfomonile tiedjei, Thermoproteus tenax or Rubrivivax gelatinosa.

According to a second aspect, there is provided a recombinant nucleic acid molecule. The recombinant nucleic acid molecule contains a nucleic acid sequence encoding one or more enzymes that allow the conversion of acetyl-CoA to one or more alkanes and/or alkenes with a main chain of 2 to 6 carbon atoms. In embodiments where the recombinant nucleic acid molecule includes more than one coding sequences or a combination of more than one coding sequences that encode one or more enzymes that catalyse the conversion of acetyl-CoA to one or more alkanes and/or alkenes with a main chain of 2 to 6 carbon atoms, the recombinant nucleic acid molecule may not include at least one of the respective coding sequences. In some embodiments the recombinant nucleic acid molecule does not include at least one coding sequence or a combination of coding sequences that encode one or more enzymes that catalyse the conversion of acetyl-CoA to one or more alkanes and/or alkenes with a main chain of 2 to 6 carbon atoms.

In some embodiments the recombinant nucleic acid molecule contains a nucleic acid sequence encoding one or more enzymes that allow the conversion of acetyl-CoA to one or more alkenes with a main chain of 2 to 5 carbon atoms. The recombinant nucleic acid molecule may include one or more coding sequences encoding one or more enzymes that catalyse the conversion of acetyl-CoA to crotonyl-CoA, and that further catalyse the conversion of crotonyl-CoA to an alkene. The recombinant nucleic acid molecule may also include one or more coding sequences encoding one or more enzymes that catalyse the conversion of acetyl-CoA to 3-methylcrotonyl-CoA, and that further catalyse the conversion of 3-methylcrotonyl-CoA to an alkene. The recombinant nucleic acid molecule may also include one or more coding sequences encoding one or more enzymes that catalyse the conversion of acetyl-CoA to propionyl-CoA, and that further catalyse the conversion of propionyl-CoA to an alkene. Each coding sequence included in the recombinant nucleic acid molecule is operationally linked to a transcriptional promoter.

In some embodiments the recombinant nucleic acid molecule contains a coding sequence that encodes a nitrogenase enzyme EC 1.18.6.1 or EC 1.19.6.1.

In some embodiments of the recombinant nucleic acid molecule according to the second aspect the one or more enzymes that catalyse the conversion of acetyl-CoA to crotonyl-CoA include acetyl-CoA carboxylase (EC 6.4.1.2); acetoacetyl-CoA synthase (EC 2.3.1.194); acetoacetyl-CoA reductase (EC 1.1.1.36); and enoyl-CoA hydratase 2 (EC 4.2.1.119).

In some embodiments of the recombinant nucleic acid molecule according to the second aspect the one or more enzymes that catalyse the conversion of acetyl-CoA to crotonyl-CoA include acetyl-CoA C-acetyltransferase (EC 2.3.1.9); 3-hydroxybutyryl-CoA dehydrogenase (EC 1.1.1.157); and 3-hydroxybutyryl-CoA dehydratase. In some embodiments the one or more enzymes that catalyse the conversion of acetyl-CoA to crotonyl-CoA include 4-aminobutyrate aminotransferase (EC 2.6.1.19); glutamate dehydrogenase (EC 1.4.1.2); 2-hydroxyglutarate dehydrogenase (EC 1.1.99.2); glutaconate CoA-transferase (EC 2.8.3.12); 2-hydroxyglutaryl-CoA dehydratase (EC 4.2.1.-); and glutaconyl-CoA decarboxylase (EC 4.1.1.70). In some embodiments the one or more enzymes that catalyse the conversion of acetyl-CoA to crotonyl-CoA include 4-aminobutyrate aminotransferase (EC 2.6.1.19); 4-hydroxybutyrate dehydrogenase (EC 1.1.1.61); 4-hydroxybutyrate CoA-transferase (EC 2.8.3.-); vinylacetyl-CoA-Delta-isomerase (EC 4.2.1.120); and 4-hydroxy-butyryl-CoA dehydratase (EC 5.3.3.3).

In some embodiments of the recombinant nucleic acid molecule according to the second aspect the one or more enzymes that catalyse the conversion of acetyl-CoA to 3-methylcrotonyl-CoA include acetyl-CoA C-acetyltransferase (EC 2.3.1.9); 3-hydroxy-3-methylglutaryl-CoA synthase (EC 2.3.3.10); 3-methylglutaconyl-CoA hydratase (EC 4.2.1.18); and 3-methylcrotonyl-CoA carboxylase (EC 6.4.1.4). In some embodiments the one or more enzymes that catalyse the conversion of acetyl-CoA to 3-methylcrotonyl-CoA include acetyl-CoA carboxylase (EC 6.4.1.2); acetoacetyl-CoA synthase (EC 2.3.1.194); 3-hydroxy-3-methylglutaryl-CoA synthase (EC 2.3.3.10); 3-methyl-glutaconyl-CoA hydratase (EC 4.2.1.18); and 3-methylcrotonyl-CoA carboxylase (EC 6.4.1.4).

In some embodiments of the recombinant nucleic acid molecule according to the second aspect the one or more enzymes that catalyse the conversion of acetyl-CoA to propionyl-CoA include acetyl-CoA carboxylase (EC 6.4.1.2); malonyl CoA reductase (EC 1.2.1.75); malonyl CoA reductase (EC 1.1.1.298); propionyl-CoA synthase (EC 6.2.1.36); propionyl-CoA synthase/acryloyl-CoA synthase (EC 4.2.1.116); and propionyl-CoA synthase (EC 1.3.1.84).

In some embodiments of the recombinant nucleic acid molecule according to the second aspect the one or more enzymes that catalyse the conversion of acetyl-CoA to propionyl-CoA include acetyl-CoA carboxylase (EC 6.4.1.2); methylmalonyl-CoA mutase (EC 5.4.99.2); and methylmalonyl-CoA decarboxylase (EC 4.1.1.41). In some embodiments the one or more enzymes that catalyse the conversion of acetyl-CoA to propionyl-CoA include 2-oxoglutarate/butyrate ferredoxin oxidoreductase (EC 1.2.7.3) and/or pyruvate/2-oxobutyrate ferredoxin oxidoreductase (EC 1.2.7.1). In some embodiments the one or more enzymes that catalyse the conversion of acetyl-CoA to propionyl-CoA include lactate CoA-transferase (EC 2.8.3.-); lactoyl-CoA dehydratase (EC 4.2.1.54); and propionyl-CoA synthase (EC 1.3.1.84).

In some embodiments of the recombinant nucleic acid molecule according to the second aspect the one or more enzymes that catalyse the conversion of acetyl-CoA to acryloyl-CoA include acetyl-CoA carboxylase (EC 6.4.1.2); malonyl CoA reductase (EC 1.2.1.75); malonyl CoA reductase (EC 1.1.1.298); propionyl-CoA synthase (EC 6.2.1.36); and propionyl-CoA synthase/acryloyl-CoA synthase (EC 4.2.1.116). In some embodiments the one or more enzymes that catalyse the conversion of acetyl-CoA to acryloyl-CoA include lactate CoA-transferase (EC 2.8.3.-); and lactoyl-CoA dehydratase (EC 4.2.1.54). In some embodiments the one or more enzymes that catalyse the conversion of acetyl-CoA to acryloyl-CoA include acyl-CoA thioesterase (EC 3.1.2.-) and (b) phenyl acrylic acid decarboxylase (EC 4.1.1.-).

In some embodiments of the recombinant nucleic acid molecule according to the second aspect the one or more enzymes that catalyse the conversion of acetyl-CoA to acryloyl-CoA include phosphate butyryltransferase (EC 2.3.1.19); butyrate kinase (EC 2.7.2.7), and phenyl acrylic acid decarboxylase (EC 4.1.1.-). In some embodiments the one or more enzymes that catalyse the conversion of acetyl-CoA to acryloyl-CoA include trans-2-enoyl-CoA reductase (EC1.1.1.36); acetyl-CoA C-acetyltransferase (EC 2.3.1.9); 3-hydroxybutyryl-CoA dehydrogenase (EC 1.1.1.157); 3-hydroxybutyryl-CoA dehydratase (EC 4.2.1.55); acyl-CoA thioesterase (EC 3.1.2.-) and/or a combination of phosphate butyryltransferase (EC 2.3.1.19) and butyrate kinase (EC 2.7.2.7); and a phenyl acrylic acid decarboxylase enzyme (EC 4.1.1.-).

In some embodiments of the recombinant nucleic acid molecule according to the second aspect the one or more enzymes that catalyse the conversion of acetyl-CoA to acryloyl-CoA include aldehyde dehydrogenase (EC 1.2.1.-) or alcohol dehydrogenase (EC 1.1.1.-); aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127). In some embodiments the one or more enzymes that catalyse the conversion of acetyl-CoA to acryloyl-CoA include aldehyde dehydrogenase (EC 1.2.1.-) or alcohol dehydrogenase (EC 1.1.1.-); aldehyde dehydrogenase (EC 1.2.1.-) and/or alcohol dehydrogenase (EC 1.1.1.-); and a combination of (i) one or more of farnesol kinase (EC 2.7.1.-), geranylgeraniol kinase (EC 2.7.1.B19), hydroxyethylthiazole kinase (EC 2.7.1.50) or undecaprenol kinase (EC 2.7.1.66), and (ii) one or more of 2-methyl-1,3-butadiene synthase (EC 4.2.3.27) or methylbutenol synthase.

In some embodiments of the recombinant nucleic acid molecule according to the second aspect the one or more enzymes that catalyse the conversion of acetyl-CoA to acryloyl-CoA include a combination of acyl-CoA thioesterase (EC 3.1.2.-) and aldehyde dehydrogenase (EC 1.2.1.-) or alcohol dehydrogenase (EC 1.1.1.-); aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127). In some embodiments the one or more enzymes that catalyse the conversion of acetyl-CoA to acryloyl-CoA include a combination of acyl-CoA thioesterase (EC 3.1.2.-) and aldehyde dehydrogenase (EC 1.2.1.-) or alcohol dehydrogenase (EC 1.1.1.-); aldehyde dehydrogenase (EC 1.2.1.-) and/or alcohol dehydrogenase (EC 1.1.1.-); and a combination of (i) one or more of farnesol kinase (EC 2.7.1.-), geranylgeraniol kinase (EC 2.7.1.B19), hydroxyethylthiazole kinase (EC 2.7.1.50) or undecaprenol kinase (EC 2.7.1.66), and (ii) one or more of 2-methyl-1,3-butadiene synthase (EC 4.2.3.27) or methylbutenol synthase.

In some embodiments of the recombinant nucleic acid molecule according to the second aspect the one or more enzymes that catalyse the conversion of acetyl-CoA to acryloyl-CoA include a combination of aldehyde ferredoxin oxidoreductase (EC 1.2.7.5) and acyl-CoA thioesterase (EC 3.1.2.-); aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127). In some embodiments the one or more enzymes that catalyse the conversion of acetyl-CoA to acryloyl-CoA include a combination of aldehyde ferredoxin oxidoreductase (EC 1.2.7.5) and acyl-CoA thioesterase (EC 3.1.2.-); aldehyde dehydrogenase (EC 1.2.1.-) and/or alcohol dehydrogenase (EC 1.1.1.-); and a combination of (i) one or more of farnesol kinase (EC 2.7.1.-), geranylgeraniol kinase (EC 2.7.1.B19), hydroxyethylthiazole kinase (EC 2.7.1.50) or undecaprenol kinase (EC 2.7.1.66), and (ii) one or more of 2-methyl-1,3-butadiene synthase (EC 4.2.3.27) or methylbutenol synthase.

In some embodiments of the recombinant nucleic acid molecule according to the second aspect the one or more enzymes that catalyse the conversion of acetyl-CoA to acryloyl-CoA include a combination of aldehyde ferredoxin oxidoreductase (EC 1.2.7.5) and acyl-CoA thioesterase (EC 3.1.2.-); aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127). In some embodiments the one or more enzymes that catalyse the conversion of acetyl-CoA to acryloyl-CoA include a combination of aldehyde ferredoxin oxidoreductase (EC 1.2.7.5) and acyl-CoA thioesterase (EC 3.1.2.-); aldehyde dehydrogenase (EC 1.2.1.-) and/or alcohol dehydrogenase (EC 1.1.1.-); and a combination of (i) one or more of farnesol kinase (EC 2.7.1.-), geranylgeraniol kinase (EC 2.7.1.B19), hydroxyethylthiazole kinase (EC 2.7.1.50) or undecaprenol kinase (EC 2.7.1.66), and (ii) one or more of 2-methyl-1,3-butadiene synthase (EC 4.2.3.27) or methylbutenol synthase.

In some embodiments of the recombinant nucleic acid molecule according to the second aspect the one or more enzymes that catalyse the conversion of acetyl-CoA to acryloyl-CoA include a combination of phosphate butyryltransferase (EC 2.3.1.19), butyrate kinase (EC 2.7.2.7) and aldehyde dehydrogenase (EC 1.2.1.-) or alcohol dehydrogenase (EC 1.1.1.-); aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127). In some embodiments the one or more enzymes that catalyse the conversion of acetyl-CoA to acryloyl-CoA include a combination of phosphate butyryltransferase (EC 2.3.1.19), butyrate kinase (EC 2.7.2.7) and aldehyde dehydrogenase (EC 1.2.1.-) or alcohol dehydrogenase (EC 1.1.1.-); aldehyde dehydrogenase (EC 1.2.1.-) and/or alcohol dehydrogenase (EC 1.1.1.-); and a combination of (i) one or more of farnesol kinase (EC 2.7.1.-), geranylgeraniol kinase (EC 2.7.1.B19), hydroxyethylthiazole kinase (EC 2.7.1.50) or undecaprenol kinase (EC 2.7.1.66), and (ii) one or more of 2-methyl-1,3-butadiene synthase (EC 4.2.3.27) or methylbutenol synthase.

In some embodiments of the recombinant nucleic acid molecule according to the second aspect the one or more enzymes that catalyse the conversion of acetyl-CoA to acryloyl-CoA include a combination of phosphate butyryltransferase (EC 2.3.1.19), butyrate kinase (EC 2.7.2.7) and aldehyde ferredoxin oxidoreductase (EC 1.2.7.5); (m) aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127). In some embodiments the one or more enzymes that catalyse the conversion of acetyl-CoA to acryloyl-CoA include a combination of phosphate butyryltransferase (EC 2.3.1.19), butyrate kinase (EC 2.7.2.7) and aldehyde ferredoxin oxidoreductase (EC 1.2.7.5); (m) aldehyde dehydrogenase (EC 1.2.1.-) and/or alcohol dehydrogenase (EC 1.1.1.-); and a combination of (i) one or more of farnesol kinase (EC 2.7.1.-), geranylgeraniol kinase (EC 2.7.1.B19), hydroxyethylthiazole kinase (EC 2.7.1.50) or undecaprenol kinase (EC 2.7.1.66), and (ii) one or more of 2-methyl-1,3-butadiene synthase (EC 4.2.3.27) or methylbutenol synthase.

In some embodiments of the recombinant nucleic acid molecule according to the second aspect the one or more enzymes that catalyse the conversion of 3-methylcrotonyl-CoA to an alkene include the enzyme phenyl acrylic acid decarboxylase (EC 4.1.1.-); and the enzyme acyl-CoA thioesterase (EC 3.1.2.-) and/or a combination of phosphate butyryltransferase (EC 2.3.1.19) and butyrate kinase (EC 2.7.2.7).

In some embodiments of the recombinant nucleic acid molecule according to the second aspect the one or more enzymes that catalyse the conversion of 3-methylcrotonyl-CoA to an alkene include a combination of acyl-CoA thioesterase (EC 3.1.2.-) and aldehyde dehydrogenase (EC 1.2.1.-) or alcohol dehydrogenase (EC 1.1.1.-); aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127). In some embodiments the one or more enzymes that catalyse the conversion of 3-methylcrotonyl-CoA to an alkene include a combination of acyl-CoA thioesterase (EC 3.1.2.-) and aldehyde dehydrogenase (EC 1.2.1.-) or alcohol dehydrogenase (EC 1.1.1.-); aldehyde dehydrogenase (EC 1.2.1.-) and/or alcohol dehydrogenase (EC 1.1.1.-); and a combination of (i) one or more of farnesol kinase (EC 2.7.1.-), geranylgeraniol kinase (EC 2.7.1.B19), hydroxyethylthiazole kinase (EC 2.7.1.50) or undecaprenol kinase (EC 2.7.1.66), and (ii) one or more of 2-methyl-1,3-butadiene synthase (EC 4.2.3.27) or methylbutenol synthase.

In some embodiments of the recombinant nucleic acid molecule according to the second aspect the one or more enzymes that catalyse the conversion of 3-methylcrotonyl-CoA to an alkene include aldehyde dehydrogenase (EC 1.2.1.-) or alcohol dehydrogenase (EC 1.1.1.-); aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127). In some embodiments the one or more enzymes that catalyse the conversion of 3-methylcrotonyl-CoA to an alkene include aldehyde dehydrogenase (EC 1.2.1.-) or alcohol dehydrogenase (EC 1.1.1.-); and a combination of (i) one or more of farnesol kinase (EC 2.7.1.-), geranylgeraniol kinase (EC 2.7.1.B19), hydroxyethylthiazole kinase (EC 2.7.1.50) or undecaprenol kinase (EC 2.7.1.66), and (ii) one or more of 2-methyl-1,3-butadiene synthase (EC 4.2.3.27) or methylbutenol synthase.

In some embodiments of the recombinant nucleic acid molecule according to the second aspect the one or more enzymes that catalyse the conversion of 3-methylcrotonyl-CoA to an alkene include a combination of aldehyde ferredoxin oxidoreductase (EC 1.2.7.5) and acyl-CoA thioesterase (EC 3.1.2.-); aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127). In some embodiments of the recombinant nucleic acid molecule according to the second aspect the one or more enzymes that catalyse the conversion of 3-methylcrotonyl-CoA to an alkene include a combination of aldehyde ferredoxin oxidoreductase (EC 1.2.7.5) and acyl-CoA thioesterase (EC 3.1.2.-); aldehyde dehydrogenase (EC 1.2.1.-) and/or alcohol dehydrogenase (EC 1.1.1.-); and a combination of (i) one or more of farnesol kinase (EC 2.7.1.-), geranylgeraniol kinase (EC 2.7.1.B19), hydroxyethylthiazole kinase (EC 2.7.1.50) or undecaprenol kinase (EC 2.7.1.66), and (ii) one or more of 2-methyl-1,3-butadiene synthase (EC 4.2.3.27) or methylbutenol synthase.

In some embodiments of the recombinant nucleic acid molecule according to the second aspect the one or more enzymes that catalyse the conversion of 3-methylcrotonyl-CoA to an alkene include a combination of phosphate butyryltransferase (EC 2.3.1.19), butyrate kinase (EC 2.7.2.7) and aldehyde dehydrogenase (EC 1.2.1.-) or alcohol dehydrogenase (EC 1.1.1.-); aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127). In some embodiments the one or more enzymes that catalyse the conversion of 3-methylcrotonyl-CoA to an alkene include a combination of phosphate butyryltransferase (EC 2.3.1.19), butyrate kinase (EC 2.7.2.7) and aldehyde dehydrogenase (EC 1.2.1.-) or alcohol dehydrogenase (EC 1.1.1.-); aldehyde dehydrogenase (EC 1.2.1.-) and/or alcohol dehydrogenase (EC 1.1.1.-); and a combination of (i) one or more of farnesol kinase (EC 2.7.1.-), geranylgeraniol kinase (EC 2.7.1.B19), hydroxyethylthiazole kinase (EC 2.7.1.50) or undecaprenol kinase (EC 2.7.1.66), and (ii) one or more of 2-methyl-1,3-butadiene synthase (EC 4.2.3.27) or methylbutenol synthase.

In some embodiments of the recombinant nucleic acid molecule according to the second aspect the one or more enzymes that catalyse the conversion of 3-methylcrotonyl-CoA to an alkene include a combination of phosphate butyryltransferase (EC 2.3.1.19), butyrate kinase (EC 2.7.2.7) and aldehyde ferredoxin oxidoreductase (EC 1.2.7.5); aldehyde dehydrogenase (EC 1.2.1.-) and/or alcohol dehydrogenase (EC 1.1.1.-); and geraniol isomerase (EC 5.4.4.4), which is also linalool dehydratase (EC 4.2.1.127). In some embodiments the one or more enzymes that catalyse the conversion of 3-methylcrotonyl-CoA to an alkene include a combination of phosphate butyryltransferase (EC 2.3.1.19), butyrate kinase (EC 2.7.2.7) and aldehyde ferredoxin oxidoreductase (EC 1.2.7.5); aldehyde dehydrogenase (EC 1.2.1.-) and/or alcohol dehydrogenase (EC 1.1.1.-); and a combination of (i) one or more of farnesol kinase (EC 2.7.1.-), geranylgeraniol kinase (EC 2.7.1.B19), hydroxyethylthiazole kinase (EC 2.7.1.50) or undecaprenol kinase (EC 2.7.1.66), and (ii) one or more of 2-methyl-1,3-butadiene synthase (EC 4.2.3.27) or methylbutenol synthase.

In some embodiments of the recombinant nucleic acid molecule according to the second aspect the one or more enzymes that catalyse the conversion of propionyl-CoA to an alkene include acetyl-CoA C-acetyltransferase (EC 2.3.1.9); a combination of acetoacetyl-CoA reductase (EC 1.1.1.36) and enoyl-CoA hydratase 2 (EC 4.2.1.119); acyl-CoA thioesterase (EC 3.1.2.-) and/or a combination of phosphate butyryltransferase (EC 2.3.1.19) and butyrate kinase (EC 2.7.2.7); and phenyl acrylic acid decarboxylase (EC 4.1.1.-). In some embodiments the one or more enzymes that catalyse the conversion of propionyl-CoA to an alkene include acetyl-CoA C-acetyltransferase (EC 2.3.1.9); a combination of 3-hydroxybutyryl-CoA dehydrogenase (EC 1.1.1.157) and 3-hydroxy-butyryl-CoA dehydratase (EC 4.2.1.55); acyl-CoA thioesterase (EC 3.1.2.-) and/or a combination of phosphate butyryltransferase (EC 2.3.1.19) and butyrate kinase (EC 2.7.2.7); and phenyl acrylic acid decarboxylase (EC 4.1.1.-).

In some embodiments of the recombinant nucleic acid molecule according to the second aspect the one or more enzymes that catalyse the conversion of acryloyl-CoA to an alkene include acyl-CoA thioesterase (EC 3.1.2.-); and phenyl acrylic acid decarboxylase (EC 4.1.1.-). In some embodiments the one or more enzymes that catalyse the conversion of acryloyl-CoA to an alkene include phosphate butyryltransferase (EC 2.3.1.19); butyrate kinase (EC 2.7.2.7); and phenyl acrylic acid decarboxylase (EC 4.1.1.-).

In some embodiments the recombinant nucleic acid molecule according to the second aspect includes coding sequences encoding the enzyme acetyl-CoA C-acetyltransferase (EC 2.3.1.9); the enzyme 3-hydroxybutyryl-CoA dehydrogenase (EC 1.1.1.157); the enzyme 3-hydroxybutyryl-CoA dehydratase (EC 4.2.1.55); an acyl-CoA thioesterase enzyme (EC 3.1.2.-); an aldehyde dehydrogenase (EC 1.2.1.-) or an alcohol dehydrogenase enzyme (EC 1.1.1.-); and the enzyme geraniol isomerase (EC 5.4.4.4), which is also linalool dehydratase (EC 4.2.1.127). In some embodiments the recombinant nucleic acid molecule includes coding sequences encoding the enzyme Acetyl-CoA carboxylase (EC 6.4.1.2); the enzyme acetoacetyl-CoA synthase (EC 2.3.1.194); the enzyme acetoacetyl-CoA reductase (EC 1.1.1.36); the enzyme enoyl-CoA hydratase 2 (EC 4.2.1.119); an acyl-CoA thioesterase enzyme (EC 3.1.2.-); an aldehyde dehydrogenase (EC 1.2.1.-) or an alcohol dehydrogenase enzyme (EC 1.1.1.-); and the enzyme geraniol isomerase (EC 5.4.4.4), which is also linalool dehydratase (EC 4.2.1.127).

In some embodiments the recombinant nucleic acid molecule according to the second aspect includes coding sequences encoding the enzyme 4-aminobutyrate aminotransferase (EC 2.6.1.19); the enzyme glutamate dehydrogenase (EC 1.4.1.2); the enzyme 2-hydroxyglutarate dehydrogenase (EC 1.1.99.2); the enzyme glutaconate CoA-transferase (EC 2.8.3.12); a 2-hydroxyglutaryl-CoA dehydratase enzyme (EC 4.2.1.-); the enzyme glutaconyl-CoA decarboxylase (EC 4.1.1.70); an acyl-CoA thioesterase enzyme (EC 3.1.2.-); an aldehyde dehydrogenase (EC 1.2.1.-) or an alcohol dehydrogenase enzyme (EC 1.1.1.-); and the enzyme geraniol isomerase (EC 5.4.4.4), which is also linalool dehydratase (EC 4.2.1.127).

In some embodiments the recombinant nucleic acid molecule according to the second aspect includes coding sequences encoding the enzyme 4-aminobutyrate aminotransferase (EC 2.6.1.19); the enzyme 4-hydroxybutyrate dehydrogenase (EC 1.1.1.61); a 4-hydroxybutyrate CoA-transferase enzyme (EC 2.8.3.-); the enzyme vinylacetyl-CoA-Delta-isomerase (EC 4.2.1.120); the enzyme 4-hydroxybutyryl-CoA dehydratase (EC 5.3.3.3); an acyl-CoA thioesterase enzyme (EC 3.1.2.-); an aldehyde dehydrogenase (EC 1.2.1.-) or an alcohol dehydrogenase enzyme (EC 1.1.1.-); and the enzyme geraniol isomerase (EC 5.4.4.4), which is also linalool dehydratase (EC 4.2.1.127).

In some embodiments the recombinant nucleic acid molecule according to the second aspect includes coding sequences encoding the enzyme acetyl-CoA C-acetyltransferase (EC 2.3.1.9) and/or the enzymes acetyl-CoA carboxylase (EC 6.4.1.2) and acetoacetyl-CoA synthase (EC 2.3.1.194), the enzyme 3-hydroxy-3-methylglutaryl-CoA synthase (EC 2.3.3.10), the enzyme 3-methylglutaconyl-CoA hydratase (EC 4.2.1.18), and the enzyme 3-methylcrotonyl-CoA carboxylase (EC 6.4.1.4); an aldehyde dehydrogenase (EC 1.2.1.-) or an alcohol dehydrogenase enzyme (EC 1.1.1.-); and the enzyme geraniol isomerase (EC 5.4.4.4), which is also linalool dehydratase (EC 4.2.1.127), and/or a combination of (i) one or more of the enzymes farnesol kinase (EC 2.7.1.-), geranylgeraniol kinase (EC 2.7.1.B19), hydroxyethylthiazole kinase (EC 2.7.1.50) or undecaprenol kinase (EC 2.7.1.66), and (ii) one or more of the enzymes 2-methyl-1,3-butadiene synthase (EC 4.2.3.27) or methylbutenol synthase.

In some embodiments the recombinant nucleic acid molecule according to the second aspect includes coding sequences encoding a combination of the enzymes acetyl-CoA C-acetyltransferase (EC 2.3.1.9), 3-hydroxybutyryl-CoA dehydrogenase (EC 1.1.1.157), and 3-hydroxybutyryl-CoA dehydratase (EC 4.2.1.55); and an aldehyde dehydrogenase (EC 1.2.1.-) or an alcohol dehydrogenase enzyme (EC 1.1.1.-); and the enzyme geraniol isomerase (EC 5.4.4.4), which is also linalool dehydratase (EC 4.2.1.127), and/or a combination of (i) one or more of the enzymes farnesol kinase (EC 2.7.1.-), geranylgeraniol kinase (EC 2.7.1.B19), hydroxyethylthiazole kinase (EC 2.7.1.50) or undecaprenol kinase (EC 2.7.1.66), and (ii) one or more of the enzymes 2-methyl-1,3-butadiene synthase (EC 4.2.3.27) or methylbutenol synthase.

In some embodiments the recombinant nucleic acid molecule according to the second aspect includes coding sequences encoding a combination of the enzymes Acetyl-CoA carboxylase (EC 6.4.1.2), acetoacetyl-CoA synthase (EC 2.3.1.194), acetoacetyl-CoA reductase (EC 1.1.1.36) and enoyl-CoA hydratase 2 (EC 4.2.1.119); and an aldehyde dehydrogenase (EC 1.2.1.-) or an alcohol dehydrogenase enzyme (EC 1.1.1.-); and the enzyme geraniol isomerase (EC 5.4.4.4), which is also linalool dehydratase (EC 4.2.1.127), and/or a combination of (i) one or more of the enzymes farnesol kinase (EC 2.7.1.-), geranylgeraniol kinase (EC 2.7.1.B19), hydroxyethylthiazole kinase (EC 2.7.1.50) or undecaprenol kinase (EC 2.7.1.66), and (ii) one or more of the enzymes 2-methyl-1,3-butadiene synthase (EC 4.2.3.27) or methylbutenol synthase.

In some embodiments the recombinant nucleic acid molecule according to the second aspect includes coding sequences encoding (i) a combination of the enzymes acetyl-CoA C-acetyltransferase (EC 2.3.1.9), 3-hydroxybutyryl-CoA dehydrogenase (EC 1.1.1.157), and 3-hydroxybutyryl-CoA dehydratase (EC 4.2.1.55), and/or a combination of the enzymes the enzymes Acetyl-CoA carboxylase (EC 6.4.1.2), acetoacetyl-CoA synthase (EC 2.3.1.194), acetoacetyl-CoA reductase (EC 1.1.1.36) and enoyl-CoA hydratase 2 (EC 4.2.1.119); (ii) a combination of the enzymes phosphate butyryltransferase (EC 2.3.1.19) and butyrate kinase (EC 2.7.2.7), and/or an acyl-CoA thioesterase enzyme (EC 3.1.2.-); and (iii) a phenyl acrylic acid decarboxylase enzyme (EC 4.1.1.-).

In some embodiments the recombinant nucleic acid molecule according to the second aspect includes coding sequences encoding (i) a combination of the enzyme acetyl-CoA C-acetyltransferase (EC 2.3.1.9) and/or the enzymes Acetyl-CoA carboxylase (EC 6.4.1.2) and acetoacetyl-CoA synthase (EC 2.3.1.194); the enzyme 3-hydroxy-3-methylglutaryl-CoA synthase (EC 2.3.3.10); the enzyme 3-methylglutaconyl-CoA hydratase (EC 4.2.1.18); and the enzyme 3-methylglutaconyl-CoA decarboxylase; (ii) a combination of the enzymes phosphate butyryltransferase (EC 2.3.1.19) and butyrate kinase (EC 2.7.2.7), and/or an acyl-CoA thioesterase enzyme (EC 3.1.2.-); and (iii) a phenyl acrylic acid decarboxylase enzyme (EC 4.1.1.-).

In some embodiments the recombinant nucleic acid molecule according to the second aspect includes coding sequences encoding the enzyme lactate CoA-transferase (EC 2.8.3.-); the enzyme lactoyl-CoA dehydratase (EC 4.2.1.54); and the enzyme acyl-CoA thioesterase (EC 3.1.2.-). In some embodiments the recombinant nucleic acid molecule includes coding sequences encoding the enzyme lactate CoA-transferase (EC 2.8.3.-), the enzyme lactoyl-CoA dehydratase (EC 4.2.1.54); the enzyme phosphate butyryltransferase (EC 2.3.1.19); the enzyme butyrate kinase (EC 2.7.2.7); and the enzyme phenyl acrylic acid decarboxylase (EC 4.1.1.-).

In some embodiments the recombinant nucleic acid molecule according to the second aspect includes coding sequences encoding the enzyme 4-aminobutyrate aminotransferase (EC 2.6.1.19); the enzyme 4-hydroxybutyrate dehydrogenase (EC 1.1.1.61); a 4-hydroxybutyrate CoA-transferase enzyme (EC 2.8.3.-); the enzyme vinylacetyl-CoA-delta-isomerase (EC 4.2.1.120); the enzyme 4-hydroxybutyryl-CoA dehydratase (EC 5.3.3.3); a combination of the enzymes phosphate butyryltransferase (EC 2.3.1.19) and butyrate kinase (EC 2.7.2.7), and/or an acyl-CoA thioesterase enzyme (EC 3.1.2.-); and the enzyme phenyl acrylic acid decarboxylase (EC 4.1.1.-). In some embodiments the recombinant nucleic acid molecule includes coding sequences encoding the enzyme 4-aminobutyrate aminotransferase (EC 2.6.1.19); the enzyme glutamate dehydrogenase (EC 1.4.1.2); the enzyme 2-hydroxyglutarate dehydrogenase (EC 1.1.99.2); the enzyme glutaconate CoA-transferase (EC 2.8.3.12); the enzyme 2-hydroxyglutaryl-CoA dehydratase (EC 4.2.1.-); the enzyme glutaconyl-CoA decarboxylase (EC 4.1.1.70); a combination of the enzymes phosphate butyryltransferase (EC 2.3.1.19) and butyrate kinase (EC 2.7.2.7), and/or an acyl-CoA thioesterase enzyme (EC 3.1.2.-); and the enzyme phenyl acrylic acid decarboxylase (EC 4.1.1.-).

In some embodiments the recombinant nucleic acid molecule according to the second aspect includes coding sequences encoding the enzyme 3-hydroxybutyryl-CoA dehydrogenase (EC 1.1.1.157); 3-hydroxybutyryl-CoA dehydratase (EC 4.2.1.55); the enzyme trans-2-enoyl-CoA reductase (EC1.1.1.36); the enzyme acetyl-CoA C-acetyltransferase (EC 2.3.1.9); the enzyme 3-hydroxybutyryl-CoA dehydrogenase (EC 1.1.1.157); the enzyme 3-hydroxybutyryl-CoA dehydratase (EC 4.2.1.55); a combination of the enzymes phosphate butyryltransferase (EC 2.3.1.19) and butyrate kinase (EC 2.7.2.7), and/or an acyl-CoA thioesterase enzyme (EC 3.1.2.-); and a phenyl acrylic acid decarboxylase enzyme (EC 4.1.1.-).

In some embodiments the recombinant nucleic acid molecule according to the second aspect includes coding sequences encoding the enzyme Acetyl-CoA carboxylase (EC 6.4.1.2); acetoacetyl-CoA synthase (EC 2.3.1.194); acetoacetyl-CoA reductase (EC 1.1.1.36); enoyl-CoA hydratase 2 (EC 4.2.1.119); the enzyme trans-2-enoyl-CoA reductase (EC1.1.1.36); the enzyme acetyl-CoA C-acetyltransferase (EC 2.3.1.9); the enzyme 3-hydroxybutyryl-CoA dehydrogenase (EC 1.1.1.157); the enzyme 3-hydroxybutyryl-CoA dehydratase (EC 4.2.1.55); a combination of the enzymes phosphate butyryltransferase (EC 2.3.1.19) and butyrate kinase (EC 2.7.2.7), and/or an acyl-CoA thioesterase enzyme (EC 3.1.2.-); and a phenyl acrylic acid decarboxylase enzyme (EC 4.1.1.-).

In some embodiments the recombinant nucleic acid molecule according to the second aspect includes coding sequences encoding the enzyme acetyl-CoA carboxylase (EC 6.4.1.2); the enzyme methylmalonyl-CoA mutase (EC 5.4.99.2); the enzyme methylmalonyl-CoA decarboxylase (EC 4.1.1.41); the enzyme acetyl-CoA C-acetyltransferase (EC 2.3.1.9); a combination of (i) the enzymes acetoacetyl-CoA reductase (EC 1.1.1.36) and enoyl-CoA hydratase 2 (EC 4.2.1.119) and/or (ii) the enzymes 3-hydroxybutyryl-CoA dehydrogenase (EC 1.1.1.157) and 3-hydroxybutyryl-CoA dehydratase (EC 4.2.1.55); (vi) an acyl-CoA thioesterase enzyme (EC 3.1.2.-); the enzyme acetyl-CoA C-acetyltransferase (EC 2.3.1.9); the enzyme acetoacetyl-CoA reductase (EC 1.1.1.36); the enzyme enoyl-CoA hydratase 2 (EC 4.2.1.119); a combination of the enzymes phosphate butyryltransferase (EC 2.3.1.19) and butyrate kinase (EC 2.7.2.7), and/or an acyl-CoA thioesterase enzyme (EC 3.1.2.-); and a phenyl acrylic acid decarboxylase enzyme (EC 4.1.1.-).

In some embodiments the recombinant nucleic acid molecule according to the second aspect includes coding sequences encoding the enzyme acetyl-CoA carboxylase (EC 6.4.1.2); the enzyme methylmalonyl-CoA mutase (EC 5.4.99.2); the enzyme methylmalonyl-CoA decarboxylase (EC 4.1.1.41); the enzyme acetyl-CoA C-acetyltransferase (EC 2.3.1.9); a combination of (i) the enzymes acetoacetyl-CoA reductase (EC 1.1.1.36) and enoyl-CoA hydratase 2 (EC 4.2.1.119) and/or (ii) the enzymes 3-hydroxybutyryl-CoA dehydrogenase (EC 1.1.1.157) and 3-hydroxybutyryl-CoA dehydratase (EC 4.2.1.55); an acyl-CoA thioesterase enzyme (EC 3.1.2.-); the enzyme acetyl-CoA C-acetyltransferase (EC 2.3.1.9); the enzyme 3-hydroxybutyryl-CoA dehydrogenase (EC 1.1.1.157); the enzyme 3-hydroxybutyryl-CoA dehydratase (EC 4.2.1.55); a combination of the enzymes phosphate butyryltransferase (EC 2.3.1.19) and butyrate kinase (EC 2.7.2.7), and/or an acyl-CoA thioesterase enzyme (EC 3.1.2.-); and a phenyl acrylic acid decarboxylase enzyme (EC 4.1.1.-).

In some embodiments the recombinant nucleic acid molecule according to the second aspect includes coding sequences encoding the enzyme 2-oxoglutarate/butyrate ferredoxin oxidoreductase (EC 1.2.7.3); the enzyme pyruvate/2-oxobutyrate ferredoxin oxidoreductase (EC 1.2.7.1); the enzyme lactoyl-CoA dehydratase (EC 4.2.1.54); the enzyme acetyl-CoA C-acetyltransferase (EC 2.3.1.9); a combination of (i) the enzymes acetoacetyl-CoA reductase (EC 1.1.1.36) and enoyl-CoA hydratase 2 (EC 4.2.1.119) and/or (ii) the enzymes 3-hydroxybutyryl-CoA dehydrogenase (EC 1.1.1.157) and 3-hydroxybutyryl-CoA dehydratase (EC 4.2.1.55); a combination of the enzymes phosphate butyryltransferase (EC 2.3.1.19) and butyrate kinase (EC 2.7.2.7), and/or an acyl-CoA thioesterase enzyme (EC 3.1.2.-); and a phenyl acrylic acid decarboxylase enzyme (EC 4.1.1.-).

In some embodiments the recombinant nucleic acid molecule according to the second aspect includes coding sequences encoding the enzyme lactate CoA-transferase enzyme (EC 2.8.3.-); the enzyme lactoyl-CoA dehydratase (EC 4.2.1.54); the enzyme propionyl-CoA synthase (EC 1.3.1.84); the enzyme lactoyl-CoA dehydratase (EC 4.2.1.54); the enzyme acetyl-CoA C-acetyltransferase (EC 2.3.1.9); a combination of (i) the enzymes acetoacetyl-CoA reductase (EC 1.1.1.36) and enoyl-CoA hydratase 2 (EC 4.2.1.119) and/or (ii) the enzymes 3-hydroxybutyryl-CoA dehydrogenase (EC 1.1.1.157) and 3-hydroxybutyryl-CoA dehydratase (EC 4.2.1.55); a combination of the enzymes phosphate butyryltransferase (EC 2.3.1.19) and butyrate kinase (EC 2.7.2.7), and/or an acyl-CoA thioesterase enzyme (EC 3.1.2.-); and a phenyl acrylic acid decarboxylase enzyme (EC 4.1.1.-).

In some embodiments the recombinant nucleic acid molecule according to the second aspect includes coding sequences encoding the enzyme acetyl-CoA carboxylase (EC 6.4.1.2); the enzyme malonyl CoA reductase (EC 1.2.1.75); the enzyme malonyl CoA reductase (EC 1.1.1.298); the enzyme propionyl-CoA synthase (EC 6.2.1.36); the enzyme propionyl-CoA synthase/acryloyl-CoA synthase (EC 4.2.1.116); the enzyme propionyl-CoA synthase (EC 1.3.1.84); the enzyme lactoyl-CoA dehydratase (EC 4.2.1.54); the enzyme acetyl-CoA C-acetyltransferase (EC 2.3.1.9); a combination of (i) the enzymes acetoacetyl-CoA reductase (EC 1.1.1.36) and enoyl-CoA hydratase 2 (EC 4.2.1.119) and/or (ii) the enzymes 3-hydroxybutyryl-CoA dehydrogenase (EC 1.1.1.157) and 3-hydroxybutyryl-CoA dehydratase (EC 4.2.1.55); a combination of the enzymes phosphate butyryltransferase (EC 2.3.1.19) and butyrate kinase (EC 2.7.2.7), and/or an acyl-CoA thioesterase enzyme (EC 3.1.2.-); and a phenyl acrylic acid decarboxylase enzyme (EC 4.1.1.-).

In some embodiments the recombinant nucleic acid molecule according to the second aspect includes coding sequences encoding the enzyme 4-aminobutyrate aminotransferase (EC 2.6.1.19); a combination of (i) the enzyme glutamate dehydrogenase (EC 1.4.1.2), the enzyme 2-hydroxyglutarate dehydrogenase (EC 1.1.99.2), the enzyme glutaconate CoA-transferase (EC 2.8.3.12), a 2-hydroxyglutaryl-CoA dehydratase enzyme (EC 4.2.1.-) and/or (ii) the enzyme 4-hydroxybutyrate dehydrogenase (EC 1.1.1.61), a 4-hydroxybutyrate CoA-transferase enzyme (EC 2.8.3.-), the enzyme vinylacetyl-CoA-delta-isomerase (EC 4.2.1.120) and the enzyme glutaconyl-CoA decarboxylase (EC 4.1.1.70); the enzyme 4-hydroxybutyryl-CoA dehydratase (EC 5.3.3.3); the enzyme trans-2-enoyl-CoA reductase (EC1.1.1.36); the enzyme acetyl-CoA C-acetyltransferase (EC 2.3.1.9); the enzyme 3-hydroxybutyryl-CoA dehydrogenase (EC 1.1.1.157); the enzyme 3-hydroxybutyryl-CoA dehydratase (EC 4.2.1.55); an acyl-CoA thioesterase enzyme (EC 3.1.2.-); and a phenyl acrylic acid decarboxylase enzyme (EC 4.1.1.-).

In some embodiments the recombinant nucleic acid molecule according to the second aspect includes coding sequences encoding the enzyme acetyl-CoA carboxylase (EC 6.4.1.2); the enzyme malonyl CoA reductase (malonate semialdehyde-forming) (EC 1.2.1.75); the enzyme 3-hydroxypropionate dehydrogenase (EC 1.1.1.298); the enzyme 3-hydroxypropionyl-CoA synthase (EC 6.2.1.36); the enzyme hydroxypropionyl-CoA dehydratase (EC 4.2.1.116); a combination of the enzymes phosphate butyryltransferase (EC 2.3.1.19) and butyrate kinase (EC 2.7.2.7), and/or an acyl-CoA thioesterase enzyme (EC 3.1.2.-); and a phenyl acrylic acid decarboxylase enzyme (EC 4.1.1.-). In some embodiments the recombinant nucleic acid molecule includes coding sequences encoding a combination of (i) the enzyme acetyl-CoA carboxylase (EC 6.4.1.2), a lactate CoA-transferase enzyme (EC 2.8.3.-), and the enzyme lactoyl-CoA dehydratase (EC 4.2.1.54) and/or (ii) a lactate CoA-transferase enzyme (EC 2.8.3.-) and the enzyme lactoyl-CoA dehydratase (EC 4.2.1.54); a combination of the enzymes phosphate butyryltransferase (EC 2.3.1.19) and butyrate kinase (EC 2.7.2.7), and/or an acyl-CoA thioesterase enzyme (EC 3.1.2.-); and a phenyl acrylic acid decarboxylase enzyme (EC 4.1.1.-).

According to a third aspect, there is provided a method of producing one or more alkenes with a main chain of 2 to 5 carbon atoms. The method includes contacting a recombinant microorganism according to the second aspect with carbon monoxide and/or carbon dioxide.

The method according to the third aspect may be a method of producing ethylene. The method may also be a method of producing 1-propene. In some embodiments the method may be a method of producing 1-butene. The method may in some embodiments be a method of producing 2-methylpropene. In some embodiments the method may be a method of producing 1,3-butadiene. In some embodiments the method may be a method of producing 1-pentene. The method may in some embodiments be a method of producing 2-methyl-1,3-butadiene.

Additional advantages will be set forth in part in the description that follows, and in part will be apparent from the description and drawings, or can be learned by practice of a method or microorganism disclosed herein. The advantages of a respective method and microorganism will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE DRAWINGS

FIG. 1A depicts four nitrogenase pathways for the generation of hydrocarbons from CO, CO₂ and/or CN, and electrons.

FIG. 1B depicts the pathways, including the key enzymes, for the formation of ethene from carbon monoxide and/or from carbon dioxide as well as from carbon dioxide and electrons and/or from carbon monoxide and electrons.

FIG. 1C depicts the pathways, including the key enzymes, for the formation of propene from carbon monoxide and/or from carbon dioxide as well as from carbon dioxide and electrons and/or from carbon monoxide and electrons.

FIG. 1D shows the pathways, including the key enzymes, for the formation of 1-butene from carbon monoxide and/or from carbon dioxide as well as from carbon dioxide and electrons and/or from carbon monoxide and electrons.

FIG. 1E depicts the pathways, including the key enzymes, for the formation of isobutene from carbon monoxide and/or from carbon dioxide as well as from carbon dioxide and electrons and/or from carbon monoxide and electrons.

FIG. 1F depicts the pathways, including the key enzymes, for the formation of 1,3-butadiene from carbon monoxide and/or from carbon dioxide as well as from carbon dioxide and electrons and/or from carbon monoxide and electrons.

FIG. 1G depicts the pathways, including the key enzymes, for the formation of 2-methyl-1,3-butadiene from carbon monoxide and/or from carbon dioxide as well as from carbon dioxide and electrons and/or from carbon monoxide and electrons. (DMAP=dimethylallyl monophosphate)

FIG. 1H depicts the pathways, including the key enzymes, for the formation of 1-pentene from carbon monoxide and/or from carbon dioxide as well as from carbon dioxide and electrons and/or from carbon monoxide and electrons.

FIG. 2A shows the basic design principle of a plasmid based system for integration of DNA into a bacterial chromosome mediated by a random cut and paste transposase.

FIG. 2B illustrates the use of positive selection in isolating mutants of interest. The 5′ITR (inverted terminal repeat recognized and cut by the transposase) and the lox66 site serve as a promoter for the erythromycin/clarythromycin antibiotic resistance marker mIsR. When located on the plasmid the expression of mIsR is deactivated by the repressor lacI, which binds to the lac operator sequence and blocks transcription. After transposition, the operator sequence the repressor lac operator sequence is gone and resistance is conferred, since mIsR is expressed and can be selected for (RBS=ribosome binding site).

FIG. 3A schematically depicts the mode of action of the implemented positive selection system. The repressor binding operator site blocks expression of antibiotic resistance gene. 32: 3-hydroxybutyryl-CoA dehydrogenase (EC 1.1.1.157); 34: 3-hydroxybutyryl-CoA dehydratase (EC 4.2.1.55); 36: acyl-CoA thioesterase [EC 3.1.2.-]; 38: aldehyde/alcohol dehydrogenase (EC 1.1.1.- and EC 1.2.1.-); 40: geraniol isomerase/linalool dehydratase (EC 5.4.4.4 and EC 4.2.1.127); 42: acetyl-CoA carboxylase (EC 6.4.1.2).

FIG. 3B schematically depicts the basic design principle of a plasmid based system for integration of DNA into a bacterial chromosome mediated by a random cut and paste transposase. 36: acyl-CoA thioesterase (EC 3.1.2.-); 38: aldehyde/alcohol dehydrogenase (EC 1.1.1.- and EC 1.2.1.-); 40: geraniol isomerase/linalool dehydratase (EC 5.4.4.4 and EC 4.2.1.127); 44: acetoacetyl-CoA synthase (EC 2.3.1.194).

FIG. 4A shows plasmid SG193 (SEQ ID NO: 1) which contains sequences encoding enzymes of a molybdenum nitrogenase pathway.

FIG. 4B shows plasmid SG211 (SEQ ID NO: 2) which contains sequences encoding enzymes of a vanadium nitrogenase pathway.

FIG. 4C shows plasmid SG278 (SEQ ID NO: 3) which contains sequences encoding enzymes of an iron nitrogenase pathway.

FIG. 4D shows plasmid SG323 (SEQ ID NO: 4) which contains a sequence encoding an engineered nitrogenase.

FIG. 5A shows plasmid SG123 (SEQ ID NO: 5) which contains sequences encoding enzymes of a MEP pathway together with kudzu 2-methyl-1,3-butadiene synthase.

FIG. 5B shows plasmid SG124 (SEQ ID NO: 6) which contains sequences encoding enzymes of a MEP pathway together with populus 2-methyl-1,3-butadiene synthase.

FIG. 6 shows plasmid SG156 (SEQ ID NO: 7) which contains sequences encoding enzymes of a MEP pathway together with kudzu 2-methyl-1,3-butadiene synthase.

FIG. 7 shows plasmid SG157 (SEQ ID NO: 8) which contains sequences encoding enzymes of a MEP pathway together with populus 2-methyl-1,3-butadiene synthase.

FIG. 8A depicts a GC-MS chromatogram in product analysis of the product 2-methyl-1,3-butadiene obtained by fermentative production with carbon dioxide and electrons as reactants using Clostridium ljungdahlii harbouring plasmid SG156.

FIG. 8B depicts a mass spectrum of the peak measured and depicted in FIG. 8A, which was identified as 2-methyl-1,3-butadiene.

FIG. 9 schematically depicts the vector pANT (SEQ ID NO: 9), a methylating-plasmid required for genetic transformation of Clostridium acetobutylicum.

FIG. 10 schematically depicts the vector BG132 (abbreviated T1, SEQ ID NO: 10), a plasmid for chromosomal integration via transposase containing the positive selection system for chromosomal integration.

FIG. 11 shows a vector map of the vector BG133 (abbreviated T2, SEQ ID NO: 11).

FIG. 12 shows a vector map of the vector BG134 (abbreviated T3, SEQ ID NO: 12).

FIG. 13 shows a vector map of the vector BG135 (abbreviated T4, SEQ ID NO: 13).

FIG. 14 shows a vector map of the vector BG136 (abbreviated H, SEQ ID NO: 14).

FIG. 15A depicts the separation of PCR by 0.8% analytic DNA agarose gel electrophoresis of Clostridium acetobutylicum DNA. Lane 1: standard; lanes 2-6: integrand 1 PCR 1-5; lanes 7-11: integrand 2 PCR 1-5; lane 12: standard; lanes 13-17: integrand 3 PCR 1-5; lanes 18-22: integrand 4 PCR 1-5; Lane 23-27: integrand 5 PCR 1-5; lane 28: standard.

FIG. 15B depicts the separation of PCR by 0.8% analytic DNA agarose gel electrophoresis. Lane 1: standard; lanes 2-6: integrand 6 PCR 1-5; lanes 7-11: integrand 7 PCR 1-5; lanes 12-16: integrand 8 PCR 1-5; lane 17: standard; lanes 18-22: wild type PCR 1-5; lanes 23-27: integrand 5 PCR 1-5; lane 28: standard.

FIG. 16 depicts fluorescence microscopy photos of integrands. FIG. 16A, 16B, 16C: Clostridium acetobutylicum integrand 1; FIG. 16D: Clostridium acetobutylicum integrand 2; FIG. 16E, 16F, 16G: Clostridium acetobutylicum integrand 2; FIG. 16H: Clostridium acetobutylicum wild type control.

FIG. 17 shows a vector map of the vector BG168 (abbreviated “I”, SEQ ID NO: 15).

FIG. 18 shows a vector map of the vector BG182 (SEQ ID NO: 16).

FIG. 19 shows a vector map of the vector BG282 (SEQ ID NO: 17).

FIG. 20 shows a vector map of the vector BG281 (SEQ ID NO: 18).

FIG. 21 shows a vector map of the vector BG287 (SEQ ID NO: 19).

FIG. 22 shows a vector map of the vector BG288 (SEQ ID NO: 20).

FIG. 23 shows a vector map of the vector BG289 (SEQ ID NO: 21).

FIG. 24 shows a vector map of the vector BG290 (SEQ ID NO: 22).

FIG. 25 shows a vector map of the vector BG291 (SEQ ID NO: 23).

FIG. 26 shows a vector map of the vector BG292 (SEQ ID NO: 24).

FIG. 27 shows a vector map of the vector BG178 (SEQ ID NO: 25).

FIG. 28 depicts the separation of PCR products by 0.8% analytic DNA agarose gel electrophoresis of Clostridium ljungdahlii DNA. Lane 1: standard; lanes 2-9: integrand LBI 105-111 PCR; lane 10: standard; lanes 11-18: integrand LBI 112-120 PCR; lane 19: standard; lanes 24-25: positive control PCR; lanes 26-27: negative control gDNA C. ljungdahlii gDNA PCR; lanes 28-29: negative control gDNA C. 3.

FIG. 29 depicts the separation of PCR products by 0.8% analytic DNA agarose gel electrophoresis of Clostridium autoethanogenum DNA. Lane 1: standard; lanes 2-5: integrand A1-A4 PCR; lanes 6-7: positive control PCR; lanes 8-9: negative control gDNA C. ljungdahlii gDNA PCR; lanes 10-11: negative control gDNA C. autoethanogenum gDNA PCR; lane 12: standard.

FIG. 30 schematically depicts the plasmid SG193 (SEQ ID NO: 26) which contains sequences encoding enzymes of a molybdenum nitrogenase pathway.

FIG. 31 schematically depicts the plasmid SG211 (SEQ ID NO: 27) which contains sequences encoding enzymes of a vanadium nitrogenase pathway.

FIG. 32 schematically depicts the plasmid SG278 (SEQ ID NO: 28) which contains sequences encoding enzymes of an iron nitrogenase pathway.

FIG. 33 schematically depicts the plasmid SG323 (SEQ ID NO: 29) which contains an engineered nitrogenase complex.

FIG. 34 schematically depicts the plasmid SG387 (SEQ ID NO: 30) which contains sequences encoding enzymes of a 1,3-butadiene pathway type 1 (crotonyl-CoA biosynthesis is derived from Clostridium acetobutylicum).

FIG. 35 schematically depicts the plasmid SG411 (SEQ ID NO: 31) which contains sequences encoding enzymes of a 1,3-butadiene pathway type 2 (crotonyl-CoA biosynthesis via malonyl-CoA is derived from Escherichia coli and other bacteria).

FIG. 36 schematically depicts the plasmid SG455 (SEQ ID NO: 32) which contains sequences encoding enzymes for propene biosynthesis.

FIG. 37 schematically depicts the plasmid SG479 (SEQ ID NO: 33) which contains sequences encoding enzymes for propene biosynthesis.

FIG. 38 schematically depicts the plasmid SG539 (SEQ ID NO: 34) which contains sequences encoding enzymes for 1-butene biosynthesis.

FIG. 39 schematically depicts the plasmid SG523 (SEQ ID NO: 35) which contains sequences encoding enzymes for 1-butene biosynthesis.

FIG. 40 schematically depicts the plasmid SG582 (SEQ ID NO: 36) which contains sequences encoding enzymes for isobutene biosynthesis.

FIG. 41 schematically depicts the plasmid SG601 (SEQ ID NO: 37) which contains sequences encoding enzymes for isobutene biosynthesis.

FIG. 42 schematically depicts the plasmid SG498 (SEQ ID NO: 38) which contains sequences encoding enzymes for 1-pentene biosynthesis.

FIG. 43 schematically depicts the plasmid SG513 (SEQ ID NO: 39) which contains sequences encoding enzymes for 1-pentene biosynthesis.

FIG. 44 schematically depicts the plasmid SG557 (SEQ ID NO: 40) which contains sequences encoding enzymes for ethene biosynthesis.

FIG. 45 schematically depicts the plasmid SG598 (SEQ ID NO: 41) which contains sequences encoding enzymes for ethene biosynthesis.

FIG. 46 schematically depicts the plasmid SG661 (SEQ ID NO: 42) which contains sequences encoding enzymes of a 1,3-butadiene pathway type 3 (crotonyl-CoA synthesis via malonyl-CoA is derived from Escherichia coli and other bacteria and the classical pathway from Clostridium acetobutylicum).

FIG. 47 schematically depicts the plasmid SG696 (SEQ ID NO: 43) which contains sequences encoding enzymes of a 1,3-butadiene pathway type 4 (crotonyl-CoA synthesis via glutamate and 4-aminobutanoate).

FIG. 48 schematically depicts the plasmid SG726 (SEQ ID NO: 44) which contains sequences encoding enzymes of a pathways for 2-methyl-1,3-butadiene biosynthesis and the positive selection system for chromosomal integration.

FIG. 49 schematically depicts the plasmid SG705 (SEQ ID NO: 45) which contains sequences encoding enzymes of a pathways for 2-methyl-1,3-butadiene biosynthesis and the positive selection system for chromosomal integration.

FIG. 50 schematically depicts the plasmid SG714 (SEQ ID NO: 46) which contains sequences encoding enzymes of a pathways for 1,3-butadiene biosynthesis and the positive selection system for chromosomal integration.

FIG. 51 schematically depicts the plasmid SG739 (SEQ ID NO: 47) which contains sequences encoding enzymes of a pathways for 1,3-butadiene biosynthesis and the positive selection system for chromosomal integration.

FIG. 52 schematically depicts the plasmid SG755 (SEQ ID NO: 48) which contains sequences encoding enzymes of pathways for propene biosynthesis and the positive selection system for chromosomal integration.

FIG. 53 schematically depicts the plasmid SG779 (SEQ ID NO: 49) which contains sequences encoding enzymes of pathways for propene biosynthesis and the positive selection system for chromosomal integration.

FIG. 54 schematically depicts the plasmid SG839 (SEQ ID NO: 50) which contains sequences encoding enzymes of pathways for 1-butene biosynthesis and the positive selection system for chromosomal integration.

FIG. 55 schematically depicts the plasmid SG823 (SEQ ID NO: 51) which contains sequences encoding enzymes of pathways for 1-butene biosynthesis and the positive selection system for chromosomal integration.

FIG. 56 schematically depicts the plasmid SG882 (SEQ ID NO: 52) which contains sequences encoding enzymes of pathways for isobutene biosynthesis and the positive selection system for chromosomal integration.

FIG. 57 schematically depicts the plasmid SG901 (SEQ ID NO: 53) which contains sequences encoding enzymes of pathways for isobutene biosynthesis and the positive selection system for chromosomal integration.

FIG. 58 schematically depicts the plasmid SG798 (SEQ ID NO: 54) which contains sequences encoding enzymes of pathways for 1-pentene biosynthesis and the positive selection system for chromosomal integration.

FIG. 59 schematically depicts the plasmid SG813 (SEQ ID NO: 55) which contains sequences encoding enzymes of pathways for 1-pentene biosynthesis and the positive selection system for chromosomal integration.

FIG. 60 schematically depicts the plasmid SG857 (SEQ ID NO: 56) which contains sequences encoding enzymes of pathways for ethene biosynthesis and the positive selection system for chromosomal integration.

FIG. 61 schematically depicts the plasmid SG898 (SEQ ID NO: 57) which contains sequences encoding enzymes of pathways for ethene biosynthesis and the positive selection system for chromosomal integration.

FIG. 62 lists the initial integration tests by serial transfer (positive selection) using plasmid N and plasmid I.

FIG. 63 shows the preparation of gDNA of the integrants obtains in the test depicted in FIG. 62. R/C: replicate/clone; Cla: Clarythromycin (μg/ml).

FIG. 64 shows the determination of loci of the integrants analysed in FIG. 63 by PCR genome walking.

FIG. 65 summarizes the protocol used for analyzing genomic DNA of integrands. GDNA concentrations were determined using a NanoDrop spectrophotometer (ThermoFisher Scientific Inc., Darmstadt, Germany).

FIG. 66 shows the enzymes encoded by exemplary plasmids disclosed herein. FIG. 66A shows the enzymes encoded by four plasmids that contains nucleic acid sequences of enzymes of pathways leading to the biosynthesis of isobutene. FIG. 66B shows the enzymes encoded by two plasmids that contains nucleic acid sequences of enzymes of pathways leading to the biosynthesis of isoprene. FIG. 66C shows the enzymes encoded by six plasmids that contains nucleic acid sequences of enzymes of pathways leading to the biosynthesis of isoprene.

FIG. 67A depicts a GC-MS chromatogram in product analysis of the product isobutene obtained by fermentative production with carbon dioxide and electrons as reactants using Clostridium ljungdahlii DSM 13528 harbouring plasmid SG601. (Inten.=intensity)

FIG. 67B depicts a mass spectrum of the peak measured and depicted in FIG. 67A, which was identified as isobutene. (Int.=intensity)

Throughout the drawings the following reference numerals apply:
20—transposase (e.g. himar1)
22—transposase recognition site (5′ ITR inverted terminal repeat)
24—antibiotic resistance marker (mlsR)
26—gene(s) of interest (cargo)
28—transposase recognition site (3′ ITR inverted terminal repeat)
30—origin of replication
31—Acetoacetyl-Coenzyme A synthase thiolase (EC 2.3.1.9)=AtoB
32—3-hydroxybutyryl-CoA dehydrogenase (EC 1.1.1.157)=hbd
34—3-hydroxybutyryl-CoA dehydratase (EC 4.2.1.55)=crt
36—acyl-CoA thioesterase (EC 3.1.2.-)=YciA, YdiI
38—phenyl acrylic acid decarboxylase (EC 4.1.1.-)=PAD1, PADC
40—geraniol isomerase/linalool dehydratase (EC 5.4.4.4 and EC 4.2.1.127)=GIM, GIT
42—acetyl-CoA carboxylase (EC 6.4.1.2)=AccABCD
44—acetoacetyl-CoA synthase (EC 2.3.1.194)=nphT7
46—acetoacetyl-CoA reductase (EC 1.1.1.36)=phaB1
48—(R)-specific enoyl-CoA hydratase (EC 4.2.1.119)=phaJ
50—4-aminobutyrate aminotransferase (EC 2.6.1.19)=gabT
52—glutamate dehydrogenase (EC 1.4.1.2)=gdh
54—2-hydroxyglutarate dehydrogenase (EC 1.1.99.2)=ygaF
56—glutaconate CoA-transferase (EC 2.8.3.12)=gctA
58—2-hydroxyglutaryl-CoA dehydratase (EC 4.2.1.-)=hdgAB
60—glutaconyl-CoA decarboxylase (EC 4.1.1.70)=gcdABCD
62—4-hydroxybutyrate dehydrogenase, also called NAD-dependent 4-hydroxybutyrate dehydrogenase (EC 1.1.1.61)=4hbD
64—4-hydroxybutyrate CoA-transferase (EC 2.8.3.-)=abfT
66—vinylacetyl-CoA-Delta-isomerase (EC 4.2.1.120)=abfD
68—4-hydroxybutyryl-CoA dehydratase (EC 5.3.3.3)=abfD
70—acetoacetyl-Coenzyme A synthase thiolase (EC 2.3.1.9)=Bktb
72—2-oxoglutarate/butyrate ferredoxin oxidoreductase, also called 2-ketoglutarate oxidoreductase (EC 1.2.7.3)=KorAB
74—pyruvate/2-oxobutyrate ferredoxin oxidoreductase, also called pyruvate/ketoisovalerate oxidoreductase (EC 1.2.7.1)=PFOR
76—methylmalonyl-CoA mutase (EC 5.4.99.2)=scpA
78—methylmalonyl-CoA decarboxylase (EC 4.1.1.41)=scpB
80—3-hydroxy-3-methylglutaryl-CoA synthase (EC 2.3.3.10)=mvaS
82—3-methylglutaconyl-CoA hydratase (EC 4.2.1.18)=liuC
84—3-methylcrotonyl-CoA carboxylase (EC 6.4.1.4)=liuBD
86—trans-2-enoyl-CoA reductase (EC1.1.1.36)=ter
88—acetyl-CoA acetyltransferase (EC 2.3.1.9)=Bktb
90—3-hydroxybutyryl-CoA dehydrogenase (EC 1.1.1.157)=pAAH1
92—acyl-CoA hydrolase (EC 3.1.2.-)=mBACH
94—phosphate butyryltransferase (EC 2.3.1.19)=ptb
96—butyrate kinase (EC 2.7.2.7)=buk
100—lactate CoA-transferase (EC 2.8.3.-)=pct
102—lactoyl-CoA dehydratase (EC 4.2.1.54)=lcdAB
104—malonyl CoA reductase (EC 1.2.1.75)=mcr
106—malonyl CoA reductase (EC 1.1.1.298)=mcr
108—propionyl-CoA synthase (EC 6.2.1.36)=per
110—propionyl-CoA synthase/acryloyl-CoA synthase (EC 4.2.1.116)=per, msed12
112—propionyl-CoA synthase (EC 1.3.1.84)=per
132—repressor (e.g. lacI)
134—operator site (e.g. lac operator)
136—transposase recognition site (5′ ITR inverted terminal repeat)
138—antibiotic resistance gene (mlsR)
140—antibiotic resistance protein (mlsR)
142—plasmid with positive selection feature
144—chromosome with randomly integrated DNA cassette
pMB1=origin of replication (E. coli)
repH=origin of replication (Clostridium)
catp=chloramphenicol/thiamphenicol resistance marker
lacI=lac repressor
tetR=tetracycline repressor
xylR=xylose repressor
himar=a transposase

### DETAILED DESCRIPTION

Before the present microorganism, composition, nucleic acid molecule, vector, host cell, and/or method is disclosed and described, it is to be understood that they are not limited to a specific synthetic method unless otherwise specified, or to particular reagents unless otherwise specified, as such may, naturally, vary. It is also to be understood that the terminology used herein is for the purpose of describing particular aspects only and is not intended to be limiting. Although any methods and materials similar or equivalent to those described herein can be used, example methods and materials are now described.

The use of a microorganism as provided herein allows a new method of obtaining one or more alkanes and/or alkenes with a chain length of 6 or less carbon atoms. The method utilizes gaseous C₁-carbon sources, including gaseous C₁-carbon sources and electrons.

Disclosed herein are, amongst others, methods of producing one or more alkanes and/or alkenes from carbon monoxide and/or carbon dioxide. The methods are based on the use of one or more non-naturally occurring microorganisms. The respective microorganisms are also disclosed herein. The microorganisms disclosed herein may have a capability of alkene biosynthesis. The microorganisms may also have a capability of alkane biosynthesis. In some embodiments the microorganisms may have a capability of alkane biosynthesis and alkene biosynthesis. The microorganisms disclosed herein are recombinant microorganisms that contain a heterologous nucleic acid sequence. The heterologous nucleic acid sequence contains one or more sequences encoding one or more enzymes. The respective enzyme(s) provide the recombinant microorganism with the capability of converting carbon monoxide and/or carbon dioxide to producing one or more alkanes and/or alkenes.

An alkene produced according to a method disclosed herein may in some embodiments be represented by the following formula:

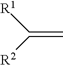

In this formula R¹ may be an aliphatic carbon chain that may contain one double bond. In some embodiments R¹ does not contain a double bond. Typically R¹ is unbranched. In some embodiments R¹ may contain 4 carbon atoms. In some embodiments R¹ may contain 2 or 3 carbon atoms. R¹ may in some embodiments contain a single carbon atom. R² may be an unbranched aliphatic carbon chain that does not contain a double bond. Generally R² may contain 1 or 2 carbon atoms. In some embodiments R² is a methyl group. R² may also be a hydrogen atom.

Generally the microorganisms have been engineered to produce one or more alkanes and/or alkenes. The microorganisms have in some embodiments been engineered to produce one or more alkanes and/or alkenes from carbon monoxide and/or carbon dioxide. A microorganism disclosed herein may have been engineered on the basis of a naturally occurring microorganism that lacks a capability of alkene biosynthesis. In some embodiments a microorganism disclosed herein may have been engineered on the basis of a naturally occurring microorganism that lacks a capability of alkene biosynthesis from carbon monoxide and/or carbon dioxide. A microorganism disclosed herein may also have been engineered on the basis of a naturally occurring microorganism that lacks a capability of alkane biosynthesis. In some embodiments a microorganism disclosed herein may have been engineered on the basis of a naturally occurring microorganism that lacks a capability of alkane biosynthesis from carbon monoxide and/or carbon dioxide. In some embodiments a microorganism disclosed herein may have been engineered on the basis of a naturally occurring microorganism that lacks a capability of both alkane biosynthesis and alkene biosynthesis. In some embodiments a microorganism disclosed herein may have been engineered on the basis of a naturally occurring microorganism that lacks a capability of both alkane biosynthesis and alkene biosynthesis from carbon monoxide and/or carbon dioxide. Disclosed are also methods of making and using such organisms for the production of one or more alkanes and/or alkenes from carbon monoxide and/or carbon dioxide, including the design and production of microorganisms that have a capability of alkane and/or alkene biosynthesis

### Definitions

Unless otherwise stated, the following terms used in this document, including the description and claims, have the definitions given below.

As used in the specification and the appended claims, the singular forms “a”, “an” and “the” include plural referents unless the context clearly dictates otherwise. The word “or” as used herein means any one member of a particular list and also includes any combination of members of that list.

The word “about” as used herein refers to a value being within an acceptable error range for the particular value as determined by one of ordinary skill in the art, which will depend in part on how the value is measured or determined, i.e., the limitations of the measurement system. For example, “about” can mean within 1 or more than 1 standard deviation, per the practice in the art. The term “about” is also used to indicate that the amount or value in question may be the value designated or some other value that is approximately the same. The phrase is intended to convey that similar values promote equivalent results or effects as described. In this context “about” may refer to a range above and/or below of up to 10%. The word “about” refers in some embodiments to a range above and below a certain value that is up to 5%, such as up to up to 2%, up to 1%, or up to 0.5% above or below that value. In one embodiment “about” refers to a range up to 0.1% above and below a given value.

The term “allelic variant” denotes herein any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene.

The term “alkane” means, unless otherwise stated, a saturated aliphatic molecule. A respective aliphatic molecule has a straight or branched hydrocarbon chain, which may include heteroatoms. In a method disclosed herein an alkane generally does not contain a heteroatom. The branches of the hydrocarbon chain may include linear chains. Generally an alkane formed in a method described herein does not contain cyclic elements. The hydrocarbon chain may, unless otherwise stated, be of any length, and contain any number of branches. A branch may be defined by an alkyl group such as a methyl, an ethyl or a propyl group. Typically, the hydrocarbon (main) chain of an alkane formed in a method described herein includes 1 to 8, such as 2 to 6 carbon atoms.

The term “alkene” means, unless otherwise stated, an unsaturated aliphatic molecule, which contains one or more double bonds. A respective aliphatic molecule has a straight or branched hydrocarbon chain, which may be mono- or poly-unsaturated and include heteroatoms. In a method disclosed herein an alkene generally does not contain a heteroatom. The presence of branches in the hydrocarbon chain has already been addressed above. Generally an alkene formed in a method described herein does not contain cyclic elements. Typically, the hydrocarbon (main) chain of an alkene formed in a method described herein includes 1 to 8, such as 2 to 6 carbon atoms

An “anaerobe” is an organism that does not require oxygen for growth. An anaerobe can be an obligate anaerobe, a facultative anaerobe, or an aerotolerant organism. An “obligate anaerobe” is an anaerobe for which atmospheric levels of oxygen can be lethal. Examples of obligate anaerobes include, but are not limited to, Clostridium, Eurobacterium, Bacteroides, Peptostreptococcus, Butyribacterium, Veillonella, and Actinomyces. A “facultative anaerobe” is an anaerobe that is capable of performing aerobic respiration in the presence of oxygen and is capable of performing anaerobic fermentation under oxygen-limited or oxygen-free conditions. Examples of facultative anaerobes include, but are not limited to, Escherichia, Pantoea, and Streptomyces.

The terms “carboxidotrophic organism” and “carboxidotroph” refers to an organism that can tolerate high concentrations of carbon monoxide, and generally capable of utilizing carbon monoxide for metabolism. A carboxydotrophic microorganism may furthermore obtain energy and carbon from the oxidation of CO. Such a carboxydotrophic microorganism may be an aerobic bacterium capable of oxidizing CO to carbon dioxide. A carboxydotrophic microorganism may also be an obligate anaerobe, which may be capable of reducing carbon dioxide to CO and/or converting CO to carbon dioxide and hydrogen. Some carboxydotrophic microorganisms that are an obligate anaerobe are acetogenic bacteria, e.g. Morella thermoacetica, and can form acetate from CO and/or CO₂.

When used herein the term “coding sequence” means a nucleotide sequence, which directly specifies the amino acid sequence of its protein product. The boundaries of the coding sequence are generally determined by an open reading frame, which usually begins with the ATG start codon or alternative start codons such as GTG and TTG and ends with a stop codon such as TAA, TAG, and TGA. The coding sequence may be a DNA, cDNA, synthetic or recombinant nucleotide sequence.

As used herein, the terms “conservative modification” and “conservative substitution” refer to a modification and a substitution, respectively, that maintains physically, biologically, chemically or functionally the properties with regard to the corresponding reference. A molecule that includes a sequence with conservative substitution for instance has a similar size, shape, electric charge, chemical properties, including a comparable ability to form covalent or hydrogen bonds, and/or comparable polarity. Such conservative modifications include, but are not limited to, one or more nucleobases and amino acid substitutions, additions and deletions.

For example, conservative amino acid substitutions include those in which the amino acid residue is replaced with an amino acid residue having a similar side chain. For example, amino acid residues being non-essential with regard to binding to an antigen can be replaced with another amino acid residue from the same side chain family, e.g. serine may be substituted for threonine Amino acid residues are usually divided into families based on common, similar side-chain properties, such as:
1. nonpolar side chains (e.g., glycine, alanine, valine, leucine, isoleucine, methionine),
2. uncharged polar side chains (e.g., asparagine, glutamine, serine, threonine, tyrosine, proline, cysteine, tryptophan),
3. basic side chains (e.g., lysine, arginine, histidine),
4. acidic side chains (e.g., aspartic acid, glutamic acid),
5. beta-branched side chains (e.g., threonine, valine, isoleucine) and
6. aromatic side chains (e.g., tyrosine, phenylalanine, tryptophan, histidine).
   This classification can be further segmented. As a further orientation, the following eight groups each contain amino acids that can typically be taken to define conservative substitutions for one another:
1) Alanine (Ala), Glycine (Gly);
2) Aspartic acid (Asp), Glutamic acid (Glu);
3) Asparagine (Asn), Glutamine (Gln);
4) Arginine (Arg), Lysine (Lys);
5) Isoleucine (Ile), Leucine (Leu), Methionine (Met), Valine (Val);
6) Phenylalanine (Phe), Tyrosine (Tyr), Tryptophan (Trp);
7) Serine (Ser), Threonine (Thr); and
8) Cysteine (Cys), Methionine (Met).
   A conservative substitution can be taken to be a substitution of a first amino acid within one of the six groups above by a further amino acid within the same group of the six groups. Conservative substitutions are generally the following substitutions, listed according to the amino acid to be mutated, each followed by one or more replacement(s) that can be taken to be conservative: Ala→Gly, Ser, Val; Arg→Lys; Asn→Gln, His; Asp→Glu; Cys→Ser; Gln→Asn; Glu→Asp; Gly→Ala; His→Arg, Asn, Gln; Ile→Leu, Val; Leu→Ile, Val; Lys→Arg, Gln, Glu; Met→Leu, Tyr, Ile; Phe→Met, Leu, Tyr; Ser→Thr; Thr→Ser; Trp→Tyr; Tyr→Trp, Phe; Val→Ile, Leu. Other substitutions are also permissible and can be determined empirically or in accord with other known conservative or non-conservative substitutions. A conservative substitution may also involve the use of a non-natural amino acid.

Non-conservative substitutions, i.e. exchanging members of one family against members of another family, may lead to substantial changes, e.g., with respect to the charge, dipole moment, size, hydrophilicity, hydrophobicity or conformation of the binding member, which may lead to a significant drop in the binding activity, in particular if amino acids are affected that are essential for binding to the target molecule. A non-conservative substitution may also involve the use of a non-natural amino acid.

Conservative and non-conservative modifications can be introduced into parental binding members by a variety of standard techniques known in the art, such as combinatorial chemistry, site-directed DNA mutagenesis, PCR-mediated and/or cassette mutagenesis, peptide/protein chemical synthesis, chemical reaction specifically modifying reactive groups in the parental binding member. The variants can be tested by routine methods for their chemical, biological, biophysical and/or biochemical properties. Preferably, the conservative amino acid substitution does not substantially change the functional, and generally also the structural characteristics of the parental sequence. Accordingly, the binding characteristics of a binding member that includes a conservative substitution are at least essentially unaltered. Furthermore, a conservative amino acid substitution does generally not substantially modify or disrupt a secondary structure of the parental sequence.

The term “endogenous” as used herein refers to a referenced molecule naturally present in the host. Accordingly, an “endogenous” enzyme, as used herein in the context of a microorganism, is an enzyme that is produced or synthesized from a nucleic acid sequence that is naturally a part of the same microorganism, such as a gene of the respective species.

The term “exogenous”, as used herein, is intended to mean that the referenced molecule, sequence or activity is or has been introduced into the host microbial organism. An exogenous nucleic acid molecule or sequence thus refers to any nucleic acid molecule or sequence that does not originate from that particular organism as found in nature. Thus, a non-naturally-occurring nucleic acid molecule or sequence is considered to be exogenous to a cell once introduced into the organism. The molecule or sequence can be introduced, for example, by introduction of an encoding nucleic acid molecule into the host genetic material such as by integration into a host chromosome or as non-chromosomal genetic material such as a plasmid. Therefore, the term as it is used in reference to expression of an encoding nucleic acid refers to introduction of the encoding nucleic acid in an expressible form into the microorganism. When used in reference to a biosynthetic activity, the term refers to an activity that is introduced into the host reference organism. The source can be, for example, a homologous or heterologous encoding nucleic acid that expresses the referenced activity following introduction into the host microorganism.

It is important to note that a non-naturally-occurring nucleic acid molecule or sequence can contain nucleic acid sequences or fragments of nucleic acid sequences that are found in nature, provided the nucleic acid as a whole does not exist in nature. For example, a nucleic acid molecule containing a genomic DNA sequence within an expression vector is a non-naturally-occurring nucleic acid molecule, and thus is exogenous to a cell once introduced into the cell, since that nucleic acid molecule as a whole (genomic DNA plus vector DNA) does not exist in nature. Thus, any vector, autonomously replicating plasmid, or virus (e.g., retrovirus, adenovirus, or herpes virus) that as a whole does not exist in nature is considered to be non-naturally-occurring nucleic acid. It follows that a genomic DNA fragment produced by PCR or restriction endonuclease treatment as well as a cDNA is considered to be non-naturally-occurring nucleic acid since they exist as separate molecules not found in nature. It also follows that any nucleic acid containing a promoter sequence and polypeptide-encoding sequence (e.g., cDNA or genomic DNA) in an arrangement not found in nature is non-naturally-occurring nucleic acid. Nucleic acid that is naturally-occurring can be exogenous to a particular organism. For example, an entire chromosome isolated from a cell of organism X is an exogenous nucleic acid with respect to a cell of organism Y once that chromosome is introduced into organism's cell.

The terms “expressing” and “expression” in reference to a protein define that a sequence included in a nucleic acid molecule and encoding a peptide/protein is converted into its peptide/protein product. Where the nucleic acid is DNA, a protein such as an enzyme is expressed by a cell via transcription of a nucleic acid into mRNA, followed by translation into a polypeptide, which is folded and possibly further processed. Where the nucleic acid is RNA, expression may include the replication of this RNA into further RNA copies and/or the reverse transcription of the RNA into DNA and optionally the transcription of this DNA into further RNA molecule(s). In any case expression of RNA includes the translation of any of the RNA species provided/produced into protein. Hence, the statement that a cell is expressing a protein implies that the protein has been synthesized by the expression machinery of the respective cell. With regard to the respective biological process itself, the terms “expression”, “gene expression” or “expressing” refer to the entirety of regulatory pathways converting the information encoded in the nucleic acid sequence of a gene first into messenger RNA (mRNA) and then to a protein. Accordingly, the expression of a gene includes its transcription into a primary hnRNA, the processing of this hnRNA into a mature RNA and the translation of the mRNA sequence into the corresponding amino acid sequence of the protein. In this context, it is also noted that the term “gene product” refers not only to a protein, including e.g. a final protein (including a splice variant thereof) encoded by that gene and a respective precursor protein where applicable, but also to the respective mRNA, which may be regarded as the “first gene product” during the course of gene expression. Expression of a protein or peptide may be carried out using an in vitro expression system. Such an expression system may include a cell extract, typically from bacteria, rabbit reticulocytes or wheat germ. Many suitable systems are commercially available. The mixture of amino acids used may include synthetic amino acids if desired, to increase the possible number or variety of proteins produced in the library. This can be accomplished by charging tRNAs with artificial amino acids and using these tRNAs for the in vitro translation of the proteins to be selected. A suitable embodiment for expression purposes is the use of a vector, in particular an expression vector.

As used herein, the term “expression cassette” refers to a nucleic acid molecule capable of directing expression of a particular nucleotide sequence in an appropriate host cell. An expression cassette includes a promoter operatively linked to the nucleotide sequence of interest, which is operatively linked to one or more termination signals. It may also include sequences required for proper translation of the nucleotide sequence. The coding region can encode a polypeptide of interest and can also encode a functional RNA of interest, including but not limited to, antisense RNA or a non-translated RNA, in the sense or antisense direction. The expression cassette contains the nucleotide sequence of interest can be chimeric, meaning that at least one of its components is heterologous with respect to at least one of its other components. The expression cassette can also be one that is naturally occurring but has been obtained in a recombinant form useful for heterologous expression. In some embodiments, however, the expression cassette is heterologous with respect to the host; i.e., the particular nucleic acid sequence of the expression cassette does not occur naturally in the host cell and was introduced into the host cell or an ancestor of the host cell by a transformation event. The expression of the nucleotide sequence in the expression cassette can be under the control of a constitutive promoter or of an inducible promoter that initiates transcription only when the host cell is exposed to some particular external stimulus. In the case of a multicellular organism such as a plant or an animal, the promoter can also be specific to a particular tissue, organ, or stage of development.

Unless the context requires otherwise, the phrases “fermenting”, “fermentation process” and “fermentation reaction” when used herein, refer to an enzymatic conversion of compounds. The respective compounds are generally organic compounds and include gaseous carbon compounds such as carbon monoxide and carbon dioxide. The enzymatic conversion of reactants to product compounds typically involves cells, which encompass and/or produce the enzymes required for the enzymatic conversion. A fermentation process includes the culture of cells, regardless of their stage, density and/or growth condition. For instance both the growth phase and the product biosynthesis phase of the process are intended to be encompassed. In this regard, in some embodiments a bioreactor may contain a first growth reactor and a second fermentation reactor. As such, the addition of metals or compositions to a fermentation reaction should be understood to include addition to either or both of these reactors.

A “fermentation broth” as used herein is a culture medium that contains a nutrient media and bacterial cells.

By “gene” is meant a unit of inheritance that occupies a specific locus on a chromosome and that is a segment of nucleic acid associated with a biological function. A gene encompasses transcriptional and/or translational regulatory sequences as well as a coding region. Besides a coding sequence a gene may include a promoter region, a cis-regulatory sequence, a non-expressed DNA segment that is a specific recognition sequence for regulatory proteins, a non-expressed DNA segment that contributes to gene expression, a DNA segment designed to have desired parameters, or combinations thereof. A gene can be obtained by a variety of methods, including cloning from a biological sample, synthesis based on known or predicted sequence information, and recombinant derivation of an existing sequence.

A “homologous” enzyme, as used herein in the context of a microorganism, is an enzyme that is produced or synthesized from a nucleic acid sequence that is naturally a part of a microorganism of the same species. A homologous enzyme may thus be an enzyme that is identical to the enzyme encoded by a gene of a microorganism, but it may have been introduced into the microorganism by way of genetic engineering. The respective enzyme can in such a case not be distinguished from the microorganism's own respective enzyme. Some enzymes exist in form of different isoforms within the same species. Hence, in such a case some members of the same species have a first isoform while other members of the species have a second isoform. A homologous enzyme may be either isoform. In other words a homologous enzyme may be an isoform of an enzyme that is different from the enzyme encoded by a gene of the particular microorganism, but it may be an isoform of the enzyme that is found in some other members of the same species.

The term “homologous sequence”, as used herein, refers to the sequence similarity between two polymeric molecules, e.g., between two nucleic acid molecules, e.g., two DNA molecules or two RNA molecules, or between two polypeptide molecules. Two sequences can be termed homologous if a tfasty search for a given sequence results in an expectancy score (E value) of less than 0.001 for a further sequence.

As used herein, the term “heterologous” refers to a molecule or activity derived from a source other than the referenced species whereas “homologous” refers to a molecule or activity derived from the host microbial organism. Accordingly, exogenous expression of an encoding nucleic acid as described herein can utilize either or both a heterologous or homologous encoding nucleic acid. A “heterologous” enzyme, as used herein in the context of a microorganism, is an enzyme that is produced or synthesized from a nucleic acid sequence that naturally occurs in a species that differs from the species of the respective microorganism.

The terms “identity” or “sequence identity” in the context of an amino acid sequence or nucleic acid sequence refers to the sequence match between two proteins or nucleic acids. The trem thus defines a property of sequences that measures their similarity or relationship. The protein or nucleic acid sequences to be compared are aligned to give maximum identity. Methods for aligning sequences for comparison are well known in the art. An illustrative example is the NCBI Basic Local Alignment Search Tool (BLAST™; Altschul et al., J. Mol. Biol. (1990) 215, 403-410), available from several sources, including the National Center for Biotechnology Information (NCBI, Bethesda, Md., U.S.A.), which can be used in connection with the sequence analysis programs blastp, blastn, blastx, tblastn and tblastx. BLAST™ can be accessed on the Internet at the NCBI website. A further example is the tool EMBOSS Needle (pair wise alignment; available at www.ebi.ac.uk). When the same position in the sequences to be compared is occupied by the same nucleobase or amino acid residue, then the respective molecules are identical at that very position. The “sequence identity” or “identity” can be taken to define the percentage of pair-wise identical residues—following (homology) alignment of a sequence of a particular polypeptide with a reference sequence—with respect to the number of residues in the longer of these two sequences. Identity is measured by dividing the number of identical residues by the total number of residues and multiplying the product by 100. For instance, if 6 out of 10 sequence positions are identical, then the identity is 60%. The percent identity between two protein sequences can, e.g., be determined using the Needleman and Wunsch algorithm (NEEDLEMAN, S. B. and Wunsch, C. D., JMB (1970,) 48, 443-453) which has been incorporated into EMBOSS Needle, using a BLOSUM62 matrix, a “gap open penalty” of 10, a “gap extend penalty” of 0.5, a false “end gap penalty”, an “end gap open penalty” of 10 and an “end gap extend penalty” of 0.5. Two molecules having the same primary amino acid or nucleic acid sequence are identical irrespective of any chemical and/or biological modification. For example, two enzymes having the same primary amino acid sequence but different glycosylation patterns are identical by this definition. In case of nucleic acids, for example, two molecules having the same sequence but different linkage components such as thiophosphate instead of phosphate are identical by this definition.

The percentage of sequence homology or sequence identity can, for example, be determined using the program BLASTP, e.g. version blastp 2.2.5 (Nov. 16, 2002; cf. Altschul, S. F. et al. (1997) Nucl. Acids Res. 25, 3389-3402). In this embodiment the percentage of homology may be based on the alignment of the entire polypeptide sequences (matrix: BLOSUM 62; gap costs: 11.1; cutoff value set to 10⁻³) including the propeptide sequences, using the known native protein as reference in a pairwise comparison. It is calculated as the percentage of numbers of “positives” (homologous amino acids) indicated as result in the BLASTP program output divided by the total number of amino acids selected by the program for the alignment. It is noted in this connection that this total number of selected amino acids can differ from the length of the known native protein.

The term “mutated” or “mutant” in reference to a nucleic acid or a polypeptide refers to the exchange, deletion, or insertion of one or more nucleotides or amino acids, respectively, compared to the naturally occurring nucleic acid molecule or polypeptide. A respective mutant may for instance include one or more substitutions in comparison to the corresponding native amino acid sequence or nucleic acid sequence.

The term “mutagenesis” as used herein means that the experimental conditions are chosen such that the amino acid naturally occurring at a given sequence position of a selected protein can be substituted by at least one amino acid that is not present at this specific position in the respective natural polypeptide sequence. As already noted above, the term “mutagenesis” also includes the (additional) modification of the length of sequence segments by deletion or insertion of one or more amino acids. Thus, for example, one amino acid at a chosen sequence position may replaced by a stretch of three random mutations, leading to an insertion of two amino acid residues compared to the length of the respective segment of the wild type protein. Such an insertion of deletion may be introduced independently from each other in any of the peptide segments that can be subjected to mutagenesis. The term “random mutagenesis” means that no predetermined single amino acid (mutation) is present at a certain sequence position but that at least two amino acids can be incorporated with a certain probability at a predefined sequence position during mutagenesis.

The coding sequence of a selected protein may be used as a starting point for mutagenesis. For the mutagenesis of a selected amino acid position, the person skilled in the art has at his disposal various established standard methods for site-directed mutagenesis. A commonly used technique is the introduction of mutations by means of PCR (polymerase chain reaction) using mixtures of synthetic oligonucleotides, which bear a degenerate base composition at the desired sequence positions. For example, use of the codon NNK or NNS (wherein N=adenine, guanine or cytosine or thymine; K=guanine or thymine; S=adenine or cytosine) allows incorporation of all 20 amino acids plus the amber stop codon during mutagenesis, whereas the codon VVS limits the number of possibly incorporated amino acids to 12, since it excludes the amino acids Cys, Ile, Leu, Met, Phe, Trp, Tyr, Val from being incorporated into the selected position of the polypeptide sequence; use of the codon NMS (wherein M=adenine or cytosine), for example, restricts the number of possible amino acids to 11 at a selected sequence position since it excludes the amino acids Arg, Cys, Gly, Ile, Leu, Met, Phe, Trp, Val from being incorporated at a selected sequence position. In this respect it is noted that codons for amino acids that differ from the regular 20 naturally occurring amino acids, such as selenocystein or pyrrolysine can also be incorporated into a nucleic acid. It is also possible, as described by Wang, L., et al. Science (2001) 292, 498-500, or Wang, L., and Schultz, P. G. Chem. Comm. (2002) 1, 1-11, to use “artificial” codons such as UAG which are usually recognized as stop codons in order to insert other unusual amino acids, for example o-methyl-L-tyrosine or p-aminophenylalanine. The use of nucleotide building blocks with reduced base pair specificity, as for example inosine, 8-oxo-2′deoxyguanosine or 6(2-deoxy-β-D-ribofuranosyl)-3,4-dihydro-8H-pyrimido-1,2-oxazine-7-one (Zaccolo et al. J. Mol. Biol. (1996) 255, 589-603), is another option for the introduction of mutations into a chosen sequence segment. A further possibility is the so-called triplet-mutagenesis. This method uses mixtures of different nucleotide triplets, each of which codes for one amino acid, for incorporation into the coding sequence (Virnekäs et al., Nucleic Acids Res (1994) 22, 5600-5607).

The term “non-naturally occurring” when used herein in the context of a microorganism is intended to mean that the microorganism has one or more genetic alterations not normally found in a naturally occurring strain of the referenced species, including wild-type strains of the referenced species. Genetic alterations include, for example, modifications introducing an expressible nucleic acid sequence that encodes metabolic polypeptides, in particular one or more enzymes. A respective expressible nucleic acid sequence may be included in the genome of the microorganism, or it may be included in a nucleic acid molecule that is different from the genome of the microorganism. Genetic alterations may also include other nucleic acid additions, nucleic acid deletions and/or other functional disruption of the microbial organism's genetic material. Such modifications include, for example, coding regions and functional fragments thereof, for heterologous, homologous or both heterologous and homologous polypeptides for the referenced species. Additional modifications include, for example, non-coding regulatory regions in which the modifications alter expression of a gene or operon. Exemplary metabolic polypeptides include enzymes or proteins within an alkene biosynthetic pathway.

A metabolic modification refers to a biochemical reaction that is altered from its naturally occurring state. Accordingly, a non-naturally occurring microorganism can have a genetic modification to a nucleic acid that encodes a metabolic polypeptide, or a functional fragment thereof

The term “nucleic acid molecule” as used herein refers to any nucleic acid in any possible configuration, such as single stranded, double stranded or a combination thereof. Examples of nucleic acids include for instance DNA molecules, RNA molecules, analogues of the DNA or RNA generated using nucleotide analogues or using nucleic acid chemistry, locked nucleic acid molecules (LNA), protein nucleic acids molecules (PNA), alkylphosphonate and alkylphosphotriester nucleic acid molecules and tecto-RNA molecules (e.g. Liu, B., et al., J. Am. Chem. Soc. (2004) 126, 4076-4077). LNA has a modified RNA backbone with a methylene bridge between C4′ and O2′, providing the respective molecule with a higher duplex stability and nuclease resistance. Alkylphosphonate and alkylphosphotriester nucleic acid molecules can be viewed as a DNA or an RNA molecule, in which phosphate groups of the nucleic acid backbone are neutralized by exchanging the P—OH groups of the phosphate groups in the nucleic acid backbone to an alkyl and to an alkoxy group, respectively. DNA or RNA may be of genomic or synthetic origin and may be single or double stranded. Such nucleic acid can be e.g. mRNA, cRNA, synthetic RNA, genomic DNA, cDNA synthetic DNA, a copolymer of DNA and RNA, oligonucleotides, etc. A respective nucleic acid may furthermore contain non-natural nucleotide analogues and/or be linked to an affinity tag or a label.

Many nucleotide analogues are known and can be used in nucleic acids used in the methods described herein. A nucleotide analogue is a nucleotide containing a modification at for instance the base, sugar, or phosphate moieties. As an illustrative example, a substitution of 2′-OH residues of siRNA with 2′F, 2′O-Me or 2′H residues is known to improve the in vivo stability of the respective RNA. Modifications at the base moiety may be a natural or a synthetic modification of A, C, G, and T/U, a different purine or pyrimidine base, such as uracil-5-yl, hypoxanthin-9-yl, and 2-aminoadenin-9-yl, as well as a non-purine or a non-pyrimidine nucleotide base. Other nucleotide analogues serve as universal bases. Examples of universal bases include 3-nitropyrrole and 5-nitroindole. Universal bases are able to form a base pair with any other base. Base modifications often can be combined with for example a sugar modification, such as for instance 2′-O-methoxyethyl, e.g. to achieve unique properties such as increased duplex stability.

The term “nucleic acid construct” as used herein refers to a nucleic acid molecule, either single- or double-stranded, which is isolated from a naturally occurring gene or which is modified to contain segments of nucleic acids in a manner that would not otherwise exist in nature or which is synthetic. The term nucleic acid construct is synonymous with the term “expression cassette” when the nucleic acid construct contains the control sequences required for expression of a particular coding sequence.

The term “operatively linked to” refers to the functional relationship of a nucleic acid sequence with another nucleic acid sequence. Promoters, enhancers, transcriptional and translational stop sites, and other signal sequences are examples of nucleic acid sequences operatively linked to other sequences. For example, operative linkage of DNA to a transcriptional control element refers to the physical and functional relationship between the DNA and promoter such that the transcription of such DNA is initiated from the promoter by an RNA polymerase that specifically recognizes, binds to and transcribes the DNA.

As used herein, the terms “polypeptide” and “protein” refer to a polymer of amino acid residues and are not limited to a certain minimum length of the product. Where both terms are used concurrently, this twofold naming accounts for the use of both terms side by side in the art.

The term “promoter” as used throughout this document, refers to a nucleic acid sequence needed for gene sequence expression. Promoter regions vary from organism to organism, but are well known to those skilled in the art for different organisms. For example, in prokaryotes, the promoter region contains both the promoter (which directs the initiation of RNA transcription) as well as the DNA sequences which, when transcribed into RNA, will signal synthesis initiation. Such regions will normally include those 5′-non-coding sequences involved with initiation of transcription and translation, such as the TATA box, capping sequence, CAAT sequence, and the like. Both constitutive and inducible promoters can be used in the context of a method disclosed herein. A large number of promoters recognized by a variety of potential host cells are well known. The selected promoter can be operably linked to cistron DNA encoding a polypeptide described herein by removing the promoter from the source DNA via restriction enzyme digestion and inserting the isolated promoter sequence into the vector of choice. Both the native promoter sequence and many heterologous promoters may be used to direct amplification and/or expression of a selected nucleic acid sequence.

The word “recombinant” is used in this document to describe a nucleic acid molecule that, by virtue of its origin, manipulation, or both is not associated with all or a portion of the nucleic acid molecule with which it is associated in nature. Generally a recombinant nucleic acid molecule includes a sequence which does not naturally occur in the respective wildtype organism or cell. Typically a recombinant nucleic acid molecule is obtained by genetic engineering, usually constructed outside of a cell. Generally a recombinant nucleic acid molecule is substantially identical and/or substantial complementary to at least a portion of the corresponding nucleic acid molecule occurring in nature. A recombinant nucleic acid molecule may be of any origin, such as genomic, cDNA, mammalian, bacterial, viral, semisynthetic or synthetic origin The term “recombinant” as used with respect to a protein/polypeptide means a polypeptide produced by expression of a recombinant polynucleotide.

“Similar” protein sequences are those which, when aligned, share similar amino acid residues and most often, but not mandatorily, identical amino acid residues at the same positions of the sequences to be compared. Similar amino acid residues are grouped by chemical characteristics of the side chains into families. These families are described below for “conservative amino acid substitutions”. The “percent similarity” between sequences is the number of positions that contain identical or similar residues at the same sequence positions of the sequences to be compared divided by the total number of positions compared and multiplied by 100%. For instance, if 6 out of 10 sequence positions have identical amino acid residues and 2 out of 10 positions contain similar residues, then the sequences have 80% similarity. The similarity between two sequences can e.g. be determined using EMBOSS Needle.

The term “stringent conditions” refers to the technique of hybridizing nucleic acid molecules. Nucleic acid hybridization reactions can be performed under conditions of different stringency. “Stringent conditions” are widely known and published in the art. Typically, during the hybridization reaction a SSC-based buffer can be used in which SSC is 0.15 M NaCl and 15 mM citrate buffer having a pH of 7.0. Increasing buffer concentrations and the presence of a denaturing agent increase the stringency of the hybridization step. For example, high stringency hybridization conditions can involve the use of (i) 50% (v/v) formamide, 5×SSC (0.75 M NaCl, 0.075 M sodium citrate), 50 mM sodium phosphate (pH 6.8), 0.1% sodium pyrophosphate, 5×Denhardt's solution, sonicated salmon sperm DNA (50 μg/mL), 0.1% SDS, and 10% dextran sulphate at 42° C. with washes at 42° C. in 0.2×SSC and 0.1% SDS; (ii) 50% (v/v) formamide with 0.1% bovine serum albumin/0.1% Ficoll/0.1% polyvinylpyrrolidone/50 mM sodium phosphate buffer at pH 6.5 with 750 mM sodium chloride, 75 mM sodium citrate at 42° C., or (iii) 10% dextran sulphate, 2×SSC, and 50% formamide at 55° C., followed by a high-stringency wash consisting of 0.1×SSC containing EDTA at 55° C. Additionally or alternatively, one, two or more washing steps using wash solutions of low ionic strength and high temperature can be included in the hybridization protocol using, for example, 0.015 M sodium chloride/0.0015 M sodium citrate/0.1% sodium dodecyl sulphate at 50° C.

The term “syngas” refers to a mixture of carbon monoxide, carbon dioxide and hydrogen.

The terms “transposon” or “transposable element”, as used herein, are meant to refer to a nucleotide sequence that is able to excise or copy from a donor polynucleotide, for instance, a vector, and integrate into a target site, for instance, a cell's genomic or extrachromosomal DNA. The process of excising or copying a defined DNA segment from one nucleic acid molecule and moving the same to another site in the same or a different nucleic acid molecule is called transposition. A transposon may be a “class 2” element, also called “DNA transposon”. Class 2 elements are excised from a donor polynucleotide and inserted into a target nucleic acid molecule. This process is catalyzed by a functional transposase protein. The transposase protein is in some cases encoded by the transposon. Such a transposon is also termed an autonomous transposon. A transposon contains a nucleic acid sequence flanked by cis-acting nucleotide sequences on the termini of the transposon. A nucleic acid sequence is “flanked by” cis-acting nucleotide sequences if at least one cis-acting nucleotide sequence is positioned 5′ to the nucleic acid sequence, and at least one cis-acting nucleotide sequence is positioned 3′ to the nucleic acid sequence. For class 2 elements the cis-acting nucleotide sequences include at least one inverted repeat (also referred to herein as an inverted terminal repeat, or ITR) at each end of the transposon, to which the transposase binds. A transposon may also be a “class 1” element, also called “retrotransposon”. A class 1 element does not encode a transposase enzyme; rather, it produces RNA transcripts and then relies upon reverse transcriptase enzymes to reverse transcribe the RNA sequence(s) into DNA, which is then inserted into the target site. A class 1 element is copied from a donor polynucleotide, i.e. it leaves behind the original copy and generates a second copy that is inserted into the target site. Class 1 elements are divided into autonomous and nonautonomous classes depending on whether they do or do not encompass a sequence that encodes proteins required for retrotransposition. The capability of a transposon to excise or copy from a donor polynucleotide and integrate into a target site can be silenced, for example by methylation and/or small interfering RNA molecules.

A “variant” refers to an amino acid or nucleic acid sequence which differs from the parental sequence by virtue of addition (including insertions), deletion and/or substitution of one or more amino acid residues or nucleobases while retaining at least one desired activity of the parent sequence disclosed herein. In the case of an enzyme such desired activity may include specific substrate binding. Similarly, a variant nucleic acid sequence may be modified when compared to the parent sequence by virtue of addition, deletion and/or substitution of one or more nucleobases, but the encoded protein, e.g. enzyme, retains the desired activity as described above. Variants may be naturally occurring, such as allelic or splice variants, or may be artificially constructed.

The term “vector”, sometimes also referred to as gene delivery system or gene transfer vehicle, relates to a macromolecule or complex of molecules that include(s) a polynucleotide to be delivered to a host cell, whether in vitro, ex vivo or in vivo. Typically a vector is a single or double-stranded circular nucleic acid molecule that allows or facilitates the transfer of a nucleic acid sequence into a cell. A vector can generally be transfected into cells and replicated within or independently of a cell genome. A circular double-stranded nucleic acid molecule can be cut and thereby linearized upon treatment with restriction enzymes. An assortment of nucleic acid vectors, restriction enzymes, and the knowledge of the nucleotide sequences cut by restriction enzymes are readily available to those skilled in the art. A nucleic acid molecule encoding a peptide, such as a sequence that encodes the amino acid sequence of GenBank accession no. WP_003251320.1 or a homolog thereof, and/or a sequence encoding the amino acid sequence of GenBank accession no. WP_003243190.1 or a homolog thereof, can be inserted into a vector by cutting the vector with restriction enzymes and ligating the two pieces together. A vector may for instance be a viral vector, such as a retroviral vector, a Lentiviral vector, a herpes virus based vector or an adenoviral vector. A vector may also be a plasmid vector, which is also a typical example of a prokaryotic vector. A respective plasmid may in some embodiments be a plasmid capable of replication in E. coli, such as, for example, pBR322, ColEl, pSC101, pACYC 184 or πVX. Bacillus plasmids include pC194, pC221 or pT127. Suitable Streptomyces plasmids include p1θ101, and streptomyces bacteriophages such as φC31. A vector may also be a liposome-based extrachromosomal vector, also called episomal vector. Two illustrative examples of an episomal vector are an oriP-based vector and a vector encoding a derivative of EBNA-1. Lymphotrophic herpes virus is a herpes virus which replicates in a lymphoblast and becomes a plasmid for a part of its natural life-cycle. A vector may also be based on an organically modified silicate. In some embodiments a vector may be a transposon-based system, as explained above.

The terms “comprising”, “including,” containing”, “having” etc. shall be read expansively or open-ended and without limitation. Singular forms such as “a”, “an” or “the” include plural references unless the context clearly indicates otherwise. Thus, for example, reference to a “vector” includes a single vector as well as a plurality of vectors, either the same—e.g. the same operon—or different. Likewise reference to “cell” includes a single cell as well as a plurality of cells. Unless otherwise indicated, the term “at least” preceding a series of elements is to be understood to refer to every element in the series. The terms “at least one” and “at least one of” include for example, one, two, three, four, or five or more elements. It is furthermore understood that slight variations above and below a stated range can be used to achieve substantially the same results as a value within the range. Also, unless indicated otherwise, the disclosure of ranges is intended as a continuous range including every value between the minimum and maximum values.

The scope and meaning of any use of a term will be apparent from the specific context in which the term is used. Certain further definitions for selected terms used throughout this document are given in the appropriate context of the detailed description, as applicable. Unless otherwise defined, all other scientific and technical terms used in the description, figures and claims have their ordinary meaning as commonly understood by one of ordinary skill in the art.

Various aspects of the disclosure are described in further detail in the following subsections. It is understood that the various embodiments, preferences and ranges may be combined at will. Further, depending of the specific embodiment, selected definitions, embodiments or ranges may not apply.

### Enzymes and Pathways

A recombinant microorganism as described herein generally contains one or more heterologous nucleic acid sequences that provide, complete or enhance a biosynthesis pathway. The A recombinant microorganism described herein is generally capableof expressing a number of enzymes that together define a biosynthesis pathway leading to the formation of an alkane and/or an alkene. One or more of the enzymes of the biosynthesis pathway are encoded by a heterologous nucleic acid sequence. Typically the enzymes of the biosynthesis pathway are expressed in a sufficient amount to allow production of an alkane and/or alkene. Thereby the recombinant microorganism may be capable of converting carbon monoxide and/or carbon dioxide to an alkane and/or alkene. A recombinant microorganism as described herein may contain nucleic acid sequences encoding enzymes of a pathway leading to the formation of methane. In some embodiments a recombinant microorganism described herein may contain nucleic acid sequences encoding enzymes of a pathway leading to the formation of ethane.

A recombinant microorganism as described herein may contain nucleic acid sequences that encode enzymes of a pathway leading to the formation of ethene. One or more of these enzymes may be encoded by a heterologous nucleic acid sequence. A respective heterologous nucleic acid sequence may originate from a heterologous nucleic acid molecule. In some embodiments the recombinant microorganism may contain nucleic acid sequences that encode all enzymes of a pathway leading to the formation of ethane. In some embodiments the recombinant microorganism does not contain nucleic acid sequences encoding all enzymes of a pathway leading to the formation of ethane. In some embodiments a recombinant microorganism as described herein may be capable of producing ethene. In some embodiments a recombinant microorganism as described herein is not capable of producing ethane. A recombinant microorganism disclosed herein may contain nucleic acid sequences encoding enzymes of a pathway leading to the formation of propane. One or more of these enzymes may be encoded by a heterologous nucleic acid sequence. In some embodiments the recombinant microorganism may contain nucleic acid sequences that encode all enzymes of a pathway leading to the formation of propane. In some embodiments the recombinant microorganism does not contain nucleic acid sequences encoding all enzymes of a pathway leading to the formation of propane. In some embodiments a recombinant microorganism as described herein is capable of producing propane.

A recombinant microorganism as described herein may contain nucleic acid sequences encoding enzymes of a pathway leading to the formation of propene. One or more of these enzymes may be encoded by a heterologous nucleic acid sequence. In some embodiments two or three of these enzymes may be encoded by a heterologous nucleic acid sequence. As noted above, a respective heterologous nucleic acid sequence may originate from a heterologous nucleic acid molecule. In some embodiments the recombinant microorganism may contain nucleic acid sequences that encode all enzymes of a pathway leading to the formation of propene. In some embodiments the recombinant microorganism does not contain nucleic acid sequences encoding all enzymes of a pathway leading to the formation of propene. A recombinant microorganism as described herein may in some embodiments be capable of producing propene. In some embodiments a recombinant microorganism as described herein is not capable of producing propene. A recombinant microorganism disclosed herein may contain nucleic acid sequences encoding enzymes of a pathway leading to the formation of n-butane. One or more of the enzymes of this pathway may be encoded by a heterologous nucleic acid sequence. In some embodiments the recombinant microorganism may contain nucleic acid sequences that encode all enzymes of a pathway leading to the formation of n-butane. In some embodiments the recombinant microorganism does not contain nucleic acid sequences encoding all enzymes of a pathway leading to the formation of n-butane. In some embodiments a recombinant microorganism as described herein is capable of producing propane. A recombinant microorganism as described herein may contain nucleic acid sequences encoding enzymes of a pathway leading to the formation of 1-butene. One or more of the enzymes of this pathway may be encoded by a heterologous nucleic acid sequence. In some embodiments two or three of the enzymes of this pathway may be encoded by a heterologous nucleic acid sequence. As noted above, a respective heterologous nucleic acid sequence may originate from a heterologous nucleic acid molecule. In some embodiments the recombinant microorganism may contain nucleic acid sequences that encode all enzymes of a pathway leading to the formation of 1-butene. In some embodiments the recombinant microorganism does not contain nucleic acid sequences encoding all enzymes of a pathway leading to the formation of 1-butene. In some embodiments a recombinant microorganism as described herein may be capable of producing 1-butene. In some embodiments a recombinant microorganism as described herein is not capable of producing 1-butene.

In some embodiments a recombinant microorganism described herein may contain nucleic acid sequences encoding enzymes of a pathway leading to the formation of isobutene. One or more of the enzymes of this pathway may be encoded by a heterologous nucleic acid sequence. In some embodiments at least two or three of the enzymes of a pathway leading to the formation of isobutene may be encoded by a heterologous nucleic acid sequence. A respective heterologous nucleic acid sequence may originate from a heterologous nucleic acid molecule. In some embodiments the recombinant microorganism may contain nucleic acid sequences that encode all enzymes of a pathway leading to the formation of isobutene. In some embodiments the recombinant microorganism does not contain nucleic acid sequences encoding all enzymes of a pathway leading to the formation of isobutene. In some embodiments a recombinant microorganism as described herein may be capable of producing isobutene. In some embodiments a recombinant microorganism as described herein is not capable of producing isobutene. A recombinant microorganism disclosed herein may also contain nucleic acid sequences encoding enzymes of a pathway leading to the formation of 1,3-butadiene. One or more of the enzymes of this pathway may be encoded by a heterologous nucleic acid sequence. In some embodiments at least two or three of the enzymes of this pathway may be encoded by a heterologous nucleic acid sequence. As indicated above, a respective heterologous nucleic acid sequence may originate from a heterologous nucleic acid molecule. In some embodiments the recombinant microorganism may contain nucleic acid sequences that encode all enzymes of a pathway leading to the formation of 1,3-butadiene. In some embodiments the recombinant microorganism does not contain nucleic acid sequences encoding all enzymes of a pathway leading to the formation of 1,3-butadiene. A recombinant microorganism as described herein may in some embodiments be capable of producing 1,3-butadiene. In some embodiments a recombinant microorganism as described herein is not capable of producing 1,3-butadiene.

A recombinant microorganism as described herein may contain nucleic acid sequences encoding enzymes of a pathway leading to the formation of n-pentane. In some embodiments the recombinant microorganism may contain nucleic acid sequences that encode all enzymes of a pathway leading to the formation of n-pentane. In some embodiments the recombinant microorganism does not contain nucleic acid sequences encoding all enzymes of a pathway leading to the formation of n-pentane. In some embodiments a recombinant microorganism as described herein is capable of producing n-pentane. A recombinant microorganism as described herein may contain nucleic acid sequences encoding enzymes of a pathway leading to the formation of 1-pentene. One or more of the enzymes of this pathway may be encoded by a heterologous nucleic acid sequence. In some embodiments at least two or three of the enzymes of this pathway may be encoded by a heterologous nucleic acid sequence. A respective heterologous nucleic acid sequence may originate from a heterologous nucleic acid molecule. In some embodiments the recombinant microorganism may contain nucleic acid sequences that encode all enzymes of a pathway leading to the formation of 1-pentene. In some embodiments the recombinant microorganism does not contain nucleic acid sequences encoding all enzymes of a pathway leading to the formation of 1-pentene. In some embodiments a recombinant microorganism as described herein may be capable of producing 1-pentene. In some embodiments a recombinant microorganism as described herein is not capable of producing 1-pentene.

In some embodiments a recombinant microorganism described herein may contain nucleic acid sequences encoding enzymes of a pathway leading to the formation of n-hexane. One or more of the enzymes of this pathway may be encoded by a heterologous nucleic acid sequence. In some embodiments the recombinant microorganism may contain nucleic acid sequences that encode all enzymes of a pathway leading to the formation of n-hexane. In some embodiments the recombinant microorganism does not contain nucleic acid sequences encoding all enzymes of a pathway leading to the formation of n-hexane. In some embodiments a recombinant microorganism as described herein is capable of producing n-hexane. A recombinant microorganism as described herein may contain nucleic acid sequences encoding enzymes of a pathway leading to the formation of 1-hexene. One or more of the enzymes of this pathway may be encoded by a heterologous nucleic acid sequence. In some embodiments at least two or three of the enzymes of this pathway may be encoded by a heterologous nucleic acid sequence. In some embodiments the recombinant microorganism may contain nucleic acid sequences that encode all enzymes of a pathway leading to the formation of 1-hexene. In some embodiments the recombinant microorganism does not contain nucleic acid sequences encoding all enzymes of a pathway leading to the formation of 1-hexene. In some embodiments a recombinant microorganism as described herein is capable of producing 1-hexene.

In some embodiments a recombinant microorganism described herein may contain nucleic acid sequences encoding enzymes of a pathway leading to the formation of isoprene, also called 2-methyl-1,3-butadiene. One or more of the enzymes of the pathway leading to the formation of isoprene may be encoded by a heterologous nucleic acid sequence. In some embodiments at least two or three of the enzymes of a pathway leading to the formation of 2-methyl-1,3-butadiene may be encoded by a heterologous nucleic acid sequence. A respective heterologous nucleic acid sequence may originate from a heterologous nucleic acid molecule. In some embodiments the recombinant microorganism may contain nucleic acid sequences that encode all enzymes of a pathway leading to the formation of 2-methyl-1,3-butadiene. The recombinant microorganism does in some embodiments not contain nucleic acid sequences encoding all enzymes of a pathway leading to the formation of 1-hexene. A recombinant microorganism as described herein may in some embodiments be capable of producing 2-methyl-1,3-butadiene. In some embodiments a recombinant microorganism as described herein is not capable of producing 2-methyl-1,3-butadiene.

The heterologous nucleic acid sequences encode one or more enzymes that allow the enzymatic conversion of acetyl-CoA to one or more alkanes and/or alkenes with a main chain of 2 to 6 carbon atoms. In some embodiments the heterologous nucleic acid sequences encode two or more enzymes that allow the enzymatic conversion of acetyl-CoA to one or more alkenes with a main chain of 2 to 5 carbon atoms. Acetyl-CoA or Acetyl coenzyme A is a thioester of acetic acid and a β-mercaptoethylamine moiety of coenzyme A. It serves as a universal component in metabolism and can be regarded as an activated carrier of an acetyl group. Acetyl-CoA is generated by the degradation of inter alia fatty acids as well as carbohydrates, and serves as a starting substance in a variety of biosynthesis pathways. Acetyl-CoA does not readily cross membranes, so that it remains within the cell and can function as a reactant.

Illustrative examples of biosynthesis pathways that may be established, completed or enhanced using the methods, nucleic acid sequences and vectors disclosed herein are depicted in the figures. In some embodiments the recombinant microorganism further expresses enzymes of the Wood-Ljungdahl-pathway. As a result the recombinant microorganism is capable of synthesizing acetyl-CoA from carbon monoxide and/or carbon dioxide. The enzymes of the Wood-Ljungdahl-pathway that the microorganism expresses may in some embodiments be encoded by and expressed from endogenous nucleic acid sequences.

A recombinant microorganism as described herein may contin a heterologous nucleic acid sequence that encodes an enzyme described in the following. The encoded protein may be any allelic variant of a respective enzyme. The respective heterologous nucleic acid sequence may in some embodiments be the nucleotide sequence found in a sequence database. The heterologous nucleic acid sequence included in the recombinant microorganism may be a homolog of a nucleic acid encoding an enzyme described below. In some embodiments the heterologous nucleic acid sequence included in the recombinant microorganism may contain a conservative modification, which essentially maintains the enzymatic activity of the peptide/protein. In same embodiments a respective conservative modification leaves the enzymatic activity of the peptide/protein essentially unchanged. A heterologous nucleic acid sequence included in the recombinant microorganism may be codon-optimized, i.e. it may have been adapted for expression in the cells of a given organism by replacing one or more, such as two or three, or a significant number, of codons with one or more codons that are more frequently used in the genes of that organism. Codon optimization is well known in the art. In general, highly expressed genes in an organism are biased towards codons that are recognized by the most abundant tRNA species in that organism. One measure of this bias is the “codon adaptation index” or “CAI”, which measures the extent to which the codons used to encode each amino acid in a particular gene are those which occur most frequently in a reference set of highly expressed genes from an organism. Many organisms display a bias for use of particular codons to code for insertion of a particular amino acid in a growing peptide chain. Codon preference or codon bias, differences in codon usage between organisms, is afforded by degeneracy of the genetic code, and is well documented among many organisms. Codon bias often correlates with the efficiency of translation of messenger RNA (mRNA), which is in turn believed to be dependent on, inter alia, the properties of the codons being translated and the availability of particular transfer RNA (tRNA) molecules. The predominance of selected tRNAs in a cell is generally a reflection of the codons used most frequently in peptide synthesis.

A heterologous nucleic acid sequence included in the recombinant microorganism may in some embodiments have a sequence identity of 90% or more when compared to the sequence of a database entry of a peptide/protein described below. In some embodiments a heterologous nucleic acid sequence in the recombinant microorganism may have a sequence identity of 95% or more, such as 97% or more, when compared to the sequence of a database entry of a peptide/protein described below. In some embodiments a heterologous nucleic acid sequence in the recombinant microorganism may have a sequence identity of 99% or more, when compared to the sequence of a database entry of a peptide/protein described below. The heterologous nucleic acid sequence in the recombinant microorganism is in some embodiments included in a double stranded nucleic acid molecule. A complementary strand to the coding strand, i.e. to the sense strand, of the heterologous nucleic acid sequence may in some embodiments be capable of binding to a strand encoding the sequence of a peptide/protein described below under stringent conditions.

In some embodiments, a recombinant microorganism as described herein is generated from a host that already contains the enzymatic capability to synthesize the respective alkane and/or alkene. In such an embodiment it can be useful to increase the synthesis or accumulation of one or more intermediate products of the pathway to, for example, drive alkane/alkene pathway reactions toward alkene production. Increased synthesis or accumulation can be accomplished by, for example, overexpression of one or more nucleic acid sequences that encode one or more of the above-described enzymes. Overexpression of the enzyme or enzymes and/or protein or proteins of an alkane or alkene pathway can occur, for example, through exogenous expression of the endogenous gene or genes, or through exogenous expression of the heterologous gene or genes. Accordingly, a naturally occurring microorganism can be readily generated to be a non-naturally occurring microorganism, for example, producing an alkane and/or alkene, through overexpression of one or more, includingall nucleic acid sequences encoding enzymes of a respective alkane/alkene pathway. In some embodiments a recombinant microorganism can be generated by mutagenesis of an endogenous gene that results in an increase in activity of an enzyme in an alkane/alkene biosynthetic pathway.

In some embodiments the recombinant microorganism contains heterologous nucleic acid sequences that encode enzymes, which allow the conversion of acetyl-CoA to ethene. In some embodiments the microorganism contains one or more heterologous nucleic acid sequences that encode the enzymes lactate CoA-transferase (EC 2.8.3.-) and lactoyl-CoA dehydratase (EC 4.2.1.54). An illustrative example of a lactoyl CoA dehydratase is the enzyme from Clostridium propionicum having an alpha subunit of SwissProt accession no. G3KIM4 (LCDA_CLOPR), version 13 of 26 Nov. 2014 (version 1 of the sequence) and a beta subunit of SwissProt accession no. Q9L3F8 (Q9L3F8_CLOPR), version 20 of 29 Oct. 2014 (version 1 of the sequence). The enzyme the enzyme lactate CoA-transferase (EC 2.8.3.-) may be an acy CoA transferase, e.g. the enzyme EC 2.8.3.1. A suitable example is the acetate CoA-transferase YdiF from Clostridium propionicum, which has a broad substrate specificity for fatty acids with up to about 4 carbon atoms, having SwissProt accession no. Q9L3F7 (Q9L3F7_CLOPR), version 38 of 1 Apr. 2015 (version 1 of the sequence).

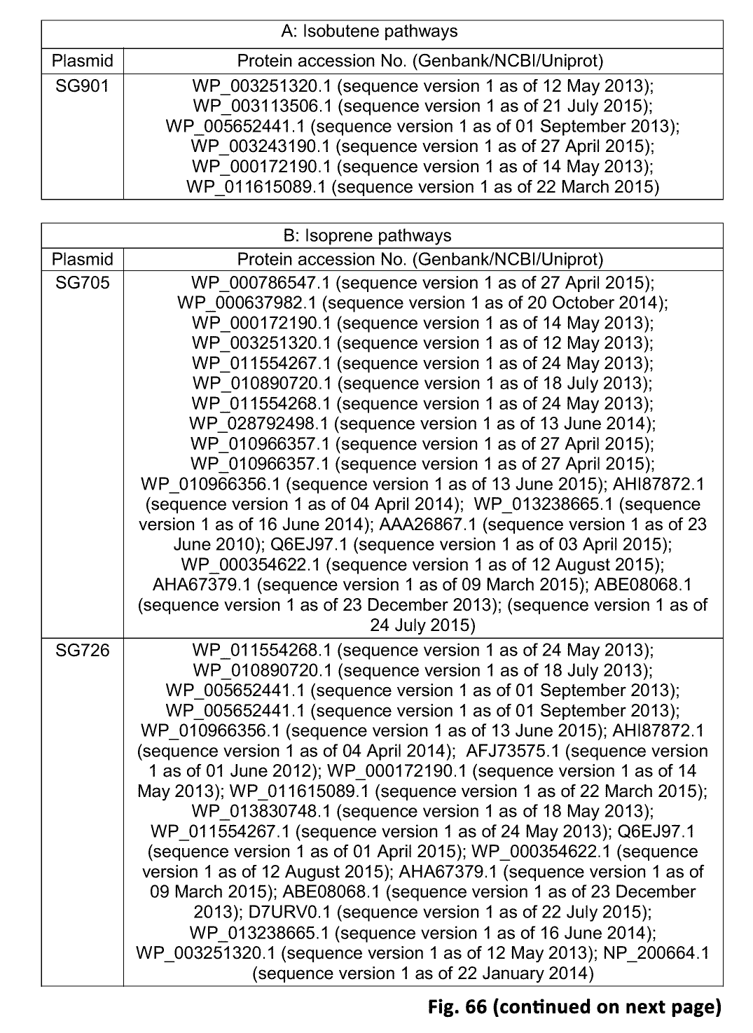

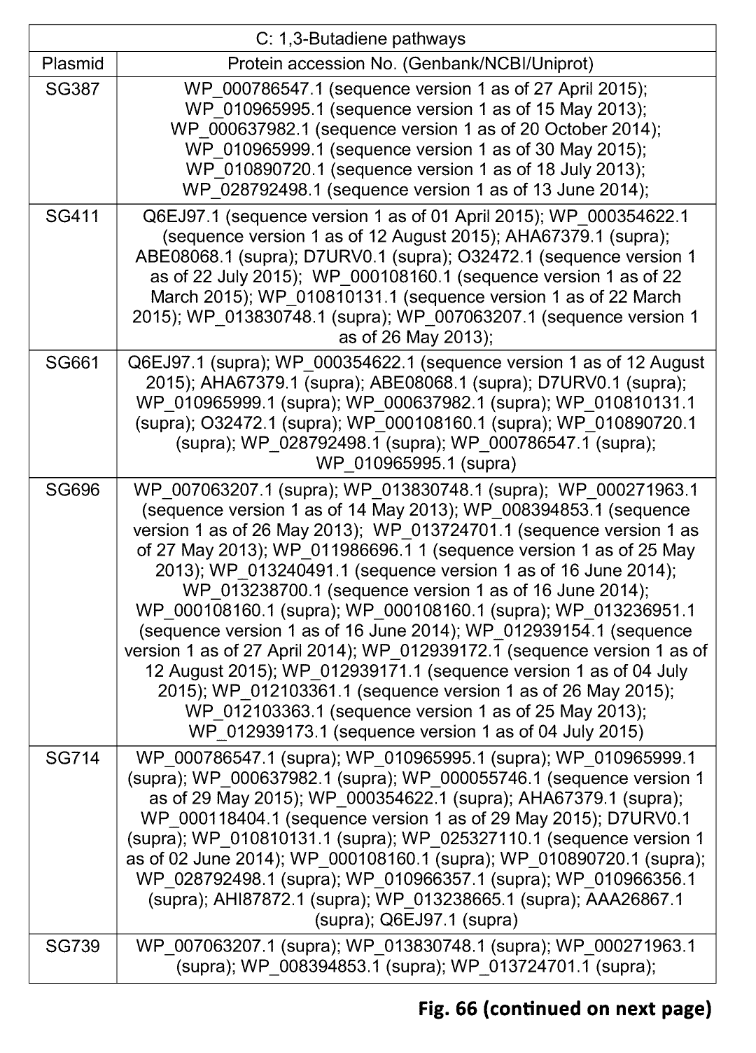

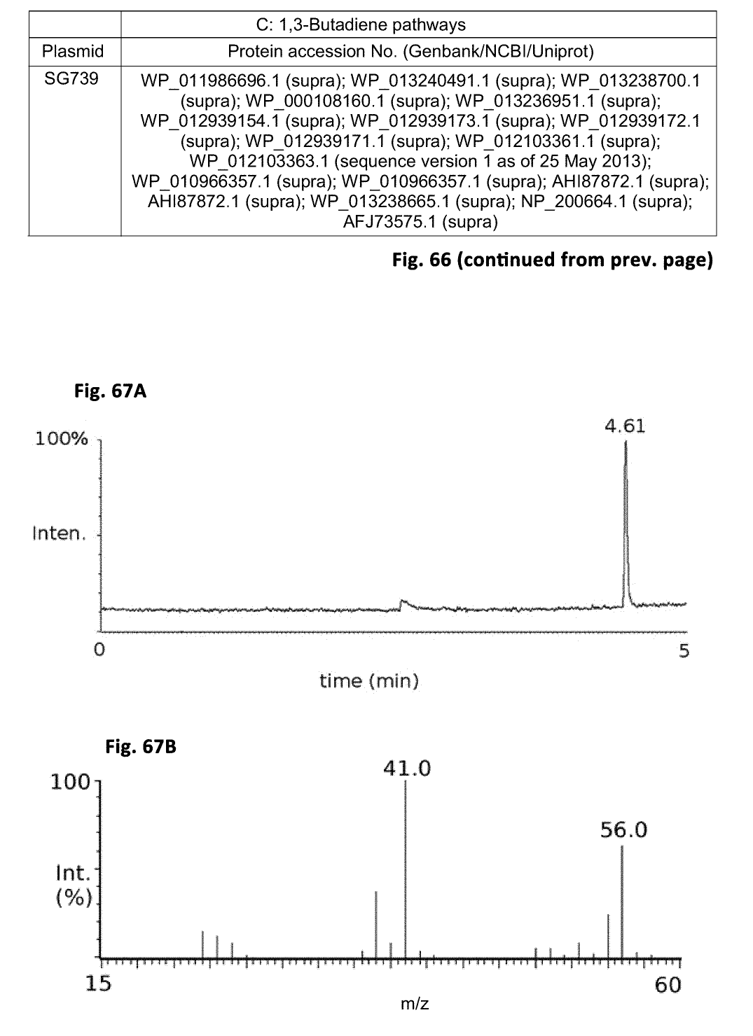

In some embodiments the microorganism contains a heterologous nucleic acid sequence that encodes the enzyme alcohol dehydrogenase. An example of a respective enzyme is alcohol dehydrogenase from Yokenella sp. WZY002 of SwissProt accession no. W6CX26, version 10 of 1 Apr. 2015 (version 1 of the sequence), which has GenBank accession no. AHI87872.1, cf. FIG. 66.

In some embodiments the microorganism contains a heterologous nucleic acid sequence that encodes the enzyme NADP-dependent malonyl CoA reductase (EC 1.2.1.75). An example of a respective enzyme is Malonyl CoA reductase from Metallosphaera sedula of SwissProt accession no. A4YEN2, version 56 of 27 May 2015 (version 1 of the sequence). A further example is malonyl-CoA reductase from Sulfolobus tokodaii with SwissProt accession no. Q96YK1, version 79 of 27 May 2015 (version 1 of the sequence).

In some embodiments the microorganism contains a heterologous nucleic acid sequence that encodes the enzyme malonyl CoA reductase (EC 1.1.1.298). An example of such an enzyme is NADP-dependent 3-hydroxy acid dehydrogenase reductase from E. coli of SwissProt accession no. P39831, version 114 of 22 Jul. 2015 (version 1 of the sequence). A further example of such an enzyme is the malonic semialdehyde reductase RutE from Cronobacter sakazakii of SwissProt accession no. A7MFX7, version 47 of 22 Jul. 2015 (version 1 of the sequence). Another suitable enzyme is the enzyme 3-hydroxy acid dehydrogenase from E. coli of SwissProt accession no. J7R133, version 22 of 22 Jul. 2015 (version 1 of the sequence).

In some embodiments the microorganism contains a heterologous nucleic acid sequence that encodes the enzyme 3-hydroxypropionyl-CoA synthetase (EC 6.2.1.36). An illustrative example of a 3-hydroxypropionyl-CoA synthetase is the enzyme from Congregibacter litoralis of SwissProt accession no. A4A7V6, version 53 of 24 Jun. 2015 (version 1 of the sequence). A further example of a 3-hydroxypropionyl-CoA synthetase is the enzyme from Metallosphaera sedula of SwissProt accession no. A4YGR1, version 49 of 22 Jul. 2015 (version 1 of the sequence). Yet a further example of a 3-hydroxypropionyl-CoA synthetase is the enzyme from Sulfolobus tokodaii of SwissProt accession no. Q973W5, version 75 of 22 Jul. 2015 (version 1 of the sequence).

In some embodiments the microorganism contains a heterologous nucleic acid sequence that encodes the enzyme 3-hydroxypropionyl-coenzyme A dehydratase (EC 4.2.1.116). An illustrative example of a 3-hydroxypropionyl-coenzyme A dehydratase is the enzyme from Sulfolobus tokodaii of SwissProt accession no. F9VNG3, version 11 of 22 Jul. 2015 (version 1 of the sequence). Another example of a 3-hydroxypropionyl-coenzyme A dehydratase is the enzyme from Metallosphaera sedula of SwissProt accession no. A4YI89, version 52 of 22 Jul. 2015 (version 1 of the sequence). Yet a further example of a 3-hydroxypropionyl-coenzyme A dehydratase is the enzyme from Bacillus thermoamylovorans of SwissProt accession no. A0A090IWI4, version 4 of 29 Apr. 2015 (version 1 of the sequence).

In some embodiments the microorganism contains a heterologous nucleic acid sequence that encodes an acyl-CoA thioesterase enzyme (EC 3.1.2.-). The enzyme may for instance be the enzyme 1,4-dihydroxy-2-naphthoyl-CoA hydrolase of E.C. 3.1.2.28 from E. coli of SwissProt accession no. P77781, version 121 of 22 Jul. 2015 (version 1 of the sequence), which is identical to GenBank accession no. WP_000637982.1 (see FIG. 66). This enzyme has activity toward a wide range of acyl-CoA thioesters. A further example of a 1,4-dihydroxy-2-naphthoyl-CoA hydrolase of E.C. 3.1.2.28 is the enzyme from Prochlorococcus marinus of SwissProt accession no. A2BNW8, version 53 of 22 Jul. 2015 (version 1 of the sequence). As a further example, the acyl-CoA thioesterase enzyme may be the enzyme acyl-CoA thioesterase 2 of E.C. 3.1.2.2 from E. coli of SwissProt accession no. P0AGG2, version 82 of 22 Jul. 2015 (version 2 of the sequence). A further example of an acyl-CoA thioesterase enzyme of EC 3.1.2.- is the acyl-CoA thioester hydrolase YbgC from E. coli of SwissProt accession no. P0A8Z3, version 87 of 22 Jul. 2015 (version 1 of the sequence). Another example of an acyl-CoA thioesterase enzyme of EC 3.1.2.- is the acyl-CoA thioesterase YbgC (EC 3.1.2.-) from Helicobacter pylori of SwissProt accession no. P94842, version 101 of 22 Jul. 2015 (version 2 of the sequence). Yet another example of an acyl-CoA thioesterase enzyme of EC 3.1.2.- is the acyl-CoA thioesterase YneP from Bacillus subtilis of SwissProt accession no. Q45061, version 81 of 24 Jun. 2015 (version 2 of the sequence). Yet another example of an acyl-CoA thioesterase enzyme of EC 3.1.2.- is the acyl-CoA thioesterase tesB (EC 3.1.2.-) from Klebsiella pneumonia of SwissProt accession no. W9BNH2, version 15 of 22 Jul. 2015 (version 1 of the sequence).

In some embodiments the microorganism contains a heterologous nucleic acid sequence that encodes a 3-hydroxypropionyl-Co A dehydratase (EC 4.2.1.116). An illustrative example of a 3-hydroxypropionyl-Co A dehydratase is the enzyme from Metallosphaera sedula of SwissProt accession no. A4YI89 version 52 of 22 Jul. 2015 (version 1 of the sequence). Another example of a 3-hydroxypropionyl-Co A dehydratase of EC 4.2.1.116 is the acyl-CoA thioesterase YbgC from Bacillus thermoamylovorans of SwissProt accession no. A0A090IWI4, version 4 of 29 Apr. 2015 (version 1 of the sequence).

In some embodiments the microorganism contains a heterologous nucleic acid sequence that encodes the enzyme acyl-CoA thioesterase of EC 3.1.2 (EC 3.1.2.-), also called acyl-CoA thioester hydrolase. An illustrative example of an acyl-CoA thioesterase is the enzyme acyl-CoA thioesterase YneP (EC: 3.1.2.-) from Bacillus subtilis of SwissProt accession no. Q45061, version 81 of 24 Jun. 2015 (version 2 of the sequence). A further example of an acyl-CoA thioesterase is the enzyme acyl-CoA thioesterase TesB (EC: 3.1.2.-) from Haemophilus influenzae of SwissProt accession no. A0A0E3JH21, version 2 of 22 Jul. 2015 (version 1 of the sequence), which has an identical sequence to GenBank accession no. WP_{—} 005652441.1, see FIG. 66. Another example of an acyl-CoA thioesterase is the enzyme acyl-CoA thioesterase TesB (EC: 3.1.2.-) from Acinetobacter baumannii of SwissProt accession no. V5V9L9, version 17 of 22 Jul. 2015 (version 1 of the sequence). A further example of a suitable acyl-CoA thioesterase is the 1,4-dihydroxy-2-naphthoyl-CoA hydrolase enzyme (EC: 3.1.2.28) from E. coli of SwissProt accession no. C8U9Q5, version 35 of 22 Jul. 2015 (version 1 of the sequence), which has an identical sequence to GenBank accession no. WP_000637982.1, see FIG. 66. Another example of an acyl-CoA thioesterase is the enzyme acyl-CoA thioesterase TesB (EC: 3.1.2.-) from Pseudoalteromonas sp. BSi20311 of SwissProt accession no. G7EN07, version 10 of 27 May 2015 (version 1 of the sequence). A further example of an acyl-CoA thioesterase is the enzyme acyl-CoA thioesterase TesB (EC: 3.1.2.-) from E. coli of SwissProt accession no. P0AGG2, version 82 of 22 Jul. 2015 (version 2 of the sequence). Yet a further example of an acyl-CoA thioesterase is the enzyme acyl-CoA thioesterase TesA (EC: 3.1.2.-) from E. coli of SwissProt accession no. P0ADA1, version 83 of 22 Jul. 2015 (version 1 of the sequence). A further example of an acyl-CoA thioesterase is the enzyme of SwissProt accession no. A0A0E1LWS5, version 3 of 22 Jul. 2015 (version 1 of the sequence), which has an identical sequence to GenBank accession no. WP_000108160.1, cf. FIG. 66.

In some embodiments the microorganism may contain a heterologous nucleic acid sequence that encodes the enzyme phosphate butyryltransferase (EC 2.3.1.19). An example of a phosphate butyryltransferase is the enzyme phosphate butyryltransferase from Clostridium acetobutylicum of SwissProt accession no. P58255, version 78 of 22 Jul. 2015 (version 1 of the sequence), which has GenBank accession no. WP_010966357.1 (cf. FIG. 66). A further example of a phosphate butyryltransferase is the respective enzyme (EC: 2.3.1.19) from Bacillus subtilis of SwissProt accession no. P54530, version 85 of 16 Oct. 2013 (version 2 of the sequence). Yet another example is the enzyme phosphate butyryltransferase (EC 2.3.1.19) from Marinithermus hydrothermalis of SwissProt accession no. F2NPE1, version 22 of 22 Jul. 2015 (version 1 of the sequence).

In some embodiments the microorganism may contain a heterologous nucleic acid sequence that encodes the enzyme butyrate kinase (EC 2.7.2.7). An illustrative example of a butyrate kinase is the enzyme from Clostridium pasteurianum of SwissProt accession no. P81337, version 51 of 7 Jan. 2015 (version 1 of the sequence). A further example of a butyrate kinase is the enzyme from Clostridium beijerinckii of SwissProt accession no. Q05619, version 88 of 22 Jul. 2015 (version 1 of the sequence). Another example is the butyrate kinase enzyme from Thermosediminibacter oceani of SwissProt accession no. Q05619, version 31 of 22 Jul. 2015 (version 1 of the sequence).

The microorganism may in some embodiments contain a heterologous nucleic acid sequence that encodes the enzyme phenylacrylic acid decarboxylase (EC 4.1.1.-), which may also act as a phenolic acid decarboxylase. An illustrative example of a phenylacrylic acid decarboxylase is the mitochondrial enzyme from Saccharomyces cerevisiae of SwissProt accession no. P33751, version 113 of 19 Feb. 2014 (version 2 of the sequence), which is identical to GenBank accession no. NP_010827.3 (see FIG. 66). Another example of a phenylacrylic acid decarboxylase is the enzyme from Aspergillus kawachii of SwissProt accession no. G7XVA3, version 24 of 22 Jul. 2015 (version 1 of the sequence). Yet a further example of a phenylacrylic acid decarboxylase is the enzyme from Pseudoalteromonas rubra of SwissProt accession no. U1LP54, version 5 of 27 May 2015 (version 1 of the sequence). A further example of a respective enzyme, currently entitled a phenolic acid decarboxylase, is the enzyme from Bacillus subtilis of SwissProt accession no. 007006, version 93 of 24 Jun. 2015 (version 1 of the sequence), which is identical to GenBank accession no. WP_003243190.1 (see FIG. 66).

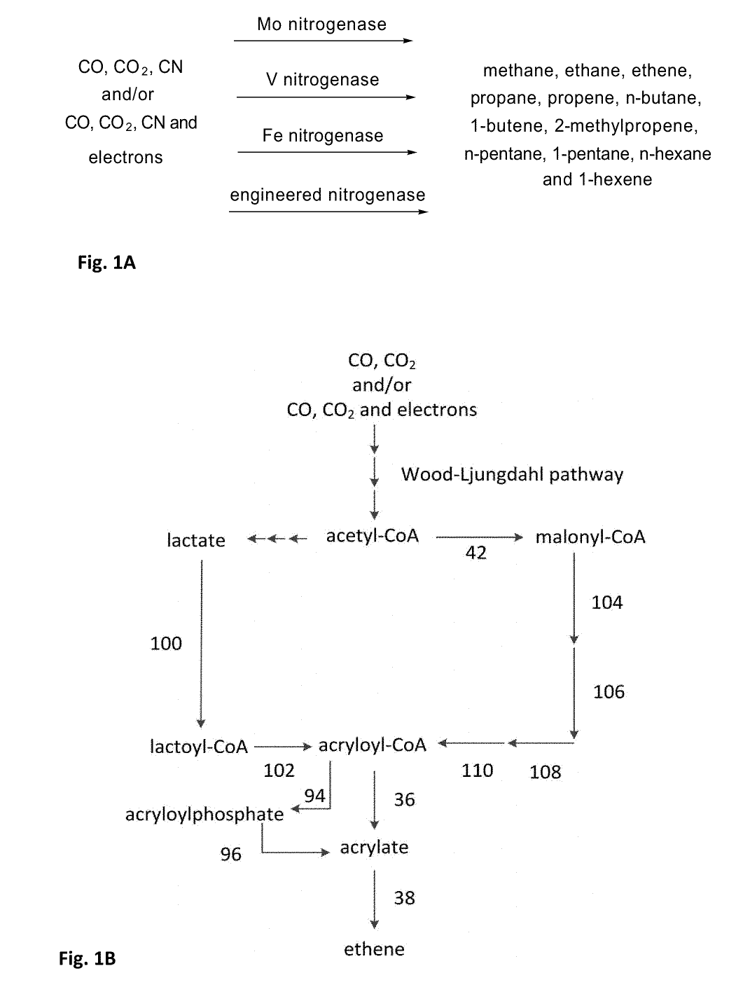

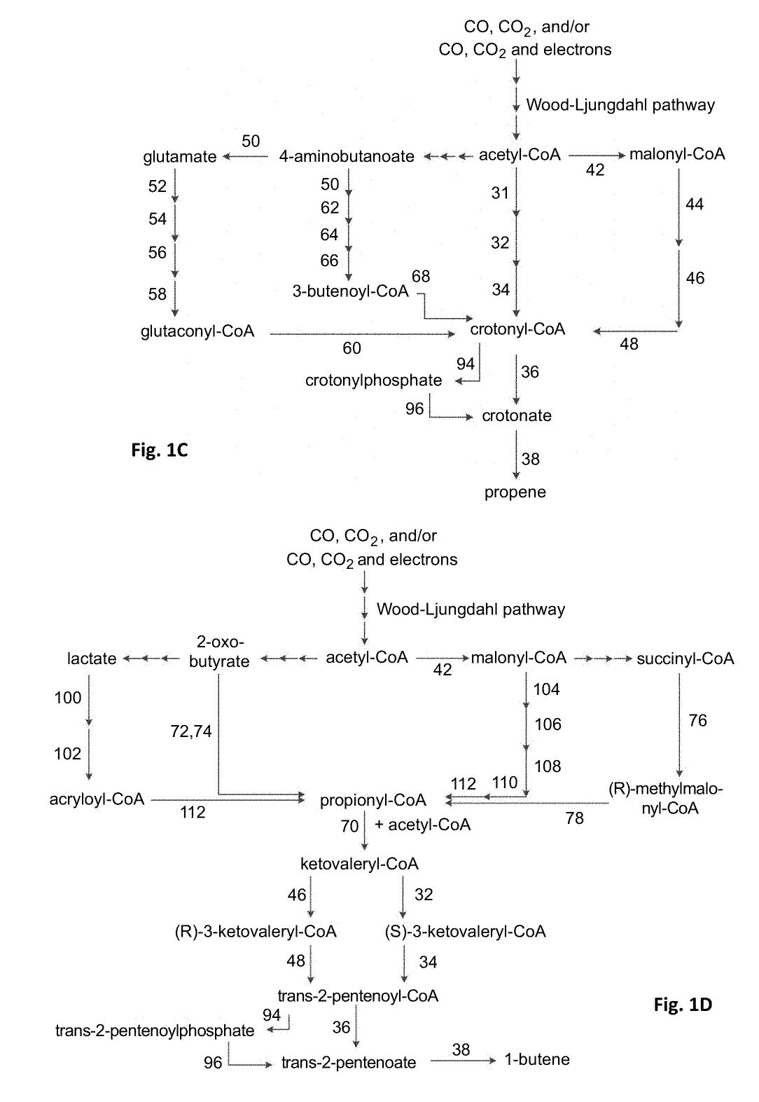

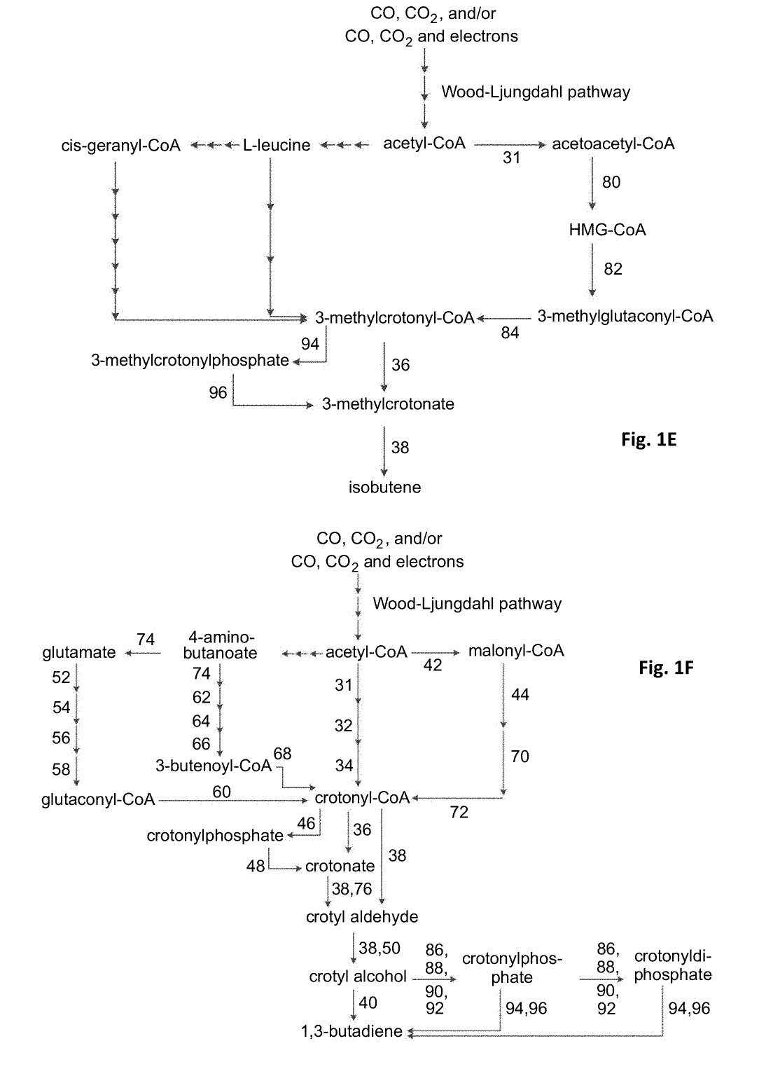

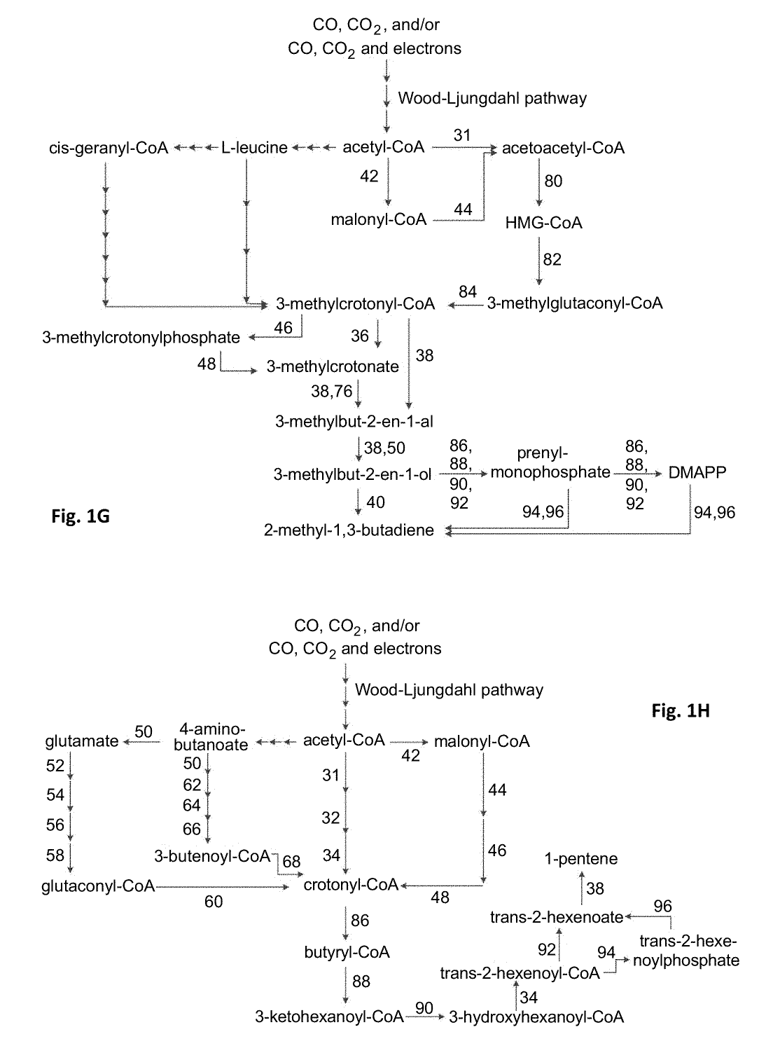

FIG. 1B depicts exemplary pathways leading to the synthesis of ethene, in which some or all of the above indicated enzymes may be present.

In some embodiments the microorganism may contain a heterologous nucleic acid sequence that encodes the enzyme 4-aminobutyrate aminotransferase (EC 2.6.1.19). An illustrative example of a 4-aminobutyrate aminotransferase is the respective enzyme from E. coli of SwissProt accession no. P22256, version 132 of 14 May 2014 (version 1 of the sequence). Another example of a 4-aminobutyrate aminotransferase is the enzyme from Thermaerobacter marianensis of SwissProt accession no. E6SLG7, version 28 of 22 Jul. 2015 (version 1 of the sequence).

In some embodiments the microorganism contains a heterologous nucleic acid sequence that encodes the enzyme glutamate dehydrogenase (EC 1.4.1.2). An illustrative example of a glutamate dehydrogenase is the enzyme from Bacillus subtilis of SwissProt accession no. P39633, version 111 of 22 Jul. 2015 (version 3 of the sequence). Another example of a glutamate dehydrogenase is the enzyme from Peptoclostridium difficile (Clostridium difficile) of SwissProt accession no. P27346, version 72 of 22 Jul. 2015 (version 1 of the sequence).

The microorganism may in some embodiments contain a heterologous nucleic acid sequence that encodes the enzyme 2-hydroxyglutarate dehydrogenase (EC 1.1.99.2). An illustrative example of a 2-hydroxyglutarate dehydrogenase is the mitochondrial enzyme Caenorhabditis elegans of SwissProt accession no. Q9N4Z0, version 84 of 22 Jul. 2015 (version 2 of the sequence). Another example of a 2-hydroxyglutarate dehydrogenase is the enzyme from Pseudomonas fluorescens of SwissProt accession no. G8Q888, version 22 of 22 Jul. 2015 (version 1 of the sequence).

In some embodiments the microorganism contains a heterologous nucleic acid sequence that encodes the enzyme glutaconate CoA-transferase (EC 2.8.3.12). An example of a respective enzyme is glutaconate CoA-transferase from Clostridium sticklandii of SwissProt accession no. E3PWJ5, version 21 of 22 Jul. 2015 (version 1 of the sequence). Another example of a glutaconate CoA-transferase is enzyme with the subunit A from Acidaminococcus fermentans of SwissProt accession no. Q59111, version 95 of 1 Apr. 2015 (version 3 of the sequence), and subunit B from Acidaminococcus fermentans of SwissProt accession no. Q59112, version 95 of 22 Jul. 2015 (version 3 of the sequence). Yet a further example is the subunit A of the enzyme from Myxococcus xanthus of SwissProt accession no. Q1D4I4, version 50 of 22 Jul. 2015 (version 1 of the sequence), which has GenBank accession no. WP_011554267.1, cf. FIG. 66. This enzyme has a subunit B of SwissProt accession no. Q1D4I3, version 46 of 22 Jul. 2015 (version 1 of the sequence), which has GenBank accession no. WP_011554268.1, cf. FIG. 66.

The microorganism may in some embodiments contain a heterologous nucleic acid sequence that encodes the enzyme 2-hydroxyglutaryl-CoA dehydratase (EC 4.2.1.-). An illustrative example of a 2-hydroxyglutaryl-CoA dehydratase is the enzyme with the subunit alpha from Acidaminococcus fermentans of SwissProt accession no. P11569, version 83 of 22 Jul. 2015 (version 3 of the sequence), and subunit beta from Acidaminococcus fermentans of SwissProt accession no. P11570, version 83 of 22 Jul. 2015 (version 3 of the sequence). Another example of a 2-hydroxyglutaryl-CoA dehydratase is the enzyme from Clostridium ljungdahlii of SwissProt accession no. D8GT18, version 19 of 22 Jul. 2015 (version 1 of the sequence).

In some embodiments the microorganism contains a heterologous nucleic acid sequence that encodes the enzyme glutaconyl-CoA decarboxylase (EC 4.1.1.70). An example of a respective enzyme is the enzyme from Acidaminococcus fermentans with the subunit alpha of SwissProt accession no. Q06700, version 82 of 1 Apr. 2015 (version 1 of the sequence), subunit beta of SwissProt accession no. Q9ZAA6, version 81 of 22 Jul. 2015 (version 1 of the sequence), subunit gamma of SwissProt accession no. Q9ZAA7, version 82 of 26 Nov. 2014 (version 1 of the sequence), and subunit delta of SwissProt accession no. Q9ZAA8, version 78 of 22 Jul. 2015 (version 1 of the sequence). Subunit delta of SwissProt accession no. Q9ZAA8 has GenBank accession no. WP_012939173.1, see FIG. 66. Another example of a glutaconyl-CoA decarboxylase is the enzyme from Rhodospirillum rubrum of SwissProt accession no. Q2RN28, version 50 of 27 May 2015 (version 1 of the sequence). Yet a further example of a glutaconyl-CoA decarboxylase is the enzyme from Clostridium phytofermentans of SwissProt accession no. A9KLQ1, version 40 of 22 Jul. 2015 (version 1 of the sequence).

The microorganism may in some embodiments contain a heterologous nucleic acid sequence that encodes the enzyme NAD-dependent 4-hydroxybutyrate dehydrogenase, also called 4-hydroxybutyrate dehydrogenase (EC 1.1.1.61). An illustrative example of a NAD-dependent 4-hydroxybutyrate dehydrogenase is the enzyme from Clostridium kluyveri of SwissProt accession no. P38945, version 72 of 22 Jul. 2015 (version 1 of the sequence). Another example of a NAD-dependent 4-hydroxybutyrate dehydrogenase is the enzyme from Acetobacterium woodii of SwissProt accession no. H6LHY2, version 16 of 22 Jul. 2015 (version 1 of the sequence).

In some embodiments the microorganism may contain a heterologous nucleic acid sequence that encodes the enzyme 4-hydroxybutyrate CoA-transferase (EC 2.8.3.-). An example of such an enzyme is Acetate CoA-transferase YdiF from E. coli of SwissProt accession no. Q8X5X6, version 81 of 22 Jul. 2015 (version 1 of the sequence). This enzyme has a broad substrate specificity for short-chain fatty acids, catalysing the reaction of propionate, acetoacetate, butyrate, isobutyrate, and 4-hydroxybutyrate. A further example of a 4-hydroxybutyrate CoA-transferase is the enzyme from Yersinia pseudotuberculosis of SwissProt accession no. A0A0E8XK97, version 2 of 22 Jul. 2015 (version 1 of the sequence). Yet another example of a 4-hydroxybutyrate CoA-transferase is the enzyme from Clostridium aminobutyricum of SwissProt accession no. Q9RM86, version 48 of 24 Jun. 2015 (version 2 of the sequence).

In some embodiments the microorganism may contain a heterologous nucleic acid sequence that encodes the enzyme vinylacetyl-CoA-Delta-isomerase (EC 5.3.3.3), which is in many embodiments also a 4-hydroxybutyryl-CoA dehydratase (EC 4.2.1.120). An example of such an enzyme with both activities is the enzyme from Clostridium aminobutyricum of SwissProt accession no. P55792, version 79 of 1 Apr. 2015 (version 3 of the sequence). Another example of a vinylacetyl-CoA-Delta-isomerase is the enzyme from Candidatus Accumulibacter sp. SK-11 of SwissProt accession no. A0A011PCK9, version 3 of 7 Jan. 2015 (version 1 of the sequence). Yet a further example of a vinylacetyl-CoA-Delta-isomerase and a 4-hydroxybutyryl-CoA dehydratase is the enzyme from Clostridium tyrobutyricum of SwissProt accession no. W6NES0, version 8 of 24 Jun. 2015 (version 1 of the sequence). Another example of a suitable enzyme with vinylacetyl-CoA delta-isomerase activity (EC 5.3.3.3) and 4-hydroxybutyryl-CoA dehydratase activity (EC 4.2.1.-) is the enzyme from Carboxydothermus hydrogenoformans of SwissProt accession no. Q3ACI6, version 67 of 22 Jul. 2015 (version 1 of the sequence). Another example of a suitable enzyme with vinylacetyl-CoA delta-isomerase activity (EC 5.3.3.3) and 4-hydroxybutyryl-CoA dehydratase activity (EC 4.2.1.-) is the enzyme from Clostridium kluyveri of SwissProt accession no. B9E5E4, version 39 of 22 Jul. 2015 (version 1 of the sequence), which has GenBank accession no. WP_012103363.1, cf. FIG. 66. An example of an enzyme that is only known to fall under EC 4.2.1.120 as a 4-hydroxybutyryl-CoA dehydratase is the enzyme from Nitrososphaera gargensis of SwissProt accession no. K0IP19, version 14 of 22 Jul. 2015 (version 1 of the sequence). A further example of an enzyme that is only known to have 4-hydroxybutyryl-CoA dehydratase activity is the enzyme from Thermoproteus tenax of SwissProt accession no. G4RJK0, version 20 of 22 Jul. 2015 (version 1 of the sequence).

In some embodiments the microorganism may contain a heterologous nucleic acid sequence that encodes the enzyme acetoacetyl-CoA thiolase or acetyl-CoA acetyltransferase (EC 2.3.1.9). An example of a respective enzyme is the enzyme from Peptoclostridium difficile (Clostridium difficile) with the SwissProt accession no. P45362, version 74 of 7 Jan. 2015 (version 2 of the sequence). Two further examples of an acetyl-CoA acetyltransferase are the enzymes from E. coli of SwissProt accession no. A0A024LB55, version 12 of 22 Jul. 2015 (version 1 of the sequence), and of SwissProt accession no. H9UUG8, version 17 of 22 Jul. 2015 (version 1 of the sequence, which includes the sequences of GenBank accession nos. AKR29896.1 and WP_000786547.1, cf. FIG. 66. Another example of a suitable acetyl-CoA acetyltransferase is the enzyme beta-ketothiolase BktB from Cupriavidus necator of SwissProt accession no. Q0KBP1, version 62 of 22 Jul. 2015 (version 1 of the sequence), which is identical to the sequences of GenBank accession no. WP_011615089.1, cf. FIG. 66. Yet a further example of an acetyl-CoA acetyltransferase is the enzyme from Bacillus thermoamylovorans of SwissProt accession no. A0A0D0F6V4, version 4 of 22 Jul. 2015 (version 1 of the sequence).

The microorganism may in some embodiments contain a heterologous nucleic acid sequence that encodes the enzyme 3-hydroxybutyryl-CoA dehydrogenase (EC 1.1.1.157). An illustrative example of a 3-hydroxybutyryl-CoA dehydrogenase is the enzyme from Clostridium acetobutylicum of SwissProt accession no. P52041, version 101 of 27 May 2015 (version 2 of the sequence). A further example of a 3-hydroxybutyryl-CoA dehydrogenase is the enzyme from Klebsiella pneumoniae of SwissProt accession no. B5XRJ5, version 52 of 22 Jul. 2015 (version 1 of the sequence). Yet another example of a 3-hydroxybutyryl-CoA dehydrogenase is the enzyme from E. coli of SwissProt accession no. B7L6B5, version 45 of 22 Jul. 2015 (version 1 of the sequence).

In some embodiments the microorganism may contain a heterologous nucleic acid sequence that encodes the enzyme 3-hydroxybutyryl-CoA dehydratase (EC 4.2.1.55). An example of a 3-hydroxybutyryl-CoA dehydratase is the enzyme from Methylobacterium extorquens with the SwissProt accession no. C5AZ74, version 34 of 29 Oct. 2014 (version 1 of the sequence). A further example is the enzyme from Peptoclostridium difficile with the SwissProt accession no. Q189Z6, version 60 of 22 Jul. 2015 (version 1 of the sequence).

In some embodiments the microorganism may contain a heterologous nucleic acid sequence that encodes the enzyme acetyl-CoA carboxylase (EC 6.4.1.2). This enzyme is typically an enzyme complex. An illustrative subunit is the beta subunit from E. coli of SwissProt accession no. W8ZLL8, version 13 of 22 Jul. 2015 (version 1 of the sequence), which corresponds to GenBank accession no. ABE08068.1 (cf. FIG. 66). Subunit alpha from E. coli of this enzyme has SwissProt accession no. W8ZDE6, version 12 of 22 Jul. 2015 (version 1 of the sequence)

In some embodiments the microorganism may contain a heterologous nucleic acid sequence that encodes the enzyme biotin carboxylase (EC 6.3.4.14). An example of a suitable biotin carboxylase is the enzyme from Shigella dysenteriae of SwissProt accession no. A0A0A7A003, version 5 of 22 Jul. 2015 (version 1 of the sequence), which corresponds to GenBank accession no. AHA67379.1 (cf. FIG. 66).

The microorganism may in some embodiments contain a heterologous nucleic acid sequence that encodes the enzyme acetoacetyl-CoA synthase (EC 2.3.1.194). An illustrative example of an acetoacetyl-CoA synthase is the enzyme from Streptomyces sp. (strain CL190) of SwissProt accession no. D7URV0, version 23 of 22 Jul. 2015 (version 1 of the sequence), which is identical to GenBank Accession no. D7URV0.1, cf. FIG. 66. Another example of an acetoacetyl-CoA synthase is the enzyme from Paenibacillus sp. P1XP2 of SwissProt accession no. A0A0B0I6S7, version 5 of 22 Jul. 2015 (version 1 of the sequence).

In some embodiments the microorganism may contain a heterologous nucleic acid sequence that encodes the enzyme acetoacetyl-CoA reductase (EC 1.1.1.36). An example of an acetoacetyl-CoA reductase is the enzyme from Acinetobacter sp. (strain RA3849) with the SwissProt accession no. P50203, version 70 of 7 Jan. 2015 (version 1 of the sequence). A further example of an acetoacetyl-CoA reductase is the enzyme from Methylobacterium extorquens with the SwissProt accession no. C5AZN8, version 41 of 22 Jul. 2015 (version 1 of the sequence). Yet another example of an acetoacetyl-CoA reductase is the enzyme from Aeromonas hydrophila with the SwissProt accession no. W0A092, version 7 of 22 Jul. 2015 (version 1 of the sequence).

The microorganism may in some embodiments contain a heterologous nucleic acid sequence that encodes an enoyl-CoA hydratase such as the enzyme (R)-specific enoyl-CoA hydratase (EC 4.2.1.119). An illustrative example of an enoyl-CoA hydratase enzyme is the enzyme from Pseudomonas putida of SwissProt accession no. Q88FM3, version 70 of 22 Jul. 2015 (version 1 of the sequence), which is identical to the enzyme of GenBank accession no. WP_003251320.1, see FIG. 66. Another example of an enoyl-CoA hydratase is the enzyme from Aeromonas hydrophila of SwissProt accession no. W0A1X1, version 9 of 22 Jul. 2015 (version 1 of the sequence), which is identical to the enzyme of GenBank accession no. WP_025327110.1, cf. FIG. 66. An example of a (R)-specific enoyl-CoA hydratase (EC 4.2.1.119) is the enzyme from Rhodospirillum rubrum of SwissProt accession no. Q2RQ36, version 59 of 24 Jun. 2015 (version 1 of the sequence). A further example of an (R)-specific enoyl-CoA hydratase (EC 4.2.1.119) is the peroxisomal enzyme from Saccharomyces cerevisiae with the SwissProt accession no. Q02207, version 123 of 27 May 2015 (version 1 of the sequence). Yet another example of an (R)-specific enoyl-CoA hydratase (EC 4.2.1.119) is the peroxisomal enzyme from Mycobacterium xenopi with the SwissProt accession no. X7YI38, version 7 of 24 Jun. 2015 (version 1 of the sequence).

FIG. 1C depicts exemplary pathways leading to the synthesis of propene, in which one or more of the above indicated enzymes may be present.

In some embodiments the microorganism may contain a heterologous nucleic acid sequence that encodes the enzyme acryloyl-coenzyme A reductase (EC 1.3.1.84), which may also be called propionyl-CoA synthase. An example of an acryloyl-coenzyme A reductase is the enzyme from Sulfolobus tokodaii with the SwissProt accession no. Q975C8, version 96 of 22 Jul. 2015 (version 1 of the sequence). Another example of an acryloyl-coenzyme A reductase is the enzyme from Rhodobacter sphaeroides with the SwissProt accession no. Q3J6K9, version 65 of 29 Apr. 2015 (version 1 of the sequence).

In some embodiments the microorganism may contain a heterologous nucleic acid sequence that encodes the enzyme methylmalonyl-CoA mutase (EC 5.4.99.2). An example of a methylmalonyl-CoA mutase is the enzyme from E. coli with the SwissProt accession no. P27253, version 124 of 22 Jul. 2015 (version 2 of the sequence). A further example of a methylmalonyl-CoA mutase is the enzyme from Streptomyces cinnamonensis with the SwissProt accession no. Q05065, version 77 of 24 Jun. 2015 (version 1 of the sequence). Another example of a methylmalonyl-CoA mutase is the enzyme from Sulfolobus islandicus with the SwissProt accession no. C3N159, version 29 of 24 Jun. 2015 (version 1 of the sequence).

The microorganism may in some embodiments contain a heterologous nucleic acid sequence that encodes the enzyme methylmalonyl-CoA decarboxylase (EC 4.1.1.41). An illustrative example of a methylmalonyl-CoA decarboxylase is the enzyme from E. coli of SwissProt accession no. P52045, version 108 of 22 Jul. 2015 (version 1 of the sequence). A further example of a methylmalonyl-CoA decarboxylase is the enzyme from Streptococcus pyogenes with the SwissProt accession no. A0A0C6FWG2, version 4 of 22 Jul. 2015 (version 1 of the sequence).

In some embodiments the microorganism may contain a heterologous nucleic acid sequence that encodes an aldehyde-alcohol dehydrogenase enzyme. Such an enzyme typically includes alcohol dehydrogenase activity (EC 1.1.1.1) and may also include acetaldehyde dehydrogenase activity (EC 1.2.1.10). An illustrative example of an aldehyde-alcohol dehydrogenase is the enzyme from Clostridium acetobutylicum of SwissProt accession no. Q7DFN2, version 24 of 27 May 2015 (version 1 of the sequence), which has GenBank accession no. WP_010890720.1 (cf. FIG. 66).

FIG. 1D depicts exemplary pathways leading to the synthesis of n-butene, in which one or more of the above indicated enzymes may be present.

In some embodiments the microorganism may contain a heterologous nucleic acid sequence that encodes the enzyme 3-hydroxy-3-methylglutaryl-CoA synthase (EC 2.3.3.10). An example of a 3-hydroxy-3-methylglutaryl-CoA synthase is the enzyme from Haloferax volcanii with the SwissProt accession no. D4GWR6, version 37 of 22 Jul. 2015 (version 1 of the sequence). An further example of a 3-hydroxy-3-methylglutaryl-CoA synthase is the enzyme from Staphylococcus aureus with the SwissProt accession no. A0A0E1XB83, version 3 of 22 Jul. 2015 (version 1 of the sequence), which is identical to GenBank accession no. WP_000172190.1. Another example of a 3-hydroxy-3-methylglutaryl-CoA synthase is the enzyme from Bacillus amyloliquefaciens Y2 with the SwissProt accession no. I2C5E4, version 18 of 22 Jul. 2015 (version 1 of the sequence).

In some embodiments the microorganism may contain a heterologous nucleic acid sequence that encodes the enzyme methylglutaconyl-CoA hydratase, also called 3-methylglutaconyl-CoA hydratase (EC 4.2.1.18). An illustrative example of a methylglutaconyl-CoA hydratase is the mitochondrial enzyme from Dictyostelium discoideum of SwissProt accession no. Q54HG7, version 67 of 22 Jul. 2015 (version 1 of the sequence). Another example of a methylglutaconyl-CoA hydratase is the enzyme from Bacillus cereus of SwissProt accession no. A0A068N7T5, version 5 of 22 Jul. 2015 (version 1 of the sequence). Yet a further example of a suitable enzyme with methylglutaconyl-CoA hydratase activity is the enzyme from Desulfatibacillum alkenivorans of SwissProt accession no. B8FC62, version 36 of 22 Jul. 2015 (version 1 of the sequence).

The microorganism may in some embodiments contain a heterologous nucleic acid sequence that encodes the enzyme methylcrotonoyl-CoA carboxylase, also called 3-methylcrotonyl-CoA carboxylase (EC 6.4.1.4). An illustrative example of a methylcrotonoyl-CoA carboxylase is the mitochondrial enzyme from Dictyostelium discoideum with an alpha subunit of SwissProt accession no. Q54KE6, version 80 of 29 May 2013 (version 1 of the sequence), and a beta subunit of SwissProt accession no. Q8T2J9, version 76 of 24 Jun. 2015 (version 2 of the sequence). Another example of a methylcrotonoyl-CoA carboxylase is the enzyme from Octadecabacter arcticus with an alpha subunit of SwissProt accession no. M9RSW8, version 14 of 22 Jul. 2015 (version 1 of the sequence), and a beta subunit of SwissProt accession no. M9RL62, version 15 of 29 Apr. 2015 (version 1 of the sequence). Yet a further example of a methylcrotonoyl-CoA carboxylase is the enzyme from Pseudomonas aeruginosa with the SwissProt accession no. Q9I299, version 91 of 24 Jun. 2015 (version 1 of the sequence), which is identical to the enzyme of GenBank accession no. WP_003113506.1, see FIG. 66. Another example of a methylcrotonoyl-CoA carboxylase is the enzyme from Pseudomonas aeruginosa with the SwissProt accession no. W1MJK9, version 8 of 22 Jul. 2015 (version 1 of the sequence), which is identical to the enzyme of GenBank accession no. WP_003100387.1, see FIG. 66.

FIG. 1E depicts exemplary pathways leading to the synthesis of isobutene, in which one or more of the above indicated enzymes may be present.

In some embodiments the microorganism may contain a heterologous nucleic acid sequence that encodes the enzyme pyruvate/2-oxobutyrate ferredoxin oxidoreductase, also called pyruvate/ketoisovalerate oxidoreductase or pyruvate synthase (EC 1.2.7.1). An illustrative example of a 2-oxobutyrate ferredoxin oxidoreductase is the pyruvate synthase enzyme from Methanosarcina barkeri with a subunit PorA of SwissProt accession no. P80521, version 83 of 22 Jul. 2015 (version 2 of the sequence), a subunit PorB of SwissProt accession no. P80522, version 81 of 22 Jul. 2015 (version 3 of the sequence), and a subunit PorC of SwissProt accession no. P80523, version 84 of 22 Jul. 2015 (version 2 of the sequence). Another example of a pyruvate/2-oxobutyrate ferredoxin oxidoreductase is the enzyme called pyruvate/ketoisovalerate oxidoreductases common subunit gamma (PorG) from Pyrococcus endeavori of SwissProt accession no. P84819, version 21 of 1 Oct. 2014 (version 1 of the sequence). Yet a further example of

In some embodiments the microorganism may contain a heterologous nucleic acid sequence that encodes the enzyme 2-oxoglutarate/butyrate ferredoxin oxidoreductase, also called 2-ketoglutarate oxidoreductase or 2-oxoglutarate synthase (EC 1.2.7.3). An illustrative example of a 2-oxoglutarate ferredoxin oxidoreductase is the pyruvate synthase enzyme from Methanocaldococcus jannaschii with a subunit KorA of SwissProt accession no. Q57724, version 88 of 24 Jun. 2015 (version 1 of the sequence), a subunit KorB of SwissProt accession no. Q57957, version 83 of 22 Jul. 2015 (version 1 of the sequence), and a subunit KorC of SwissProt accession no. Q57956, version 85 of 24 Jun. 2015 (version 1 of the sequence). Another example of a 2-oxoglutarate/butyrate ferredoxin oxidoreductase is the enzyme 2-ketoglutarate:ferredoxin oxidoreductase (KGOR) from Thermococcus sibiricus with a subunit alpha of SwissProt accession no. C6A4H7, version 32 of 27 May 2015 (version 1 of the sequence), a subunit beta of SwissProt accession no. C6A4H6, version 32 of 27 May 2015 (version 1 of the sequence), and a subunit gamma of SwissProt accession no. C6A4H5, version 31 of 27 May 2015 (version 1 of the sequence).

In some embodiments the microorganism may contain a heterologous nucleic acid sequence that encodes an aldehyde ferredoxin oxidoreductase enzyme (EC 1.2.7.5). An illustrative example of an aldehyde ferredoxin oxidoreductase is the enzyme from Clostridium autoethanogenum with the SwissProt accession no. U5RP02, version 10 of 22 Jul. 2015 (version 1 of the sequence), which has GenBank accession no. WP_013238665.1, cf. FIG. 66. The enzyme may also be the butyrate kinase from Clostridium acetobutylicum with GenBank accession no. WP_010966356.1 (cf. FIG. 66), which is completely included in the aldehyde ferredoxin oxidoreductase of SwissProt accession no. U5RP02.

In some embodiments the microorganism may contain a heterologous nucleic acid sequence that encodes the enzyme geraniol isomerase (EC 5.4.4.4), also called geraniol hydroxy-mutase. The enzyme is bifunctional and also acts as a linalool dehydratase (EC 4.2.1.127), also called linalool dehydratase-isomerase. An example of a geraniol isomerase/linalool dehydratase is the enzyme from Castellaniella defragrans with the SwissProt accession no. E1XUJ2, version 12 of 1 Apr. 2015 (version 1 of the sequence). A further example of a geraniol isomerase/linalool dehydratase is the enzyme from Colletotrichum gloeosporioides with the SwissProt accession no. L2FRH3, version 11 of 4 Mar. 2015 (version 1 of the sequence). Yet another example of a geraniol isomerase/linalool dehydratase is the enzyme from Mycobacterium triplex with the SwissProt accession no. A0A024JQR5, version 5 of 24 Jun. 2015 (version 1 of the sequence).

The microorganism may in some embodiments contain a heterologous nucleic acid sequence that encodes the enzyme trans-2-enoyl-CoA reductase, also called acetoacetyl-CoA reductase (EC1.1.1.36). An illustrative example of an acetoacetyl-CoA reductase is the enzyme from Paracoccus denitrificans with the SwissProt accession no. P50204, version 76 of 24 Jun. 2015 (version 1 of the sequence). Another example of an acetoacetyl-CoA reductase is the enzyme from Achromobacter xylosoxidans with the SwissProt accession no. E3HWI9, version 31 of 22 Jul. 2015 (version 1 of the sequence).

In some embodiments the microorganism may contain a heterologous nucleic acid sequence that encodes the enzyme butyrate kinase (EC 2.7.2.7). An example of a butyrate kinase is the enzyme butyrate kinase 1 from Clostridium acetobutylicum with the SwissProt accession no. Q45829, version 90 of 27 Mar. 2015 (version 2 of the sequence). Another example of a butyrate kinase is the enzyme butyrate kinase 2 from Clostridium acetobutylicum with the SwissProt accession no. Q97111, version 80 of 22 Jul. 2015 (version 1 of the sequence). Yet another example of a butyrate kinase is the enzyme from Thermotoga maritima with the SwissProt accession no. G4FGE0, version 34 of 22 Jul. 2015 (version 1 of the sequence).

In some embodiments the microorganism may contain a heterologous nucleic acid sequence that encodes an isoprene synthase enzyme (EC 4.2.3.27). An illustrative example of an isoprene synthase is the enzyme from Pueraria montana var lobate of SwissProt accession no. Q6EJ97, version 42 of 1 Apr. 2015 (version 1 of the sequence), which also has GenBank accession no. Q6EJ97.1, cf. FIG. 66. A further example of an isoprene synthase is the enzyme from Populus alba of SwissProt accession no. Q50L36, version 38 of 27 May 2015 (version 1 of the sequence).

In some embodiments the microorganism may contain a heterologous nucleic acid sequence that encodes a diacylglycerol kinase enzyme. An illustrative example of a diacylglycerol kinase is the enzyme from Streptococcus mutans GS-5 with the SwissProt accession no. Q05888, version 106 of 24 Jun. 2015 (version 2 of the sequence) of GenBank accession no. AAA26867, version 5 of the entry of 23 Jun. 2010 (version 2 of the sequence), cf. FIG. 66. This enzyme may for example be an undecaprenol kinase (EC 2.7.1.66), which has diacylglycerol kinase activity. An example of a respective undecaprenol kinase is the enzyme from Streptococcus mutans serotype c with the SwissProt accession no. Q05888, version 106 of 24 Jun. 2015 (version 2 of the sequence), which 98% identical to the diacylglycerol kinase of GenBank accession no. AAA26867.1.

The microorganism may in some embodiments contain a heterologous nucleic acid sequence that encodes the enzyme 2-methyl-3-buten-2-ol synthase. An illustrative example of a 2-methyl-3-buten-2-ol synthase is the enzyme from Pinus muricata of SwissProt accession no. I3QDT7, version 14 of 1 Apr. 2015 (version 1 of the sequence). Another example of a 2-methyl-3-buten-2-ol synthase is the enzyme from Pinus radiata with the SwissProt accession no. I3QDV8, version 14 of 1 Apr. 2015 (version 1 of the sequence), which has GenBank accession no. AFJ73575.1, cf. FIG. 66.

FIG. 1F depicts exemplary pathways leading to the synthesis of butadiene, in which one or more of the above indicated enzymes may be present. FIG. 1G depicts exemplary pathways leading to the synthesis of 2-methyl-1,3-butadiene, in which one or more of the above indicated enzymes may be present. FIG. 1H depicts exemplary pathways leading to the synthesis of 1-pentene, in which one or more of the above indicated enzymes may be present.

FIG. 66 depicts exemplary enzymes encoded by plasmids described below. Any of the enzymes shown in this figure may be encoded by a heterologous nucleic acid sequence included in a recombinant microorganism and expressed by the same. In some embodiments the recombinant microorganism may include one or more heterologous nucleic acid sequences expressing two or more enzymes that are shown in FIG. 66.

In some embodiments exogenous expression of the encoding nucleic acid sequence(s) is employed. Exogenous expression confers the ability to custom tailor the expression and/or regulatory elements to the host and application to achieve a desired expression level that is controlled by the user. In some embodiments endogenous expression is utilized, for instance by removing a negative regulatory effector or induction of the promoter of a gene when linked to an inducible promoter or other regulatory element. An endogenous gene having a naturally occurring inducible promoter can be up-regulated by providing the appropriate inducing agent, or the regulatory region of an endogenous gene can be engineered to incorporate an inducible regulatory element, thereby allowing the regulation of increased expression of an endogenous gene at a desired time. Similarly, an inducible promoter can be included as a regulatory element for an exogenous gene introduced into a non-naturally occurring microorganism.

For some enzymes known to have one particular activity, it has been reported that the enzyme also catalyzes a very different function. As an example, methylcrotonoyl-CoA carboxylase from Octadecabacter arcticus of SwissProt accession no. M9RSW8 has both methylcrotonoyl-CoA carboxylase activity and biotin carboxylase activity. As two further examples, methylglutaconyl-CoA hydratase from Desulfatibacillum alkenivorans of SwissProt accession no. B8FC62 has both methylglutaconyl-CoA hydratase activity and enoyl-CoA isomerase activity, and methylmalonyl-CoA decarboxylase from Streptococcus pyogenes with the SwissProt accession no. A0A0C6FWG2 has methylmalonyl-CoA decarboxylase activity, enoyl-CoA isomerase activity and enoyl-CoA hydratase activity. On the other hand, enzymes can also be modified using directed evolution to broaden their specificity towards non-natural substrates. Alternatively, the substrate preference of an enzyme can also be changed using directed evolution. Thereby a given enzyme can be engineered for efficient function on a natural, for example, improved efficiency, or a non-natural substrate, for example, increased efficiency. In this regard, algorithms have been used to design a retro-aldolase that could be used to catalyze the carbon-carbon bond cleavage in a non-natural and non-biological substrate, 4-hydroxy-4-(6-methoxy-2-naphthyl)-2-butanone (Jiang, L., et al., Science (2008) 319, 1387-1391). These algorithms used different combinations of four different catalytic motifs to design a new enzyme. Twenty of the selected designs for experimental characterization had four-fold improved rates compared to the uncatalyzed reaction (Jiang et al., 2008, supra). In this way not only the array of potential substrates that can be used by an enzyme can be changed, but the design and construction of an enzyme can also be performed. As an example, a method of DNA shuffling (random chimeragenesis on transient templates or RACHITT) was reported to lead to an engineered monooxygenase that had an improved rate of desulfurization on complex substrates as well as 20-fold faster conversion of a non-natural substrate (Coco, W M, et al., Nat. Biotechnol. (2001) 19, 4, 354-359). Similarly, the specific activity of a sluggish mutant triosephosphate isomerase enzyme was improved up to 19-fold from 1.3 fold (Hermes, J D, et al., Proc. Nat. Acad. Sci U.S.A. (1990) 87, 2, 696-700). This enhancement in specific activity was accomplished by using random mutagenesis over the whole length of the protein and the improvement could be traced back to mutations in six amino acid residues.

As noted above, the recombinant microorganism may contain the endogenous enzymes of the Wood-Ljungdahl pathway. The recombinant microorganism may have been engineered on the basis of a naturally occurring microorganism as the host that already included functional endogenous enzymes of the Wood-Ljungdahl pathway. The Wood-Ljungdahl pathway, also called the reductive acetyl-CoA pathway, is an enzymatic reaction path that allows the conversion of CO and/or CO₂ to acetyl-CoA. This pathway is indicated in any of FIG. 1B to FIG. 1F. Where CO₂ is used as the reactant, one molecule of CO₂ is reduced to CO via the enzyme carbon monoxide (CO) dehydrogenase/acetyl-CoA synthase. Another molecule of CO₂ is reduced to create a carbonyl group. The recombinant microorganism may be an acetogen, i.e. an obligately anaerobic bacterium, in which the reductive acetyl-CoA or Wood-Ljungdahl pathway serves as the bacterium's main mechanism for energy conservation. With CO₂ as the substrate, in an acetogenic bacterium molecular hydrogen is typically required for the formation of acetyl-CoA. A respective microorganism is thus capable of utilizing CO and syngas as well as generally also of utilizing CO₂ and CO₂/H₂ mixtures.

### Obtaining a Recombinant Microorganism

A number of prokaryotes are suitable for use in obtaining a recombinant microorganism as described herein, such as gram-negative bacteria. In some embodiments the microorganism used is a bacterium that can ferment one carbon reactants such as carbon monoxide or carbon dioxide. Suitable species may be in a genus of Acetobacterium, Acetohalobium, Acetoanaerobium, Acetitomaculum, Acetonema, Alkalibaculum, Anoxybacillus, Ammonifex, Archaeoglobus, Bacillus, Blautia, Bradyrhizobium, Bryanella, Butyribacterium, Caloramator, Calderihabitans, Caloribacterium, Caldanaerobacter, Candidatus, Carbophilus, Carboxydobrachium, Carboxydocella, Carboxydothermus, Cloacibacillus, Clostridium, Desulfomonile, Desulfovirgula, Holophaga, Eubacterium, Desulfitbacterium, Desulfotomaculum, Desulfurispora, Desulfosporosinus, Dehalococcoides, Desulfatibacillum, Desulfobacterium, Desulfobacula, Desulfospira, Desulfovermiculus, Desulfovibrio, Dictyoglomus, Hydrogenophilus, Hydrogenophaga, Moorella, Methanobrevibacter, Mycobacterium, Natroniella, Natronincola, Oligotropha, Oxobacter, Orenia, Paenibacillus, Peptostreptococcus, Pseudomonas, Ralstonia, Rhodopseudomonas, Rhodospirillum, Rubrivivax, Ruminococcus, Scalindua, Syntrophococcus, Streptomyces, Sporomusa, Thermoanaerobacter, Thermincola, Thermoacetogenium, Thermosinus, Tindallia, Thermosediminibacter, Treponema, Thermolithobacter, Thermococcus, Thermofilum or Thermoproteus. Typically, a strain selected for development as an alkane/alkene production strain is first analysed to determine which alkene production genes are endogenous to the strain and which genes are not present. Genes for which an endogenous counterpart is not present in the strain are assembled in one or more recombinant constructs, which are then transformed into the strain in order to supply the missing function(s). Genes for which an endogenous counterpart is present in the strain can, if desired, be modified or supplemented with one or more recombinant genes, for example in order to enhance flux through particular pathways or particular steps.

In some embodiments, the recombinant microorganism is a cell that is derived from a naturally occurring carboxidotrophic cell. The respective host cell may thus be carboxidotrophic. In some embodiments, the recombinant cell is derived from a naturally occurring thermotolerant host cell. The respective host cell may thus be thermotolerant. Thermotolerant host cells are useful in process applications in biotechnology due to their ability to grow at relatively high temperatures. In some embodiments the host cell may be thermophilic, i.e. a cell that thrives at a temperature of about 45° C. or higher. In some embodiments the host cell may be mesophilic, i.e. a cell that thrives in the range from about 20 to about 45° C.

A large number of well-established techniques of genetic engineering are available in the art that can be employed in order to obtain a microorganism as disclosed herein. Such techniques include molecular methods such as heterologous gene expression, genome insertion or deletion, altered gene expression, inactivation of genes, or enzyme engineering methods.

In a method disclosed herein a nucleic acid molecule may be introduced into a host cells by any suitable technique of nucleic acid delivery for transformation of a cell available in the art. Examples of suitable techniques include, but are not limited to, direct delivery of DNA, e.g. via transfection, injection, including microinjection, electroporation, calcium phosphate precipitation, by using DEAE-dextran followed by polyethylene glycol, direct sonic loading, liposome mediated transfection, receptor-mediated transfection, microprojectile bombardment, agitation with silicon carbide fibers, Agrobacterium-mediated transformation, desiccation/inhibition-mediated DNA uptake or any combination thereof

An exogenous nucleic acid molecule may be delivered to a microorganism as a naked nucleic acid molecule or it may be formulated with one or more agents to facilitate the transformation process. As an illustrative example, a liposome-conjugated nucleic acid may be provided. A microorganism as described herein may be obtained by introducing one or more exogenous nucleic acids into a microorganism. A variety of techniques of introducing a nucleic acid molecule into a cell are available in the art. For example, transformation, including transduction or transfection, may be achieved by electroporation, ultrasonication, polyethylene glycol-mediated transformation, chemical or natural competence, protoplast transformation, prophage induction or conjugation.

Electroporation has been described for several carboxydotrophic acetogens as C. ljungdahlii (Köpke et al., Poc. Nat. Acad. Sci. U.S.A. (2010) 107, 13087-13092; WO 2012/053905; Leang et al., Appl Environ Microbiol. (2013) 79, 4, 1102-1109), C. autoethanogenum (WO 2012/053905), Acetobacterium woodii (Straetz et al., Appl. Environ. Microbiol. (1994) 60, 1033-37) or Moorella thermoacetica (Kita et al., J Biosci Bioeng. (2013) 115, 4, 347-352) and is a standard method used in many Clostridia such as C. acetobutylicum (Mermelstein et al., Biotechnology (1992) 10, 190-195), C. cellulolyticum (Jennert et al., 2000, Microbiology, 146, 3071-3080) or C. thermocellum (Tyurin et al., Appl. Environ. Microbiol. (2004) 70, 883-890). Conjugation has been described as method of choice for many Clostridia including Clostridium difficile (Herbert et al., FEMS Microbiol. Lett. (2003) 229, 103-110) or C. acetobuylicum (Williams et al., J. Gen. Microbiol. (1990) 136, 819-826) and could be used in a similar fashion for carboxydotrophic acetogens.

In certain embodiments, due to the restriction systems which are active in the microorganism to be transformed, it is necessary to methylate the nucleic acid to be introduced into the microorganism. This can be done using a variety of techniques known in the art.

### Vectors and Gene Integration

A microorganism described herein may in some embodiments be a bacterium. In some embodiments a bacterium as described herein may contain a bacterial chromosome and an additional nucleic acid molecule. The additional nucleic acid molecule may have been introduced as described above. In some embodiments a bacterium as described herein contains a bacterial chromosome and no additional nucleic acid molecule. The bacterial chromosome may in some embodiments be modified and contain an exogenous nucleic acid sequence as described herein. The exogenous nucleic acid sequence may be a heterologous nucleic acid sequence. In one embodiment the bacterial chromosome contains exogenous, including a heterologous, nucleic acid sequence as described herein, and does not contain an additional nucleic acid molecule.

A microorganism described herein may in some embodiments contain two or more heterologous nucleic acid sequences encoding an enzyme as described herein. A microorganism described herein may in some embodiments contain three or more heterologous nucleic acid sequences encoding an enzyme as described herein. A microorganism described herein may also contain a heterologous nucleic acid sequence encoding two or more enzymes as described herein. A microorganism described herein may also contain a heterologous nucleic acid sequence encoding three or more enzymes as described herein.

In some embodiments a nucleic acid molecule as disclosed herein includes an expression cassette capable of inducing and/or regulating the expression of an enzyme. In some embodiments a nucleic acid molecule as disclosed herein is encompassed by a vector that contains a promoter effective to initiate transcription in the respective host cell (whether of endogenous or exogenous origin).

Expression of an enzyme in a microorganism may include the generation of a vector that has a construct with a sequence encoding the respective enzyme. A vast number of suitable vectors are known that can be employed. Once the vector or nucleic acid molecule that contains the construct(s) has been prepared for expression, the nucleic acid construct(s) may be introduced into a selected suitable host cell by any of a variety of suitable means, i.e., transformation, transfection, conjugation, protoplast fusion, electroporation, particle gun technology, calcium phosphate-precipitation, direct microinjection, and the like. In some embodiments, the vector contains a selective marker. Examples of selectable markers include, but are not limited to, antibiotic resistance nucleic acids (e.g., kanamycin, ampicillin, carbenicillin, gentamicin, hygromycin, phleomycin, bleomycin, neomycin, or chloramphenicol) and/or nucleic acids that confer a metabolic advantage, such as a nutritional advantage on the host cell. In such embodiments after the introduction of the vector, recipient cells can be grown in a selective medium, which selects for the growth of vector-containing microorganism. Expression of the cloned gene(s) results in the production of the respective enzyme. This can take place in the transformed cells as such, or following the induction of these cells to differentiate. A variety of incubation conditions can be used to form a peptide as disclosed herein. In some embodiments it may be desired to use conditions that mimic physiological conditions.

Generating a microorganism as described herein may involve the use of various recombinant nucleic acid constructs such as cloning vectors, expression vectors or synthetic operons. Both cloning and expression vectors contain nucleotide sequences that allow the vectors to replicate in one or more suitable recombinant microorganisms. In a cloning vector, this sequence is generally one that enables the vector to replicate independently of the recombinant microorganism chromosomes and also includes either origins of replication or autonomously replicating sequences. Various bacterial and viral origins of replication are in use in the art. Generally, any vector suitable to maintain, propagate or express polynucleotides to express a polypeptide in a recombinant microorganism can be used for expression in this regard. To name a few examples, chromosomal, episomal and virus-derived vectors, including vectors derived from bacterial plasmids, bacteriophages, yeast episomes, yeast chromosomal elements, viruses, such as baculoviruses, papoviruses, such as SV40, vaccinia viruses, adenoviruses, fowl pox viruses, pseudorabies viruses and retroviruses, and vectors derived from combinations thereof, such as those derived from plasmid and bacteriophage genetic elements, such as cosmids and phagemids, are used in the art.

The appropriate DNA sequence is inserted into the respective vector by any of a variety of well-known and routine techniques. In general, a DNA sequence for expression is joined to an expression vector by cleaving the DNA sequence and the expression vector with one or more restriction endonucleases and then joining the restriction fragments together using e.g. T4-DNA ligase. Linking is achieved by conventional techniques such as SOE PCR, DNA synthesis, blunt end ligation, or ligation at restriction enzyme sites. If suitable restriction sites are not available, synthetic oligonucleotide adapters or linkers can be used. The assembly of multiple sequences, including large numbers of genes, into a single nucleic acid molecule can be achieved by in vitro recombination using the combination of an exonuclease, a polymerase immobilized DNA polymerase, as described by Gibson et al. (Gibson, D. G., et al. Nature Methods (2009) 6, 5, 343-345; incorporated herein by reference in its entirety).

Numerous promoters are functional in bacterial cells and have been described in the literature, including constitutive, inducible, developmentally regulated, and environmentally regulated promoters. Of particular interest is the use of promoters (also referred to as transcriptional initiation regions) that are functional in the appropriate microbial recombinant microorganism. For example, if E. coli is used as a recombinant microorganism, then exemplary promoters that can be used include, but are not limited to, the phage lambda PL promoter, the E. coli lac, trp and tac promoters, the SV40 early and late promoters, promoters of retroviral LTRs or the CaMV 35S promoter.

A polynucleotide sequence encoding one or more enzymes described herein also can be part of an expression cassette that at a minimum includes a promoter, one or more nucleic acids encoding an enzyme disclosed herein, and a transcriptional termination signal sequence functional in a recombinant microorganism. The promoter can be any types of promoter as desired. For example, an inducible promoter or constitutive promoter and the expression cassette can further include an operably linked targeting sequence or transit or secretion peptide coding region capable of directing transport of the protein produced. The expression cassette can also further include a nucleotide sequence encoding a selectable marker.

In some embodiments a shuttle vector is used, which is capable of propagating in two or more different host species. Exemplary shuttle vectors are able to replicate in E. coli and/or Bacillus subtilis and in an obligate anaerobe, such as Clostridium. Upon insertion of an enzyme into the shuttle vector, the shuttle vector can be introduced into an E. coli host cell for amplification and selection of the vector. Such shuttle vector, e.g., a shuttle vector containing one or more sequences that encode enzymes, may also be introduced into a host cell that contains methyltransferase (e.g., an E. coli host cell expressing a methyltransferase) for the purpose of obtaining a methylated vector.

The vector can then be isolated and introduced into a selected microorganism for expression of the one or more enzymes. Any suitable shuttle vector or plasmid may be used, such as any of the shuttle plasmids described in the present disclosure below.

In some embodiments any of the cells described herein are introduced with a single vector, e.g., a shuttle plasmid DNA molecule, harbouring one or more nucleic acid sequences encoding an enzyme. A dual plasmid system may also be used. Two different plasmids carrying one or more sequences encoding an enzyme may be used. Each of the two different plasmids may carry a different selection marker. In some embodiments, the plasmid(s) are stably transformed in anaerobic cells.

For the production of an alkane and/or alkene it may be desirable to use a recombinant microorganism in which the heterologous nucleic acid sequence is stably maintained. In this regard a replicative plasmid may be used. This is a plasmid that has an origin of replication that is recognized by the host and that allows the plasmid to replicate as a stable, autonomous, extrachromosomal element that is partitioned during cell division into daughter cells.

In some embodiments one or more sequences encoding one or more enzymes are being integrated into the genome of a selected microorganism. Integration may be performed together with an expression cassette. In some embodiments integration into a host genome is a stable integration. Integration of a nucleic acid into a genome can be carried out using a transposase enzyme. The respective host genome may be a bacterial chromosome.

In embodiments where one or more nucleic acid sequences encoding an enzyme are being integrated into a host genome, one or more transposable elements may be used for this purpose. As an example, a class 2 transposable element, which may encode a transposase, may be included in a vector and encompass one or more coding sequences that encode one or more enzymes as disclosed herein. Transposases from a variety of organisms, ranging from bacteria to humans, are known. An example of a human transposase is the transposase THAP9, which contains a THAP-type zinc finger. Most transposases are classified under EC number EC 2.7.7.

A frequently used and well-examined transposase is the transposase of the transposon Tn10, which is found in bacteria such as E. coli. The transposon contains five genes, including the transposase and two proteins that confer resistance to the antibiotic tetracycline. The transposase of Tn10 is for example included in the plasmid pWH1(A31V)-mRFP or the plasmid pXT107. A further example of a suitable transposase is the transposase of the bacterial transposon Tn5 (EC:3.1.-.-), found in E. coli and Shewanella. The transposon encodes two proteins, the transposase Tn5 and a transposition inhibitor. Transposase Tn5 is a member of the RNase superfamily of proteins which includes retroviral integrases. The transposon codes for antibiotic resistance to kanamycin and other aminoglycoside antibiotics. The transposase Tn5 is for example included in the plasmid pO86A1. Yet a further example is the Tn3 transposase of the Tn3 transposon, found in prokaryotes. The transposon encodes β-lactamase, an enzyme that confers resistance to β-lactam antibiotics, the Tn3 transposase and Tn3 resolvase. Another example of a transposase that may be used is a Tc1/mariner-type transposase such as the Sleeping Beauty transposon system. The sleeping Beauty transposon system is a synthetic DNA transposon designed to introduce precisely defined DNA sequences into the chromosomes of vertebrates. It contains the Sleeping Beauty (SB) transposase. A large number of SB transposons have been developed as non-viral vectors, in particular for introduction of genes into genomes of vertebrate animals. Two further transposon-based systems that may be employed are the Frog Prince transposon/transposase system described in e.g. international patent application WO 2003/100070, or the TTAA-specific transposon piggyBac system. Yet a further example is the transposase of the transposon TniA from E. coli. The transposase of TniA is included in the plasmids pEC279, pEC54 and peH4H. A further example, the transposon Tn1000 transposase, also called “Transposase for transposon gamma-delta”, found in E. coli, is included e.g. in the E. coli F Plasmid. As further examples may serve the IS186A transposase, the REP-associated tyrosine transposase or the InsH transposase from E. coli.

Integration into a host genome may be performed in combination with positive selection, for example by providing a nucleic acid molecule that encodes a particular property or function that is required for survival of the recombinant host, e.g. bacteria, depends upon the presence or absence of a particular function encoded by the DNA that is introduced into the organism. The respective property or function may reside in resistance against a particular antibiotic, see also above. As an illustrative example, a resistance marker may be expressed and thus active when integrated in the host genome, e.g. chromosomal DNA, so that positive selection for host cells that carry the integrated nucleic acid, also called “integrand”, can be carried out. In some embodiments the respective resistance marker may not be expressed and thus active when encompassed by a vector and thus not yet integrated into a genome. The respective vector, e.g. plasmid, may include a repressor capable of binding in close proximity to the promoter of the antibiotic resistance gene. As a result, while included in the vector, the resistance marker is inactive. Once inserted into a bacterial genome, e.g. via transposition, the operator sequence to which the repressor is capable to bind is not present. The resistance marker is therefore functional in the host genome.

### Determining the Expression of an Enzyme

Where desired, the expression of a protein may be verified by detecting the amount of a nucleic acid or a protein or, in the case of an enzyme, by detecting the enzymatic activity. Any available method can be used to detect the presence of a nucleic acid or a protein in the context of a method disclosed herein. Such a method may include established standard procedures well known in the art. Examples of such techniques include, but are not limited to, RT-PCR, RNAse protection assay, Northern analysis, Western analysis, ELISA, radioimmunoassay or fluorescence titration assay. For an enzyme, the enzymatic activity may be used to assess the presence and to estimate the amount of protein present. A respective activity test may be carried out in vivo in the cell using a dye, where a dye is available that can be introduced into the cell.

Detection of a respective dye may be carried out using Fluorescence Microscopy, including Ratio Fluorescence Microscopy. Integrated optical imaging systems are commercially available that are capable of processing an entire assay from the biological preparation to the final data. Sensitive fluorescent indicators of a variety of physiologically important properties are available, and fluorescent reagents can be used for sensitively and specifically characterizing the intracellular distribution of proteins, nucleotides, ions, and lipids. In ratio fluorescence microscopy two fluorescence images are collected and the parameter of interest is quantified as a ratio of the fluorescence in one image to that in the other image. Fluorescence ratio images may be collected by sequentially exciting the sample with two different wavelengths of light and sequentially collecting two different images, by exciting the sample with a single wavelength of light and collecting images formed from light of two different emission wavelengths, or by exciting the sample with two wavelengths and collecting emissions of two wavelengths.

Assessing the amount of a protein in a cell may include assessing the amount of a nucleic acid, e.g. RNA, in a cell encoding the respective protein. A method as disclosed herein may further include measuring the expression of a sequence that encodes the sequence of GenBank accession no. WP_003113506.1 or a homolog thereof and/or a sequence of GenBank accession no. NP_010827.3 or a homolog thereof. This can for instance be achieved by determining the number of RNA molecules transcribed from an encoding nucleic acid molecule that is under the control of a selected promoter. A method commonly used in the art is the subsequent copy of RNA to cDNA using reverse transcriptase and the coupling of the cDNA molecules to a fluorescent dye. The analysis may for example be performed in form of a DNA microarray. Numerous respective services and kits are commercially available, for instance GeneChip® expression arrays from Affymetrix. Other means of determining gene expression of a transcription factor include, but are not limited to, oligonucleotide arrays, and quantitative Real-time Polymerase Chain Reaction (RT-PCR).

A nucleic acid probe may be used to probe a sample by any common hybridization method to detect the amount of nucleic acid molecules of the protein. In order to obtain nucleic acid probes chemical synthesis can be carried out. The synthesized nucleic acid probes may be first used as primers in a polymerase chain reaction (PCR) carried out in accordance with recognized PCR techniques, essentially according to standard PCR protocols utilizing the appropriate template, in order to obtain the respective probe. One skilled in the art will readily be able to design such a probe based on the sequences available for the biomarker. The hybridization probe can be labeled by standard labeling techniques such as with a radiolabel, enzyme label, fluorescent label, biotin-avidin label, chemiluminescence or a nanoparticle. After hybridization, the probes may be visualized using a standard technique.

The most frequently used methods for determining the concentration of nucleic acids include the detection by autoradiography, fluorescence, chemiluminescence or bioluminescence as well as electrochemical and electrical techniques. A further suitable technique is the electrical detection of a target nucleic acid molecule as disclosed in international patent applications WO 2009/041917 and WO 2008/097190, both being incorporated herein by reference in their entirety. In case of conflict, the present specification, including definitions, will control. A technique for the specific detection of a selected nucleic acid well established in the art is based on the hybridisation between a nucleic acid binding partner and a target nucleic acid. Typically the respective nucleic acid binding partner is immobilised onto a solid support, and subsequently one of the above mentioned detection methods is employed.

In some embodiments determining the level of expression of the gene of interest includes determining the level of transcription into mRNA. RNA encoding the protein of interest may be amplified using any available amplification technique, such as polymerase chain reaction (PCR), including multiplex PCR, nested PCR and amplification refractory mutation specific (ARMS) PCR (also called allele-specific PCR (AS-PCR), rolling circle amplification (RCA), nucleic acid sequence based amplification (NASBA), ligase chain reaction (LCR), QB replicase chain reaction, loop-mediated isothermal amplification (LAMP), transcription mediated amplification (TMA) and strand displacement amplification (SDA), including genome strand displacement amplification (WGSDA), multiple strand displacement amplification (MSDA), and gene specific strand displacement amplification (GS-MSDA). Detection of the obtained amplification products may be performed in numerous ways known in the art. Examples include, but are not limited to, electrophoretic methods such as agarose gel electrophoresis in combination with a staining such as ethidium bromide staining. In other embodiments a method as disclosed herein is accompanied by real time detection, such as real time PCR. In these embodiments the time course of the amplification process is monitored. A means of real time detection commonly used in the art involves the addition of a dye before the amplification process. An example of such a dye is the fluorescence dye SYBR Green, which emits a fluorescence signal only when bound to double-stranded nucleic acids.

As an illustrative example, real-time PCR may be used to determine the level of RNA encoding the protein of interest, such as an enzyme. Such a PCR procedure is carried out under real time detection, so that the time course of the amplification process is monitored. PCR is characterised by a logarithmic amplification of the target sequences. For the amplification of RNA, a reverse transcriptase-PCR is used. Design of the primers and probes required to detect expression of a protein is within the skill of a practitioner of ordinary skill in the art. In some embodiments RNA from the sample is isolated under RNAse free conditions and then converted to DNA via the use of a reverse transcriptase. Reverse transcription may be performed prior to RT-PCR analysis or simultaneously, within a single reaction vessel. RT-PCR probes are oligonucleotides that have a fluorescent moiety, also called reporter dye, attached to the 5′ end and a quencher moiety coupled to the 3′ end (or vice versa). These probes are typically designed to hybridize to an internal region of a PCR product. In the unhybridized state, the proximity of the fluor and the quench molecules prevents the detection of fluorescent signal from the probe. During PCR amplification, when the polymerase replicates a template on which an RT-PCR probe is bound, the 5′-3′ nuclease activity of the polymerase cleaves the probe. Thereby the fluorescent and quenching moieties are decoupled. Fluorescence increases then in each cycle, in a manner proportional to the amount of probe cleavage. Fluorescence signal emitted from the reaction can be measured or followed over time using equipment which is commercially available using routine and conventional techniques. Quantitation of biomarker RNA in a sample being evaluated may be performed by comparison of the amplification signal to that of one or more standard curves where known quantities of RNA were evaluated in a similar manner In some embodiments, the difference in biomarker expression is measured as the difference in PCR cycle time to reach a threshold fluorescence, or “dCT.”

### Producing Alkenes Using a Recombinant Microorganism

Using a recombinant microorganism as described herein, one or more alkene and/or alkane products may be produced. The production of one or more alkenes and/or alkanes may involve providing a substrate that can be converted into acetyl-CoA by the respective microorganism. As explained above, acetyl-CoA is a central metabolic building stone. A large variety of substrates, depending on the microorganism used, may potentially be suitable. In some embodiments the production may involve providing a substrate that includes CO. In some embodiments a substrate may be provided that includes CO₂. The substrate may be gaseous and contain a certain amount of CO and/or CO₂. A substrate may for example be provided that contains 25% (v/v) or more of carbon monoxide. A substrate may also be provided that contains 25% (v/v) or more of carbon dioxide. In some embodiments a substrate may for be provided that contains 35% (v/v) or more of carbon monoxide. A substrate may in some embodiments be provided that contains 35% (v/v) or more of carbon dioxide. CO may be a component that is included syngas, also called synthesis gas, a gaseous mixture essentially consisting of hydrogen and carbon monoxide. In some embodiments both CO and CO₂ may be included in syngas.

A method of producing an alkane and/or alkene as disclosed herein generally involves culturing the non-natural microorganism under suitable conditions in a suitable medium in the presence of CO and/or CO₂, for instance under an atmosphere that contains CO and CO₂. In some embodiments culturing a non-natural microorganism described herein may be carried out in substantially anaerobic culture conditions. The culture medium may contain vitamins and minerals sufficient to permit growth of the micro-organism used. Anaerobic media suitable for fermentation using CO are known in the art.

Producing one or more alkenes and/or alkanes may include a fermentation process. The fermentation process may be carried out in an aqueous culture medium, for example in a bioreactor. Culture in a bioreactor using culture media is a well-established technique in the art. Carbon monoxide and/or carbondioxide, including a substrate containing the same, as well as media, may be fed to a respective bioreactor in a continuous, batch or batch fed fashion.

The produced alkane or alkene may be secreted into the surrounding medium by the recombinant microorganism. The alkane or alkene may thus be recovered from the fermentation broth. In embodiments where the alkane or alkene is gaseous, a portion of the alkane or alkene may dissolve in the culture medium. As an example, butadiene has a solubility in water of about 7.4 g/L. Butene has a solubility in water of about 2.2/L, while propene has a solubility in water of about 0.6 g/L. Where the alkane or alkene is gaseous, typically the respective product is collected from the gas phase above the culture medium.

The produced alkane or alkene may be isolated and enriched, including purified, using any suitable separation and/or purification technique known in the art. In some embodiments the produced alkane or alkene is gaseous, such as n-butene or 1,3-butadiene. In some embodiments the produced alkane or alkene is a liquid such as isoprene. In case of a gaseous product the alkane or alkene may pass a filter, a gas separation membrane and/or a gas purifier such as an absorbent column, and then be stored in one or more cylinders. In some embodiments distillation may be employed to purify a product gas. Gas-liquid extraction (stripping) may in some embodiments be employed. In case of a liquid product isolation of the alkane or alkene may include centrifugation. The product may also be enriched via extraction using an organic phase. A respective organic phase containing the alkane and/or alkene may be separated from the fermentation liquid by adding a deemulsifier and/or a nucleating agent into the fermentation liquid. Illustrative examples of a deemulsifier include flocculants and coagulants. Illustrative examples of a nucleating agent include droplets of the acetyl-CoA derived compound itself and organic solvents such as dodecane, isopropyl myristrate, and methyl oleate. In some embodiments, an organic phase comprising the acetyl-CoA derived compound separates from the fermentation spontaneously. Methods of purifying the obtained alkane or alkene may also involve other standard techniques such as ultrafiltration, and one or more chromatographic techniques.

The listing or discussion of a previously published document in this specification should not necessarily be taken as an acknowledgement that the document is part of the state of the art or is common general knowledge.

The invention illustratively described herein may suitably be practiced in the absence of any element or elements, limitation or limitations, not specifically disclosed herein. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by exemplary embodiments and optional features, modification and variation of the inventions embodied therein herein disclosed may be resorted to by those skilled in the art, and that such modifications and variations are considered to be within the scope of this invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

Other embodiments are within the appending claims. In addition, where features or aspects of the invention are described in terms of Markush groups, those skilled in the art will recognize that the invention is also thereby described in terms of any individual member or subgroup of members of the Markush group.

In order that the invention may be readily understood and put into practical effect, particular embodiments will now be described by way of the following non-limiting examples.

### EXAMPLES

The examples illustrate techniques that can be used in methods disclosed herein and in obtaining a recombinant microorganism as described above.

### Example 1: Production of Methane, Ethane, Ethene, Propane, Propene, n-butane, 1-butene, 2-methylpropene, n-pentane, 1-pentene, n-hexane and 1-hexene

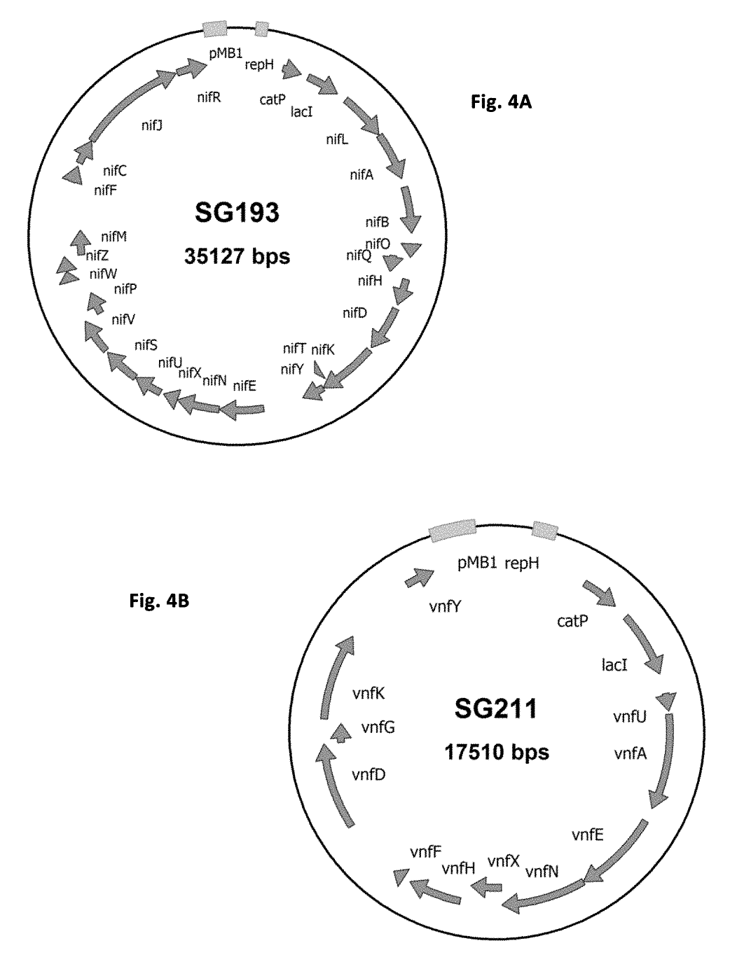

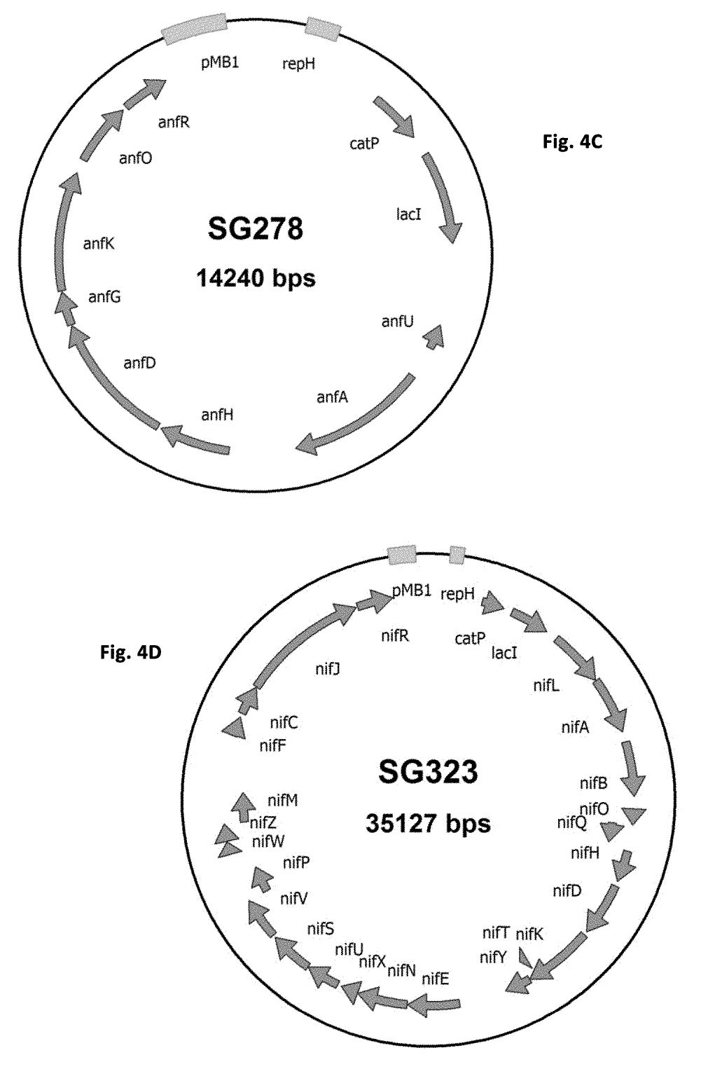

The construction of the expression plasmid SG323 (and all other plasmids described herein) was (were) performed by standard recombinant DNA and molecular cloning techniques using genes from Azotobacter vinelandii CA. FIG. 4D shows the plasmid SG323 (SEQ ID No: 4) which contains an engineered molybdenum nitrogenase pathway. This plasmid was electroporated into Clostridium Ijungdahlii cells by the following protocol:

### Genetic Transformation of Clostridium ljungdahlii Cells

Preparation of electrocompetent C. ljungdahlii cells: The procedure for making C. ljungdahlii electro competent cells was modified from a protocol reported previously (Kopke, M., al. Proc Natl Acad Sci USA. (2010) 107, 29, 13087-13092). All manipulations except centrifugation were carried out on ice in an anaerobic chamber. All buffers and centrifuge tubes were made ice cold and anoxic before use. All plastic ware was placed in the anaerobic chamber at least 24 h before use in order to eliminate any residual oxygen. Competent cells were maintained in SMP buffer (270 mM sucrose, 1 mM MgCl₂, 7 mM sodium phosphate, pH 6) with 10% DMSO at −80° C. until use. Electrocompetent C. ljungdahlii cells were prepared from cultures that were freshly inoculated from frozen stocks and then transferred twice in YTF liquid medium. About 15 to 16 h before preparation of competent cells, a mid- to late-log-phase culture was transferred into two serum bottles containing 100 mL fresh YTF medium supplemented with 40 mM DL-threonine (final optical density at 600 nm [OD₅₀₀]=0.004). After overnight growth at 37° C., early-log phase cells (OD₆₀₀=0.2 to 0.3; 200 mL) were harvested by centrifugation at 10,000 rpm for 10 min at 4° C. The cells were washed twice with 200 ml of SMP wash buffer and resuspended in the same buffer at a final concentration of 10¹⁰ to 10¹¹ cells/mL. Antifreezing buffer (60% DMSO—40% SMP, pH 6) was added to the competent cells a tone-fifth of the final resuspension volume to achieve a final concentration of 10% DMSO. The resulting competent cells (25 μL/tube) were stored at −80° C. for future use. The competence of these frozen competent cells remained stable for about 1 month.

Electrotransformation procedures for C. ljungdahlii: All procedures were carried out in an anaerobic chamber. Electrocompetent C. ljungdahlii cells (25 μl) were quickly transferred on ice from a −80° C. freezer to an anaerobic chamber. After thawing on ice (about 1 min), the cells were mixed with 1 to 5 μg DNA and transferred to a prechilled, 0.1-cm-gap cuvette. Cells were pulsed at 0.625 kV with resistance at 600 Ohm and a capacitance of 25 μF by using a microbial electroporation system. Immediately after the pulse, cells were recovered with 0.5 mL of fresh YTF medium, transferred to a pressure tube containing 10 mL of YTF medium supplemented with 5 mM L-cysteine (pH 7), and incubated at 37° C. The electroporated cells were allowed to recover at 37° C. until their cell densities were higher than immediately after the electroporation (about 9 to 12 h). Five-milliliter volumes of the outgrowth cultures or appropriately diluted cultures were mixed with 20 mL of YTF molten agar (1.5%) containing an appropriate antibiotic and poured into a petri dish. After the agar mixtures were solidified, plates were incubated upside down. After 2-3 days colonies became visible and could be inoculated in liquid culture.

YTF Medium:

10 g/L yeast extract

16 g/L Bacto tryptone

4 g/L sodium chloride

5 g/L fructose

Supplemented with 2 mM L-cysteine

The final pH is 6.0.

Methane, ethane, ethene, propane, propene, n-butane, 1-butene, 2-methylpropene, n-pentane, 1-pentene, n-hexane and 1-hexene production from syngas (mixture of CO, CO₂, and H₂) and electrons by gas fermentation with electron supply by electrode was performed using said genetically engineered C. ljungdahlii cells harboring the plasmid SG323. The materials and methods used are described in the following:

Used Medium for Growth with Gas:

| | | |
|---|---|---|
| NH₄Cl: | 1.0 | g |
| KCl: | 0.1 | g |
| MgSO₄ × 7H₂O | 0.2 | g |
| NaCl | 0.8 | g |
| KH₂PO₄ | 0.1 | g |
| CaCl₂ × 2H₂O | 20.0 | mg |
| Trace Elements (see below) | 10.0 | mL |
| Wolfe's Vitamin Solution (see below) | 10.0 | mL |
| Reducing Agent (see below) | 10.0 | mL |
| Distilled water | 980.0 | mL |

The final pH is 6.0.

Trace Elements:

| | | |
|---|---|---|
| Nitrilotriacetic acid | 2.0 | g |
| MnSO₄ × H₂O | 1.0 | g |
| Fe(SO₄)₂(NH₄)₂ × 6H₂O | 0.8 | g |
| CoCl₂ × 6H₂O | 0.2 | g |
| ZnSO₄ × 7H₂O | 0.2 | mg |
| CuCl₂ × 2H₂O | 20.0 | mg |
| NiCl₂ × 6H₂O | 20.0 | mg |
| Na₂MoO₄ × 2H₂O | 20.0 | mg |
| Na₂SeO₄ | 20.0 | mg |
| Na₂WO₄ | 20.0 | mg |
| Distilled water | 1.0 | L |

Add nitrilotriacetic acid to water and adjust to pH 6.0 with KOH. Add remainder of ingredients.

Wolfe's Vitamin Solution:

| | | |
|---|---|---|
| Biotin | 2.0 | mg |
| Folic acid | 2.0 | mg |
| Pyridoxine hydrochloride | 10.0 | mg |
| Thiamine × HCl | 5.0 | mg |
| Riboflavin | 5.0 | mg |
| Nicotinic acid | 5.0 | mg |
| Calcium D-(+)-pantothenate | 5.0 | mg |
| Vitamin B12 | 0.1 | mg |
| p-Aminobenzoic acid | 5.0 | mg |
| Thioctic acid | 5.0 | mg |
| Distilled water | 1.0 | L. |

Reducing Agent:

| | |
|---|---|
| L-Cysteine (free base) | 4.0 g |
| Distilled water | 100.0 mL. |

Used gas: 60% CO, 10% CO₂, 30% H₂.

The transformed cells were cultivated in the medium above supplied the 10 μg/mL thiamphenicol (antibiotic) at 37° C. in CSTR bioreactor provided with syngas (60% CO, 10% CO₂, 30% H₂) at 1 bar pressure and an electrode with power supply for electricity supply. Gene expression was induced by 10 mM IPTG when a cell density of 0.3 OD₆₀₀ was reached. Product analysis was performed by GC-MS (Shimadzu GC-MS QP5050A). Headspace gas sampling was conducted using a 500 iL Hamilton gastight syringe. Methane, ethane, ethene, propane, propene, n-butane, 1-butene, 2-methylpropene, n-pentane, 1-pentene, n-hexane and 1-hexene production could be detected by GC-MS.

### Alternative Embodiments

Plasmid SG193 (FIG. 4A and SEQ ID NO: 1), Plasmid SG211 (FIG. 4B and SEQ ID NO: 2) and Plasmid SG278 (FIG. 4C and SEQ ID NO: 3) were also used to engineer Clostridium ljungdahlii as well as Clostridium autoethanogenum cells to produce methane, ethane, ethene, propane, propene, n-butane, 1-butene, 2-methylpropene, n-pentane, 1-pentene, n-hexane. The same basic protocol and process like described in the preferred embodiment was performed. Different substrates were used as listed in the following:

For all substrates used, also those of the subsequent working examples:

60% CO, 10% CO₂, 30% H₂

100% CO

30% CO₂ and 60% H2

60% CO, 10% CO₂, 30% H2 and electrons

100% CO and electrons

100% CO₂ and electrons

30% CO₂ and 60% H₂ and electrons

### Example 2: Positive Selection of Chromosomal Integrations and Multicopy Chromosomal Integrations in Clostridia Via Transposase

The following example illustrates a method of integrating heterologous DNA (metabolic gene clusters or any other functional or non-functional nucleic acid sequence) into a giving host or chassis microorganism. Positive selection (or direct genetic selection) of mutant bacteria is possible whenever survival of the recombinant bacteria depends upon the presence or absence of a particular function encoded by the DNA that is introduced into the organism. The advantage of a selection method over a screening method is that growth of bacteria with the specific desired mutation is greatly favored over bacteria lacking that specific mutation, thus facilitating the identification of the preferred mutants. Since all functions that are required for transposition such as the transposase and the integration cassette flanked by the recognized base sequences for transposition, are introduced on plasmids, direct positive selection via an antibiotic selection marker as part of the integration cassette is not possible. The resistance marker is functional on the plasmid and therefor (most plasmids also have high copy numbers) no growth advantage arises from an integration event. However, when the marker is only expressed and thus active when integrated in the chromosomal DNA, positive selection for integrands is possible.

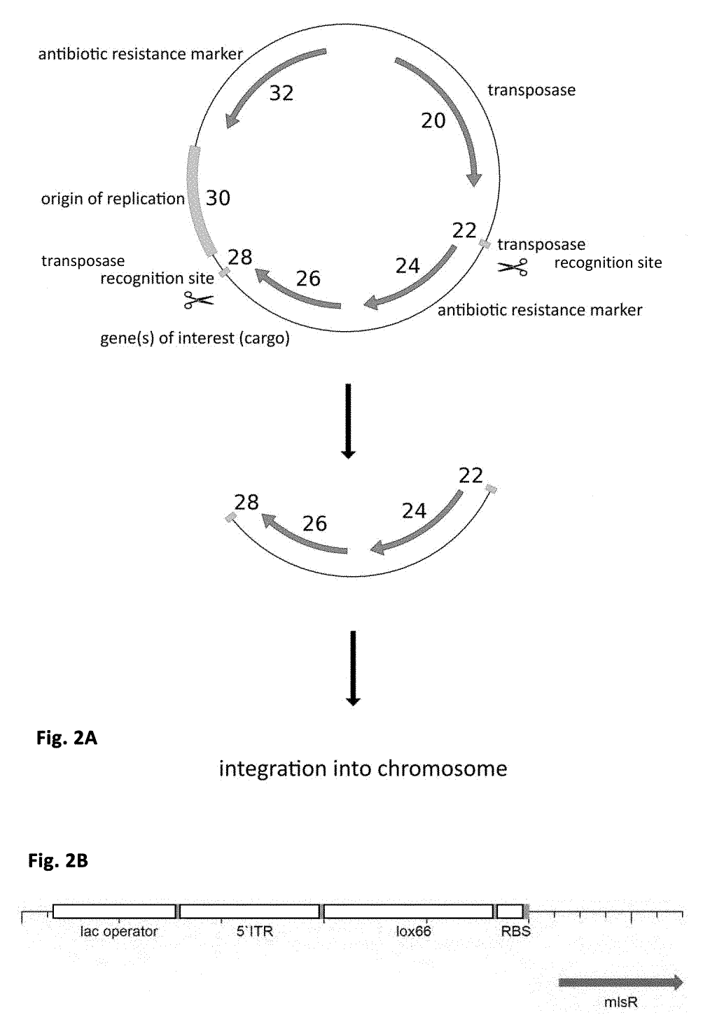

FIG. 2A provides an overview of the basic system for integration of a nucleic acid sequence into a bacterial chromosome by a cut and paste transposase 20. All functions necessary are located on a single plasmid. A backbone antibiotic resistance marker 32 and an origin of replication 30 for propagation of the vector in the transformed bacterial cell. The integration cassette is flanked by transposase recognition sites 22 and 28; the integration cassette also comprises a second antibiotic resistance gene. The transposase 20 cuts the DNA at the two recognition sites and integrates this nucleic acid sequence into the bacterial chromosome.

The expression of the resistance marker is deactivated when located on the plasmid by a repressor binding in close proximity to the promoter of antibiotic gene. After transposition the operator sequence the repressor binds to is gone and resistance is conferred. This principle is shown in more detail in FIG. 2B.

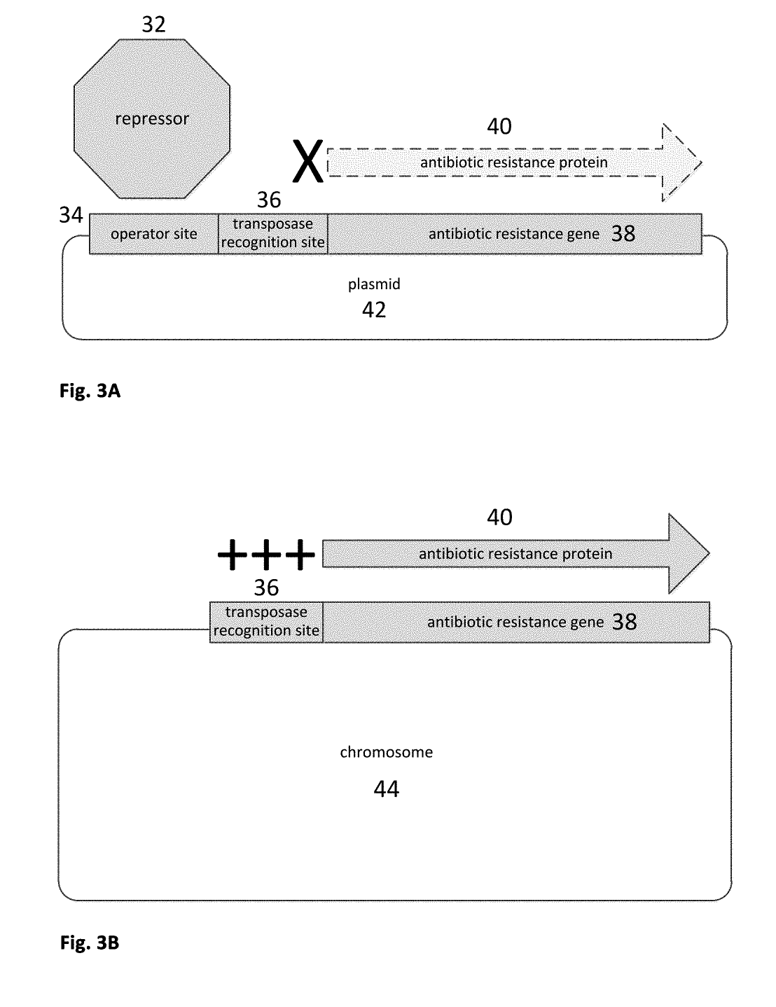

FIG. 3A depicts a positive selection system implemented on top of the minimal system of FIG. 2A. Here an additional repressor 32 binds to the operator site 34, blocking expression of an antibiotic resistance gene 38. The corresponding antibiotic resistance protein is not expressed and no resistance is conferred. FIG. 3B illustrates the situation where the cassette has been integrated into the bacterial chromosome 44 by action of the transposase. The operator site 34 is no longer present, and expression of the antibiotic resistance gene 38 leads to antibiotic resistance. Thus it is possible to select for insertional chromosomal mutants.

The 5′ITR (inverted terminal repeat recognized and cut by the transposase) and the lox66 site are also a promoter for the erythromycin/clarythromycin antibiotic resistance marker mlsR, cf. FIG. 2B. The repressor (lacI) binds to the lac operator sequence and blocks transcription (this can also be reversed by the addition of IPTG). After transposition the lac operator sequence is gone and mlsR is expressed and can be selected for (RBS=ribosome binding site). Surprisingly, the respective process also facilitates the generation of multicopy integrations and the transposition of high cargo. Both positive selection of integrands and a prolonged timeframe for transposition events to take place can be achieved.

### Initial System for Random Chromosomal Integration in Clostridia Via Transposase

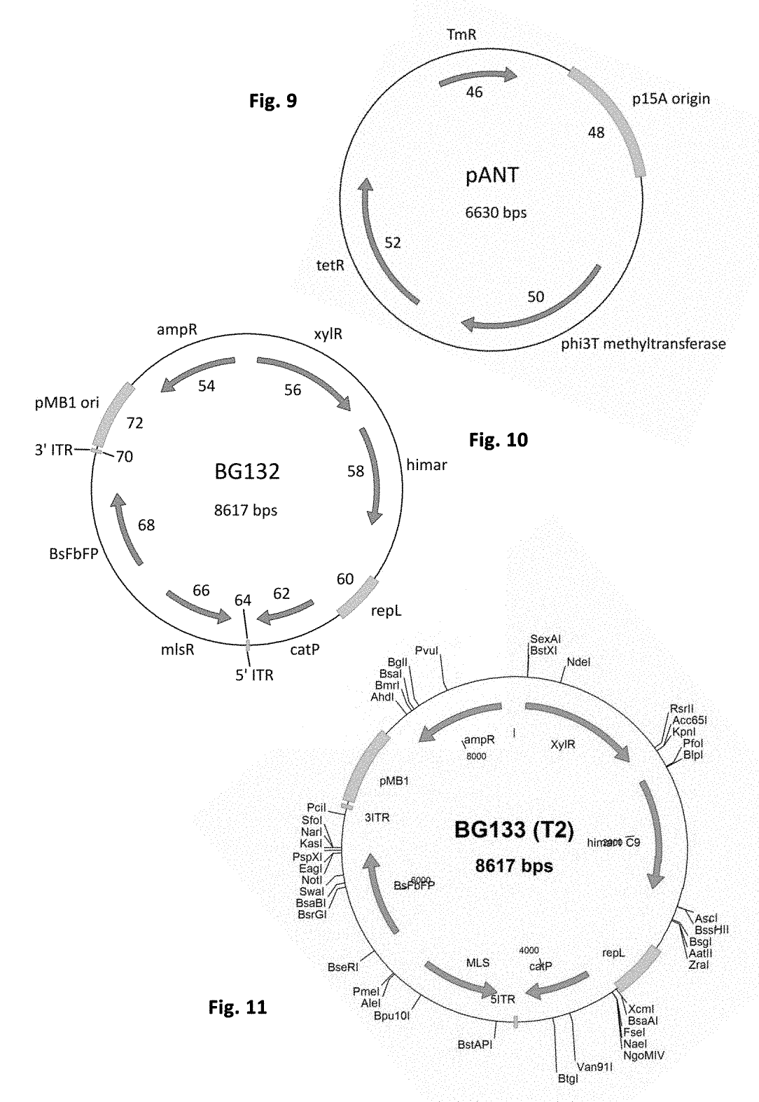

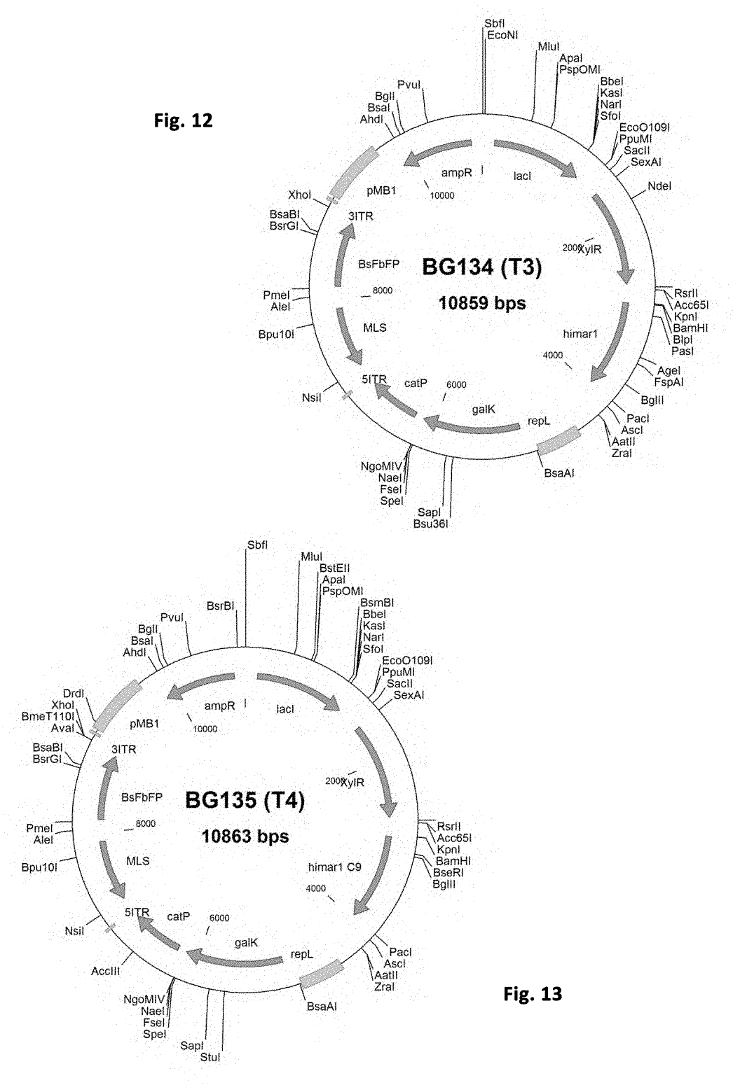

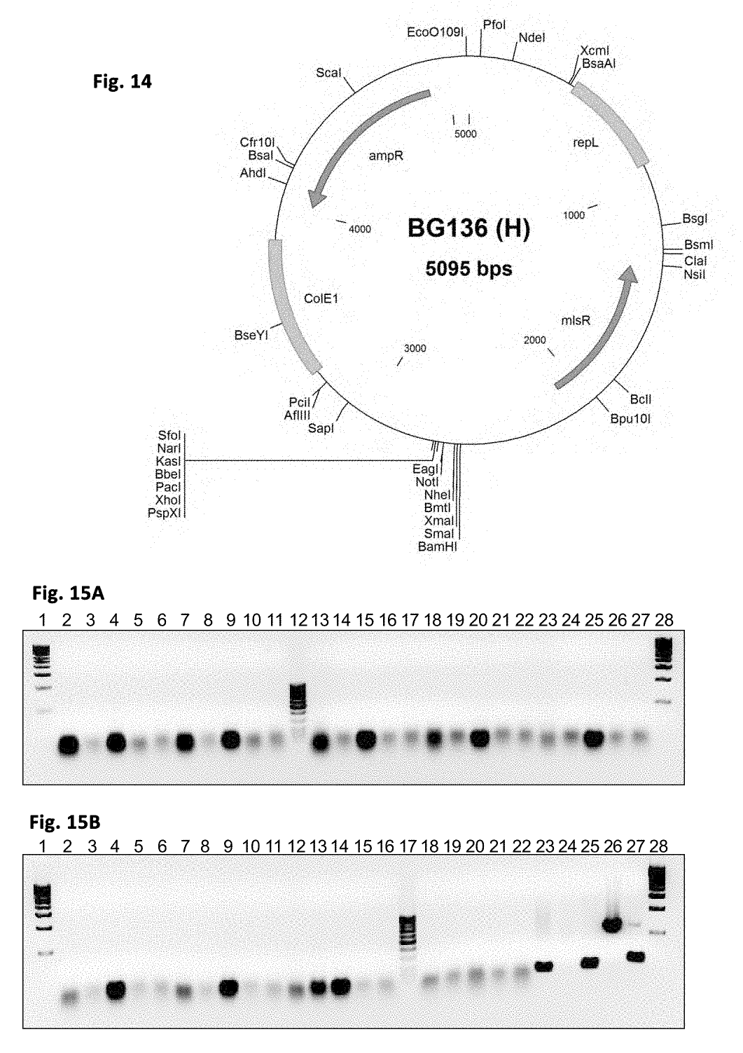

The chromosomal integration of a anaerobic fluorescence protein (BsFbFP) was established by testing four different E. coli-clostridial shuttle plasmids harboring everything necessary for transposition (integration cassette with antibiotic resistance marker and BsFbFP flanked by the recognized base sequences for transposition also called inverted terminal repeats and a transposase under control of an xylose inducible promoter) and a negative control which consists of only a E. coli-clostridial shuttle plasmid with no additional functions. The wild type transposase himar1 and also a hyperactive variant himar1 C9 were tested. For genetic transformation of Clostridium acetobutylicum the plasmids are methylated in vivo by the plasmid pANT. The vectors used are of SEQ ID NOs: 9 to 14. Their vector maps can be taken from FIG. 9 to FIG. 14.

Transformation of Clostridium acetobutylicum was performed according to the following protocol:

1. Methylation of Plasmid DNA
Transformation of plasmids in E. coli XL1 Blue MR together with plasmid pANT (Φ3T methyltransferase)
plates with antibiotics: ampicillin, chloramphenicol and tetracycline
Midipreperation (concentration of DNA should be not too low).

2. 5 ml Starter Liquid Culture C. acetobutylicum with 2×YTG Medium

In the evening 50 ml 2×YTG medium inoculated with 50-2001 pre-culture and cultured at 36° C. over night.
Harvest cells at OD₆₀₀=0.8-1
Centrifuge cells at 2200 g for 10 min (4° C.)
Wash the cells with 40 ml ice cold HEB⁺ buffer
Centrifugation at 2200×g for 10 min (4° C.)
Washing of cells with HEB+ buffer three times
Suspend in 2 ml HEB buffer; aliquot to 600 μL (one transformation)
Use cold 4 mm electroporation cuvette and up to 4 μL plasmid (600 μL cells)
Incubate for 2 min on ice
Electroporation at 2500 V
Addition of 1.4 mL 2×YTG medium and incubation at 36° C. for 4 hours
Plate on selection plates with appropriate antibiotics e.g. clarythromycin (Cla) or thiamphenicol (Tm)
Incubate for 3 days at 36° C.
Observe colonies and inoculate liquid culture

3. Medium:
2×YTG: 5 g/L glucose
16 g/L tryptone
10 g/L yeast extract 5 g/L NaCl
pH 7.0
HEB⁺ buffer:
7 mM HEPES
272 mM sucrose
5 mM MgCl₂
pH 7.4
HEB buffer contains no MgCl₂
Concentrations of the used, methylated plasmids:
   pANT methylated plasmid BG00132: 350 ng/μL
   pANT methylated plasmid BG00133: 300 ng/μL
   pANT methylated plasmid BG00134: 280 ng/μL
   pANT methylated plasmid BG00135: 385 ng/μL
   pANT methylated plasmid BG00136: 500 ng/μL

Electroporation of all constructs was successful and 10 to 50 colonies on each plate were observed.

Integration was performed as follows:

4 colonies of each transformation (1,2,3,4,5) were inoculate in fresh medium (5 mL) as follows:
Medium 1=2×YTG 5 g/L glucose
Medium 2=2×YTGX 5 g/L glucose+2.5 g/L xylose
Medium 3=2×YTG 5 g/L glucose+10 g/L xylose.

Observation: pre cultures with plasmids BG132, BG134 BG135 have been grown faster than the others.

2 μL of each pre culture were plated on 2×YT plates containing
5 g/L glucose
5 g/L glucose with 2.5 g/L xylose—10 g/L xylose

22 distinct colonies were struck out on the following plates (1st round):
1. 2×YTG plates
2. 2×YTGX with 5 g/L glucose and 2.5 g/L xylose
3. 2×YTX with 10 g/L xylose

22 colonies were struck out as follows (2nd round):
1. 2×YTG plates
2. 2×YTGX with 5 g/L glucose and 2.5 g/L xylose
3. 2×YTX with 10 g/L xylose

22 colonies taken from the plates were struck out as follows (3rd round):
1. 2×YTG plates
2. 2×YTGX with 5 g/L glucose and 2.5 g/L xylose
3. 2×YTX with 10 g/L xylose

22 colonies taken from the plates were struck out as follows (4th round):
1. 2×YTG plates without antibiotics
2. 2×YTG plates with 10 μg/mL clarythromycin (Cla)
3. 2×YTG plates with 20 μg/mL thiamphenicol (Tm)

Plates of round 4 were examined:

| Integrand | plate | clone | medium | vector |
|---|---|---|---|---|
| 1 | t2_X27B_9 | 1 | 2xYTX with 10 g/L | BG133 |
| | | | xylose | |
| 2 | t2_X27B_9 | 2 | 2xYTX with 10 g/L | BG133 |
| | | | xylose | |
| 3 | t2_X27B_2 | 1 | 2xYTX with 10 g/L | BG133 |
| | | | xylose | |
| 4 | t2_X27B_2 | 2 | 2xYTX with 10 g/L | BG133 |
| | | | xylose | |
| 5 | t4_X19B | 17 | 2xYTX with 10 g/L | BG135 |
| | | | xylose | |
| 6 | t2_GX19B | 5 | 2xYTGX with glucose | BG133 |
| | | | and 2, 5 g/L xylose | |
| 7 | t2_GX19B_4 | 1 | 2xYTGX with glucose | BG133 |
| | | | and 2, 5 g/L xylose | |
| 8 | t2_GX19B_4 | 2 | 2xYTGX with glucose | BG133 |
| | | | and 2, 5 g/L xylose | |
| 9 | t2_GX4.5B_21 | 1 | 2xYTGX with glucose | BG133 |
| | | | and 2, 5 g/L xylose | |
| 10 | t2_GX4.5B_21 | 2 | 2xYTGX with glucose | BG133 |
| | | | and 2, 5 g/L xylose | |
| 11 | t2_GX4.5B_21 | 3 | 2xYTGX with glucose | BG133 |
| | | | and 2, 5 g/L xylose | |
| 12 | t2_X4.5B | 9 | 2xYTX with 10 g/L | BG133 |
| | | | xylose | |

Summary; integrands could be isolated, the controls were all negative, but very few integrands compared to the huge number of tested clones. A number of clones still had not lost the plasmid.

Determination of the Loci of Integration was Performed as Follows:

Preparation of genomic DNA of the Clostridium acetobutylicum integration strains (see table) using a kit from Macherey-Nagel (special protocol for difficult to lyse bacteria) approx. 20 mL liquid culture was used. Concentration of gDNA was determined by nanodrop.
Integrand 1=470 ng/μL
Integrand 2=460 ng/μL
Integrand 3=520 ng/μL
Integrand 4=710 ng/μL
Integrand 5=380 ng/μL
Integrand 6=460 ng/μL
Integrand 7=440 ng/μL
Integrand 8=450 ng/μL
Integrand 9=420 ng/μL
Integrand 10=400 ng/μL
Integrand 11=570 ng/μL
Integrand 12=370 ng/μL

The samples were sent to LGC Genomics GmbH (Berlin, Germany) to perform PCR walking for loci determination.

Additionally PCR was Performed to Verify the Integrands:

Polymerase Chain Reaction (PCR) Integration Check

PCR 1: Test Presence of Fluorescence Protein BsFBFP
Template: genomic DNA of integrands (Clostridium acetobutylicum) and wild type gDNA, positive control vector BG132

(SEQ ID NO: 58)

| | |
|---|---|
| Primer BG272 forward: | GCACTTCCTCTTGTTGGAAAT |

(SEQ ID NO: 59)

| | |
|---|---|
| Primer BG273 reverse: | ACTTGTGCAAGTCCACTTAAA |

PCR 2: Check Integration Marker
Template: genomic DNA of integrands (Clostridium acetobutylicum) and wild type gDNA, positive control vector BG132

(SEQ ID NO: 60)

| | |
|---|---|
| Primer BG274 forward: | CTATGTGGCGCGGTATTATC |

(SEQ ID NO: 61)

| | |
|---|---|
| BG275 reverse: | GCATTTAAGCGTCAGAGCATGG |

PCR 3: Check Integration Marker 2
Template: genomic DNA of integrands (Clostridium acetobutylicum) and wild type gDNA, positive control vector BG132

(SEQ ID NO: 62)

| | |
|---|---|
| Primer BG276 forward: | TTTAATCGTGGAATACGGGTTTG |

(SEQ ID NO: 63)

| | |
|---|---|
| Primer BG277 reverse: | GTGAGCTATTCACTTTAGGTTTAGG |

PCR 4: Check Backbone
Template: genomic DNA of integrands (Clostridium acetobutylicum) and wild type gDNA, positive control vector BG132

| (SEQ ID NO: 64) | |
|---|---|
| Primer BG278 forward: | CAAAAGGCCAGGAACCGTAA |
| (SEQ ID NO: 65) | |
| Primer BG279 reverse: | GCGTCAGACCCCGTAGAAAA |

PCR 5: Check Backbone 2
Template: genomic DNA of integrands (Clostridium acetobutylicum) and wild type gDNA, positive control vector BG132

(SEQ ID NO: 66)

| | |
|---|---|
| Primer BG280 forward: | ATTGTAAACCGCCATTCAGAG |

(SEQ ID NO: 67)

| | |
|---|---|
| Primer BG281 reverse: | ATACCGTTGCGTATCACTTTC |

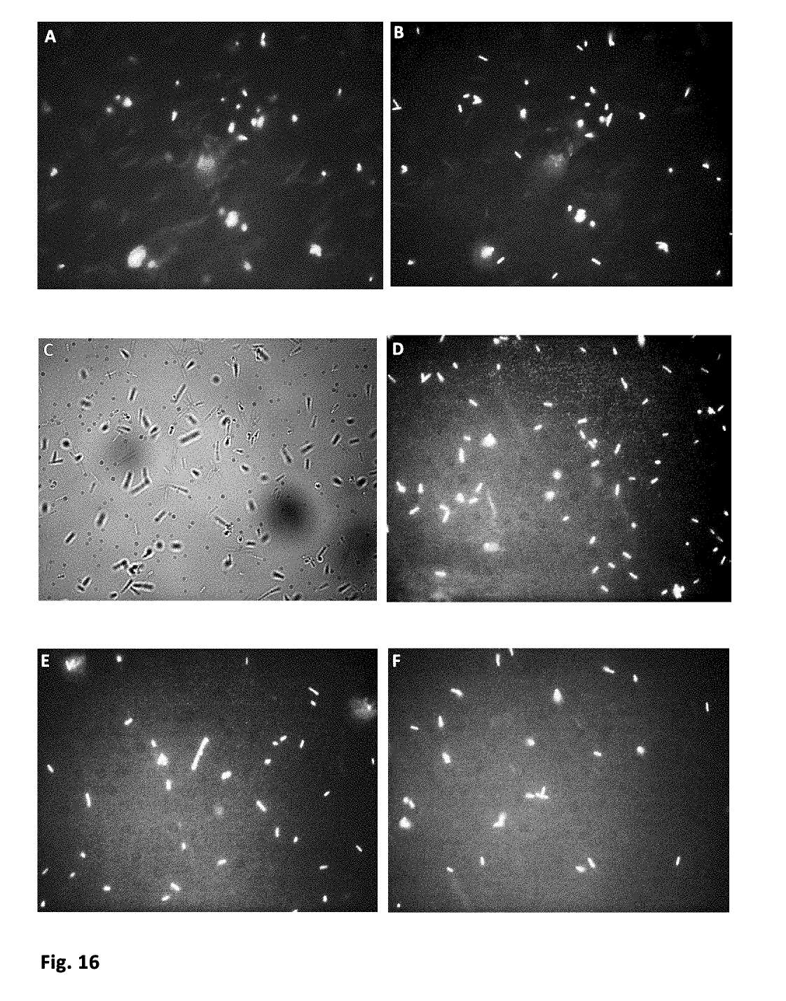

PCR 1-5 were performed with 30 cycles, 1 min elongation, 55° C. annealing temperature, 50 μL total size, phusion polymerase. The PCR products were then examined by 0.8% analytic DNA agarose gel electrophoresis. The results are depicted in FIGS. 15A and 15B.

Gel 1, FIG. 15A: loading of 10 μL each; allocation; name; result were as follows:
Lane 1: 3 μL ladder 1 kB purchased from Roth (Carl Roth GmbH & CO. KG, Karlsruhe, Germany)
Lane 2-6: integrand 1 PCR 1-5; result: 1 and 3 positive 2,4,5 negative
Lane 7-11: integrand 2 PCR 1-5; result: 1 and 3 positive 2,4,5 negative
Lane 12: 3 μL ladder 100 bp purchased from Roth
Lane 13-17: integrand 3 PCR 1-5; 1 and 3 positive 2,4,5 negative
Lane 18-22: integrand 4 PCR 1-5; 1 and 3 positive 2,4,5 negative
Lane 23-27: integrand 5 PCR 1-5; 1 and 3 positive 2,4,5 negative
Lane 28: 3 μL ladder 1 kB purchased from Roth

Gel 2, FIG. 15B: loading of 10 μL each; allocation; name; result were as follows:
Lane 1: 3 μL ladder 1 kB purchased from Roth
Lane 2-6: integrand 6 PCR 1-5; result: 1 and 3 positive 2,4,5 negative
Lane 7-11: integrand 7 PCR 1-5; result: 1 and 3 positive 2,4,5 negative
Lane 12-16: integrand 8 PCR 1-5; 1 and 3 positive 2,4,5 negative
Lane 17: 3 μL ladder 100 bp purchased from Roth
Lane 18-22: wild type PCR 1-5; all negative
Lane 23-27: integrand 5 PCR 1-5; 1,3,4,5 positive 2 negative
Lane 28: 3 μL ladder 1 kB purchased from Roth

Genome walking—first results from LGC Genomics. The reads (first number corresponds to the integrand vide supra) can be taken from SEQ ID NOs: 68 to 78.

Table with Initial Analysis:

| integrand | name | locus 1 | nomenclature | locus 2 | Nomencl. | locus 3 |
|---|---|---|---|---|---|---|
| 1 | t2_X27B_{—} | Predicted Fe—S oxidoreductase, | CA_C1838 | | | |
| | | YMCB B. subtilis ortholog | | | | |
| 2 | t2_X27B_9 | Predicted Fe—S oxidoreductase, | CA_C1838 | — | | |
| | | YMCB B. subtilis ortholog | | | | |
| 3 | t2_X27B_{—} | | | | | |
| 4 | t2_X27B_2 | | | | | |
| 5 | t4_X19B | Uracyl DNA glycosilase | CA_C219 | — | | |
| 6 | t2_GX19B | | | | | |
| 7 | t2_GX19B | Predicted Fe—S oxidoreductase, | | — | | |
| | | YMCB B. subtilis ortholog | | | | |
| 8 | t2_GX19B_4 | | | vor | CA_C3587 | Beta |
| | | | | DnaD | | glucosidase |
| 9 | t2_GX4.5B_21 | Predicted Fe—S oxidoreductase, | | — | | |
| | | YMCB B. subtilis ortholog | | | | |
| 10 | t2_GX4.5B_21 | Predicted Fe—S oxidoreductase, | | vor | CA_C3587 | |
| | | YMCB B. subtilis ortholog | | DnaD | | |
| 11 | t2_GX4.5B_21 | Predicted Fe—S oxidoreductase, | | — | | |
| | | YMCB B. subtilis ortholog | | | | |
| 12 | t2_X4.5B | Predicted Fe—S oxidoreductase, | CA_C1838 | | | |
| | | YMCB B. subtilis ortholog | | | | |

Result: PCR genome walking performed by lgc genomics was successful but not all loci could be determined. Lgc performed a second run with optimized setup.

Fluorescence Microscopy of Integrands

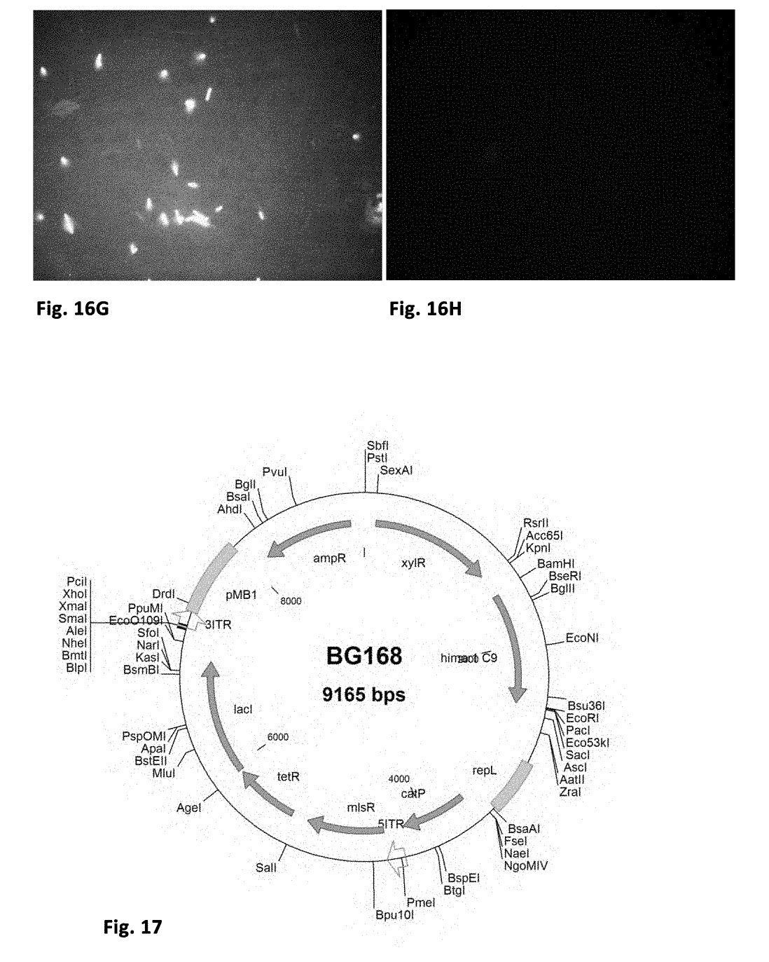

Integrands were analysed on a Leica fluorescence microscope. Images of Clostridium acetobutylicum Integrand 1 are shown in FIGS. 16A, 16B and 16C, magnification is 1000×. An image of Clostridium acetobutylicum Integrand 2 is shown in FIG. 16D. Images of Clostridium acetobutylicum Integrand 10 are shown in FIGS. 16E, 16F and 16G. An image of a Clostridium acetobutylicum wild type control is shown in FIG. 16H. All examined integrands show fluorescence. The wild type control is negative.

Genome walking—the second round of PCR Genome walking by LGC Genomics gave reads that can be taken from SEQ ID NOs: 79 to 85.

### Example 3: Development of a Positive Selection System for Random Chromosomal and Random Multicopy Chromosomal Integrations in Clostridia Via Transposase

To further improve the initial, laborious chromosomal integration system a new plasmid with a repressor blocking transcription of the integration marker was constructed. Subsequently, to test the new system, the following E. coli-Clostridium acetobutylicum shuttle vectors were transformed into Clostridium acetobutylicum. To transform the plasmids into Clostridium acetobutylicum it was first methylated in vivo with the help of plasmid pANT as described above. To achieve this, the plasmids were transformed into E. coli XL1-Blue cells (see above). Vector BG133 (abbreviated “N”) the best performing vector from Example 2, vide supra, had the sequence of SEQ ID NO: 11. Vector BG168 (abbreviated “I”, cf. FIG. 17) had the sequence of SEQ ID NO: 15. The temperature-sensitive plasmid BG182 (cf. FIG. 18) had the sequence of SEQ ID NO: 16. After plasmid preparation Clostridium acetobutylicum can be genetically transformed with e.g. plasmid BG168 by the above protocol: C. acetobutylicum was cultivated anaerobically in 2×YTG medium (16 g/L tryptone, 10 g/L yeast extract, 5 g/L NaCL, 5 g/L Glucose) until an optical density of 0.6 to 0.8 is reached. All following steps were also conducted anaerobically. The cells were centrifuged for 10 min at 2000 g (4° C.) and subsequently washed with electroporation buffer (7 mM Hepes, 270 mM sucrose, 5 mM MgCl₂, pH 7.4). This was repeated another two times. Then, a cold electroporation cuvette was prepared with 5 kg plasmid DNA, 500 μL competent cells and electroporated at 2500V for more than 3 ms. After 2 hours of regeneration with fresh 2×YTG medium the cells were plated on 2×YTG agar plates supplemented with suitable antibiotics. After 2 days genetically transformed colonies became visible. The chromosomal integrands were isolated via the positive selection system: cells were cultivated with addition of xylose for transposase induction and with addition of the second antibiotic. After two serial transfers cells were plated again and then picked for individual testing. Integrands were identified by loss of resistance for the first (backbone) maker which is thiamphenicol while remaining resistant for the second (integration) marker clarithromycin. More than 100 integrands could be isolated. Only a fraction was further analyzed. Preparation of genomic DNA of the Clostridium acetobutylicum integration strains was performed using a kit from Macherey-Nagel (special protocol for difficult to lyse bacteria); approx. 20 mL liquid culture was used.

Comparison of Chromosomal Integration by Serial Transfer (Positive Selection)

Serial Transfer Scheme:

| Name | | (μg/mL) | | |
|---|---|---|---|---|
| N(I)C5V#Ü# | 5 Vks (2xYTX medium) | Cla 5 | Transfer: | 3 times |
| | 7 mL | | 5 μL | |
| N(I)C20V#Ü# | 5 Vks (2xYTX medium) | Cla 20 | Transfer: | 3 times |
| | 7 mL | | 5 μL | |
| N(I)C100V#Ü# | 5 Vks (2xYTX medium) | Cla 100 | Transfer: | 3 times |
| | 7 mL | | 5 μL | |

Legend:
N=plasmid N (vide supra)
I=plasmid I (vide supra)
V=liquid culture (replicate) 7 mL size
Ü=round of serial transfer
#=number
Cla=clarythromycin

Isolation of Integrands:

After serial transfer in liquid culture 5 μL of each replicate was plated on 2×YTG. 28 colonies were then struck out on the following plates:
1. 2×YTG without antibiotics
2. 2×YTG with 5 μg/mL clarythromycin
3. 2×YTG with 20 μg/mL thiamphenicol
   When the plates were compared all clones from the “N” series showed thiamphenicol resistance (backbone marker) meaning that plasmid is still there. However, all clones from the “I” series were only clarythromycin resistant (integration marker) but not thiamphenicol resistant (backbone marker) meaning they are all chromosomal integrands.

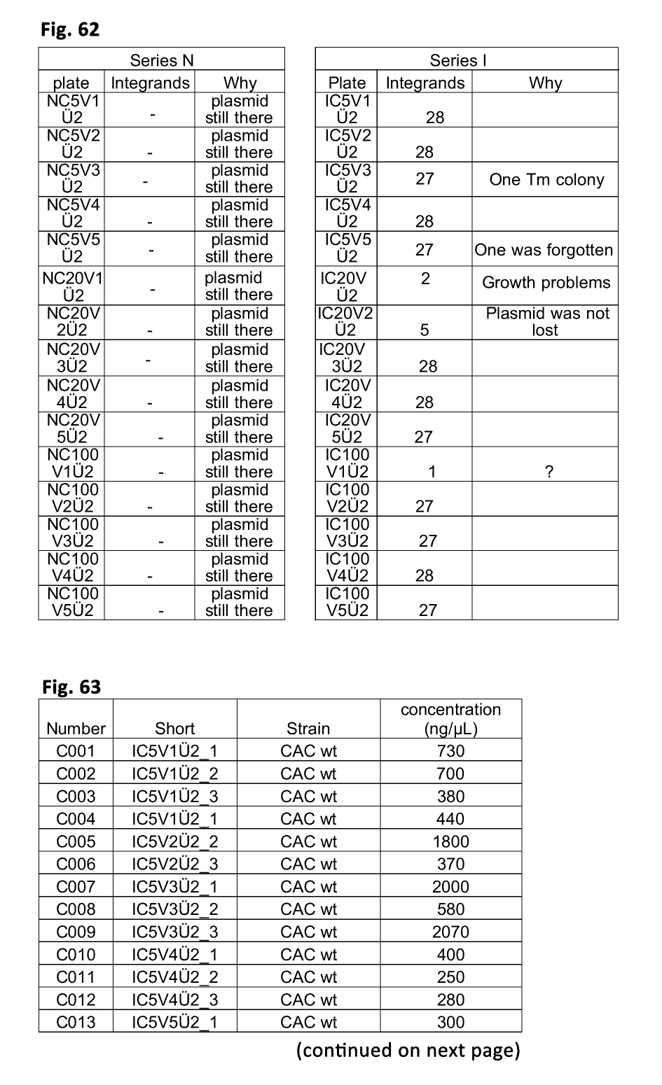

The results are summarized in the table shown in FIG. 62.

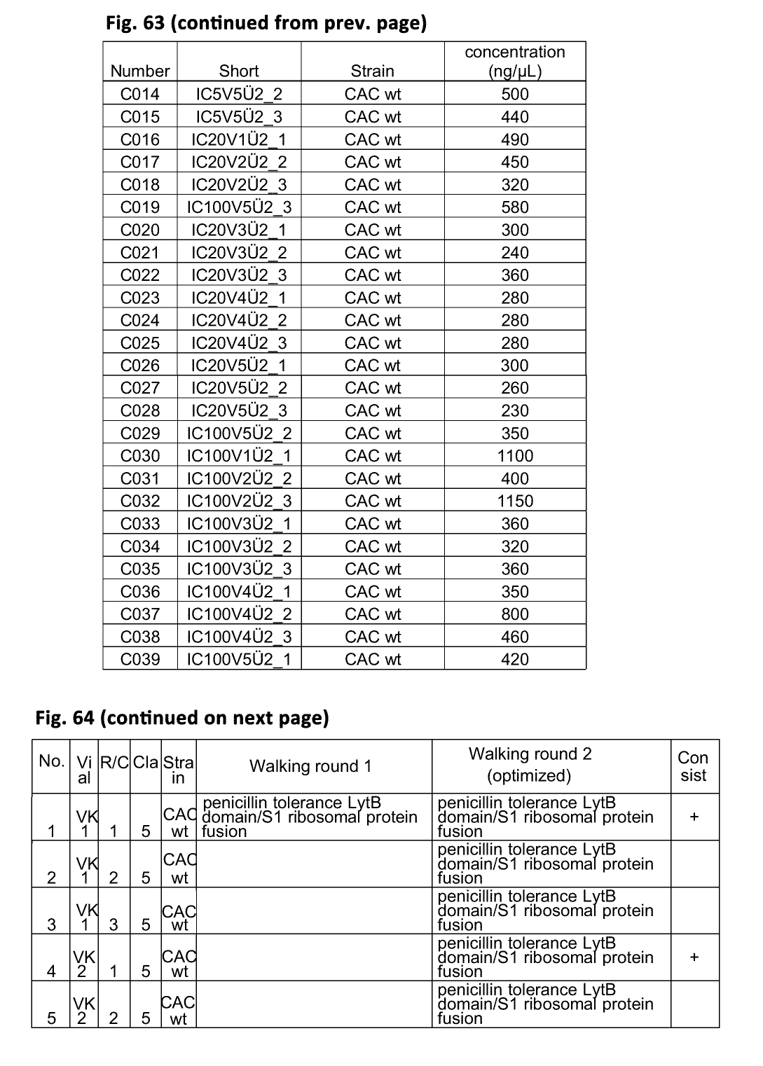

Subsequently gDNA was prepared as depicted in FIG. 63 (protocol vide supra).

Determination of Loci:

The first round of PCR genome walking by LGC Genomics provided the following results:

Not determinable:

6, 8, 10, 11, 12, 13, 14, 15, 17, 18, 20, 21, 22, 23, 24, 25, 26, 27, 28, 29, 32, 35

guent01: 4-hydroxy-3-methylbut-2-enyl diphosphate reductase/S1 RNA-binding domain protein

1, 2, 3, 4, 5

guent07: pploop-atpase

7, 9

guent16: Conserved membrane protein, probable transporter, YPAA B. subtilis ortholog

16

guent19: Uncharacterised protein, DegV family

19

guent30: fibronectin type III domain-containing protein

30, 31

guent34: Staphylococcus aureus subsp. aureus ST228 plasmid pI5S5

34

The obtained reads were as follows: >guent_07_gw2 bk_261112.0.10 had the sequence of SEQ ID NO: 86; >guent01.gw2 bk_261112.0.2 had the sequence of SEQ ID NO: 87; >guent_19_gw2 bk_261112.0.15 had the sequence of SEQ ID NO: 88; >guent_30_gw2 bk_261112.0.18 had the sequence of SEQ ID NO: 89; >guent_34_gw2 bk_261112.0.21 had the sequence of SEQ ID NO: 90; >guent_16_gw2 bk_261112.0.14 had the sequence of SEQ ID NO: 91.

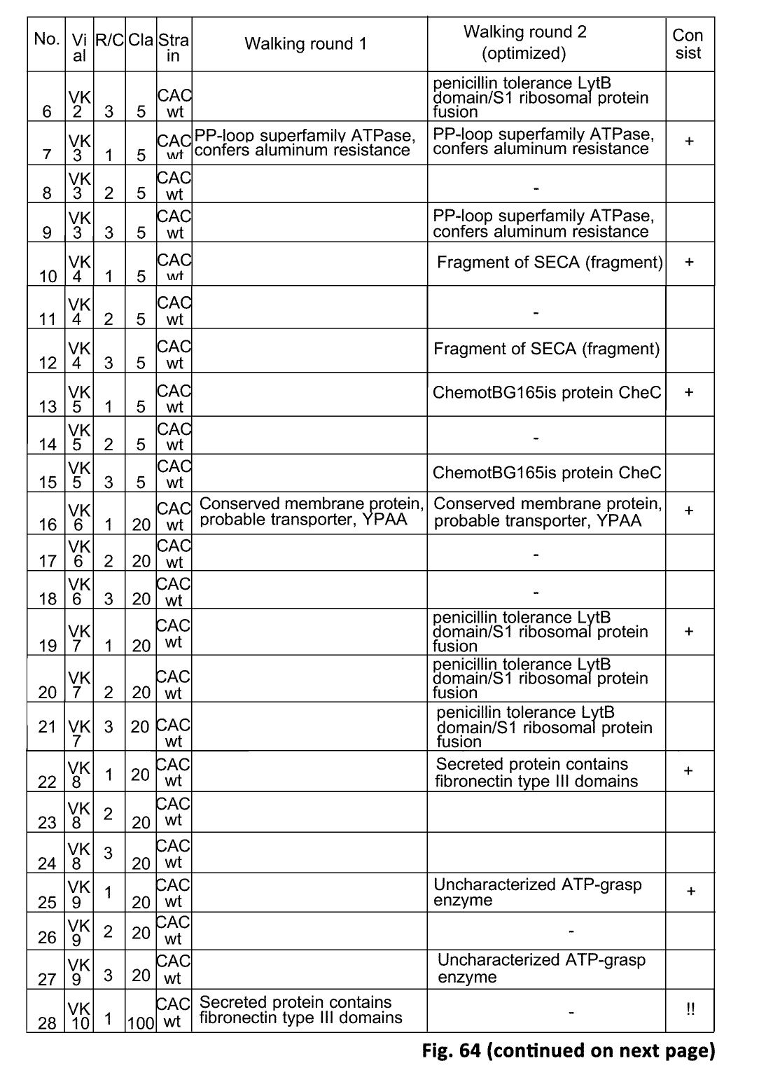

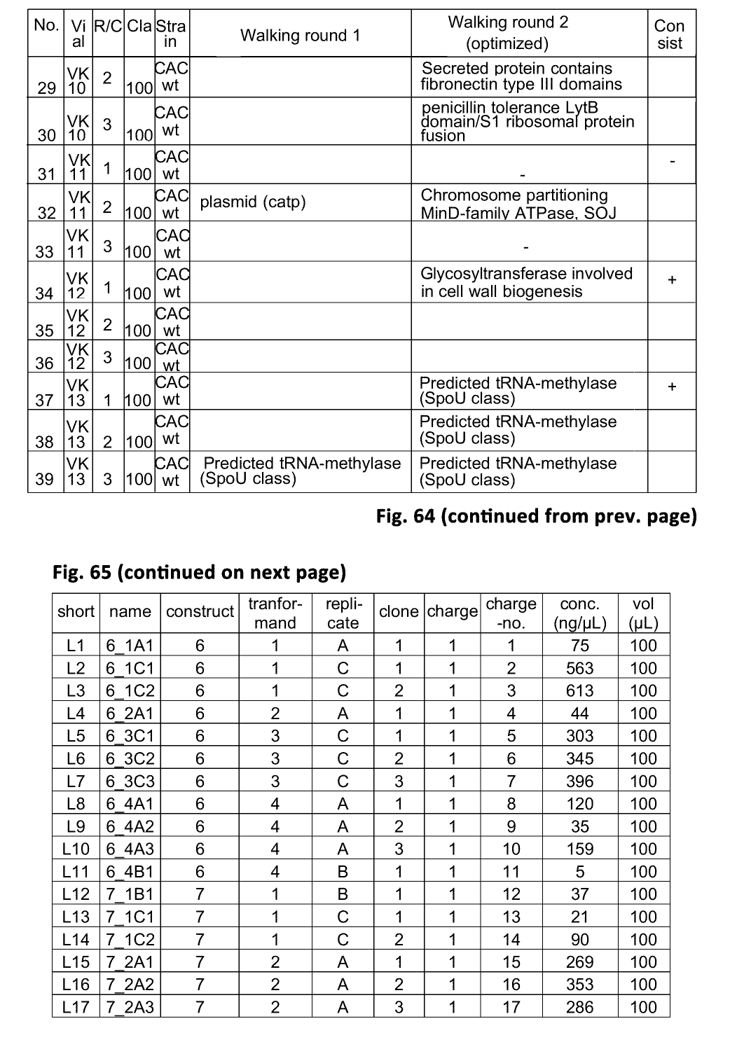

A second round of loci determination provided the results depicted in FIG. 64. Almost all loci were determined. The results are also very consistent. One out of the 4 clones generated with the highest selection pressure shows multicopy integration.

### Example 4: Application of the Positive Selection System for Random Chromosomal and Random Multicopy Chromosomal Integrations Via Transposase in Clostridium ljungdahlii and Clostridium autoethanogenum

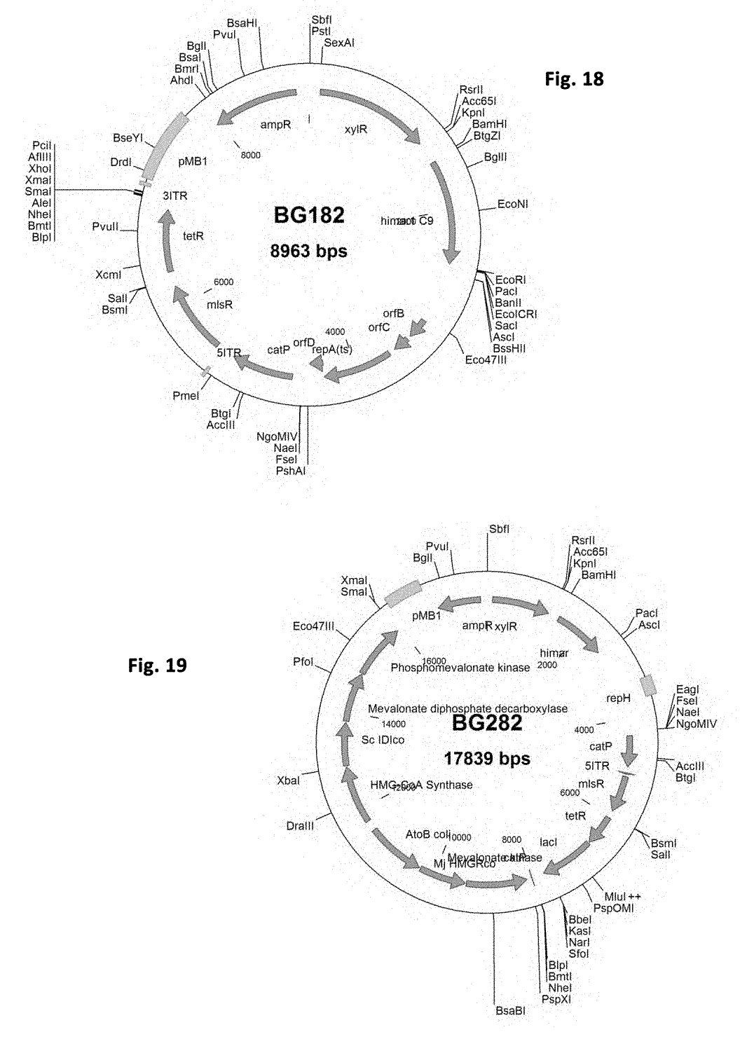

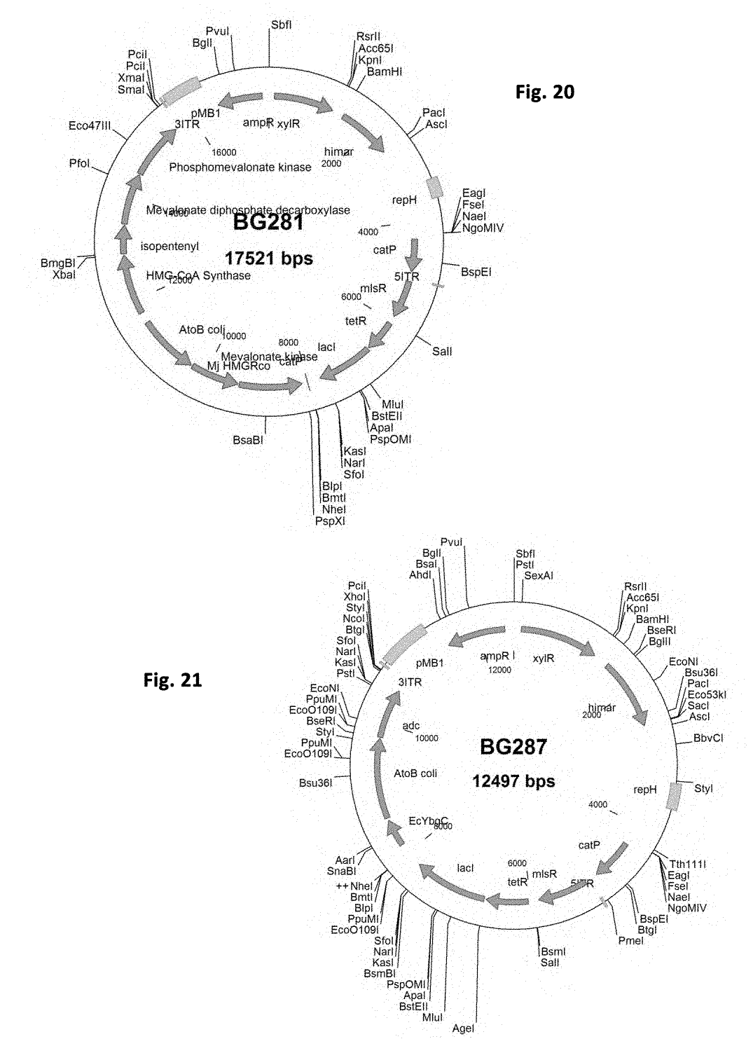

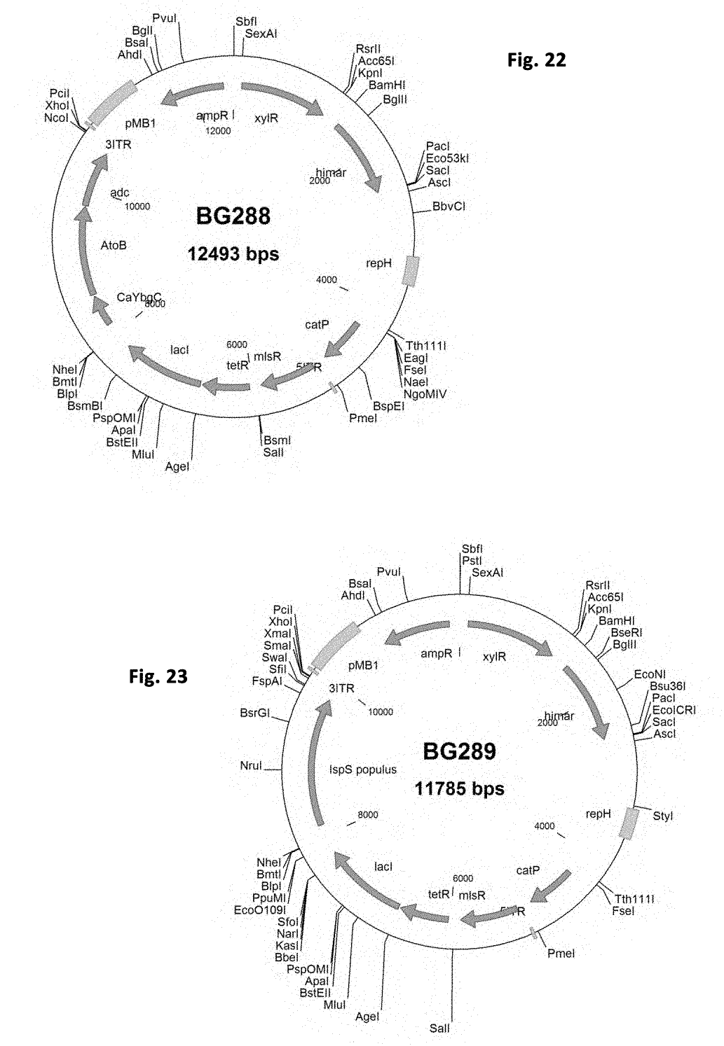

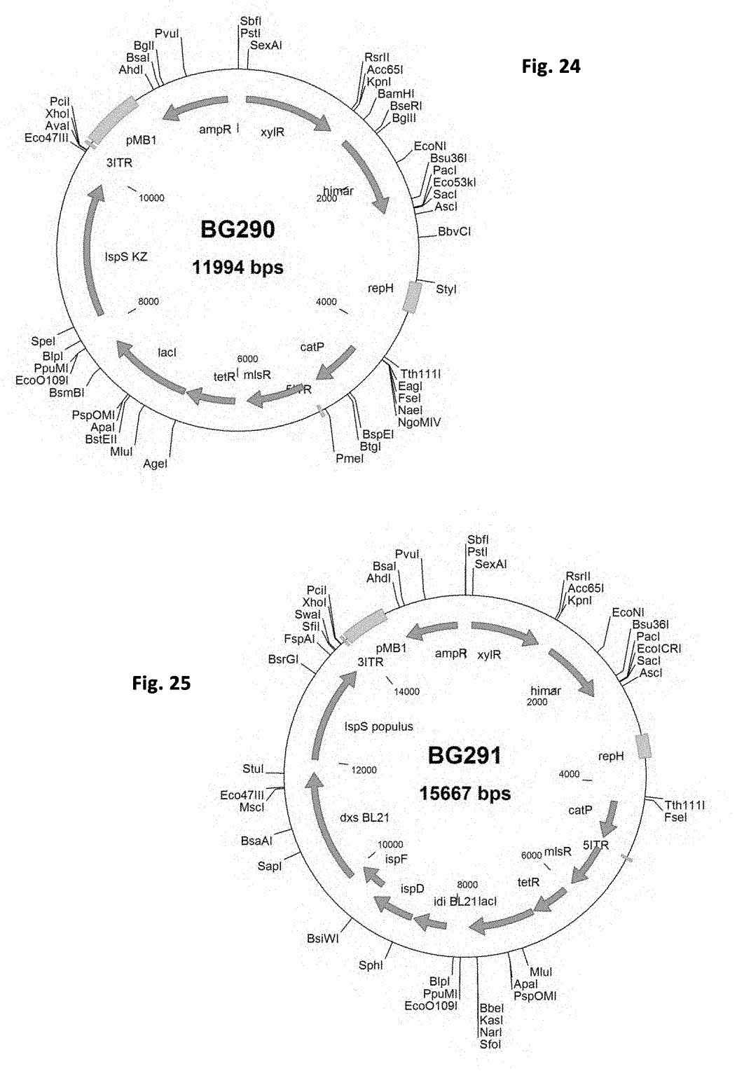

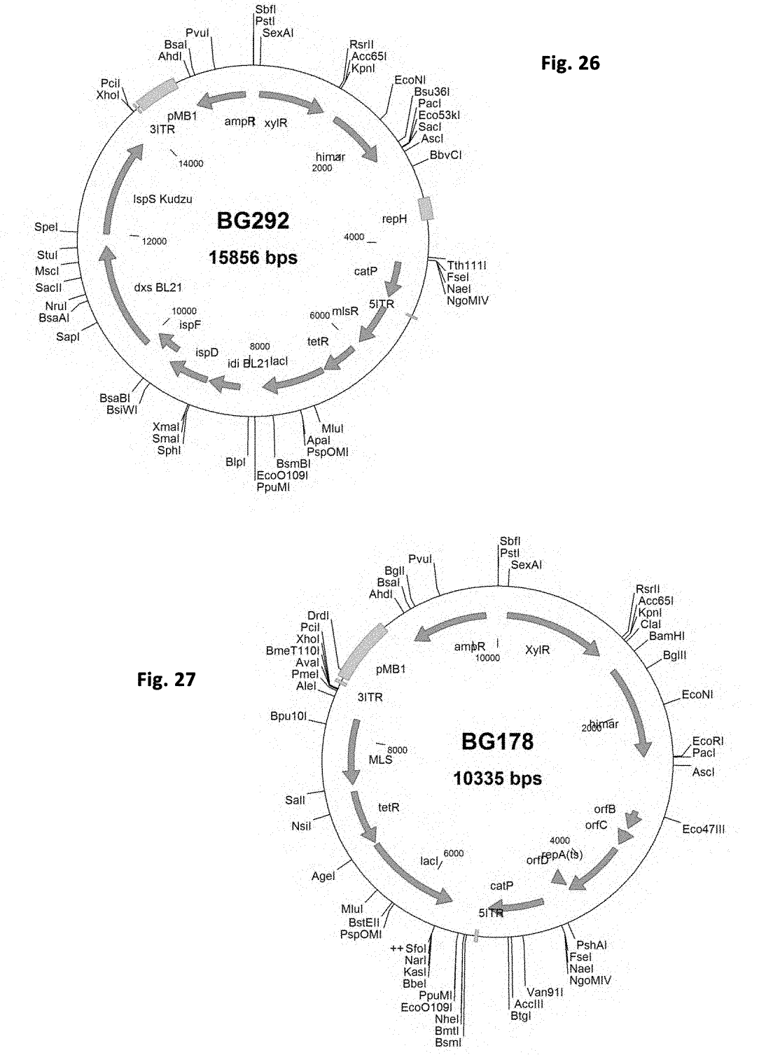

The following plasmids with the same positive selection system as depicted in example 2 (vide supra) were used with Clostridium ljungdahlii and Clostridium autoethanogenum: Vector BG282 (FIG. 19) with the sequence of SEQ ID NO: 17; vector BG281 (FIG. 20) with the sequence of SEQ ID NO: 18; vector BG287 (FIG. 21) with the sequence of SEQ ID NO: 19; vector BG288 (FIG. 22) with the sequence of SEQ ID NO: 20; vector BG289 (FIG. 23) with the sequence of SEQ ID NO: 21; vector BG290 (FIG. 24) with the sequence of SEQ ID NO: 22; vector BG291 (FIG. 25) with the sequence of SEQ ID NO: 23; vector BG292 (FIG. 26) with the sequence of SEQ ID NO: 24; vector BG178 (FIG. 27) with the sequence of SEQ ID NO: 25. Plasmid BG281 was for example used to integrate a big mevalonate biosynthetic gene cluster in the chromosome of Clostridium ljungdahlii. Plasmid BG282, as another example, was used to integrate a slightly different MVA pathway into the chromosome of Clostridium autoethanogenum.

### Genetic Transformation of Clostridium ljungdahlii

The transformation was performed according to Leang, L., et al., Appl Environ Microbiol. (2013) 79, 4, 1102-1109. Instead of NEB express cells E. coli BL21, and YTF medium instead of PETC medium was used. The protocol was as follows:

Preparation of Electrocompetent C. ljungdahlii Cells.

All manipulations except centrifugation were carried out on ice in an anaerobic chamber. All buffers and centrifuge tubes were made ice cold and anoxic before use. All plastic ware was placed in the anaerobic chamber at least 24 h before use in order to eliminate any residual oxygen. Competent cells were maintained in SMP buffer (270 mM sucrose, 1 mM MgCl₂, 7 mM sodium phosphate, pH 6) with 10% DMSO at 80° C. until use. Electro competent C. ljungdahlii cells were prepared from cultures that were freshly inoculated from frozen stocks and then transferred twice in PETC liquid medium. PETC medium can be replaced with YTF medium without significant loss of transformation efficiencies. About 15 to 16 h before preparation of competent cells, a mid- to late-log-phase culture was transferred into two serum bottles containing 100 ml fresh PETC medium supplemented with 40 mM DL-threonine (final optical density at 600 nm [OD₆₀₀], =0.004). After overnight growth at 37° C., early-log phase cells (OD₆₀₀ 0.2 to 0.3; 200 ml) were harvested by centrifugation at 10,000 rpm for 10 min at 4° C. The cells were washed twice with 200 ml of SMP wash buffer and resuspended in the same buffer at a final concentration of 10¹⁰ to 10¹¹ cells/ml. Antifreezing buffer (60% DMSO—40% SMP, pH 6) was added to the competent cells a tone-fifth of the final resuspension volume to achieve a final concentration of 10% DMSO. The resulting competent cells (25 μl/tube) were stored at 80° C. for future use. The competence of these frozen competent cells remained stable for about 1 month.

Electro-Transformation Procedures for C. ljungdahlii

All procedures were carried out in an anaerobic chamber. Electro competent C. ljungdahlii cells (25 μL) were quickly transferred on ice from a 80° C. freezer to an anaerobic chamber. After thawing on ice (about 1 min), the cells were mixed with 1 to 5 μg DNA and transferred to a pre chilled, 0.1-cm-gap Gene Pulser cuvette (Bio-Rad). Cells were pulsed at 0.625 kV with resistance at 600 Ohm and a capacitance of 25 μF by using a Gene Pulser Xcellmicrobial electroporation system (Bio-Rad). Immediately after the pulse, cells were recovered with 0.5 ml of fresh PETC medium, transferred to a pressure tube containing 10 ml of PETC medium supplemented with 5 mM L-cysteine (pH 7), and incubated at 37° C. The electroporated cells were allowed to recover at 37° C. until their cell densities were higher than immediately after the electroporation (about 9 to 12 h). Five-milliliter volumes of the outgrowth cultures or appropriately diluted cultures were mixed with 20 ml of RCM molten agar (1.5%) containing an appropriate antibiotic and poured into a petri dish. After the agar mixtures were solidified, plates were incubated upside down.

Transformed Plasmids:

| Nr. | Name (Sequence) |
|---|---|
| 1 | BG282 (SEQ ID NO: 17) |
| 2 | BG281 (SEQ ID NO: 18) |
| 3 | BG287 (SEQ ID NO: 19) |
| 4 | BG288 (SEQ ID NO: 20) |
| 5 | BG289 (SEQ ID NO: 21) |
| 6 | BG290 (SEQ ID NO: 22) |
| 7 | BG291 (SEQ ID NO: 23) |
| 8 | BG292 (SEQ ID NO: 24) |
| 9 | BG178 (SEQ ID NO: 25) |

All plasmids except BG178 (9) could be transformed in Clostridium ljungdahlii and Clostridium autoethanogenum.

Chromosomal Integration in Clostridium ljungdahlii

Transformands were cultured in liquid triplicates using YTX (5 g/L xylose) with 5 μg/mL clarithromycin. Serial Transfer with fresh medium (YTX containing 5 g/L xylose and 5 μg/mL clarithromycin) was performed 3 times. After that 5 μL were spotted on YTG plates without antibiotics. 4 colonies of each plate were picked and struck simultaneously on YTG plates with 5 μg/mL clarithromycin, YTG plates with 5 μg/mL thiamphenicol and on plates without antibiotics.

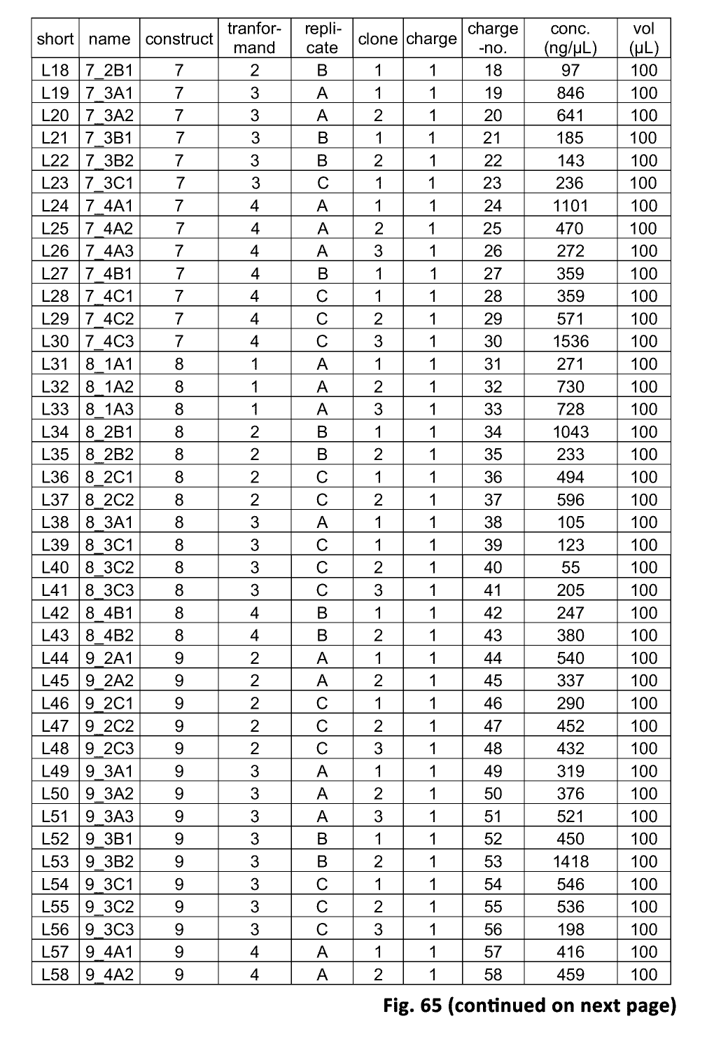

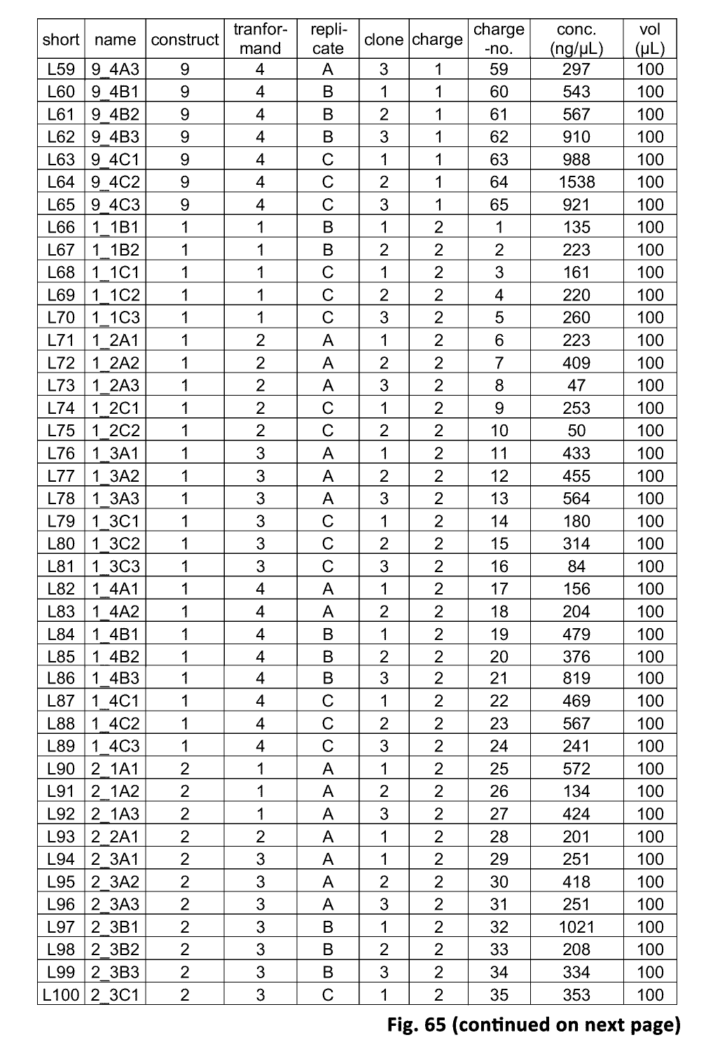

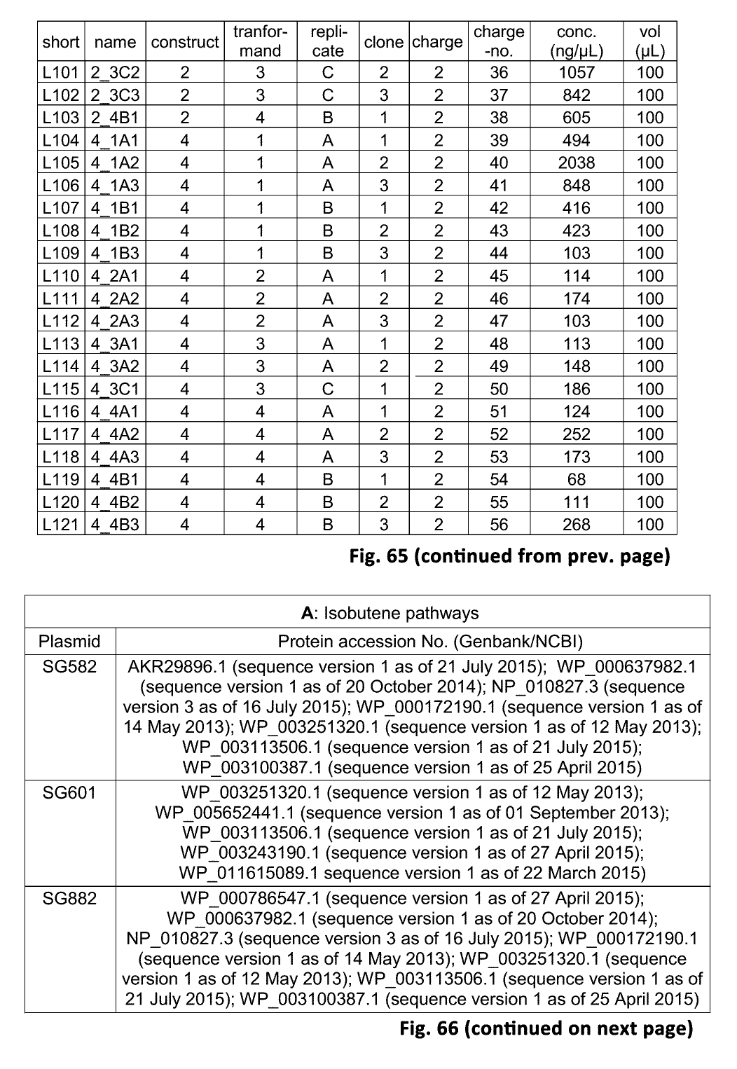

The integrands (those who were clarithromycin but not thiamphenicol resistant) were cultivated for the preparation of genomic DNA. Preparation of genomic DNA of the Clostridium ljungdahlii integration strains was performed using a kit from Macherey-Nagel (special protocol for difficult to lyse bacteria). Approx. 10 mL liquid culture were used. Concentration of gDNA was determined by nanodrop. Results are depicted in the table of FIG. 65.

PCR Verification of Integrands

PCR on gDNA of putative integrands was performed with annealing temperature 55° C., elongation time 5 min and 30 cycles using the following primers (amplifying the integration cassette):

Primer forward:
(SEQ ID NO: 92)
CTGTCTCTTATACACATCTGCTGATAAGTCCCCGGTC.
Primer reverse:
(SEQ ID NO: 93)
CTGTCTCTTATACACATCTGCTGATAAGTCCCCGGTC.

The PCR products were then examined by 0.8% analytic DNA agarose gel electrophoresis.

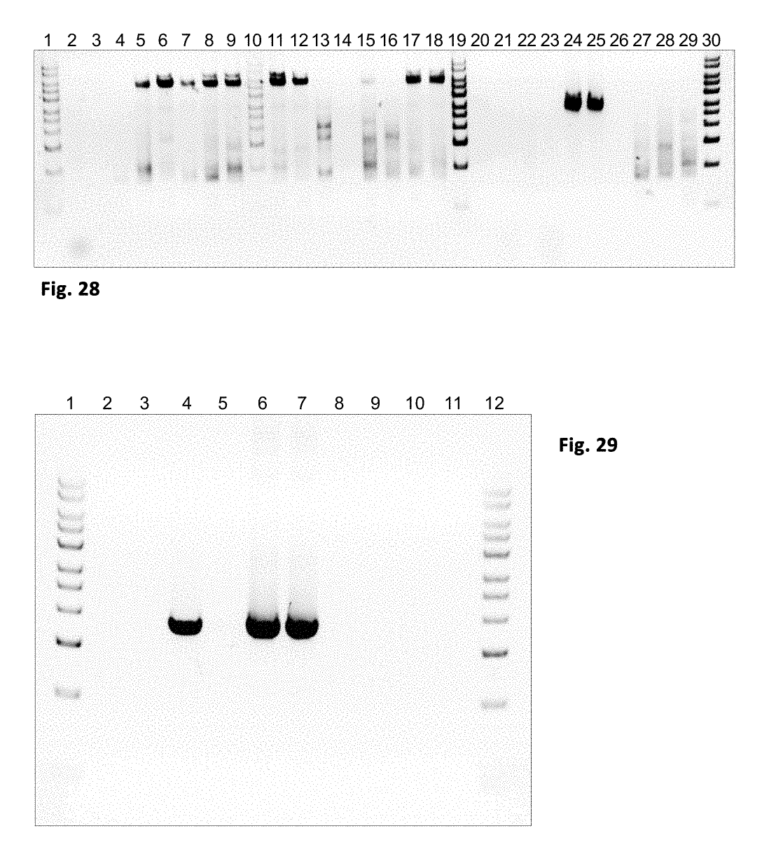

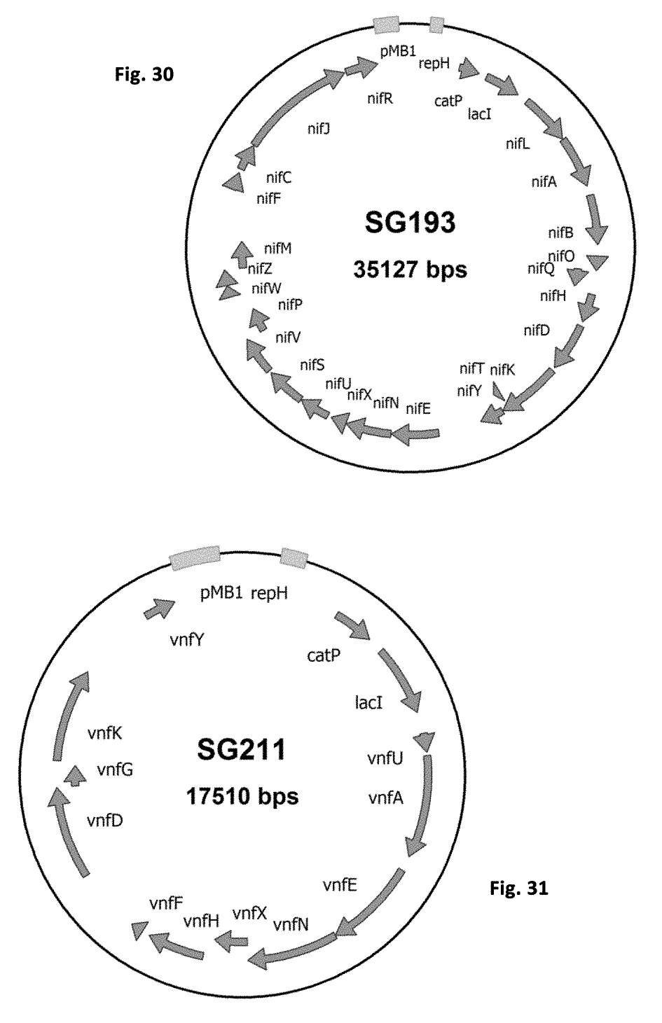

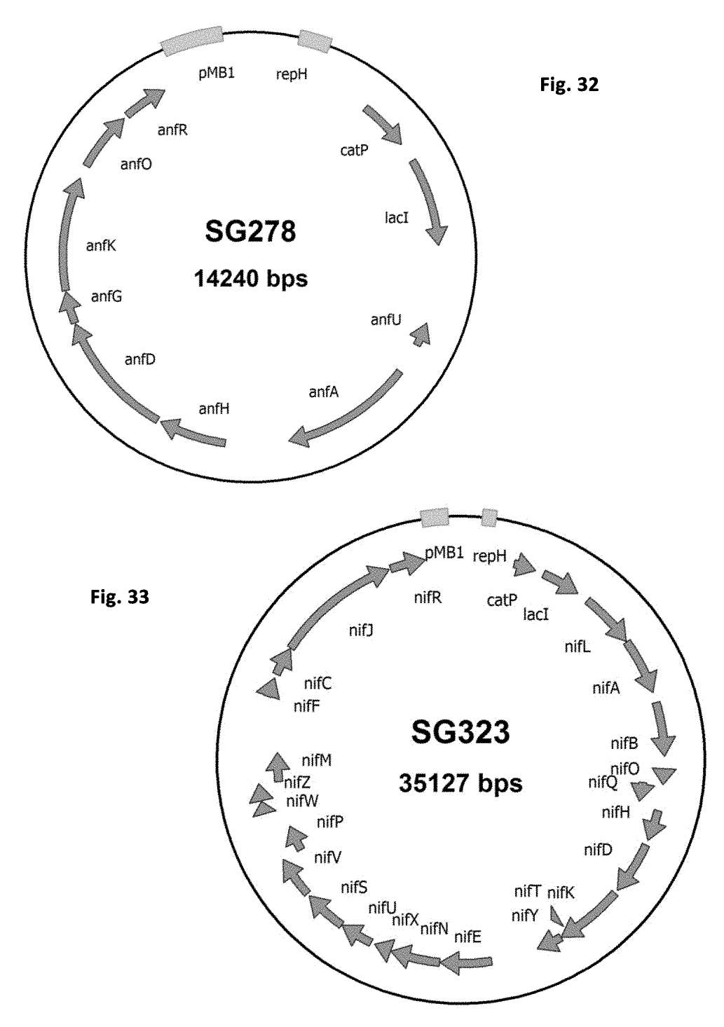

The result can be taken from FIG. 28. Lane 1: 3 μL ladder 1 kB purchased from Roth; Lane 2-9: integrand LBI 105-111 PCR; result: 4,5,6,7,8 positive 1,2,3 negative; Lane 10: 3 μL ladder 1 kB purchased from Roth; Lane 11-18: integrand LBI 112-120 PCR; result: 1,2,5,7,8 positive 3,4,6 negative; Lane 19: 3 μL ladder 1 kB purchased from Roth; Lane 24-25: positive control PCR; result: 1,2 positive; Lane 26-27: negative control gDNA C. ljungdahlii gDNA PCR; negative; Lane 28-29: negative control gDNA C. autoethanogenum gDNA PCR; negative; Lane 30: 3 μL ladder 1 kB purchased from Roth.

Analysis: the integration cassette of at least some of the integrands can be amplified by PCR. This shows that with the help of a positive selection system cargo in this case up to 10 kb could be integrated.

### Chromosomal Integration in Clostridium autoethanogenum

A single construct, BG289, was selected for integration. Transformands of BG289 were cultured YTX (5 g/L xylose) with 5 μg/mL clarithromycin. Serial Transfer with fresh medium (YTX containing 5 g/L xylose and 5 μg/mL clarithromycin) was performed 3 times. After that 5 μL were spotted on YTG plates without antibiotics. 10 colonies were picked and struck simultaneously on YTG plates with 5 μg/mL clarithromycin, YTG plates with 5 μg/mL thiamphenicol and on plates without antibiotics. This resulted in four integrands. The integrands (those who were clarithromycin but not thiamphenicol resistant) were cultivated for the preparation of genomic DNA. Preparation of genomic DNA of the Clostridium autoethanogenum integrands was performed using a kit from Macherey-Nagel (special protocol for difficult to lyse bacteria). Approx. 10 mL liquid culture was used. Concentration of gDNA was determined by nanodrop.

PCR Verification of Integrands

PCR on gDNA of putative integrands was performed with annealing temperature 55° C., elongation time 60 sec and 30 cycles using the following primers (binding to the enzyme in the integration cassette):

| SEQ ID NO: 94 | |
|---|---|
| Primer forward: | GGGTTGCCTTACTGGTTAG. |
| SEQ ID NO: 95 | |
| Primer reverse: | GCAGTATCGGTTCGGTAATC. |

The PCR products were then examined by 0.8% analytic DNA agarose gel electrophoresis. The result is shown in FIG. 29. Lane 1: 3 μL ladder 1 kB purchased from Roth; Lane 2-5: integrand A1-A4 PCR; result: 3 positive 1,2,4 negative; Lane 6-7: positive control PCR; result: 1,2 positive; Lane 8-9: negative control gDNA C. ljungdahlii gDNA PCR; negative; Lane 10-11: negative control gDNA C. autoethanogenum gDNA PCR, negative; Lane 12: 3 μL ladder 1 kB purchased from Roth.

Analysis: The integrated enzyme can be amplified from gDNA sample A3. This indicates that the system works with broad host range within the genus Clostridium.

### Example 5: Synthesis of 2-methyl-1,3-butadiene

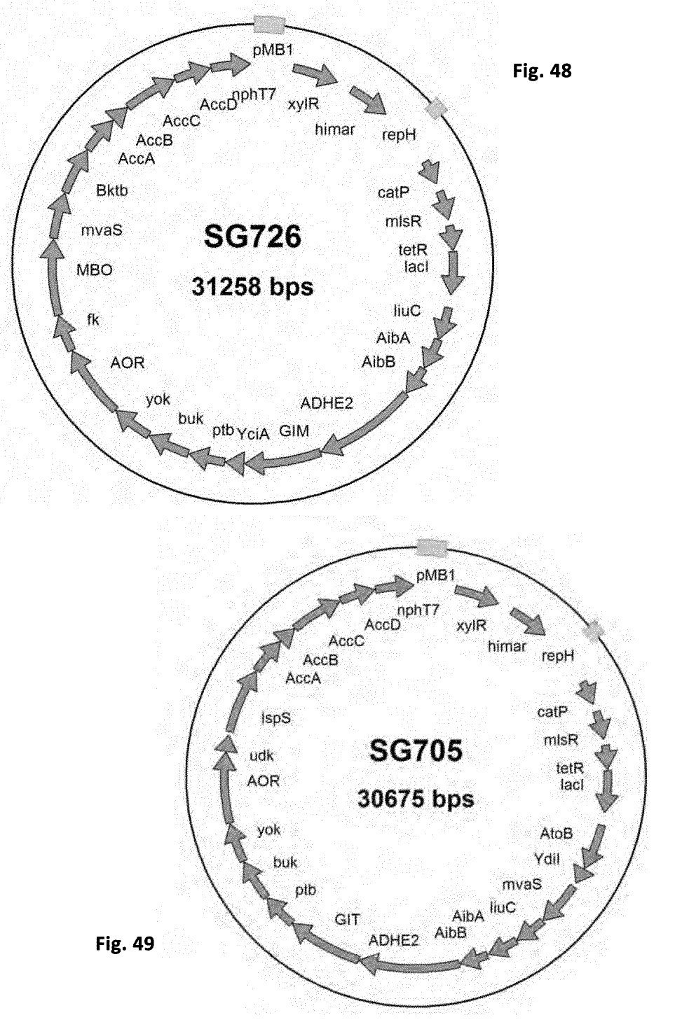

FIG. 48 and FIG. 49 show the plasmids SG726 (SEQ ID NO: 44) and SG705 (SEQ ID NO: 45), respectively, which each contain sequences encoding enzymes of a 2-methyl-1,3-butadiene pathway (for genes see also reference numerals). FIG. 49 shows the plasmid SG705 (SEQ ID NO: 45) which contains the sequences encoding enzymes of an engineered 2-methyl-1,3- butadiene pathway (for genes see also reference numerals). Preparation of electrocompetent C. ljungdahlii cells and electrotransformation were carried out as described in Example 1 (see also Example 3). 2-methyl-1,3-butadiene production was performed as described in Example 1. 2-methyl-1,3-butadiene formation could be detected by GC-MS as described above.

The two plasmids were also used to engineer Clostridium ljungdahlii and Clostridium autoethanogenum cells chromosomally to produce 2-methyl-1,3-butadiene. The protocol and process described in Example 1 was performed. Additionally, CO and a mixture of CO₂ and CO, as well as CO and electrons and/or CO₂ and electrons were also successfully used as substrates.

### Example 6: Synthesis of 2-methyl-1,3-butadiene

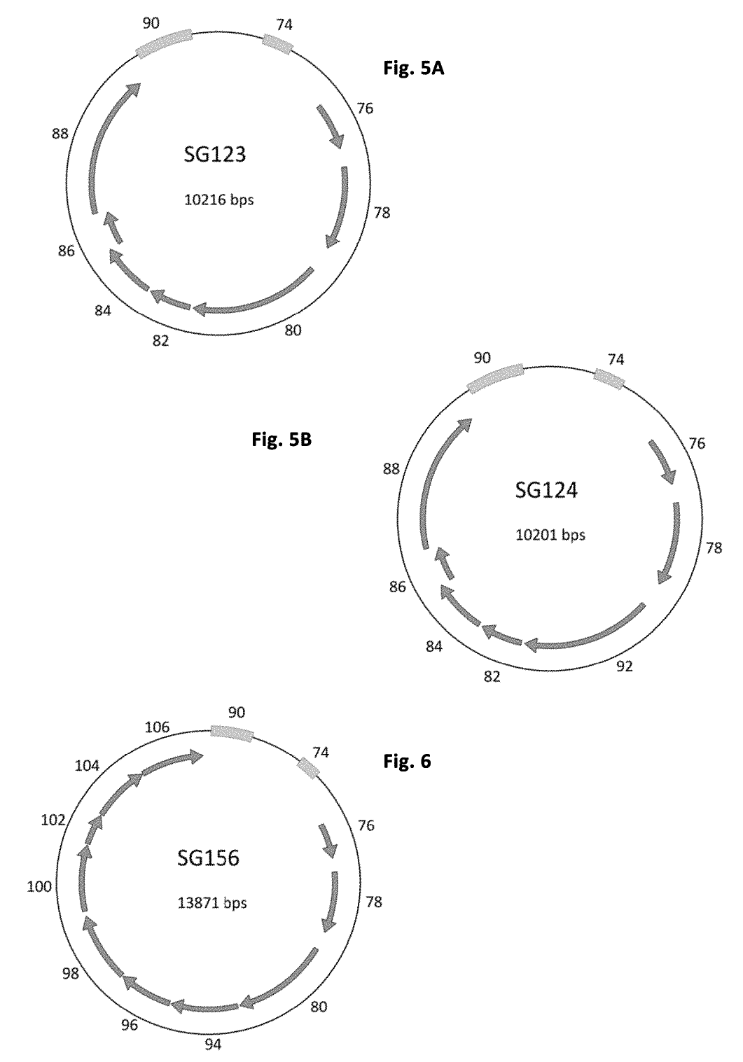

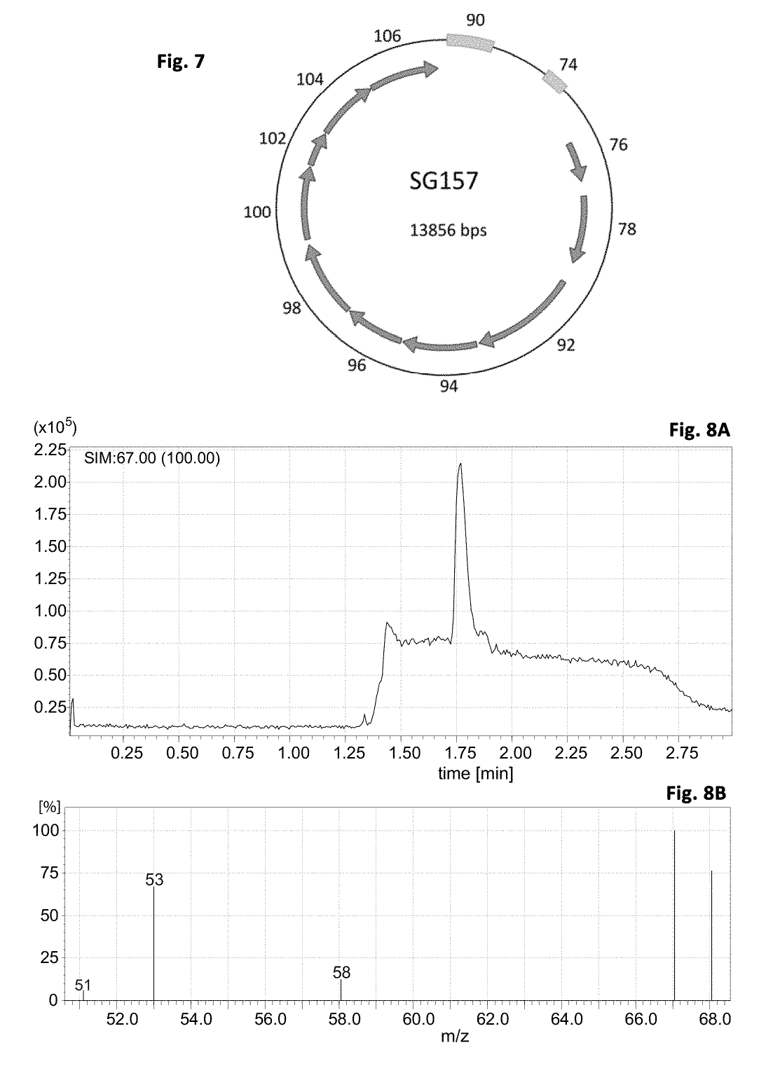

FIG. 6 and FIG. 5A show the plasmids SG156 (SEQ ID NO: 7) and SG123 (SEQ ID NO: 5), respectively, which each contain sequences encoding enzymes of a 2-methyl-1,3-butadiene pathway (for genes see also reference numerals). FIG. 5B and FIG. 7 show the plasmids SG124 (SEQ ID NO: 6) and SG157 (SEQ ID NO: 8), respectively, which each contain the sequences encoding enzymes of further engineered 2-methyl-1,3- butadiene pathways (for genes see also reference numerals). Preparation of electrocompetent C. ljungdahlii cells and electrotransformation were carried out as described in Example 1 (see also Example 3). 2-Methyl-1,3-butadiene production was performed as described in Example 1 with 100% CO₂. FIGS. 8A and 8B show the product analysis by GC-MS using a Shimadzu GC-MS QP5050A. Samples were taken via a 500 μL Hamilton Gastight syringe. 500 μL headspace were collected and loaded onto a RXI-5 ms column 30 m, 0.25 mm ID and 0.25 μm DF (Restek), using a split of −1. Separation was carried out isocratically at 30° C. (inject-temperature 60° C.; SIM recorded with m/z=67). The spectrum shows a perfect match with 2-methyl-1,3-butadiene.

The four plasmids were also used to engineer Clostridium ljungdahlii and Clostridium autoethanogenum cells chromosomally to produce 2-methyl-1,3-butadiene. The protocol and process described in Example 1 was performed. Both 100% CO₂ and a mixture of 50% CO₂ and 50% CO were also successfully used as substrates.

### Example 7: Synthesis of 1,3-butadiene

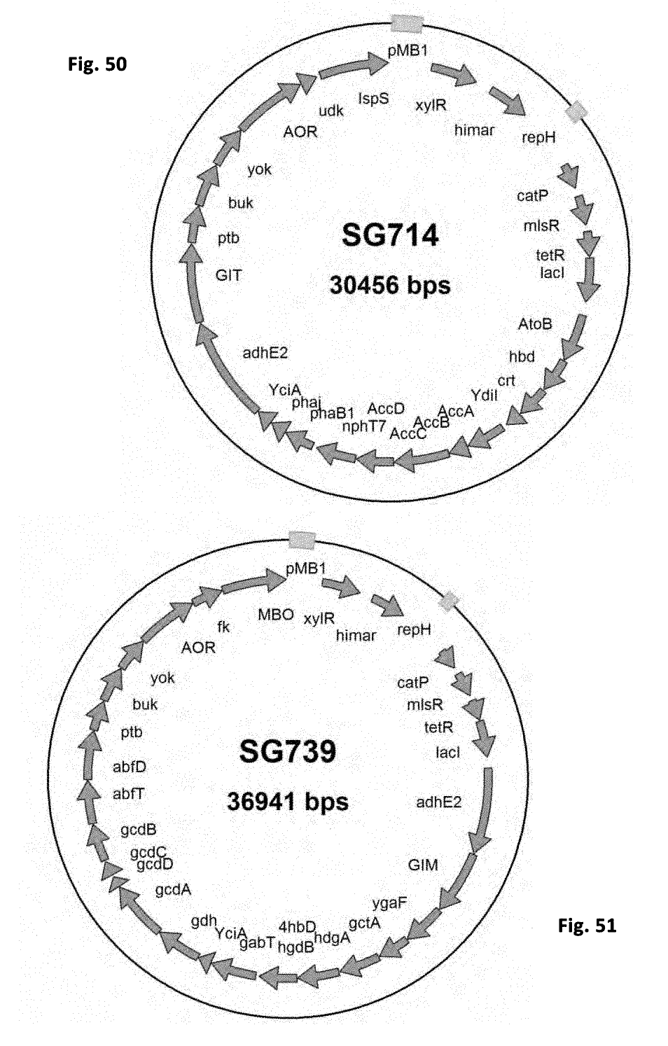

FIG. 50 shows the plasmid SG714 (SEQ ID NO: 46) which contains the sequences encoding enzymes of an engineered 1,3-butadiene pathway (for genes see also reference numerals). FIG. 51 shows the plasmid SG739 (SEQ ID NO: 47) which contains the sequences encoding enzymes of a second engineered 1,3-butadiene pathway (for genes see also reference numerals). Preparation of electrocompetent C. ljungdahlii cells and electrotransformation were carried out as described in Example 1 (see also Example 3). 1,3-butadiene production was performed as described in Example 1. 1,3-butadiene production could be detected by GC-MS as described above.

The two plasmids were also used to engineer Clostridium ljungdahlii and Clostridium autoethanogenum cells chromosomally to produce 1,3-butadiene. The same protocol and process like described in Example 1 was performed. Additionally, CO and a mixture of CO₂ and CO, as well as CO and electrons and/or CO₂ and electrons were also successfully used as substrates.

### Example 8: Synthesis of 1,3-butadiene

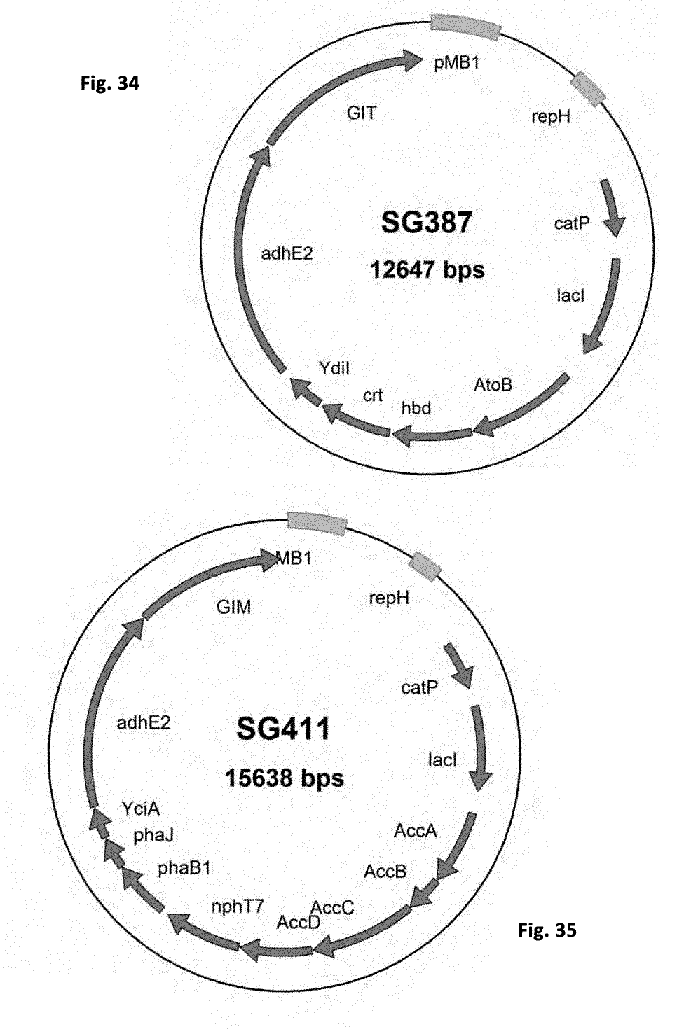

FIG. 34 shows the plasmid SG387 (SEQ ID NO: 30) which contains the sequences encoding enzymes of an engineered 1,3-butadiene pathway (for genes see also reference numerals). FIG. 35 shows the plasmid SG411 (SEQ ID NO: 31) which contains the sequences encoding enzymes of a second engineered 1,3-butadiene pathway (for genes see also reference numerals).

The construction of the expression plasmids was performed by standard recombinant DNA and molecular cloning techniques using genes from Clostridium acetobutylicum ATCC 824, Clostridium carboxidivorans, Escherichia coli, Ralstonia eutropha H16, Aeromonas caviae, Streptomyces sp. strain CL190, Saccharomyces cerevisiae S288c, Bacillus subtilis, Clostridium ljungdahlii DSM 13528 DSM 13528, Escherichia coli BL21(DE3), Clostridium sp. M62/1, Clostridium botulinum BKT015925, Clostridium kluyveri DSM 555, Acidaminococcus fermentans DSM 20731, Clostridium propionicum, Chloroflexus aurantiacus J-10-fl, Haemophilus influenzae R2866, Pseudomonas putida KT2440, Treponema denticola ATCC 35405, Thauera linaloolentis and Mycobcaterium JDM601.

Preparation of electrocompetent C. ljungdahlii cells and electrotransformation were carried out as described in Example 1 (see also Example 3). For plasmid SG714 1,3-butadiene production was performed as described in Example 1 with 100% CO₂. For plasmid SG739 1,3-butadiene production was performed as described in Example 1 with 30% CO₂, and 60% H₂. 1,3-butadiene production could in both cases be detected by GC-MS as described above.

The two plasmids were also used to engineer Clostridium ljungdahlii and Clostridium autoethanogenum cells chromosomally to produce 1,3-butadiene. The same protocol and process like described in Example 1 was performed. Additionally, CO and a mixture of CO₂ and CO, as well as CO and electrons and/or CO₂ and electrons were also successfully used as substrates.

### Example 9: Synthesis of Propene

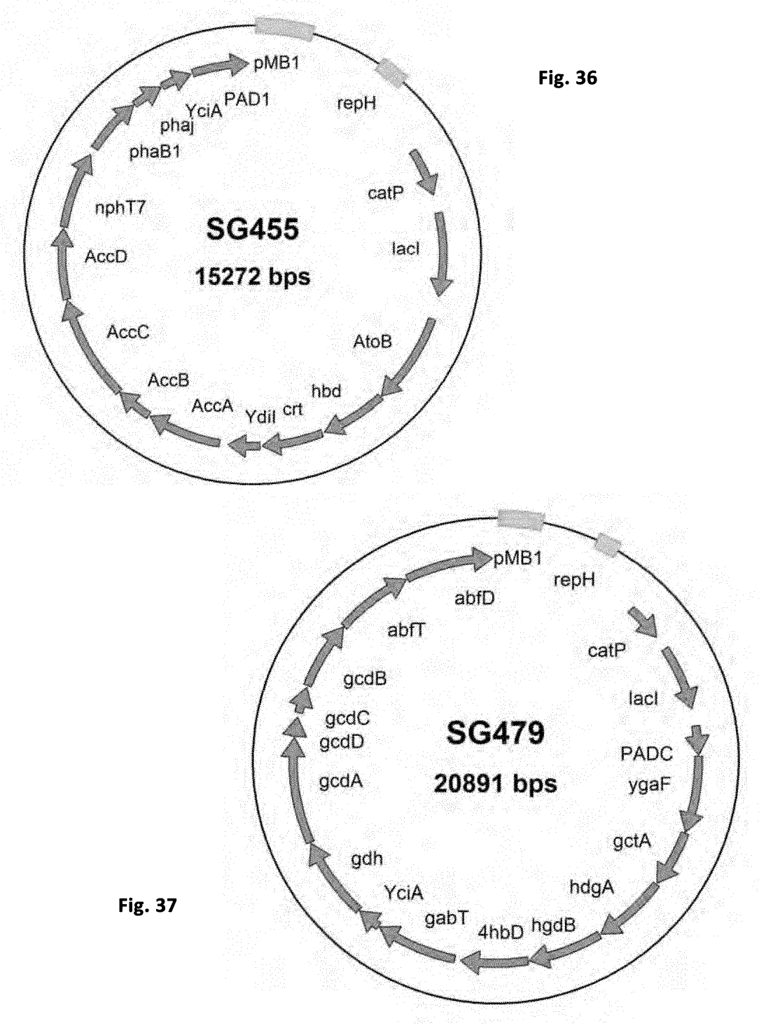

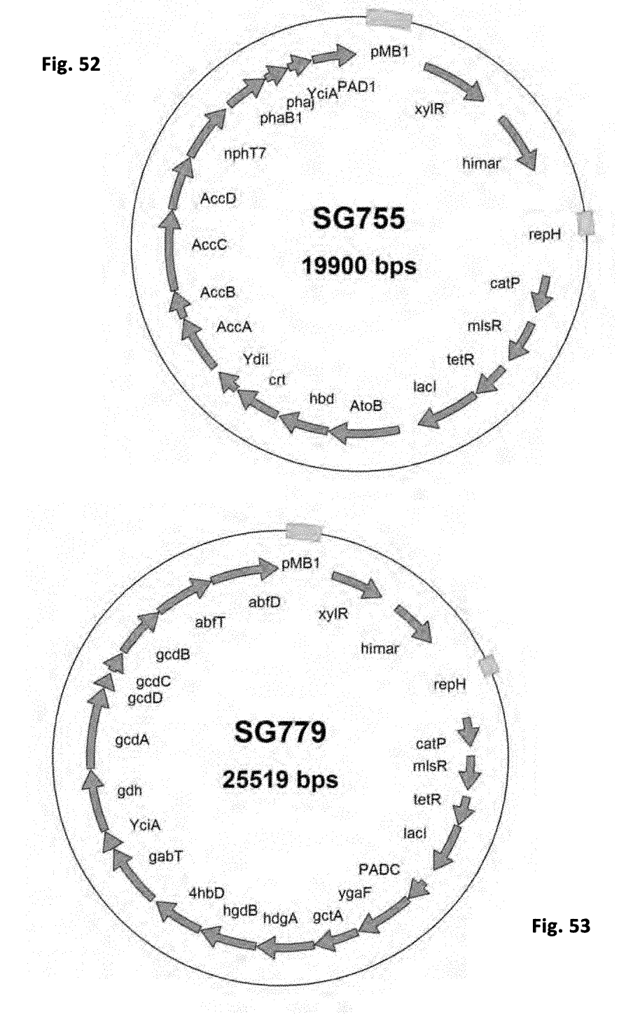

FIG. 36 shows the plasmid SG455 (SEQ ID NO: 32) which contains the sequences encoding enzymes of an engineered propene pathway (for genes see also reference numerals). FIG. 52 shows the plasmid SG755 (SEQ ID NO: 48) which contains the sequences encoding enzymes of a further engineered propene pathway. FIG. 53 shows the plasmid SG779 (SEQ ID NO: 49) which contains the sequences encoding enzymes of another engineered propene pathway. FIG. 37 shows the plasmid SG479 (SEQ ID NO: 33) which contains the sequences encoding enzymes of yet another engineered propene pathway.

The construction of the expression plasmids was performed by standard recombinant DNA and molecular cloning techniques using genes from Clostridium acetobutylicum ATCC 824, Clostridium carboxidivorans, Escherichia coli, Ralstonia eutropha H16, Aeromonas caviae, Streptomyces sp. strain CL190, Saccharomyces cerevisiae S288c, Bacillus subtilis, Clostridium ljungdahlii DSM 13528 DSM 13528, Escherichia coli BL21(DE3), Clostridium sp. M62/1, Clostridium botulinum BKT015925, Clostridium kluyveri DSM 555, Acidaminococcus fermentans DSM 20731, Clostridium propionicum, Chloroflexus aurantiacus J-10-fl, Haemophilus influenzae R2866, Pseudomonas putida KT2440, Treponema denticola ATCC 35405, Mus musculus, Metallosphaera sedula DSM 5348 and Staphylococcus aureus.

Preparation of electrocompetent C. ljungdahlii DSM 13528 cells and electrotransformation were carried out as described in Example 1. Propene production was performed as described in Example 1. Propene production could be detected by GC-MS as described above.

The three plasmids were also used to engineer Clostridium autoethanogenum DSM 10061 and Clostridium aceticum cells to produce propene. The same protocol and process as described in Example 1 was performed. Additionally, CO and a mixture of CO₂ and CO, as well as CO and electrons and/or CO₂ and electrons were also successfully used as substrates.

### Example 10: Synthesis of 1-butene

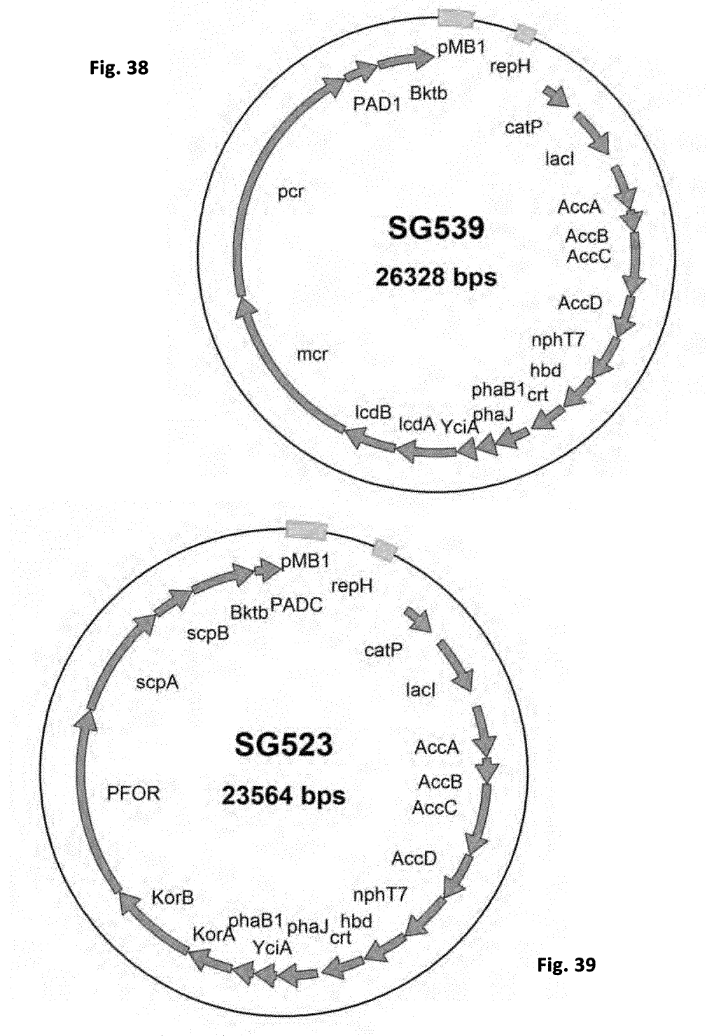

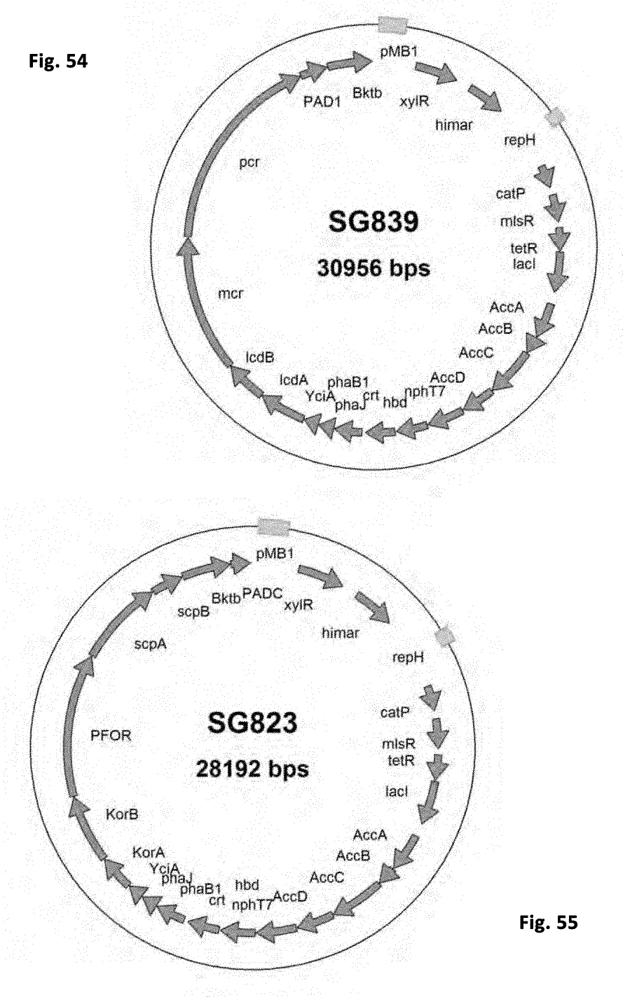

FIG. 38 shows the plasmid SG539 (SEQ ID NO: 34) which contains the sequences encoding enzymes of an engineered 1-butene pathway (for genes see also reference numerals). FIG. 39 shows the plasmid SG523 (SEQ ID NO: 35) which contains the sequences encoding enzymes of another engineered 1-butene pathway. FIG. 54 shows the plasmid SG839 (SEQ ID NO: 50) which contains the sequences encoding enzymes of another engineered 1-butene pathway. FIG. 55 shows the plasmid SG823 (SEQ ID NO: 51) which contains the sequences encoding enzymes of yet a further engineered 1-butene pathway.

The construction of the expression plasmids was performed by standard recombinant DNA and molecular cloning techniques using genes from Clostridium acetobutylicum ATCC 824, Clostridium carboxidivorans, Escherichia coli, Ralstonia eutropha H16, Aeromonas caviae, Streptomyces sp. strain CL190, Saccharomyces cerevisiae S288c, Bacillus subtilis, Clostridium ljungdahlii DSM 13528 DSM 13528, Escherichia coli BL21(DE3), Clostridium sp. M62/1, Clostridium botulinum BKT015925, Clostridium kluyveri DSM 555, Acidaminococcus fermentans DSM 20731, Clostridium propionicum, Chloroflexus aurantiacus J-10-fl, Haemophilus influenzae R2866, Pseudomonas putida KT2440, Treponema denticola ATCC 35405, Mus musculus, Metallosphaera sedula DSM 5348 and Staphylococcus aureus.

Preparation of electrocompetent C. autoethanogenum DSM 10061 cells and electrotransformation were carried out as described in Example 1 (see also Example 3). 1-Butene production was performed as described in Example 1. 1-Butene production could be detected by GC-MS as described above.

The three plasmids were also used to engineer Clostridium ljungdahlii DSM 13528 and Clostridium aceticum cells to produce 1-butene. The same protocol and process as described in Example 1 was performed. Additionally, CO and a mixture of CO₂ and CO, as well as CO and electrons and/or CO₂ and electrons were also successfully used as substrates.

### Example 11: Synthesis of Isobutene (2-methylpropene)

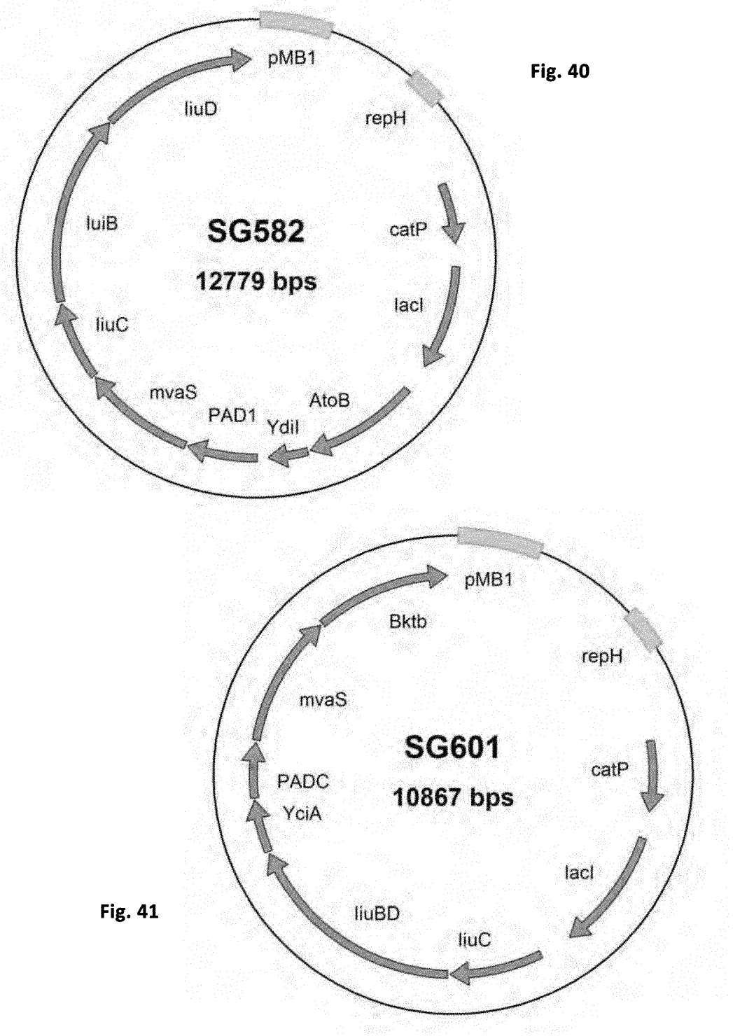

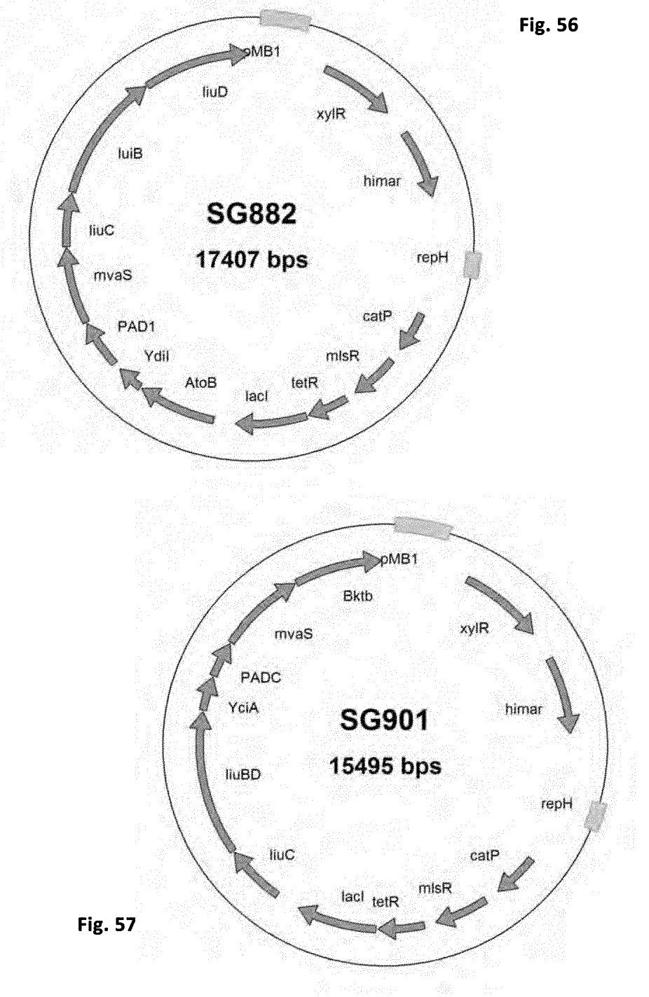

FIG. 41 shows the plasmid SG601 (SEQ ID NO: 37), which contains the sequences encoding enzymes of an engineered isobutene pathway (for genes see also reference numerals). FIG. 40 shows the plasmid SG582 (SEQ ID NO: 36), which contains the sequences encoding enzymes of a further engineered isobutene pathway (for genes see also reference numerals). FIG. 56 shows the plasmid SG882 (SEQ ID NO: 52), which contains the sequences encoding enzymes of yet another engineered isobutene pathway (for genes see also reference numerals). FIG. 57 shows the plasmid SG901 (SEQ ID NO: 53), which contains the sequences encoding enzymes of yet another engineered isobutene pathway (for genes see also reference numerals).

The construction of the expression plasmids was performed by standard recombinant DNA and molecular cloning techniques using genes from Clostridium acetobutylicum ATCC 824, Clostridium carboxidivorans, Escherichia coli, Ralstonia eutropha H16, Aeromonas caviae, Streptomyces sp. strain CL190, Saccharomyces cerevisiae S288c, Bacillus subtilis, Clostridium ljungdahlii DSM 13528 DSM 13528, Escherichia coli BL21(DE3), Clostridium sp. M62/1, Clostridium botulinum BKT015925, Clostridium kluyveri DSM 555, Acidaminococcus fermentans DSM 20731, Clostridium propionicum, Chloroflexus aurantiacus J-10-fl, Haemophilus influenzae R2866, Pseudomonas putida KT2440, Treponema denticola ATCC 35405, Mus musculus, Metallosphaera sedula DSM 5348 and Staphylococcus aureus.

Preparation of electrocompetent C. ljungdahlii DSM 13528 cells and electrotransformation were carried out as described in Example 1 (see also Example 3). Isobutene production was performed as described in Example 1 with 30% CO₂, and 60% H2. Isobutene production could be detected by GC-MS as described above. FIG. 67 shows the analysis of the obtained product via GC-MS, measured on an Agilent 5977E GCMSD. The column used was a Restek Rt-Alumina BOND/MAPD, 30 m×0.32 mm; df 5 μm. Carriers gas: helium. SIM 56 m/z. 500 μL headspace injection using GERSTEL-MultiPurposeSampler 2XL-XT. The spectrum shows a perfect match to isobutene (2-methylpropene).

The two plasmids were also used to engineer Clostridium autoethanogenum DSM 10061 and Clostridium aceticum cells to produce ethene. The same protocol and process as described in Example 1 was performed. Additionally, CO and a mixture of CO₂ and CO, as well as CO and electrons and/or CO₂ and electrons were also successfully used as substrates.

### Example 12: Synthesis of 1-pentene

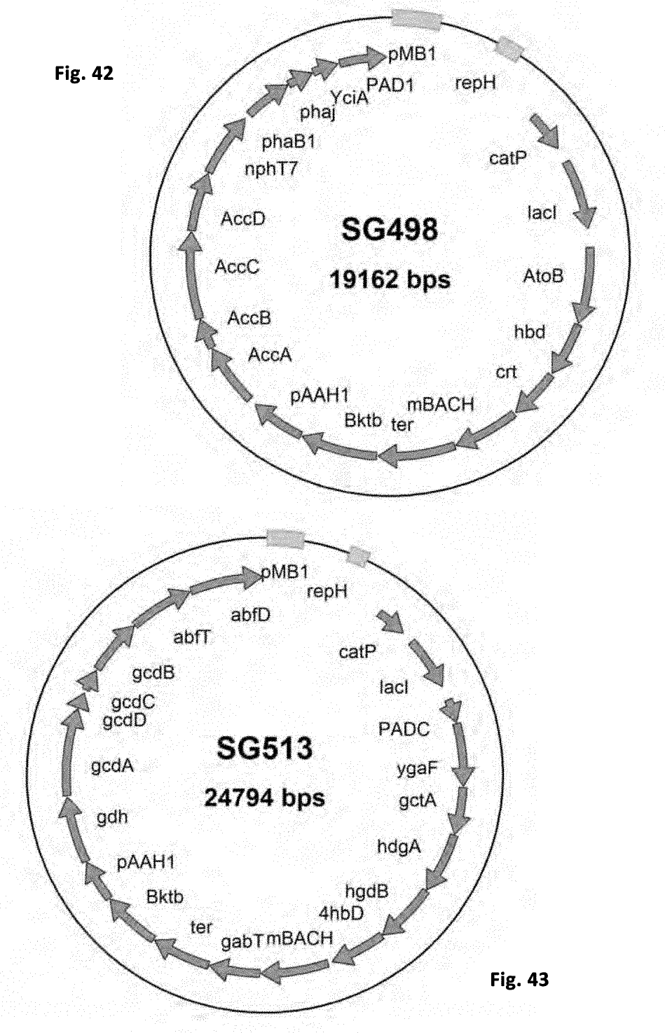

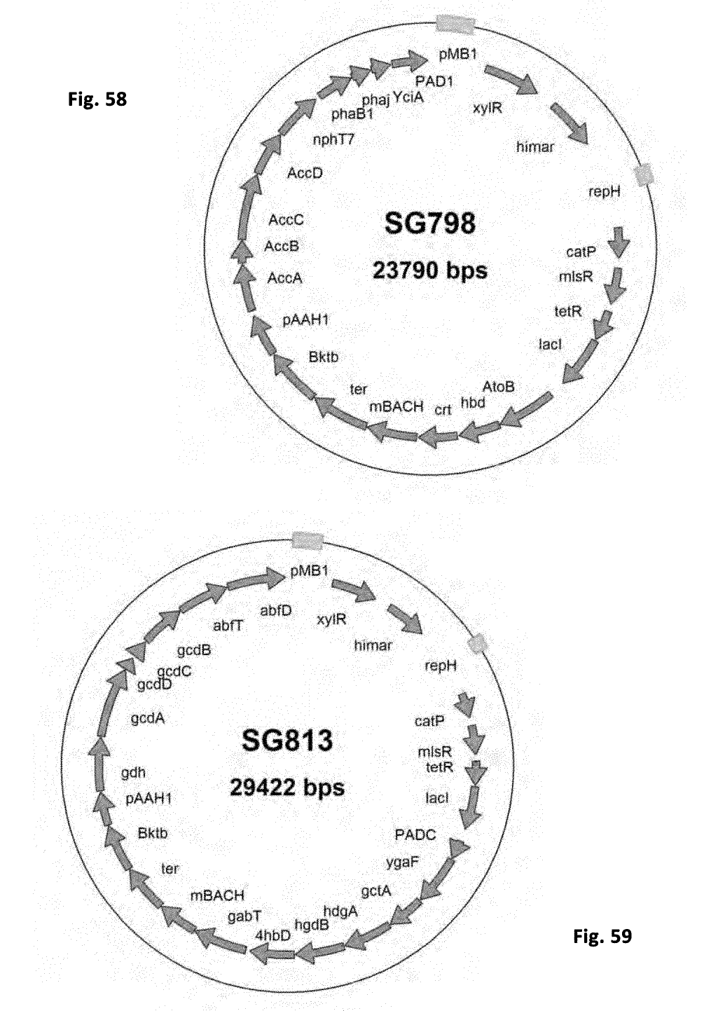

FIG. 42 and FIG. 43 show the plasmids SG498 (SEQ ID NO: 38) and SG513 (SEQ ID NO: 39), respectively, which each contain sequences encoding enzymes of an engineered 1-pentene pathway (for genes see also reference numerals). FIG. 58 and FIG. 59 show the plasmids SG798 (SEQ ID NO: 54) and SG813 (SEQ ID NO: 55), respectively, which each contain the sequences encoding enzymes of another engineered 1-pentene pathway.

The construction of the expression plasmids was performed by standard recombinant DNA and molecular cloning techniques using genes from Clostridium acetobutylicum ATCC 824, Clostridium carboxidivorans, Escherichia coli, Ralstonia eutropha H16, Aeromonas caviae, Streptomyces sp. strain CL190, Saccharomyces cerevisiae S288c, Bacillus subtilis, Clostridium ljungdahlii DSM 13528 DSM 13528, Escherichia coli BL21(DE3), Clostridium sp. M62/1, Clostridium botulinum BKT015925, Clostridium kluyveri DSM 555, Acidaminococcus fermentans DSM 20731, Clostridium propionicum, Chloroflexus aurantiacus J-10-fl, Haemophilus influenzae R2866, Pseudomonas putida KT2440, Treponema denticola ATCC 35405, Mus musculus, Metallosphaera sedula DSM 5348 and Staphylococcus aureus.

Preparation of electrocompetent Clostridium autoethanogenum DSM 10061 cells and electrotransformation were carried out as described in Example 1. 1-Pentene production was performed as described in Example 1 with 100% CO₂. 1-Pentene production could be detected by GC-MS as described above.

The two plasmids were also used to engineer Clostridium ljungdahlii DSM 13528 and Clostridium aceticum cells to produce 1-pentene. The same protocol and process as described in Example 1 was performed. Additionally, CO and a mixture of CO₂ and CO, as well as CO and electrons and/or CO₂ and electrons were also successfully used as substrates.

### Example 13: Synthesis of Ethene

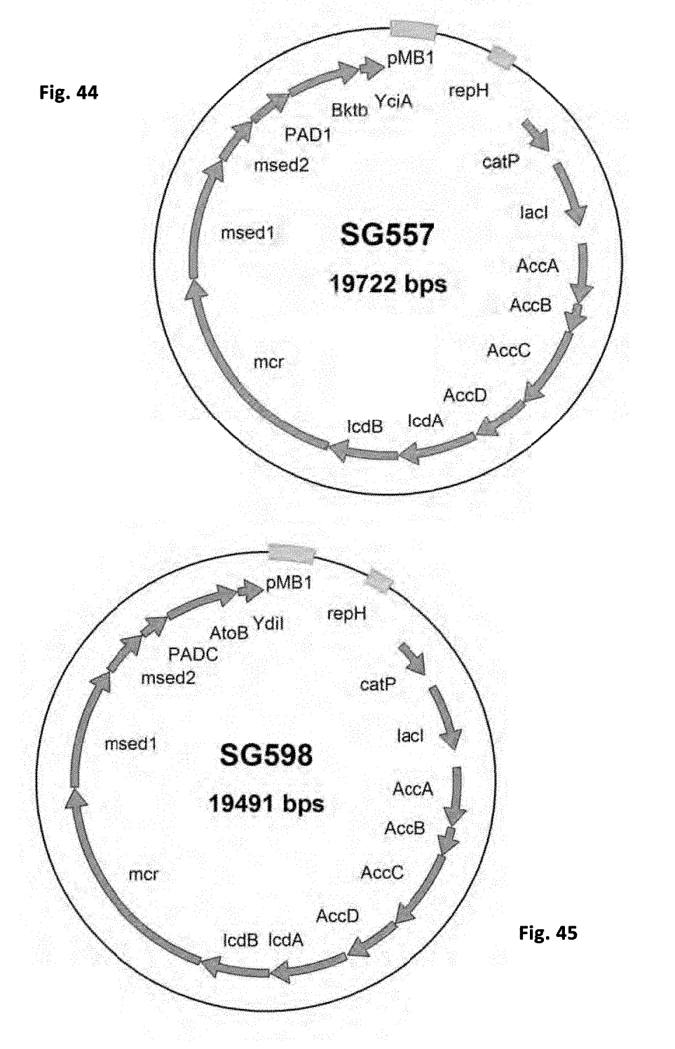

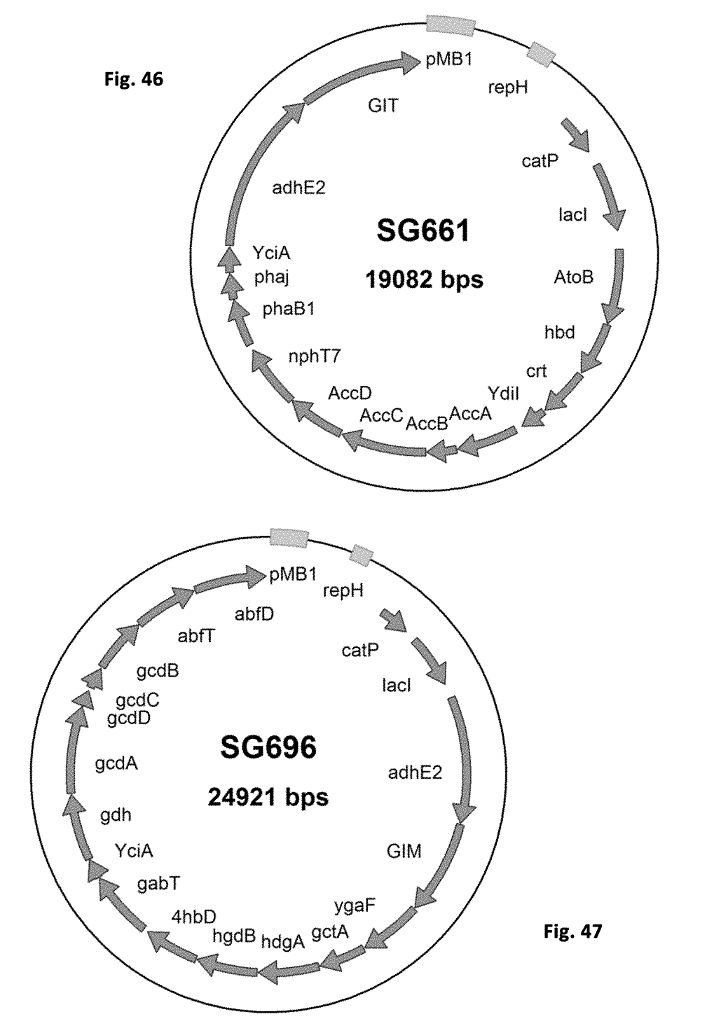

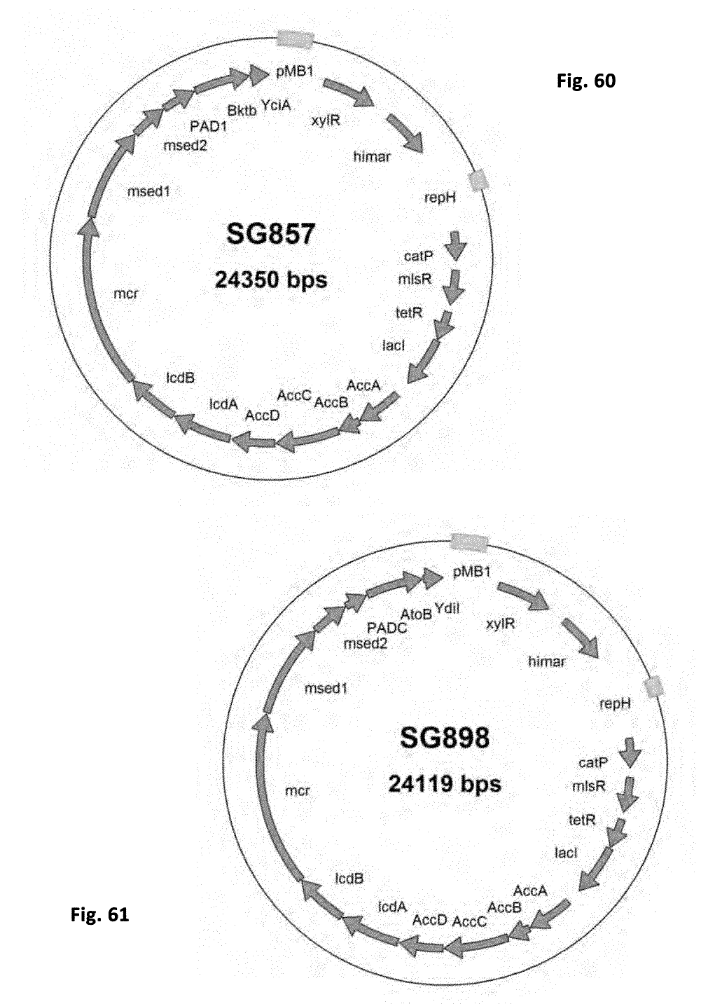

FIG. 44, FIG. 45, FIG. 60, and FIG. 61 show the plasmids SG557 (SEQ ID NO: 40), SG598 (SEQ ID NO: 41), SG857 (SEQ ID NO: 56), and SG898 (SEQ ID NO: 57), respectively, which each contain sequences encoding enzymes of an engineered ethene pathway (for genes see also reference numerals).

The construction of the expression plasmids was performed by standard recombinant DNA and molecular cloning techniques using genes from Clostridium acetobutylicum ATCC 824, Clostridium carboxidivorans, Escherichia coli, Ralstonia eutropha H16, Aeromonas caviae, Streptomyces sp. strain CL190, Saccharomyces cerevisiae S288c, Bacillus subtilis, Clostridium ljungdahlii DSM 13528 DSM 13528, Escherichia coli BL21(DE3), Clostridium sp. M62/1, Clostridium botulinum BKT015925, Clostridium kluyveri DSM 555, Acidaminococcus fermentans DSM 20731, Clostridium propionicum, Chloroflexus aurantiacus J-10-fl, Haemophilus influenzae R2866, Pseudomonas putida KT2440, Treponema denticola ATCC 35405, Mus musculus, Metallosphaera sedula DSM 5348 and Staphylococcus aureus.

Preparation of electrocompetent Clostridium ljungdahlii DSM 13528 cells and electrotransformation were carried out as described in Example 1. Ethene production was performed as described in Example 1 with 30% CO₂, and 60% H₂. Ethene production could be detected by GC-MS as described above.

The two plasmids were also used to engineer Clostridium autoethanogenum DSM 10061 and Clostridium aceticum cells to produce ethene. The same protocol and process as described in Example 1 was performed. Additionally, CO and a mixture of CO₂ and CO, as well as CO and electrons and/or CO₂ and electrons were also successfully used as substrates.

All publications mentioned herein are incorporated herein by reference to disclose and describe the methods and/or materials in connection with which the publications are cited, as if each individual publication, including patent application, were specifically and individually indicated to be incorporated by reference. In case of conflict, the present specification, including definitions, will prevail. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

Unless otherwise expressly stated, it is in no way intended that any method or aspect set forth herein be construed as requiring that its steps be performed in a specific order. Accordingly, where for a claimed method it is not specifically stated in the claims or descriptions that the steps are to be limited to a specific order, it is no way intended that an order be inferred, in any respect. This holds for any possible non-express basis for interpretation, including matters of logic with respect to arrangement of steps or operational flow, plain meaning derived from grammatical organization or punctuation, or the number or type of aspects described in the specification.

1. A recombinant microorganism comprising a heterologous nucleic acid sequence encoding one or more enzymes that allow the conversion of acetyl-CoA to one or more alkenes with a main chain of 2 to 5 carbon atoms, wherein the heterologous nucleic acid sequence comprises:
(a) one or more coding sequences encoding one or more enzymes that catalyse the conversion of acetyl-CoA to crotonyl-CoA, and that further catalyse the conversion of crotonyl-CoA to an alkene; or
(b) one or more coding sequences encoding one or more enzymes that catalyse the conversion of acetyl-CoA to 3-methylcrotonyl-CoA, and that further catalyse the conversion of 3-methylcrotonyl-CoA to an alkene; or
(c) one or more coding sequences encoding one or more enzymes that catalyse the conversion of acetyl-CoA to propionyl-CoA, and that further catalyse the conversion of propionyl-CoA to an alkene; or
(d) one or more coding sequences encoding one or more enzymes that catalyse the conversion of acetyl-CoA to acryloyl-CoA, and that further catalyse the conversion of acryloyl-CoA to an alkene;
wherein each coding sequence is operationally linked to a transcriptional promoter.

2. The recombinant microorganism of claim 1, further comprising endogenous enzymes that convert one or both of carbon monoxide and carbon dioxide to acetyl-CoA.

3. The recombinant microorganism of claim 1 or 2, wherein
(i) the one or more enzymes that catalyse the conversion of acetyl-CoA to crotonyl-CoA comprise:
acetyl-CoA carboxylase (EC 6.4.1.2); acetoacetyl-CoA synthase (EC 2.3.1.194); acetoacetyl-CoA reductase (EC 1.1.1.36); and enoyl-CoA hydratase 2 (EC 4.2.1.119); or
acetyl-CoA C-acetyltransferase (EC 2.3.1.9); 3-hydroxybutyryl-CoA dehydrogenase (EC 1.1.1.157); and 3-hydroxybutyryl-CoA dehydratase; or
4-aminobutyrate aminotransferase (EC 2.6.1.19); glutamate dehydrogenase (EC 1.4.1.2); 2-hydroxyglutarate dehydrogenase (EC 1.1.99.2); glutaconate CoA-transferase (EC 2.8.3.12); 2-hydroxyglutaryl-CoA dehydratase (EC 4.2.1.-); and glutaconyl-CoA decarboxylase (EC 4.1.1.70); or
4-aminobutyrate aminotransferase (EC 2.6.1.19); 4-hydroxybutyrate dehydrogenase (EC 1.1.1.61); 4-hydroxybutyrate CoA-transferase (EC 2.8.3.-); vinylacetyl-CoA-Delta-isomerase (EC 4.2.1.120); and 4-hydroxybutyryl-CoA dehydratase (EC 5.3.3.3);
(ii) the one or more enzymes that catalyse the conversion of acetyl-CoA to 3-methylcrotonyl-CoA comprise (a) one or both of (I) acetyl-CoA C-acetyltransferase (EC 2.3.1.9) and (II) a combination of acetyl-CoA carboxylase (EC 6.4.1.2) and acetoacetyl-CoA synthase (EC 2.3.1.194); (b) 3-hydroxy-3-methylglutaryl-CoA synthase (EC 2.3.3.10); (c) 3-methylglutaconyl-CoA hydratase (EC 4.2.1.18) and (d) 3-methylcrotonyl-CoA carboxylase (EC 6.4.1.4);
(iii) the one or more enzymes that catalyse the conversion of acetyl-CoA to propionyl-CoA comprise:
acetyl-CoA carboxylase (EC 6.4.1.2); malonyl CoA reductase (EC 1.2.1.75); malonyl CoA reductase (EC 1.1.1.298); propionyl-CoA synthase (EC 6.2.1.36); propionyl-CoA synthase/acryloyl-CoA synthase (EC 4.2.1.116); and propionyl-CoA synthase (EC 1.3.1.84); or
acetyl-CoA carboxylase (EC 6.4.1.2); methylmalonyl-CoA mutase (EC 5.4.99.2) and methylmalonyl-CoA decarboxylase (EC 4.1.1.41); or
2-oxoglutarate/butyrate ferredoxin oxidoreductase (EC 1.2.7.3) and/or pyruvate/2-oxobutyrate ferredoxin oxidoreductase (EC 1.2.7.1); or
lactate CoA-transferase (EC 2.8.3.-); lactoyl-CoA dehydratase (EC 4.2.1.54); and propionyl-CoA synthase (EC 1.3.1.84); and
(iv) the one or more enzymes that catalyse the conversion of acetyl-CoA to acryloyl-CoA comprise:
acetyl-CoA carboxylase (EC 6.4.1.2); malonyl CoA reductase (EC 1.2.1.75); malonyl CoA reductase (EC 1.1.1.298); propionyl-CoA synthase (EC 6.2.1.36); and propionyl-CoA synthase/acryloyl-CoA synthase (EC 4.2.1.116); or
lactate CoA-transferase (EC 2.8.3.-); and lactoyl-CoA dehydratase (EC 4.2.1.54).

4. The recombinant microorganism of claim 1, wherein
(i) the one or more enzymes that catalyse the conversion of crotonyl-CoA to an alkene comprise:
(a) acyl-CoA thioesterase (EC 3.1.2.-) and (b) phenyl acrylic acid decarboxylase (EC 4.1.1.-); or
(c) phosphate butyryltransferase (EC 2.3.1.19); (d) butyrate kinase (EC 2.7.2.7), and (e) phenyl acrylic acid decarboxylase (EC 4.1.1.-); or
(f) trans-2-enoyl-CoA reductase (EC1.1.1.36); (g) acetyl-CoA C-acetyltransferase (EC 2.3.1.9); (h) 3-hydroxybutyryl-CoA dehydrogenase (EC 1.1.1.157); (i) 3-hydroxybutyryl-CoA dehydratase (EC 4.2.1.55); (j) acyl-CoA thioesterase (EC 3.1.2.-) and/or a combination of phosphate butyryltransferase (EC 2.3.1.19) and butyrate kinase (EC 2.7.2.7); and (k) a phenyl acrylic acid decarboxylase enzyme (EC 4.1.1.-); or
(l) (i) aldehyde dehydrogenase (EC 1.2.1.-) or alcohol dehydrogenase (EC 1.1.1.-) or (ii) a combination of acyl-CoA thioesterase (EC 3.1.2.-) and aldehyde dehydrogenase (EC 1.2.1.-) or alcohol dehydrogenase (EC 1.1.1.-), or (iii) a combination of aldehyde ferredoxin oxidoreductase (EC 1.2.7.5) and acyl-CoA thioesterase (EC 3.1.2.-); or (iv) a combination of phosphate butyryltransferase (EC 2.3.1.19), butyrate kinase (EC 2.7.2.7) and aldehyde dehydrogenase (EC 1.2.1.-) or alcohol dehydrogenase (EC 1.1.1.-), or (v) a combination of phosphate butyryltransferase (EC 2.3.1.19), butyrate kinase (EC 2.7.2.7) and aldehyde ferredoxin oxidoreductase (EC 1.2.7.5); (m) aldehyde dehydrogenase (EC 1.2.1.-) and/or alcohol dehydrogenase (EC 1.1.1.-); and (n) geraniol isomerase (EC 5.4.4.4) or linalool dehydratase (EC 4.2.1.127), or a combination of (i) one or more of farnesol kinase (EC 2.7.1.-), geranylgeraniol kinase (EC 2.7.1.619), hydroxyethylthiazole kinase (EC 2.7.1.50) or undecaprenol kinase (EC 2.7.1.66), and (ii) one or more of 2-methyl-1,3-butadiene synthase (EC 4.2.3.27) or methylbutenol synthase;
(ii) the one or more enzymes that catalyse the conversion of 3-methylcrotonyl-CoA to an alkene comprise
(a) phenyl acrylic acid decarboxylase enzyme (EC 4.1.1.-); and (b) acyl-CoA thioesterase enzyme (EC 3.1.2.-) and/or a combination of phosphate butyryltransferase (EC 2.3.1.19) and butyrate kinase (EC 2.7.2.7); or
(c) (i) aldehyde dehydrogenase (EC 1.2.1.-) or alcohol dehydrogenase (EC 1.1.1.-) or (ii) a combination of acyl-CoA thioesterase (EC 3.1.2.-) and aldehyde dehydrogenase (EC 1.2.1.-) or alcohol dehydrogenase (EC 1.1.1.-), or (iii) a combination of aldehyde ferredoxin oxidoreductase (EC 1.2.7.5) and acyl-CoA thioesterase (EC 3.1.2.-); or (iv) a combination of phosphate butyryltransferase (EC 2.3.1.19), butyrate kinase (EC 2.7.2.7) and aldehyde dehydrogenase (EC 1.2.1.-) or alcohol dehydrogenase (EC 1.1.1.-), or (v) a combination of phosphate butyryltransferase (EC 2.3.1.19), butyrate kinase (EC 2.7.2.7) and aldehyde ferredoxin oxidoreductase (EC 1.2.7.5); (m) aldehyde dehydrogenase (EC 1.2.1.-) and/or alcohol dehydrogenase (EC 1.1.1.-); and (n) geraniol isomerase (EC 5.4.4.4) or linalool dehydratase (EC 4.2.1.127), or a combination of (i) one or more of farnesol kinase (EC 2.7.1.-), geranylgeraniol kinase (EC 2.7.1.619), hydroxyethylthiazole kinase (EC 2.7.1.50) or undecaprenol kinase (EC 2.7.1.66), and (ii) one or more of 2-methyl-1,3-butadiene synthase (EC 4.2.3.27) or methylbutenol synthase;
(iii) the one or more enzymes that catalyse the conversion of propionyl-CoA to an alkene comprise (a) acetyl-CoA C-acetyltransferase (EC 2.3.1.9); (b) a combination of (I) acetoacetyl-CoA reductase (EC 1.1.1.36) and enoyl-CoA hydratase 2 (EC 4.2.1.119) and/or (II) 3-hydroxybutyryl-CoA dehydrogenase (EC 1.1.1.157) and 3-hydroxybutyryl-CoA dehydratase (EC 4.2.1.55); (c) acyl-CoA thioesterase (EC 3.1.2.-) and/or a combination of phosphate butyryltransferase (EC 2.3.1.19) and butyrate kinase (EC 2.7.2.7); and (d) phenyl acrylic acid decarboxylase (EC 4.1.1.-); and
(iv) the one or more enzymes that catalyse the conversion of acryloyl-CoA to an alkene comprise (a) acyl-CoA thioesterase (EC 3.1.2.-) and/or a combination of phosphate butyryltransferase (EC 2.3.1.19) and butyrate kinase (EC 2.7.2.7); and (b) phenyl acrylic acid decarboxylase (EC 4.1.1.-).

5. The recombinant microorganism of claim 1, wherein the heterologous nucleic acid sequence is comprised in a heterologous nucleic acid molecule or in the microorganism's chromosome.

6. The recombinant microorganism of claim 2, wherein the endogenous enzymes that convert one or both of carbon monoxide and carbon dioxide to acetyl-CoA are enzymes of the Wood-Ljungdahl pathway.

7. The recombinant microorganism of claim 1, being a species of one of Clostridium ljungdahlii, Clostridium autoethanogenum, Clostridium carboxidivorans, Clostridium aceticum, Clostridium drakei, Clostridium scatologenes, Clostridium ragsdalei, Clostridium formicoaceticum, Clostridium magnum, Butyribacterium methylotrophicum, Acetobacterium woodii, Alkalibaculum bacchii, Aecetoariaerobium riotera, Blautia producta, Eubacterium limosum, Desulfitbacterium hafhierise, Moorella thermoacetica, Moorella thermautotrophica, Peptostreptococcus productus, Rhodospirillum rubrum, Sporomusa ovata, Sporomusa silvacetica, Sporomusa sphaeroides, Thermoanaerobacter kiuvi, Oxobacter pfennigii, Acetobacterium fimetarium, Acetohalobium arabaticu, Blautia wexlerae, Carbophilus carboxidus, Cloacibacillus evryensis, Hydrogenophaga pseudoflava, Rhodopseudomonas palustris, Pseudomonas gazotropha, Ralstonia eutropha, Calderihabitans maritimus, Caloribacterium cisternae, Carboxydobrachium pacificum, Carboxydocella ferrireducens, Carboxydocella sporoproducens, Carboxydocella thermautotrophica, Carboxydothermus ferrireducens, Carboxydothermus hydrogenoformans, Carboxydothermus islandicus, Carboxydothermus pertinax, Carboxydothermus siderophilus, Clostridium fervidus, Clostridium thermocellum, Desulfotomaculum carboxydivorans, Desulfotomaculum kuznetsovii, Desulfotomaculum thermobenzoicum subsp. thermosyntrophicum, Desulfotomaculum thermocisternum, Desulfurispora thermophila, Dictyoglomus thermophilum, Hydrogenophilus islandicus, Moorella mulderi, Moorella stamsii, Thermincola carboxydiphila, Thermincola ferriacetica, Thermincola potens, Thermoacetogenium phaeum, Thermoanaerobacter kivui, Thermoanaerobacter thermohydrosulfuricus subsp. carboxydovorans, Thermosinus carboxydivorans, Sporomusa termitida, Clostridium formicaceticum, Oligotropha carboxidovorans, Desulfotomaculum gibsoniae, Desulfosporosinus meridiei, Blautia hydrogenotrophica, Dehalococcoides mccartyi, Desulfatibacillum aliphaticivorans, Desulfobacterium autotrophicum, Desulfobacula toluolica, Desulfospira joergensenii, Desulfosporosinus orientis, Desulfosporosinus youngiae, Desulfotomaculum acetoxidans, Desulfotomaculum alcoholivorax, Desulfotomaculum carboxydivorans, Desulfotomaculum sapomandens, Desulfotomaculum thermocisternum, Desulfovermiculus halophilus, Desulfovibrio alaskensis, Desulfovibrio desulfuricans, Desulfovibrio frigidus, Desulfurispora thermophila, Holophaga foetida, Methanobrevibacter arboriphilus, Orenia salinaria, Paenibacillus polymyxa, Tindallia califomiensis, Anoxybacillus flavithermus, Desulfovirgula thermocuniculi, Thermosediminibacter oceani, Acetobacterium bakii, Acetobacterium carbinolicum, Acetobacterium dehalogenans, Acetobacterium malicum, Acetobacterium paludosum, Acetobacterium tundrae, Acetobacterium wieringae, Candidatus Scalindua brodae, Acetoanaerobium noterae, Blautia hansenii, Ammonifex degensii, Acetitomaculum ruminis, Acetoanaerobium romashkovil, Acetobacterium psammolithicum, Acetonema longum, Bryanella formatexigens, Caloramator fervidus, Clostridium coccoides, Clostridium difficile, Moorella glycerini, Natroniella acetigena, Natronincola histinovorans, Ruminococcus hydrogenotrophicus, Ruminococcus productus, Ruminococcus schinkii, Sporomusa acidovorans, Sporomusa aerivorans, Sporomusa malonica, Sporomusa paucivorans, Syntrophococcus sucromutans, Treponema primitia, Pseudomonas carboxydohyrogena, Pseudomonas thermocarboxydovorans, Bradyrhizobium japonicum, Streptomyces thermoautotrophicus, Mycobacterium smegmatis, Mycobacterium gordonae, Mycobacterium tuberculosis, Bacillus schlegelii, Caldanaerobacter subterraneus, Thermolithobacter carboxydivorans, Thermococcus onnurineus, Thermofilum carboxyditrophus, Archaeoglobus fulgidus, Desulfomonile tiedjei, Desulfovibrio vulgaris, Thermoproteus tenax or Rubrivivax gelatinosa.

8. The recombinant microorganism of claim 1, wherein the heterologous nucleic acid sequence comprises:
(a) a combination of coding sequences encoding (i) the enzyme acetyl-CoA C-acetyltransferase (EC 2.3.1.9); (ii) the enzyme 3-hydroxybutyryl-CoA dehydrogenase (EC 1.1.1.157); (iii) the enzyme 3-hydroxybutyryl-CoA dehydratase (EC 4.2.1.55); (iv) an acyl-CoA thioesterase enzyme (EC 3.1.2.-); (v) an aldehyde dehydrogenase (EC 1.2.1.-) or an alcohol dehydrogenase enzyme (EC 1.1.1.-); and (vi) the enzyme geraniol isomerase (EC 5.4.4.4) or the enzyme linalool dehydratase (EC 4.2.1.127); or
(b) a combination of coding sequences encoding (i) the enzyme Acetyl-CoA carboxylase (EC 6.4.1.2); (ii) the enzyme acetoacetyl-CoA synthase (EC 2.3.1.194); (iii) the enzyme acetoacetyl-CoA reductase (EC 1.1.1.36); (iv) the enzyme enoyl-CoA hydratase 2 (EC 4.2.1.119); (v) an acyl-CoA thioesterase enzyme (EC 3.1.2.-); (vi) an aldehyde dehydrogenase (EC 1.2.1.-) or an alcohol dehydrogenase enzyme (EC 1.1.1.-); and (vii) the enzyme geraniol isomerase (EC 5.4.4.4) or the enzyme linalool dehydratase (EC 4.2.1.127); or
(c) a combination of coding sequences encoding the enzyme 4-aminobutyrate aminotransferase (EC 2.6.1.19); the enzyme glutamate dehydrogenase (EC 1.4.1.2); the enzyme 2-hydroxyglutarate dehydrogenase (EC 1.1.99.2); the enzyme glutaconate CoA-transferase (EC 2.8.3.12); a 2-hydroxyglutaryl-CoA dehydratase enzyme (EC 4.2.1.-); the enzyme glutaconyl-CoA decarboxylase (EC 4.1.1.70); an acyl-CoA thioesterase enzyme (EC 3.1.2.-); an aldehyde dehydrogenase (EC 1.2.1.-) or an alcohol dehydrogenase enzyme (EC 1.1.1.-); and the enzyme geraniol isomerase (EC 5.4.4.4) or the enzyme linalool dehydratase (EC 4.2.1.127); or
(d) a combination of coding sequences encoding the enzyme 4-aminobutyrate aminotransferase (EC 2.6.1.19); the enzyme 4-hydroxybutyrate dehydrogenase (EC 1.1.1.61); a 4-hydroxybutyrate CoA-transferase enzyme (EC 2.8.3.-); the enzyme vinylacetyl-CoA-Delta-isomerase (EC 4.2.1.120); the enzyme 4-hydroxybutyryl-CoA dehydratase (EC 5.3.3.3); an acyl-CoA thioesterase enzyme (EC 3.1.2.-); an aldehyde dehydrogenase (EC 1.2.1.-) or an alcohol dehydrogenase enzyme (EC 1.1.1.-); and the enzyme geraniol isomerase (EC 5.4.4.4) or the enzyme linalool dehydratase (EC 4.2.1.127); or
(e) a combination of coding sequences encoding
(i) (I) the enzyme acetyl-CoA C-acetyltransferase (EC 2.3.1.9) and/or the enzymes acetyl-CoA carboxylase (EC 6.4.1.2) and acetoacetyl-CoA synthase (EC 2.3.1.194), the enzyme 3-hydroxy-3-methylglutaryl-CoA synthase (EC 2.3.3.10), the enzyme 3-methylglutaconyl-CoA hydratase (EC 4.2.1.18), and the enzyme 3-methylcrotonyl-CoA carboxylase (EC 6.4.1.4), or (II) a combination of coding sequences encoding (i) a combination of (I) the enzymes acetyl-CoA C-acetyltransferase (EC 2.3.1.9), 3-hydroxybutyryl-CoA dehydrogenase (EC 1.1.1.157), and 3-hydroxybutyryl-CoA dehydratase (EC 4.2.1.55) and/or (II) the enzymes Acetyl-CoA carboxylase (EC 6.4.1.2), acetoacetyl-CoA synthase (EC 2.3.1.194), acetoacetyl-CoA reductase (EC 1.1.1.36) and enoyl-CoA hydratase 2 (EC 4.2.1.119); and
(ii) an aldehyde dehydrogenase (EC 1.2.1.-) or an alcohol dehydrogenase enzyme (EC 1.1.1.-); and
(iii) the enzymes geraniol isomerase (EC 5.4.4.4) and/or linalool dehydratase (EC 4.2.1.127), and/or a combination of (I) one or more of the enzymes farnesol kinase (EC 2.7.1.-), geranylgeraniol kinase (EC 2.7.1.619), hydroxyethylthiazole kinase (EC 2.7.1.50) or undecaprenol kinase (EC 2.7.1.66), and (II) one or more of the enzymes 2-methyl-1,3-butadiene synthase (EC 4.2.3.27) or methylbutenol synthase; or
(f) a combination of coding sequences encoding
(i) (I) a combination of the enzymes acetyl-CoA C-acetyltransferase (EC 2.3.1.9), 3-hydroxybutyryl-CoA dehydrogenase (EC 1.1.1.157), and 3-hydroxybutyryl-CoA dehydratase (EC 4.2.1.55), and/or a combination of the enzymes the enzymes Acetyl-CoA carboxylase (EC 6.4.1.2), acetoacetyl-CoA synthase (EC 2.3.1.194), acetoacetyl-CoA reductase (EC 1.1.1.36) and enoyl-CoA hydratase 2 (EC 4.2.1.119), or (II) a combination of the enzyme acetyl-CoA C-acetyltransferase (EC 2.3.1.9) and/or the enzymes Acetyl-CoA carboxylase (EC 6.4.1.2) and acetoacetyl-CoA synthase (EC 2.3.1.194); the enzyme 3-hydroxy-3-methylglutaryl-CoA synthase (EC 2.3.3.10); the enzyme 3-methylglutaconyl-CoA hydratase (EC 4.2.1.18); and the enzyme 3-methylglutaconyl-CoA decarboxylase; and
(ii) a combination of the enzymes phosphate butyryltransferase (EC 2.3.1.19) and butyrate kinase (EC 2.7.2.7), and/or an acyl-CoA thioesterase enzyme (EC 3.1.2.-); and (iii) a phenyl acrylic acid decarboxylase enzyme (EC 4.1.1.-); or
(g) a combination of coding sequences encoding (i) the enzyme lactate CoA-transferase (EC 2.8.3.-), (ii) the enzyme lactoyl-CoA dehydratase (EC 4.2.1.54), (iii) one or both of (I) the enzyme acyl-CoA thioesterase (EC 3.1.2.-) or (II) a combination of the enzyme phosphate butyryltransferase (EC 2.3.1.19) and the enzyme butyrate kinase (EC 2.7.2.7); and (iv) the enzyme phenyl acrylic acid decarboxylase (EC 4.1.1.-); or
(h) a combination of coding sequences encoding
(i) (I) a combination of the enzyme 4-aminobutyrate aminotransferase (EC 2.6.1.19), the enzyme 4-hydroxybutyrate dehydrogenase (EC 1.1.1.61), a 4-hydroxybutyrate CoA-transferase enzyme (EC 2.8.3.-), the enzyme vinylacetyl-CoA-delta-isomerase (EC 4.2.1.120), and the enzyme 4-hydroxybutyryl-CoA dehydratase (EC 5.3.3.3), or (II) the enzyme 4-aminobutyrate aminotransferase (EC 2.6.1.19), the enzyme glutamate dehydrogenase (EC 1.4.1.2), the enzyme 2-hydroxyglutarate dehydrogenase (EC 1.1.99.2), the enzyme glutaconate CoA-transferase (EC 2.8.3.12), the enzyme 2-hydroxyglutaryl-CoA dehydratase (EC 4.2.1.-), and the enzyme glutaconyl-CoA decarboxylase (EC 4.1.1.70); and
(ii) a combination of the enzymes phosphate butyryltransferase (EC 2.3.1.19) and butyrate kinase (EC 2.7.2.7), and/or an acyl-CoA thioesterase enzyme (EC 3.1.2.-); and
(iii) the enzyme phenyl acrylic acid decarboxylase (EC 4.1.1.-); or
(i) a combination of coding sequences encoding (i) a combination of (I) 3-hydroxybutyryl-CoA dehydrogenase (EC 1.1.1.157), and 3-hydroxybutyryl-CoA dehydratase (EC 4.2.1.55) and/or (II) the enzymes Acetyl-CoA carboxylase (EC 6.4.1.2), acetoacetyl-CoA synthase (EC 2.3.1.194), acetoacetyl-CoA reductase (EC 1.1.1.36) and enoyl-CoA hydratase 2 (EC 4.2.1.119); (ii) the enzyme trans-2-enoyl-CoA reductase (EC1.1.1.36); (iii) the enzyme acetyl-CoA C-acetyltransferase (EC 2.3.1.9); (iv) the enzyme 3-hydroxybutyryl-CoA dehydrogenase (EC 1.1.1.157); (v) the enzyme 3-hydroxybutyryl-CoA dehydratase (EC 4.2.1.55); (vi) a combination of the enzymes phosphate butyryltransferase (EC 2.3.1.19) and butyrate kinase (EC 2.7.2.7), and/or an acyl-CoA thioesterase enzyme (EC 3.1.2.-); and (vii) a phenyl acrylic acid decarboxylase enzyme (EC 4.1.1.-); or
(j) a combination of coding sequences encoding (i) the enzyme acetyl-CoA carboxylase (EC 6.4.1.2); (ii) the enzyme methylmalonyl-CoA mutase (EC 5.4.99.2); (iii) the enzyme methylmalonyl-CoA decarboxylase (EC 4.1.1.41); (iv) the enzyme acetyl-CoA C-acetyltransferase (EC 2.3.1.9); (v) a combination of (I) the enzymes acetoacetyl-CoA reductase (EC 1.1.1.36) and enoyl-CoA hydratase 2 (EC 4.2.1.119) and/or (II) the enzymes 3-hydroxybutyryl-CoA dehydrogenase (EC 1.1.1.157) and 3-hydroxybutyryl-CoA dehydratase (EC 4.2.1.55); (vi) an acyl-CoA thioesterase enzyme (EC 3.1.2.-); (vii) the enzyme acetyl-CoA C-acetyltransferase (EC 2.3.1.9); (iix) a combination of (I) the enzymes acetoacetyl-CoA reductase (EC 1.1.1.36) and enoyl-CoA hydratase 2 (EC 4.2.1.119) and/or (II) the enzymes 3-hydroxybutyryl-CoA dehydrogenase (EC 1.1.1.157) and 3-hydroxybutyryl-CoA dehydratase (EC 4.2.1.55); (ix) a combination of the enzymes phosphate butyryltransferase (EC 2.3.1.19) and butyrate kinase (EC 2.7.2.7), and/or an acyl-CoA thioesterase enzyme (EC 3.1.2.-); and (x) a phenyl acrylic acid decarboxylase enzyme (EC 4.1.1.-); or
(k) a combination of coding sequences encoding
(i)(I) a combination of the enzyme 2-oxoglutarate/butyrate ferredoxin oxidoreductase (EC 1.2.7.3), and the enzyme pyruvate/2-oxobutyrate ferredoxin oxidoreductase (EC 1.2.7.1); (II) a combination of a lactate CoA-transferase enzyme (EC 2.8.3.-), the enzyme lactoyl-CoA dehydratase (EC 4.2.1.54), and the enzyme propionyl-CoA synthase (EC 1.3.1.84); or (III) a combination of the enzyme acetyl-CoA carboxylase (EC 6.4.1.2), the enzyme malonyl CoA reductase (EC 1.2.1.75), the enzyme malonyl CoA reductase (EC 1.1.1.298), the enzyme propionyl-CoA synthase (EC 6.2.1.36), the enzyme propionyl-CoA synthase/acryloyl-CoA synthase (EC 4.2.1.116), and the enzyme propionyl-CoA synthase (EC 1.3.1.84); and (ii) the enzyme lactoyl-CoA dehydratase (EC 4.2.1.54); and
(iii) the enzyme acetyl-CoA C-acetyltransferase (EC 2.3.1.9); and (iv) a combination of (I) the enzymes acetoacetyl-CoA reductase (EC 1.1.1.36) and enoyl-CoA hydratase 2 (EC 4.2.1.119) and/or (II) the enzymes 3-hydroxybutyryl-CoA dehydrogenase (EC 1.1.1.157) and 3-hydroxybutyryl-CoA dehydratase (EC 4.2.1.55); (iv) a combination of the enzymes phosphate butyryltransferase (EC 2.3.1.19) and butyrate kinase (EC 2.7.2.7), and/or an acyl-CoA thioesterase enzyme (EC 3.1.2.-); and (v) a phenyl acrylic acid decarboxylase enzyme (EC 4.1.1.-); or
(l) a combination of coding sequences encoding (i) the enzyme 4-aminobutyrate aminotransferase (EC 2.6.1.19); (ii) a combination of (I) the enzyme glutamate dehydrogenase (EC 1.4.1.2), the enzyme 2-hydroxyglutarate dehydrogenase (EC 1.1.99.2), the enzyme glutaconate CoA-transferase (EC 2.8.3.12), a 2-hydroxyglutaryl-CoA dehydratase enzyme (EC 4.2.1.-) and/or (II) the enzyme 4-hydroxybutyrate dehydrogenase (EC 1.1.1.61), a 4-hydroxybutyrate CoA-transferase enzyme (EC 2.8.3.-), the enzyme vinylacetyl-CoA-delta-isomerase (EC 4.2.1.120) and the enzyme glutaconyl-CoA decarboxylase (EC 4.1.1.70); (iii) the enzyme 4-hydroxybutyryl-CoA dehydratase (EC 5.3.3.3); (iv) the enzyme trans-2-enoyl-CoA reductase (EC1.1.1.36); (v) the enzyme acetyl-CoA C-acetyltransferase (EC 2.3.1.9); (vi) the enzyme 3-hydroxybutyryl-CoA dehydrogenase (EC 1.1.1.157); (vii) the enzyme 3-hydroxybutyryl-CoA dehydratase (EC 4.2.1.55); (iix) an acyl-CoA thioesterase enzyme (EC 3.1.2.-); and (x) a phenyl acrylic acid decarboxylase enzyme (EC 4.1.1.-); or
(m) a combination of coding sequences encoding
(i)(I) a combination of the enzyme acetyl-CoA carboxylase (EC 6.4.1.2), the enzyme malonyl CoA reductase (malonate semialdehyde-forming) (EC 1.2.1.75), the enzyme 3-hydroxypropionate dehydrogenase (EC 1.1.1.298), the enzyme 3-hydroxypropionyl-CoA synthase (EC 6.2.1.36), and the enzyme hydroxypropionyl-CoA dehydratase (EC 4.2.1.116), or (II) a combination of (□) the enzyme acetyl-CoA carboxylase (EC 6.4.1.2), a lactate CoA-transferase enzyme (EC 2.8.3.-), and the enzyme lactoyl-CoA dehydratase (EC 4.2.1.54) and/or (□) a lactate CoA-transferase enzyme (EC 2.8.3.-) and the enzyme lactoyl-CoA dehydratase (EC 4.2.1.54);
(ii) (I) a combination of the enzymes phosphate butyryltransferase (EC 2.3.1.19) and butyrate kinase (EC 2.7.2.7), and/or (II) an acyl-CoA thioesterase enzyme (EC 3.1.2.-); and
(iii) a phenyl acrylic acid decarboxylase enzyme (EC 4.1.1.-).

9. A method of producing one or more alkenes with a main chain of 2 to 5 carbon atoms, the method comprising contacting a recombinant microorganism according to claim 1 with one or both of carbon monoxide or carbon dioxide.

10. The method of claim 9, being a method of producing one or more of ethylene, 1-propene, 1-butene, 2-methylpropene, 1,3-butadiene, 1-pentene, or 2-methyl-1,3-butadiene.
